SEQUENCE LISTING (17 sequences)

SEQ ID NO: 1 - protein, 400 aa, Artificial Sequence
  1-392 of the Helicobacter pylori alpha(1,3)-fucosyltransferase (GenBank Accession NO. AAD07710.1) fused with a C-terminal His tag
MFQPLLDAFIESASIEKMVSKSPPPPLKIAVANWWGDEEIKEFKKSVLYFILSQRYAITLHQNPNESSDLVFSNPLGAARKILSYQNTKRVFYTGENESPNFNLFDYAIGFDELDFNDRYLRMPLYYAHLHYEAELVNDTTAPYKLKDNSLYALKKPSHHFKENHPNLCAVVNDESDLLKRGFASFVASNANAPMRNAFYDALNSIEPVTGGGSVRNTLGYKVGNKSEFLSQYKFNLCFENSQGYGYVTEKILDAYFSHTIPIYWGSPSVAKDFNPKSFVNVHDFNNFDEAIDYIKYLHTHPNAYLDMLYENPLNTLDGKAYFYQDLSFKKILDFFKTILENDTIYHNNPFIFYRDLHEPLISIDDLRVNYDDLRVNYDDLRVNYDDLRVNYLEHHHHHH

SEQ ID NO: 2 - protein, 392 aa, Artificial Sequence
  1-392 of the Helicobacter pylori alpha(1,3)-fucosyltransferase (GenBank Accession NO. AAD07710.1)
MFQPLLDAFIESASIEKMVSKSPPPPLKIAVANWWGDEEIKEFKKSVLYFILSQRYAITLHQNPNESSDLVFSNPLGAARKILSYQNTKRVFYTGENESPNFNLFDYAIGFDELDFNDRYLRMPLYYAHLHYEAELVNDTTAPYKLKDNSLYALKKPSHHFKENHPNLCAVVNDESDLLKRGFASFVASNANAPMRNAFYDALNSIEPVTGGGSVRNTLGYKVGNKSEFLSQYKFNLCFENSQGYGYVTEKILDAYFSHTIPIYWGSPSVAKDFNPKSFVNVHDFNNFDEAIDYIKYLHTHPNAYLDMLYENPLNTLDGKAYFYQDLSFKKILDFFKTILENDTIYHNNPFIFYRDLHEPLISIDDLRVNYDDLRVNYDDLRVNYDDLRVNY

SEQ ID NO: 3 - protein, 287 aa, Artificial Sequence
  Bovine beta1,4-GalT1catalytic domain (130-402), with Y289L, C342T mutations and a N-terminal tag.
MASMTGGQQMGRGSSLTACPEESPLLVGPMLIEFNIPVDLKLVEQQNPKVKLGGRYTPMDCISPHKVAIIIPFRNRQEHLKYWLYYLHPILQRQQLDYGIYVINQAGESMFNRAKLLNVGFKEALKDYDYNCFVFSDVDLIPMNDHNTYRCFSQPRHISVAMDKFGFSLPYVQLFGGVSALSKQQFLSINGFPNNYWGWGGEDDDIYNRLAFRGMSVSRPNAVIGKTRMIRHSRDKKNEPNPQRFDRIAHTKETMLSDGLNSLTYMVLEVQRYPLYTKITVDIGTPS

SEQ ID NO: 4 - protein, 273 aa, Artificial Sequence
  Bovine beta1,4-GalT1catalytic domain 130-402, with Y289L and C342T mutations
SLTACPEESPLLVGPMLIEFNIPVDLKLVEQQNPKVKLGGRYTPMDCISPHKVAIIIPFRNRQEHLKYWLYYLHPILQRQQLDYGIYVINQAGESMFNRAKLLNVGFKEALKDYDYNCFVFSDVDLIPMNDHNTYRCFSQPRHISVAMDKFGFSLPYVQLFGGVSALSKQQFLSINGFPNNYWGWGGEDDDIYNRLAFRGMSVSRPNAVIGKTRMIRHSRDKKNEPNPQRFDRIAHTKETMLSDGLNSLTYMVLEVQRYPLYTKITVDIGTPS

SEQ ID NO: 5 - protein, 273 aa, Artificial Sequence
  Human beta1,4-GalT1 catalytic domain 126-398 with a Y285L mutation
SLPACPEESPLLVGPMLIEFNMPVDLELVAKQNPNVKMGGRYAPRDCVSPHKVAIIIPFRNRQEHLKYWLYYLHPVLQRQQLDYGIYVINQAGDTIFNRAKLLNVGFQEALKDYDYTCFVFSDVDLIPMNDHNAYRCFSQPRHISVAMDKFGFSLPYVQLFGGVSALSKQQFLTINGFPNNYWGWGGEDDDIFNRLVFRGMSISRPNAVVGRCRMIRHSRDKKNEPNPQRFDRIAHTKETMLSDGLNSLTYQVLDVQRYPLYTQITVDIGTPS

SEQ ID NO: 6 - protein, 1006 aa, Artificial Sequence
  37-995 of Streptococcus pyogenes Endoglycosidase S fused with a N-terminal tag
MHHHHHHSSGLVPRGSGMKETAAAKFERQHMDSPDLGTGGGSGIEGREEKTVQVQKGLPSIDSLHYLSENSKKEFKEELSKAGQESQKVKEILAKAQQADKQAQELAKMKIPEKIPMKPLHGPLYGGYFRTWHDKTSDPTEKDKVNSMGELPKEVDLAFIFHDWTKDYSLFWKELATKHVPKLNKQGTRVIRTIPWRFLAGGDNSGIAEDTSKYPNTPEGNKALAKAIVDEYVYKYNLDGLDVDVEHDSIPKVDKKEDTAGVERSIQVFEEIGKLIGPKGVDKSRLFIMDSTYMADKNPLIERGAPYINLLLVQVYGSQGEKGGWEPVSNRPEKTMEERWQGYSKYIRPEQYMIGFSFYEENAQEGNLWYDINSRKDEDKANGINTDITGTRAERYARWQPKTGGVKGGIFSYAIDRDGVAHQPKKYAKQKEFKDATDNIFHSDYSVSKALKTVMLKDKSYDLIDEKDFPDKALREAVMAQVGTRKGDLERFNGTLRLDNPAIQSLEGLNKFKKLAQLDLIGLSRITKLDRSVLPANMKPGKDTLETVLETYKKDNKEEPATIPPVSLKVSGLTGLKELDLSGFDRETLAGLDAATLTSLEKVDISGNKLDLAPGTENRQIFDTMLSTISNHVGSNEQTVKFDKQKPTGHYPDTYGKTSLRLPVANEKVDLQSQLLFGTVTNQGTLINSEADYKAYQNHKIAGRSFVDSNYHYNNFKVSYENYTVKVTDSTLGTTTDKTLATDKEETYKVDFFSPADKTKAVHTAKVIVGDEKTMMVNLAEGATVIGGSADPVNARKVFDGQLGSETDNISLGWDSKQSIIFKLKEDGLIKHWRFFNDSARNPETTNKPIQEASLQIFNIKDYNLDNLLENPNKFDDEKYWITVDTYSAQGERATAFSNTLNNITSKYWRVVFDTKGDRYSSPVVPELQILGYPLPNADTIMKTVTTAKELSQQKDKFSQKMLDELKIKEMALETSLNSKIFDVTAINANAGVLKDCIEKRQLLKK

SEQ ID NO: 7 - protein, 959 aa, Artificial Sequence
  37-995 of Streptococcus pyogenes Endoglycosidase S fused
EEKTVQVQKGLPSIDSLHYLSENSKKEFKEELSKAGQESQKVKEILAKAQQADKQAQELAKMKIPEKIPMKPLHGPLYGGYFRTWHDKTSDPTEKDKVNSMGELPKEVDLAFIFHDWTKDYSLFWKELATKHVPKLNKQGTRVIRTIPWRFLAGGDNSGIAEDTSKYPNTPEGNKALAKAIVDEYVYKYNLDGLDVDVEHDSIPKVDKKEDTAGVERSIQVFEEIGKLIGPKGVDKSRLFIMDSTYMADKNPLIERGAPYINLLLVQVYGSQGEKGGWEPVSNRPEKTMEERWQGYSKYIRPEQYMIGFSFYEENAQEGNLWYDINSRKDEDKANGINTDITGTRAERYARWQPKTGGVKGGIFSYAIDRDGVAHQPKKYAKQKEFKDATDNIFHSDYSVSKALKTVMLKDKSYDLIDEKDFPDKALREAVMAQVGTRKGDLERFNGTLRLDNPAIQSLEGLNKFKKLAQLDLIGLSRITKLDRSVLPANMKPGKDTLETVLETYKKDNKEEPATIPPVSLKVSGLTGLKELDLSGFDRETLAGLDAATLTSLEKVDISGNKLDLAPGTENRQIFDTMLSTISNHVGSNEQTVKFDKQKPTGHYPDTYGKTSLRLPVANEKVDLQSQLLFGTVTNQGTLINSEADYKAYQNHKIAGRSFVDSNYHYNNFKVSYENYTVKVTDSTLGTTTDKTLATDKEETYKVDFFSPADKTKAVHTAKVIVGDEKTMMVNLAEGATVIGGSADPVNARKVFDGQLGSETDNISLGWDSKQSIIFKLKEDGLIKHWRFFNDSARNPETTNKPIQEASLQIFNIKDYNLDNLLENPNKFDDEKYWITVDTYSAQGERATAFSNTLNNITSKYWRVVFDTKGDRYSSPVVPELQILGYPLPNADTIMKTVTTAKELSQQKDKFSQKMLDELKIKEMALETSLNSKIFDVTAINANAGVLKDCIEKRQLLKK

SEQ ID NO: 8 - protein, 561 aa, Artificial Sequence
  1-344 of Lactobacillus casei alpha1,6-fucosidase fused with a CPD tag and a his tag in the C-terminus
MNDNVAWFKQAKYGMMIHWGLYSLLAGEYRGESSSAYAEWIQSKFQIPNAEYGNLATAFNPLYFDAKKIVALAKQCGMQYLVVTTKHHDGFAMYHSKVDAYNVYDATPFHRDIIGELAEACQKAGLKFGLYYSQDLDWHDPNGGGYKSNDVETAGTTWDNSWDFPDEDQKNFDLCFDNKILPQIKEIMSNYGDIATAWFDVPMTLSEAQSQTIYDTVRELQPNCLINSRLGNGKYDFVSLGDNEIPKNKEDMNKTDVDYNEITGFKPSPLGLYETAGTINDSWGFSYHDQNWKTPRTLYRYKQHLNDFGINYLLNVGLDPLGRVPMMAEENLLAAKALEDEANRALADGKILHNQNVNSWGPITVTPTTDGGETRFDGQIIVQMENDPVVAKAAANLAGKHAESSVVVQLDSDGNYRVVYGDPSKLDGKLRWQLVGHGRDHSETNNTRLSGYSADELAVKLAKFQQSFNQAENINNKPDHISIVGCSLVSDDKQKGFGHQFINAMDANGLRVDVSVRSSELAVDEAGRKHTKDANGDWVQKAENNKVSLSWDAQGHHHHHH

SEQ ID NO: 9 - protein, 344 aa, Artificial Sequence
  Lactobacillus casei alpha1,6-fucosidase (1-344)
MNDNVAWFKQAKYGMMIHWGLYSLLAGEYRGESSSAYAEWIQSKFQIPNAEYGNLATAFNPLYFDAKKIVALAKQCGMQYLVVTTKHHDGFAMYHSKVDAYNVYDATPFHRDIIGELAEACQKAGLKFGLYYSQDLDWHDPNGGGYKSNDVETAGTTWDNSWDFPDEDQKNFDLCFDNKILPQIKEIMSNYGDIATAWFDVPMTLSEAQSQTIYDTVRELQPNCLINSRLGNGKYDFVSLGDNEIPKNKEDMNKTDVDYNEITGFKPSPLGLYETAGTINDSWGFSYHDQNWKTPRTLYRYKQHLNDFGINYLLNVGLDPLGRVPMMAEENLLAAKALEDEANR

SEQ ID NO: 10 - protein, 214 aa, Artificial Sequence
  trastuzumab LC
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 11 - protein, 450 aa, Artificial Sequence
  trastuzumab HC
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 12 - protein, 213 aa, Artificial Sequence
  rituximab LC
QIVLSQSPAILSASPGEKVTMTCRASSSVSYIHWFQQKPGSSPKPWIYATSNLASGVPVRFSGSGSGTSYSLTISRVEAEDAATYYCQQWTSNPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 13 - protein, 451 aa, Artificial Sequence
  rituximab HC
QVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVASASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKAEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 14 - protein, 214 aa, Artificial Sequence
  bevacizumab LC
DIQMTQSPSSLSASVGDRVTITCSASQDISNYLNWYQQKPGKAPKVLIYFTSSLHSGVPSRFSGSGSGTDFTLTISSLQPEDFATYYCQQYSTVPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 15 - protein, 453 aa, Artificial Sequence
  bevacizumab HC
EVQLVESGGGLVQPGGSLRLSCAASGYTFTNYGMNWVRQAPGKGLEWVGWINTYTGEPTYAADFKRRFTFSLDTSKSTAYLQMNSLRAEDTAVYYCAKYPHYYGSSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 16 - protein, 214 aa, Artificial Sequence
  hRS7 LC
DIQLTQSPSSLSASVGDRVSITCKASQDVSIAVAWYQQKPGKAPKLLIYSASYRYTGVPDRFSGSGSGTDFTLTISSLQPEDFAVYYCQQHYITPLTFGAGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 17 - protein, 450 aa, Artificial Sequence
  hRS7 HC
VQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYWYFDVWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

(19) United States
(12) Patent Application Publication
(10) Pub. No.: US 2024/0350658 A1
(43) Pub. Date: Oct. 24, 2024

(54) SITE-SPECIFIC GLYCOPROTEIN CONJUGATES AND METHODS FOR MAKING THE SAME

(71) Applicant: GLYCO-THERAPY BIOTECHNOLOGY CO., LTD., Zhejiang (CN)

(72) Inventors: Yi YANG, Hangzhou (CN); Zhentao SONG, Hangzhou (CN); Ji CHEN, Hangzhou (CN)

(73) Assignee: GLYCO-THERAPY BIOTECHNOLOGY CO., LTD., Hangzhou (CN)

(21) Appl. No.: 18/697,632

(22) PCT Filed: Oct. 20, 2021
(86) PCT No.: PCT/CN2021/124891

§ 371 Date: Apr. 1, 2024

(51) Int. Cl. A61K 47/68; A61K 47/54; A61K 47/64

(52) U.S. Cl. CPC A61K 47/6889; A61K 47/545; A61K 47/549; A61K 47/64

(57) ABSTRACT

A method for preparing a protein conjugate, including contacting a fucose derivative donor Q-Fuc*′ with a protein containing an oligosaccharide in a presence of a catalyst to obtain the protein conjugate. A protein conjugate obtained by the method. A method for preventing or treating disease, including administering the protein conjugate.

### BACKGROUND OF THE INVENTION

Antibody-drug conjugates (ADC) have been considered as promising drug candidates, as they enable targeted delivery of effective drug payloads, providing significantly improved therapeutic index. However, development of effective and safe ADCs remains challenging as many factors, including the chosen of the antibodies and the payloads, the linkage stability, the number and site of the installed payloads, and the homogeneity of the conjugates are difficult to control. Current nonspecific conjugation methods such as cysteine or lysine conjugation yield heterogeneous mixtures of products that are differ in the sites and stoichiometry of modification, resulting in heterogenous pharmacological properties, Although various approaches have been developed to generate site-specific, homogeneous and stable ADCs, the majority of these methods still requires antibodies to be genetically modified either by site-directed mutations or by the introduction of genetically encoded tags that remains to be a laborious task and may compromise the yield of the antibodies. Therefore, genetic-engineering-free approaches that enable site-specific drug conjugation to antibodies would bring much-needed advance to the field.

Recently, the modification of the pendant glycans of antibodies' constant domain has become a convenient way for site-specific conjugation without additional genetically engineering. The glycans of antibodies consists of diverse monosaccharide building blocks that provide multiple potential sites for conjugation. However, glycosylation is a highly heterogeneous post-translational modification, rendering the generation of homogeneous glycans for chemical modification a formidable challenge.

Most of therapeutic mAbs have a single N-linked biantennary carbohydrate structures at Asn297 with heterogeneity in core α-1,6-fucosylation, terminal sialylation and galactosylation, which is located in both heavy chains in the Fc region of the molecule. Several attempts have been made to remodel the N297 glycan part to construct antibody conjugates on native antibodies (Bertozzi C. R. et al., Bioconjugate Chem. 2015, 26, 176-192).

Through a metabolic incorporating strategy, Okeley N. M. et al. were able to incorporate 6-thiofucose onto IgG glycans at a percentage of 60-70% (Okeley N. M. et al., Bioconjugate Chem. 2013, 24, 1650-1655). Following conjugation with a maleimide-linked MMAE yielded a conjugate with an average DAR of 1.3. However, the incorporation ratio of the unnatural 6-thiofucose were difficult to control, leading to heterogeneous antibodies conjugates.

Several strategies were developed by using endoglycosidases and their mutants to install reaction handles on the N-Glycan of the Fc domains of antibodies. In general, N297 glycans were first trimmed by a endoglycosidase to leave the core N-acetylglucosamine (GlcNAc) moiety with or without core-fucoslylation. Then, endoglycosidases mutants were used to transfer oligosaccharide bearing alkyne or azido groups to the trimmed antibody. Wang L. et al. reported the transfer of a tetrasaccharide oxazoline containing two 6-azido mannose moieties on rituximab by using EndoS and its mutant, resulting in a antibody molecule containing four azido groups (Wang L. et al., J. Am. Chem. Soc. 2012, 134, 12308-12318). Similarly, Huang W. et al. reported the transfer of non-natural egg-yolk sialylglycopeptide (SGP) derivatives carrying azido tags to trastuzumab, following reaction with DBCO conjugated cytotoxin enables the generation of homogeneous antibody drug conjugates with a DAR of 4 (Huang W. et al., Org. Biomol. Chem., 2016, 14, 9501-9518). However, a disadvantage of the glycosynthase strategies is the lengthy and complex synthesis of the required oligosaccharide derivatives.

Zhou Q. et al. using galactosyltransferases and sialyltransferases to introduce terminal sialic acids on the native glycans of N297 on the antibody (Zhou Q. et al., Bioconjugate Chem. 2014, 25, 510-520). Periodate oxidation of these sialic acids yielded aldehyde groups which were subsequently used to conjugate aminooxy functionalized cytotoxins via oxime ligation. This strategy enables the incorporation of an average of 1.6 cytotoxins per antibody molecule. Similarly, by treating co-fucosylated IgG with sodium periodate, Neri D. et al. were able to oxidize the core-fucose residues to an aldehyde, which were further used to prepare hydrazone conjugates with fluorophores and a dolastatin analogue (Neri D. et al., Chem. Commun., 2012, 48, 7100-7102). However, the use of periodate oxidation may lead to the oxidative damage to the antibodies.

Dimitrov D. S et al. employed the bovine GalT-Y289L galatosyltransferase mutant to transfer the a galactose moiety comprising a C2-substituted keto group onto the terminal GlcNAc of a degalatosylated G₀F glycoform of an intact antibody (Dimitrov D. S et al., mAbs, 2014, 6, 1190-1200). Following oxime ligation reaction enables the installation of cytotoxins to the modified antibody. Boons G. et al. exploited the ST6Gal1 to incorporate a sialic acid derivative modified with an azide at the C9 position into the terminal galactose of an intact antibody, leading to a four azido groups modified antibody (Boons G. et al., Angew. Chem. Int. Ed. 2014, 53, 7179-7182). van Delft F. L. et al. employed a mutant of bovine galactosyltransferase (GalT-Y289L) to transfer azido-tagged N-Acetylgalactosamine (UDP-GalNAz) onto a core-fucosylated as well as nonfucosylated core-GlcNAc-Fc domain of intact antibodies, resulted in a antibody molecule containing two azido groups, following reaction with the bicyclononyne (BCN) conjugated cytotoxins enables the generation of homogeneous ADCs with a DAR of 2 (van Delft F. L. et al., Bioconjugate Chem. 2015, 26, 2233-2242). However, only very limited groups (typically azido group and ketone group) were modified to the antibodies by these strategies, leading to very finite reactions could be applied to introduce a payload to the antibodies by a second ligation step. Thus, exploring novel building blocks on the glycan of the antibody that enables the introducing of multiple functional reaction groups into the antibodies, or the directly introducing of a payload into the antibodies without a second step ligation reaction is highly desirable. However, development of such a robust conjugation strategy to generate effective and safe ADCs remains high challenging due many factors, including the minimal disturbance of affinity of the antibody, the number and the sites of the installed payloads, the linkage stability, and the homogeneity of the conjugates are difficult to control.

In another aspect, combination therapies have become increasingly necessary to overcome multidrug resistance. Combination of different payloads has been proved to improve the efficacy of ADCs (Senter, P. D, et. al Angew. Chem. Int. Ed. 2019, 129, 751-755). However, the controlled site-specific conjugation of different payloads onto a single antibody molecule remains hard to achieve by most of the current conjugation strategies. In addition, current glyco-editing strategies generally conjugated on one kind of building blocks of the glycan of the antibodies, which in somehow limit the combination of different payloads. Exploring more building blocks on the glycan that enables the conjugation would significantly facilitate the combination of payloads.

Above all, site-specific antibody conjugates with well-controlled payload number, high homogeneity and high stability, are urgently needed, especially when the conjugates have multiple and specific conjugation sites. Therefore, a site-specific glyco-conjugation method for preparation of such a conjugate is highly needed.

### SUMMARY OF THE INVENTION

The present disclosure provides a protein conjugate and a method for making the same. The present disclosure explores new building blocks on the oligosaccharide of a protein for making a protein conjugate. The protein conjugate of the present disclosure has at least one of the following characteristics: (a) well-controlled and defined conjugation sites on the oligosaccharide of the protein; (b) multiple conjugation site on different building blocks of the oligosaccharide of the protein; (c) well defined molecule of interest (MOI)-to-antibody ratio (MAR); (d) high homogeneity. (e) negligible influence of the binding affinity of the antibody; (f) high stability (for example, the conjugation linkage between the fucose or fucose derivative (Fuco) of Fuc* and the GlcNAc of the -GlcNAc(Fuc)_{b}-GalX is stable in plasma (e.g. human plasma) for at least 1 day, as measured in mass spectrometry analysis, e.g., two days, three days, four days, five days, six days, seven days, eight days or more).

With the method developed in this invention, a variety of functional groups could be transferred to the antibodies using an α-1,3-fucotrasferase and a GDP-Fuc*′ to generated the antibody-functional-group-conjugates with high reactivity. Multiple ligation reaction could be applied to install a biologically and/or pharmaceutically active moiety (e.g. a cytotoxin) to the antibody to generate the antibody conjugates (the “two-step” process). In addition, a biologically and/or pharmaceutically active moiety (e.g. a cytotoxin) could be directly transferred to the antibodies using an α-1,3-fucotrasferase and the GDP-Fuc*′ to make the antibody conjugates (the “one-step” process).

Furthermore, by utilizing two building blocks of the oligosaccharide of a protein, we were able to specifically install MOIs at different sites of the protein. The multiple and specific conjugation sites provide the flexibility in payload combination. For example, the protein conjugates could have a M₁AR of 2, a M₁AR of 4, a M₁AR of 2 and M₂AR of 2 (MAR 2+2), or a M₁AR₁ of 4 and a M₂AR of 4 (MAR 4+4).

In one aspect, the present disclosure provides a protein conjugate, which comprises a protein and an oligosaccharide, wherein the oligosaccharide comprises Formula (1):

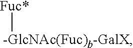

wherein, the GlcNAc is directly or indirectly linked to an amino acid of the protein, the GalX is a substituted galactose, the Fuc in parentheses is a fucose, and b is 0 or 1, the Fuc* comprises a fucose or fucose derivative (Fuco) and a molecule of interest (MOI₁).

In some embodiments, the protein comprises an antigen binding fragment and/or a Fc fragment.

In some embodiments, the oligosaccharide is an N-linked oligosaccharide. In some embodiments, the oligosaccharide is an O-linked oligosaccharide.

In some embodiments, the oligosaccharide is linked to an Asparagine (Asn) residue of the protein.

In some embodiments, the GlcNAc of Formula (1) is directly linked to an Asn residue of the protein. In some embodiments, the GlcNAc of Formula (1) is directly linked to an Asn residue of the protein and b is 1. In some embodiments, the GlcNAc of Formula (1) is directly linked to an Asn residue of the protein and b is 0.

In some embodiments, the GlcNAc of Formula (1) is linked to a saccharide of the oligosaccharide.

In some embodiments, the GlcNAc of Formula (1) is linked to a mannose of the oligosaccharide. In some embodiments, the GlcNAc of Formula (1) is linked to a mannose of the oligosaccharide, preferably b is 0.

In some embodiments, the protein comprises a Fc fragment.

In some embodiments, the protein comprises a Fc fragment and the oligosaccharide is linked to the Fc fragment.

In some embodiments, the oligosaccharide is linked to the CH₂ domain of the Fc fragment.

In some embodiments, the oligosaccharide is linked to Asn297 of the Fc fragment, numbered according to the Kabat numbering system.

In some embodiments, the protein has one or more oligosaccharides on other position not mentioned above.

In some embodiments, the protein is an antibody.

In some embodiments, the antibody is a monoclonal antibody.

In some embodiments, the antibody is an IgG antibody. In some embodiments, the antibody is an IgA antibody. In some embodiments, the antibody is an IgE antibody. In some embodiments, the antibody is an IgM antibody.

In some embodiments, the antibody is a humanized antibody. In some embodiments, the antibody is a fully human antibody. In some embodiments, the antibody is a chimeric antibody.

In some embodiments, the Fuco of the Fuc* is linked to the GlcNAc through an Fuc*α1,3GlcNAc linkage.

In some embodiments, the GalX is linked to the GlcNAc through a GalXβ1,4GlcNAc linkage.

In some embodiments, the b is 1. In some embodiments, the GalX is directly linked to an Asn of the protein, and b is 1. And in some embodiments, the Fuc is linked to the GlcNAc of Formula (1) through an α1,6 linkage.

In some embodiments, the MOI₁ of Fuc* comprises an active moiety.

In some embodiments, the active moiety of MOI₁ is a chemically active moiety, an enzymatically active moiety, a biologically active moiety, and/or a pharmaceutically active moiety.

In some embodiments, the active moiety of MOI₁ is a chemically active moiety and/or an enzymatically active moiety.

In some embodiments, the active moiety of MOI₁ comprises a X₁, and X₁ is a functional group capable of participating in a ligation reaction.

In some embodiments, the X₁ comprises a functional moiety capable of participating in a bioorthogonal ligation reaction. For example, the X₁ comprises one or more functional moieties.

In some embodiments, the X₁ comprises a functional moiety selected from the group consisting of azido group, terminal alkynyl group, cyclic alkynyl group, tetrazinyl group, 1,2,4-trazinyl group, terminal alkenyl group, cyclic alkenyl group, ketone group, aldehyde group, hydroxyl amino group, sulfydryl group, N-maleimide group and their functional derivatives. In some embodiments, the X₁ comprises a functional moiety derived from a group selected from the group consisting of azido group, terminal alkynyl group, cyclic alkynyl group, tetrazinyl group, 1,2,4-trazinyl group, terminal alkenyl group, cyclic alkenyl group, ketone group, aldehyde group, hydroxyl amino group, sulfydryl group and N-maleimide group. For example, the X₁ comprises one or more functional moieties.

In some embodiments, the X₁ comprises a functional moiety selected from the group consisting of

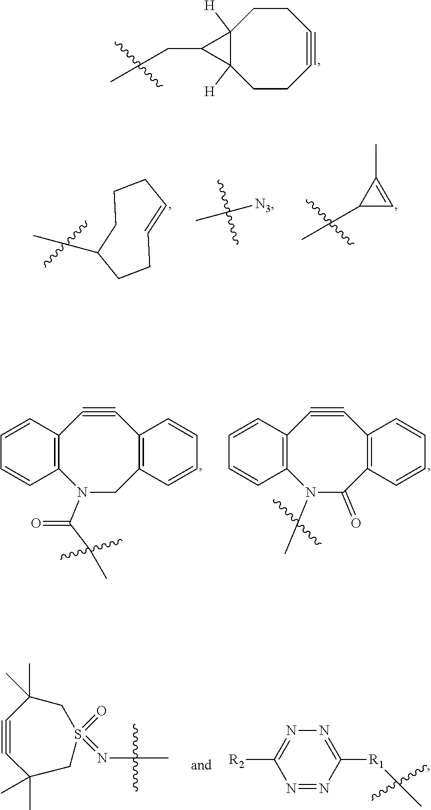

wherein R₁ is selected from the group consisting of C₁-C₂₂ alkylene group, C₅-C₂₂ (hetero)arylene group, C₆-C₂₂ alkyl(hetero)arylene group and C₆-C₂₂ (hetero)arylalkylene group, wherein the alkylene group, the (hetero)arylene group, the alkyl(hetero)arylene group or the (hetero)arylalkylene group is optionally substituted. R² is selected from the group consisting of hydrogen, halogen, C₁-C₂₂ alkyl group, C₅-C₂₂ (hetero)aryl group, C₆-C₂₂ alkyl(hetero)aryl group and C₆-C₂₂ (hetero)arylalkyl group, wherein the alkyl group, the (hetero)aryl group, the alkyl(hetero)aryl group or the (hetero)arylalkyl group is optionally substituted. For example, the alkylene group, the (hetero)arylene group, the alkyl(hetero)arylene group or the (hetero)arylalkylene group is optionally substituted by one or more halogen, —OH, —NH₂, —COOH or —CN. For example, the alkyl group, the (hetero)aryl group, the alkyl(hetero)aryl group or the (hetero)arylalkyl group is optionally substituted by one or more halogen, —OH, —NH₂, —COOH or —CN.

In some embodiments, the X₁ comprises a functional moiety selected from the group consisting of

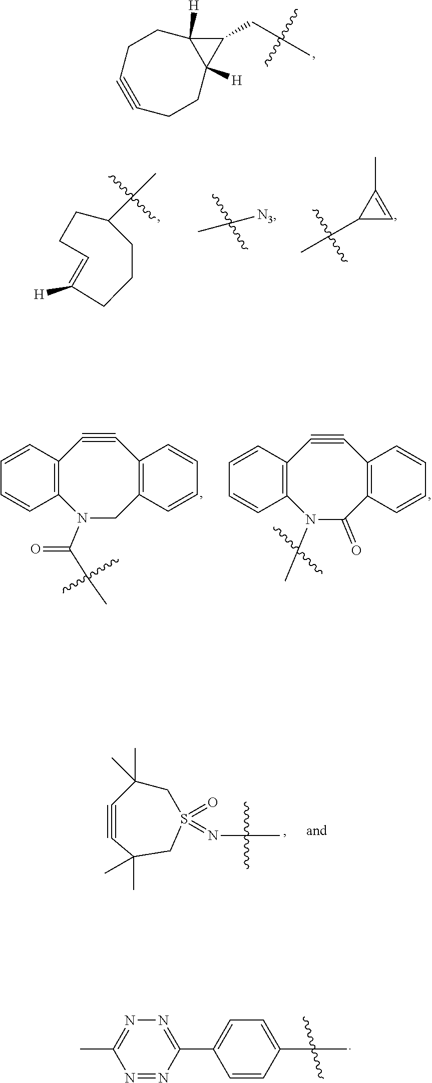

In some embodiments, the active moiety of MOI₁ comprises a P₁, and P₁ is a biologically active moiety and/or a pharmaceutically active moiety.

In some embodiments, the P₁ comprises a cytotoxin, an agonist, an antagonist, an antiviral agent, an antibacterial agent, a radioisotope or a radionuclide, a metal chelator, a fluorescent dye, a biotin, an oligonucleotide, a peptide, a protein, or any combination thereof.

In some embodiments, the P₁ is a pharmaceutically active moiety.

In some embodiments, the P₁ comprises a cytotoxin, an agonist, an antagonist, an antiviral agent, an antibacterial agent, an oligonucleotide, a peptide or any combination thereof.

In some embodiments, the P₁ comprises a cytotoxin. For example, the P₁ may comprise one or more cytotoxin molecules.

In some embodiments, the P₁ comprises a nucleic acid (e.g., DNA and/or RNA) damaging agent, a topoisomerase inhibitor and/or a microtubule inhibitor.

In some embodiments, the P₁ comprises a cytotoxin selected from the group consisting of pyrrolobenzodiazepine, auristatin, maytansinoids, duocarmycin, tubulysin, enediyene, doxorubicin, pyrrole-based kinesin spindle protein inhibitor, calicheamicin, amanitin and camptothecin.

In some embodiments, the P₁ comprises a cytotoxin selected from the group consisting of MMAE, MMAF, DXd, DM4 and seco-DUBA.

In some embodiments, the MOI₁ of Fuc* comprises a X₁Y₁, and X₁Y₁ is a remaining group after a ligation reaction between the functional group X₁ and a functional group Y₁.

In some embodiments, the X₁Y₁ is between the Fuco of Fuc* and the P₁.

In some embodiments, the Y₁ comprises a functional moiety capable of participating in a bioorthogonal reaction. In some embodiments, the Y₁ does not react with an amino acid of the protein.

In some embodiments, the Y₁ comprises a functional moiety selected from the group consisting of azido group, terminal alkynyl group, cyclic alkynyl group, tetrazinyl group, 1,2,4-trazinyl group, terminal alkenyl group, cyclic alkenyl group, ketone group, aldehyde group, hydroxyl amino group, sulfydryl group, N-maleimide group and their functional derivatives. In some embodiments, the Y₁ comprises a functional moiety derived from the group consisting of azido group, terminal alkynyl group, cyclic alkynyl group, tetrazinyl group, 1,2,4-trazinyl group, terminal alkenyl group, cyclic alkenyl group, ketone group, aldehyde group, hydroxyl amino group, sulfydryl group and N-maleimide group.

In some embodiments, the Y₁ comprises a functional moiety selected from the group consisting of

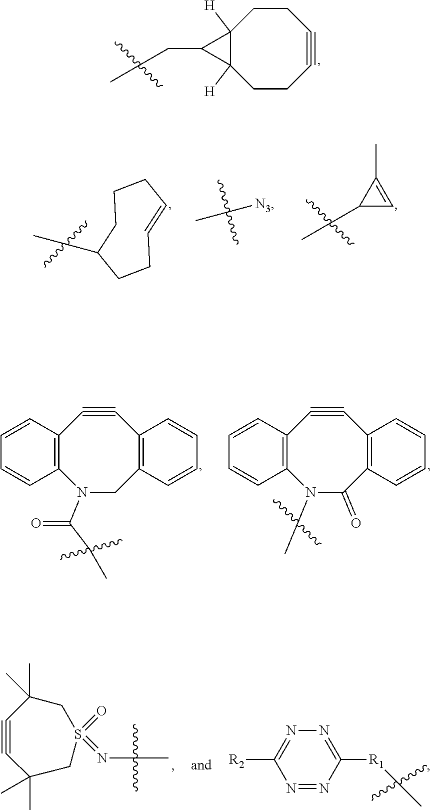

wherein each of R₁ is selected from the group consisting of C₁-C₂₂ alkylene group, C₅-C₂₂ (hetero)arylene group, C₆-C₂₂ alkyl(hetero)arylene group and C₆-C₂₂ (hetero)arylalkylene group, wherein the alkylene group, the (hetero)arylene group, the alkyl(hetero)arylene group or the (hetero)arylalkylene group is optionally substituted. R² is selected from the group consisting of hydrogen, halogen, C₁-C₂₂ alkyl group, C₅-C₂₂ (hetero)aryl group, C₆-C₂₂ alkyl(hetero)aryl group and C₆-C₂₂ (hetero)arylalkyl group, wherein the alkyl group, the (hetero)aryl group, the alkyl(hetero)aryl group or the (hetero)arylalkyl group is optionally substituted. For example, the alkylene group, the (hetero)arylene group, the alkyl(hetero)arylene group or the (hetero)arylalkylene group is optionally substituted by one or more halogen, —OH, —NH₂, —COOH or —CN. For example, the alkyl group, the (hetero)aryl group, the alkyl(hetero)aryl group or the (hetero)arylalkyl group is optionally substituted by one or more halogen, —OH, —NH₂, —COOH or —CN.

In some embodiments, the Y₁ comprises a functional moiety selected from the group consisting of

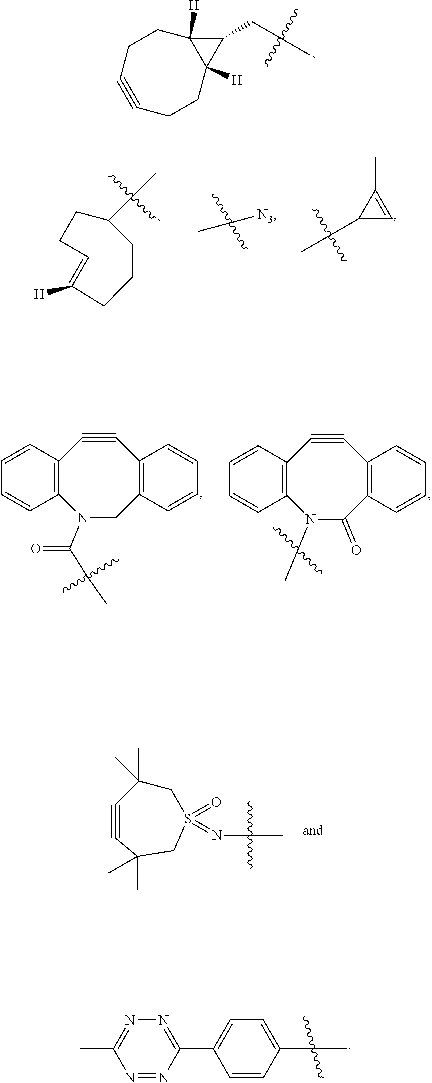

In some embodiments, the X₁Y₁ is selected from the group consisting of

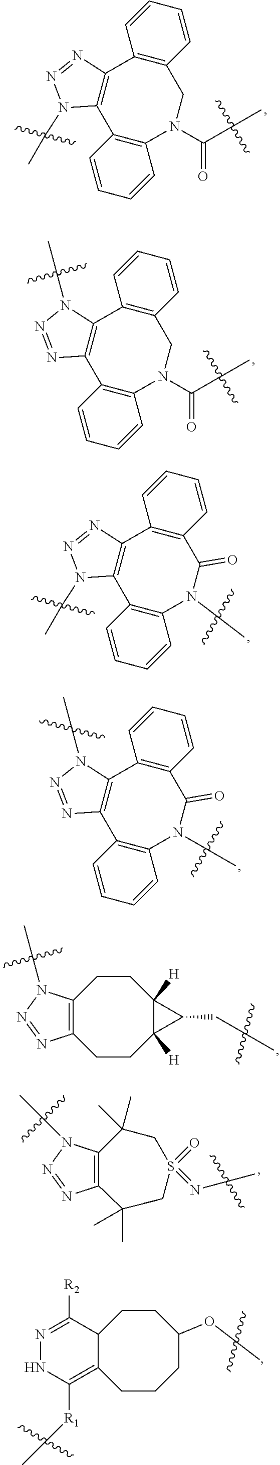

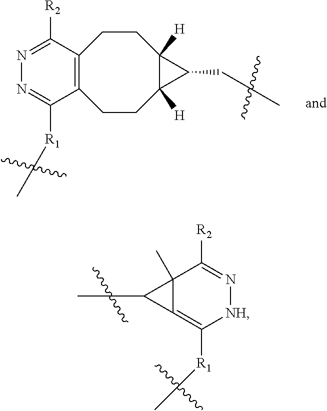

wherein R₁ is selected from the group consisting of C₁-C₂₂ alkylene group, C₅-C₂₂ (hetero)arylene group, C₆-C₂₂ alkyl(hetero)arylene group and C₆-C₂₂ (hetero)arylalkylene group, wherein the alkylene group, the (hetero)arylene group, the alkyl(hetero)arylene group or the (hetero)arylalkylene group is optionally substituted, R² is selected from the group consisting of hydrogen, halogen, C₁-C₂₂ alkyl group, C₅-C₂₂ (hetero)aryl group, C₆-C₂₂ alkyl(hetero)aryl group and C₆-C₂₂ (hetero)arylalkyl group, wherein the alkyl group, the (hetero)aryl group, the alkyl(hetero)aryl group or the (hetero)arylalkyl group is optionally substituted. For example, the alkylene group, the (hetero)arylene group, the alkyl(hetero)arylene group or the (hetero)arylalkylene group is optionally substituted by one or more halogen, —OH, —NH₂, —COOH or —CN. For example, the alkyl group, the (hetero)aryl group, the alkyl(hetero)aryl group or the (hetero)arylalkyl group is optionally substituted by one or more halogen, —OH, —NH₂, —COOH or —CN.

In some embodiments, the X₁ and the Y₁ comprise the functional moieties selected from the group consisting of:
a) X₁ comprises

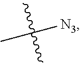 [SMILES: *N=[N+]=[N-]]

and Y₁ comprises

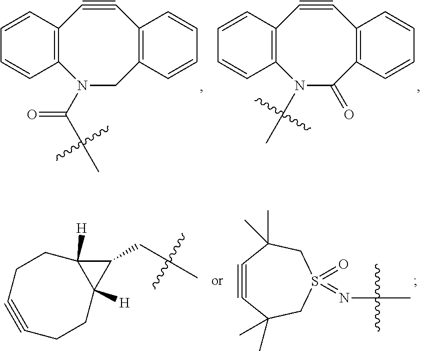

b) X₁ comprises

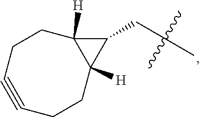 [SMILES: *C[C@H]1[C@H]2CCC#CCC[C@@H]12]

and Y₁ comprises

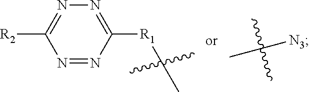

c) X₁ comprises

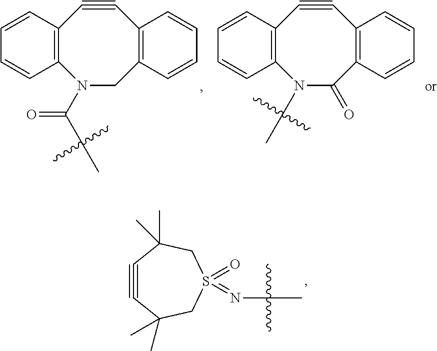

and Y₁ comprises

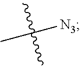 [SMILES: *N=[N+]=[N-]]

d) X₁ comprises

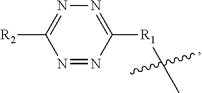

and Y₁ comprises

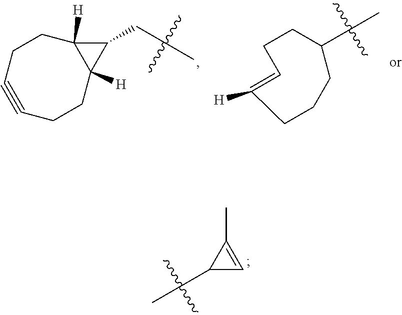

and e) X₁ comprises

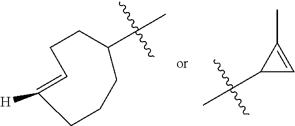

and Y₁ comprises

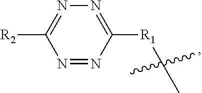

herein R₁ is selected from the group consisting of C₁-C₂₂ alkylene group, C₅-C₂₂ (hetero)arylene group, C₆-C₂₂ alkyl(hetero)arylene group and C₆-C₂₂ (hetero)arylalkylene group, wherein the alkylene group, the (hetero)arylene group, the alkyl(hetero)arylene group or the (hetero)arylalkylene group is optionally substituted, R² is selected from the group consisting of hydrogen, halogen, C₁-C₂₂ alkyl group, C₅-C₂₂ (hetero)aryl group, C₆-C₂₂ alkyl(hetero)aryl group and C₆-C₂₂ (hetero)arylalkyl group, wherein the alkyl group, the (hetero)aryl group, the alkyl(hetero)aryl group or the (hetero)arylalkyl group is optionally substituted. For example, the alkylene group, the (hetero)arylene group, the alkyl(hetero)arylene group or the (hetero)arylalkylene group is optionally substituted by one or more halogen, —OH, —NH₂, —COOH or —CN. For example, the alkyl group, the (hetero)aryl group, the alkyl(hetero)aryl group or the (hetero)arylalkyl group is optionally substituted by one or more halogen, —OH, —NH₂, —COOH or —CN.

In some embodiments, the MOI₁ of the Fuc* comprises a L₁, and L₁ is a linker.

In some embodiments, the L₁ is a cleavable linker.

In some embodiments, the L₁ is an acid-labile linker, a redox-active linker, a photo-active linker and/or a proteolytically cleavable linker.

In some embodiments, the L₁ is a vc-PAB-linker, a disulfo linker, or a GGFG-linker.

In some embodiments, the L₁ is between the Fuco of Fuc* and the P₁.

In some embodiments, the L₁ is between the Fuco of Fuc* and the X₁.

In some embodiments, the L₁ is between the Fuco of Fuc* and the X₁Y₁.

In some embodiments, the MOI₁ of the Fuc* comprises a F, and F is a connector which links X₁, P₁, L₁, or X₁Y₁ to the Fuco of the Fuc*. In some embodiments, the F is between the Fuco of Fuc* and the P₁. In some embodiments, the F is between the Fuco of Fuc* and the X₁. In some embodiments, the F is between the Fuco of Fuc* and the X₁Y₁. In some embodiments, the F is between the Fuco of Fuc* and the L₁.

In some embodiments, the F is according to Formula (2): (J)_{q}-(FL)ₛ, J is a jointer, FL is a spacer, q is 0 or 1 and s is 0 or 1. For example, q is 1. In some embodiments, J is a chemical structure that connects the Fuco of Fuc* and the FL. In some embodiments, J is directly linked to the Fuco of Fuc*.

In some embodiments, the J is a

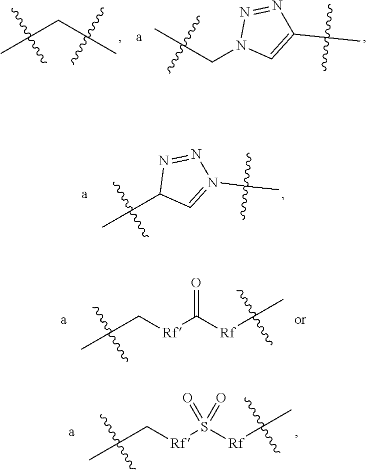

wherein Rf′ is —CH₂—, —NH— or —O—, and Rf is —CH₂—, —NH— or —O—.

In some embodiments, the J is

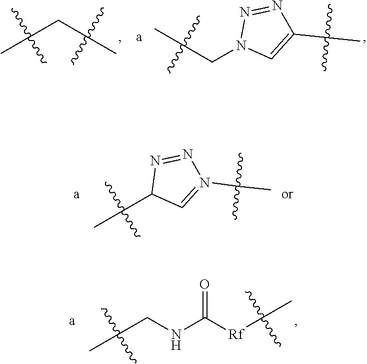

wherein Rf is —CH₂—, —NH— or —O—.

In some embodiments, the J is a

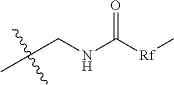

wherein Rf is —CH₂—, —NH— or —O—.

For example, the J is a

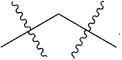 [SMILES: *C*]

For example, the J is a

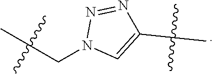 [SMILES: *Cn1cc(*)nn1]

For example, the J is a

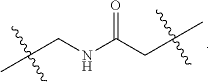 [SMILES: *CNC(=O)C*]

For example, the J is a

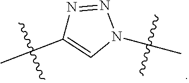 [SMILES: *c1cn(*)nn1]

For example, the J is a

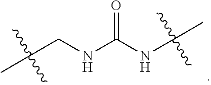 [SMILES: *CNC(=O)N*]

For example, the J is a

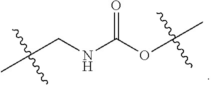 [SMILES: *CNC(=O)O*]

For example, the J is a

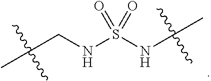 [SMILES: *CNS(=O)(=O)N*]

In some embodiments, in Formula (2), the q is 1 and s is 0 or 1. In some embodiments, Formula (2), the q is 1 and s is 1.

In some embodiments, the FL is selected from the group consisting of C₃-C₂₀₀ peptide, C₂-C₂₀₀ PEG, C₁-C₂₀₀ alkylene group, C₃-C₂₀₀ cycloalkylene group, C₂-C₂₀₀ alkenylene group, C₅-C₂₀₀ cycloalkenylene group, C₂-C₂₀₀ alkynylene group, C₅-C₂₀₀ cycloalkynylene group, C₂-C₂₄ (hetero)arylene group, C₃-C₂₀₀ (hetero)arylalkylene group, C₃-C₂₀₀ alkynyl(hetero)arylene group, their derivatives and any combination thereof, wherein said the peptide, the PEG, the alkylene group, the cycloalkylene group, the alkenylene group, the cycloalkenylene group, the alkynylene group, the cycloalkynylene group, the (hetero)arylene group, the (hetero)arylalkylene group, or the alkynyl(hetero)arylene group is optionally substituted by one or more Rs¹ and/or is optionally interrupted by one or more Rs², wherein Rs¹ is selected from the group consisting of halogen, —OH, —NH₂ and —COOH, Rs² is independently selected from the group consisting of —O—, —S—,

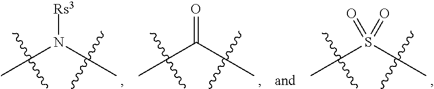

wherein Rs³ is selected from the group consisting of hydrogen, C₁-C₂₄ alkyl group, C₂-C₂₄ alkenyl group, C₂-C₂₄ alkynyl group and C₃-C₂₄ cycloalkyl group.

In some embodiments, the Fuc* is Fuco-(F)ₘ-(L₁)ₙ-X₁, Fuco-(F)ₘ-(L₁)ₙ-P₁, or Fuco-(F)ₘ-(L₁)ₙ-X₁Y₁-(FL′)_{m′}-(L₁′)_{n′}-P₁, wherein Fuco is the fucose or fucose derivative of the Fuc*, F is the connector, L₁ is the linker, P₁ is the biologically and/or pharmaceutically active moiety, X₁ is the functional group, X₁Y₁ is the remaining group, FL′ is a spacer, the L₁′ is a linker, m is 0 or 1, n is 0 or 1, m′ is 0 or 1, and n′ is 0 or 1.

In some embodiments, FL′ is selected from the group consisting of C₃-C₂₀₀ peptide, C₂-C₂₀₀ PEG, C₁-C₂₀₀ alkylene group, C₃-C₂₀₀ cycloalkylene group, C₂-C₂₀₀ alkenylene group, C₅-C₂₀₀ cycloalkenylene group, C₂-C₂₀₀ alkynylene group, C₅-C₂₀₀ cycloalkynylene group, C₂-C₂₄ (hetero)arylene group, C₃-C₂₀₀ (hetero)arylalkylene group, C₃-C₂₀₀ alkynyl(hetero)arylene group, their derivatives and any combination thereof, wherein said the peptide, the PEG, the alkylene group, the cycloalkylene group, the alkenylene group, the cycloalkenylene group, the alkynylene group, the cycloalkynylene group, the (hetero)arylene group, the (hetero)arylalkylene group, or the alkynyl(hetero)arylene group is optionally substituted by one or more Rs¹ and/or is optionally interrupted by one or more Rs², wherein Rs¹ is selected from the group consisting of halogen, —OH, —NH₂ and —COOH, Rs² is independently selected from the group consisting of —O—, —S—,

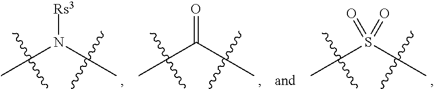

wherein Rs³ is selected from the group consisting of hydrogen, C₁-C₂₄ alkyl group, C₂-C₂₄ alkenyl group, C₂-C₂₄ alkynyl group and C₃-C₂₄ cycloalkyl group.

In some embodiments, the L₁′ is a cleavable linker.

In some embodiments, the L₁′ is an acid-labile linker, a redox-active linker, a photo-active linker and/or a proteolytically cleavable linker.

In some embodiments, the L₁′ is a vc-PAB-linker, a disulfo linker, or a GGFG-linker.

In some embodiments, the Fuc* is Fuco-X₁, Fuco-F-X₁, Fuco-F-L1-P1, Fuco-F-P₁, Fuco-X₁Y₁-FL′-L₁′-P₁, Fuco-X₁Y₁-FL′-P₁, Fuco-X₁Y₁-L₁′-P₁, Fuco-F-X₁Y₁-L₁′-P₁, Fuco-F-X₁Y₁-FL′-P₁ or Fuco-F-X₁Y₁-FL′-L₁′-P₁. For example, the Fuc* is Fuco-X₁. For example, the Fuc* is Fuco-F-X₁. For example, the Fuc* is Fuco-F-L1-P₁. For example, the Fuc* is Fuco-F-P₁. For example, the Fuc* is Fuco-X₁Y₁-FL′-L₁′-P₁. For example, the Fuc* is Fuco-X₁Y₁-FL′-P₁. For example, the Fuc* is Fuco-X₁Y₁-L₁′-P₁. For example, the Fuc* is Fuco-F-X₁Y₁-L₁′-P₁. For example, the Fuc* is Fuco-F-X₁Y₁-FL′-P₁. For example, the Fuc* is Fuco-F-X₁Y₁-FL′-L₁′-P₁.

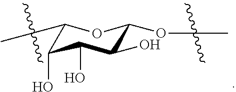

In some embodiments, the Fuco of Fuc* is according to Formula (3). In some embodiments, the Fuc* is according to Formula (4)

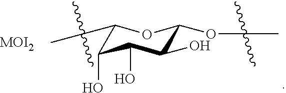

In the protein conjugate, the right part of Formula (3) and Formula (4) is linked to the GlcNAc.

In some embodiments, the GalX is linked to the GlcNAc through a GalXβ1,4GlcNAc linkage.

In some embodiments, the GalX is a substituted galactose, and is substituted on one or more positions selected from the C2 position, the C3 position, the C4 position and the C6 position of the galactose. For example, the hydroxyl group on one or more positions selected from the C2 position, the C3 position, the C4 position and the C6 position of the galactose, is substituted.

In some embodiments, the GalX is a substituted galactose, and is substituted on the C2 position and/or the C6 position. In some embodiments, the GalX is a substituted galactose, and is substituted on the C2 position. For example, the hydroxyl group on the C2 position of the galactose is substituted.

In some embodiments, the GalX is a substituted galactose, and is substituted on the C6 position. For example, the hydroxyl group on the C6 position of the galactose is substituted.

In some embodiments, the GalX is a monosaccharide.

In some embodiments, the GalX is substituted by a substitution Rg and the Rg is according to Formula (5):

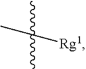

wherein Rg¹ is selected from the group consisting of hydrogen, halogen, —NH₂, —SH, —N₃, —COOH, —CN, C₁-C₂₄ alkyl group, C₃-C₂₄ cycloalkyl group, C₂-C₂₄ alkenyl group, C₅-C₂₄ cycloalkenyl group, C₂-C₂₄ alkynyl group, C₇-C₂₄ cycloalkynyl group, C₂-C₂₄ (hetero)aryl group, C₃-C₂₄ alkyl(hetero)aryl group, C₃-C₂₄ (hetero)arylalkyl group and any combination thereof, wherein the alkyl group, the cycloalkyl group, the alkenyl group, the cycloalkenyl group, the alkynyl group, the cycloalkynyl group, the (hetero)aryl group, the alkyl(hetero)aryl group, the (hetero)arylalkyl group may be substituted by one or more Rs⁴ and may be interrupted by one or more Rs², wherein Rs⁴ is selected from the group of halogen, —OH, —NH₂, —SH, —N₃, —COOH and —CN, Rs² is selected from the group consisting of —O—, —S—,

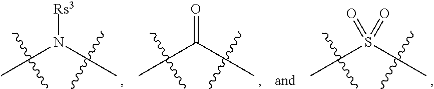

wherein Rs³ is selected from the group consisting of hydrogen, C₁-C₂₄ alkyl group, C₂-C₂₄ alkenyl group, C₂-C₂₄ alkynyl group and C₃-C₂₄ cycloalkyl group.

In some embodiments, the GalX is substituted by a substitution Rg and the Rg is according to Formula (6):

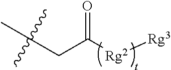

### or Formula (7):

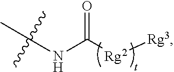

wherein t is 0 or 1, Rg² is selected from the group consisting of C₁-C₂₄ alkylene group, C₃-C₂₄ cycloalkylene group, C₂-C₂₄ alkenylene group, C₅-C₂₄ cycloalkenylene group, C₂-C₂₄ alkynylene group, C₇-C₂₄ cycloalkynylene group, C₂-C₂₄ (hetero)arylene group, C₃-C₂₄ alkyl(hetero)arylene group and C₃-C₂₄ (hetero)arylalkylene group, wherein the alkylene group, the cycloalkylene group, the alkenylene group, the cycloalkenylene group, the alkynylene group, the cycloalkynylene group, the (hetero)arylene group, the alkyl(hetero)arylene group or the (hetero)arylalkylene group may be substituted by one or more Rs⁴ and may be interrupted by one or more Rs², Rg³ is selected from the group consisting of hydrogen, halogen, —OH, —NH₂, —SH, —N₃, —COOH, —CN, C₁-C₂₄ alkyl group, C₃-C₂₄ cycloalkyl group, C₂-C₂₄ alkyne group, C₅-C₂₄ cycloalkyne group, C₂-C₂₄ alkynyl group, C₅-C₂₄ cycloalkynyl group, C₂-C₂₄ (hetero)aryl group and any combination thereof, wherein the C₁-C₂₄ alkyl group, the C₃-C₂₄ cycloalkyl group, the C₂-C₂₄ alkyne group, the C₅-C₂₄ cycloalkyne group, the C₂-C₂₄ alkynyl group, the C₅-C₂₄ cycloalkynyl group, or the C₂-C₂₄ (hetero)aryl group is optionally substituted by one or more Rs⁴, Rs⁴ is selected from the group of halogen, —OH, —NH₂, —SH, —N₃, —COOH and —CN, Rs² is selected from the group consisting of —O—, —S—,

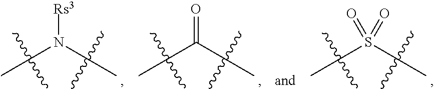

wherein Rs³ is selected from the group consisting of hydrogen, C₁-C₂₄ alkyl group, C₂-C₂₄ alkenyl group, C₂-C₂₄ alkynyl group and C₃-C₂₄ cycloalkyl group.

In some embodiments, the GalX comprises a X₂, and X₂ is functional group which comprising a functional moiety capable of participating in a ligation reaction. In this case, GalX is represented by GalX₂.

In some embodiments, the GalX comprises a X₂, and X₂ is functional group which comprising a functional moiety capable of participating in a bioorthogonal ligation reaction. In this case, GalX is represented by GalX₂.

In some embodiments, the X₂ comprises a functional moiety selected from the group consisting of azido group, terminal alkynyl group, cyclic alkynyl group, tetrazinyl group, 1,2,4-trazinyl group, terminal alkenyl group, cyclic alkenyl group, ketone group, aldehyde group, hydroxyl amino group, sulfydryl group, N-maleimide group and their functional derivatives. In some embodiments, the X₂ comprises a functional moiety selected from the group consisting of azido group, terminal alkynyl group, cyclic alkynyl group, tetrazinyl group, 1,2,4-trazinyl group, terminal alkenyl group, cyclic alkenyl group, ketone group, aldehyde group, hydroxyl amino group, sulfydryl group, and N-maleimide group. For example, the X₂ comprises one or more functional moieties.

In some embodiments, the X₂ comprises a functional moiety selected from the group consisting of

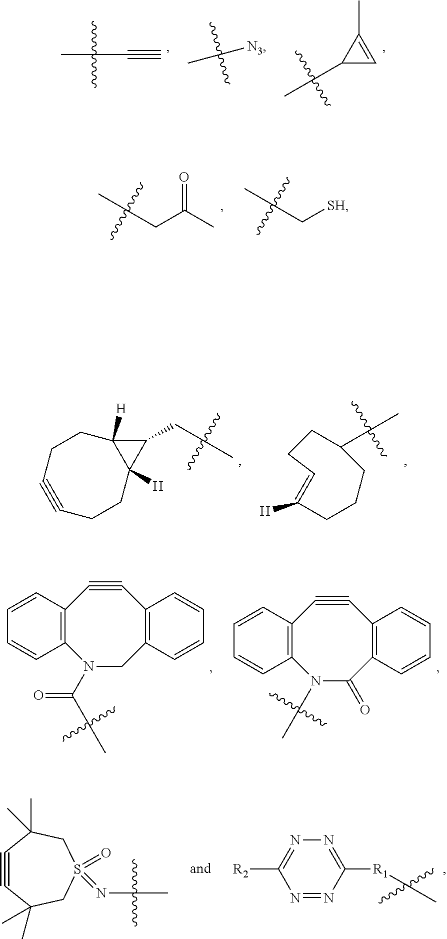

wherein R₁ is selected from the group consisting of C₁-C₂₂ alkylene group, C₅-C₂₂ (hetero)arylene group, C₆-C₂₂ alkyl(hetero)arylene group and C₆-C₂₂ (hetero)arylalkylene group, wherein the alkylene group, the (hetero)arylene group, the alkyl(hetero)arylene group or the (hetero)arylalkylene group is optionally substituted. R₂ is selected from the group consisting of hydrogen, halogen, C₁-C₂₂ alkyl group, C₅-C₂₂ (hetero)aryl group, C₆-C₂₂ alkyl(hetero)aryl group and C₆-C₂₂ (hetero)arylalkyl group, wherein the alkyl group, the (hetero)aryl group, the alkyl(hetero)aryl group or the (hetero)arylalkyl group is optionally substituted. For example, the alkylene group, the (hetero)arylene group, the alkyl(hetero)arylene group or the (hetero)arylalkylene group is optionally substituted by one or more halogen, —OH, —NH₂, —COOH or —CN. For example, the alkyl group, the (hetero)aryl group, the alkyl(hetero)aryl group or the (hetero)arylalkyl group is optionally substituted by one or more halogen, —OH, —NH₂, —COOH or —CN.

In some embodiments, the X₂ comprises a functional moiety selected from the group consisting of

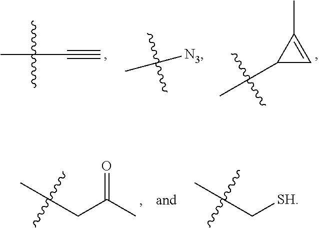

For example, the X₂ comprises

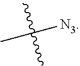 [SMILES: *N=[N+]=[N-]]

In some embodiments, the GalX comprises a P₂, and P₂ is a biologically and/or pharmaceutically active moiety.

In some embodiments, the P₂ comprises a cytotoxin, an agonist, an antagonist, an antiviral agent, an antibacterial agent, a radioisotope or a radionuclide, a metal chelator, a fluorescent dye, a biotin, an oligonucleotide, a peptide, a protein, or any combination thereof.

In some embodiments, the P₂ is a pharmaceutically active moiety.

In some embodiments, the P₂ comprises a cytotoxin, an agonist, an antagonist, an antiviral agent, an antibacterial agent, an oligonucleotide, a peptide or any combination thereof.

In some embodiments, the P₂ comprises a cytotoxin. For example, the P₂ comprises one or more cytotoxin.

In some embodiments, the P₂ comprises a cytotoxin selected from the group consisting of DNA and/or RNA damaging agent, topoisomerase inhibitor and microtubule inhibitor.

In some embodiments, the P₂ comprises a cytotoxin selected from the group consisting of pyrrolobenzodiazepine, auristatin, maytansinoids, duocarmycin, tubulysin, enediyene, doxorubicin, pyrrole-based kinesin spindle protein inhibitor, calicheamicin, amanitin and camptothecin.

In some embodiments, the P₂ comprises a cytotoxin selected from the group consisting of MMAE, MMAF, DXd, DM4 and seco-DUBA.

In some embodiments, the GalX optionally comprises a X₂Y₂, and X₂Y₂ is a remaining group after a ligation reaction between the X₂ and Y₂, and Y₂ is a functional group.

In some embodiments, the Y₂ comprises a functional moiety capable of participating in a bioorthogonal ligation reaction.

In some embodiments, Y₂ comprises a functional moiety selected from the group consisting of azido group, terminal alkynyl group, cyclic alkynyl group, tetrazinyl group, 1,2,4-trazinyl group, terminal alkenyl group, cyclic alkenyl group, ketone group, aldehyde group, hydroxyl amino group, sulfydryl group, N-maleimide group and their functional derivatives. In some embodiments, Y₂ comprises a functional moiety derived from the group consisting of azido group, terminal alkynyl group, cyclic alkynyl group, tetrazinyl group, 1,2,4-trazinyl group, terminal alkenyl group, cyclic alkenyl group, ketone group, aldehyde group, hydroxyl amino group, sulfydryl group and N-maleimide group.

In some embodiments, the Y₂ comprises a functional moiety selected from the group consisting of

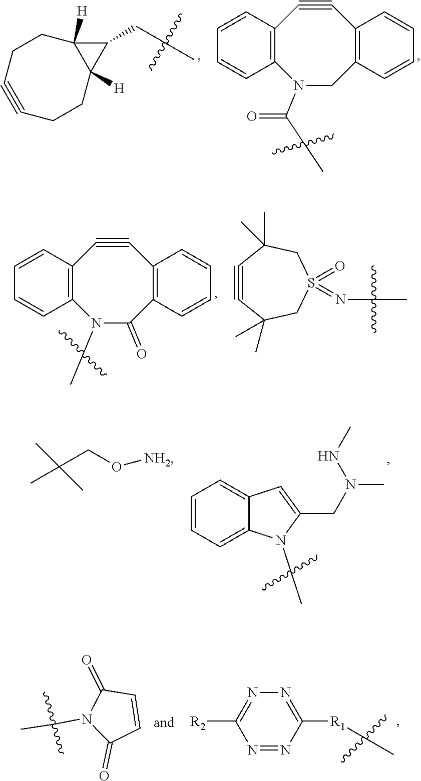

wherein R₁ is selected from the group consisting of C₁-C₂₂ alkylene group, C₅-C₂₂ (hetero)arylene group, C₆-C₂₂ alkyl(hetero)arylene group and C₆-C₂₂ (hetero)arylalkylene group, wherein the alkylene group, the (hetero)arylene group, the alkyl(hetero)arylene group or the (hetero)arylalkylene group is optionally substituted. R₂ is selected from the group consisting of hydrogen, halogen, C₁-C₂₂ alkyl group, C₅-C₂₂ (hetero)aryl group, C₆-C₂₂ alkyl(hetero)aryl group and C₆-C₂₂ (hetero)arylalkyl group, wherein the alkyl group, the (hetero)aryl group, the alkyl(hetero)aryl group or the (hetero)arylalkyl group is optionally substituted. For example, the alkylene group, the (hetero)arylene group, the alkyl(hetero)arylene group or the (hetero)arylalkylene group is optionally substituted by one or more halogen, —OH, —NH₂, —COOH or —CN. For example, the alkyl group, the (hetero)aryl group, the alkyl(hetero)aryl group or the (hetero)arylalkyl group is optionally substituted by one or more halogen, —OH, —NH₂, —COOH or —CN.

In some embodiments, the Y₂ comprises a functional moiety selected from the group consisting of

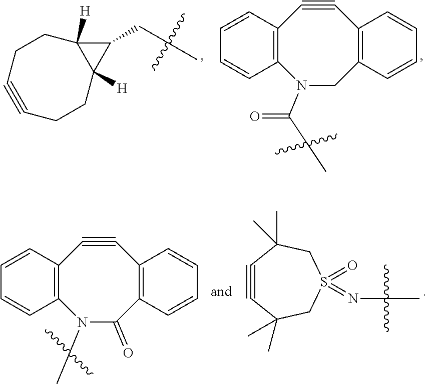 [SMILES: *C[C@H]1[C@H]2CCC#CCC[C@@H]12 ; *C(=O)N1Cc2ccccc2C#Cc2ccccc21 ; *N1C(=O)c2ccccc2C#Cc2ccccc21 ; *N=S1(=O)CC(C)(C)C#CC(C)(C)C1]

In some embodiments, the X₂Y₂ is selected from the group consisting of

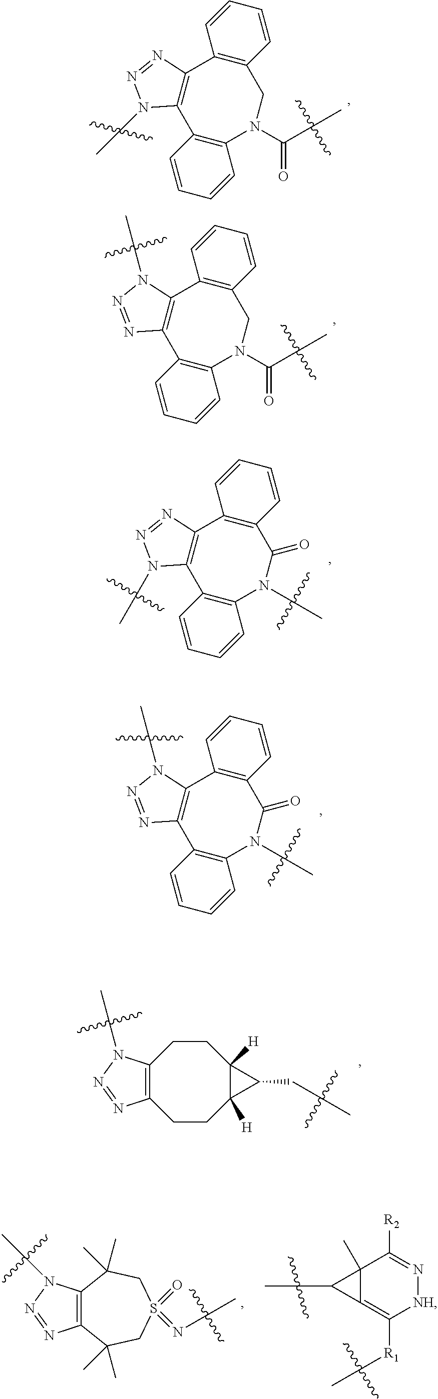

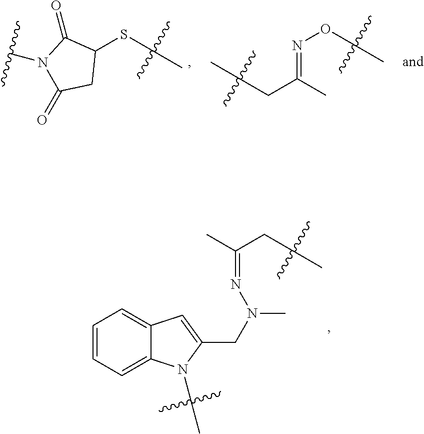

wherein each of R₁ is selected from the group consisting of C₁-C₂₂ alkylene group, C₅-C₂₂ (hetero)arylene group, C₆-C₂₂ alkyl(hetero)arylene group and C₆-C₂₂ (hetero)arylalkylene group, wherein the alkylene group, the (hetero)arylene group, the alkyl(hetero)arylene group or the (hetero)arylalkylene group is optionally substituted. R² is selected from the group consisting of hydrogen, halogen, C₁-C₂₂ alkyl group, C₅-C₂₂ (hetero)aryl group, C₆-C₂₂ alkyl(hetero)aryl group and C₆-C₂₂ (hetero)arylalkyl group, wherein the alkyl group, the (hetero)aryl group, the alkyl(hetero)aryl group or the (hetero)arylalkyl group is optionally substituted. For example, the alkylene group, the (hetero)arylene group, the alkyl(hetero)arylene group or the (hetero)arylalkylene group is optionally substituted by one or more halogen, —OH, —NH₂, —COOH or —CN. For example, the alkyl group, the (hetero)aryl group, the alkyl(hetero)aryl group or the (hetero)arylalkyl group is optionally substituted by one or more halogen, —OH, —NH₂, —COOH or —CN.

In some embodiments, the X₂ and the Y₂ comprise functional moieties selected from the group consisting of: a) X₂ comprises

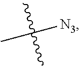 [SMILES: *N=[N+]=[N-]]

and Y₂ comprises

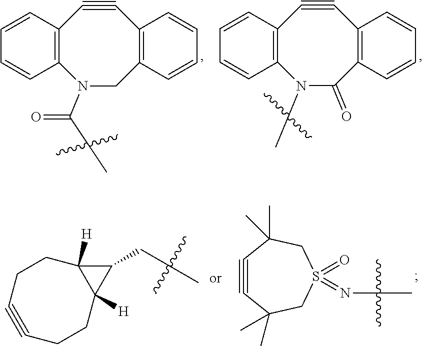

b) X₂ comprises

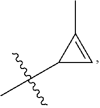 [SMILES: *C1C=C1C]

and Y₂ comprises

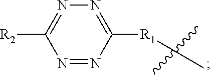

c) X₂ comprises

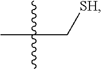 [SMILES: *CS]

and Y₂ comprises

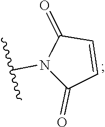 [SMILES: *N1C(=O)C=CC1=O]

and d) X₂ comprises

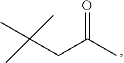

and Y₂ comprises

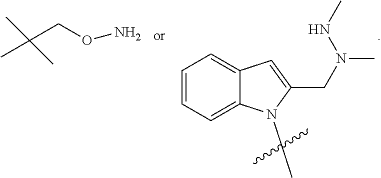

In some embodiments, the X₁ substantially does not react with the X₂.

In some embodiments, the X₁ and the X₂ comprise the same functional moiety, and the Y₁ and the Y₂ comprise the same functional moiety.

In some embodiments, the X₁ and the X₂ comprise different functional moieties, and the Y₁ and the Y₂ comprise the same functional moiety. In some embodiments, the X₁ comprises

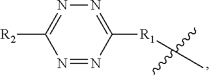

X₂ comprises

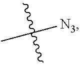 [SMILES: *N=[N+]=[N-]]

Y₁ comprises

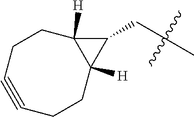 [SMILES: *C[C@H]1[C@H]2CCC#CCC[C@@H]12]

and Y₂ comprises

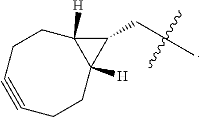 [SMILES: *C[C@H]1[C@H]2CCC#CCC[C@@H]12]

In some embodiments, the X₁ and the X₂ comprise different functional moieties, and the Y₁ and the Y₂ comprise different functional moieties.

In some embodiments, the reaction between the X₁ and Y₁ substantially does not affect on the reaction between the X₂ and the Y₂.

In some embodiments, the X₁, the Y₁, the X₂ and the Y₂ comprise functional moieties selected from the group consisting of:
a) X₁ comprises

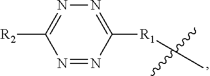

Y₁ comprises

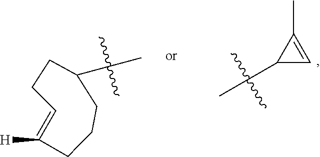

X₂ comprises

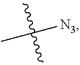 [SMILES: *N=[N+]=[N-]]

Y₂ comprises

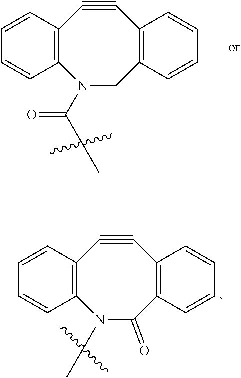

b) X₁ comprises

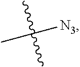 [SMILES: *N=[N+]=[N-]]

Y₁ comprises

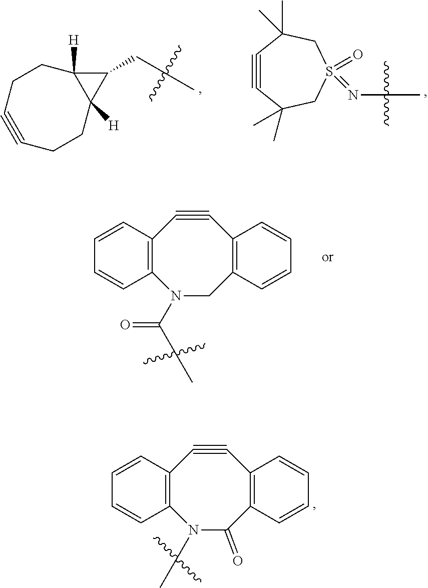

X₂ comprises

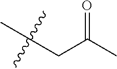 [SMILES: *CC(C)=O]

and Y₂ comprises

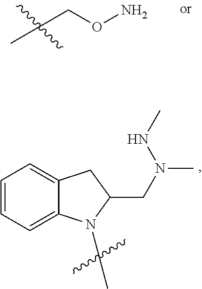

c) X₁ comprises

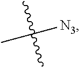 [SMILES: *N=[N+]=[N-]]

Y₁ comprises

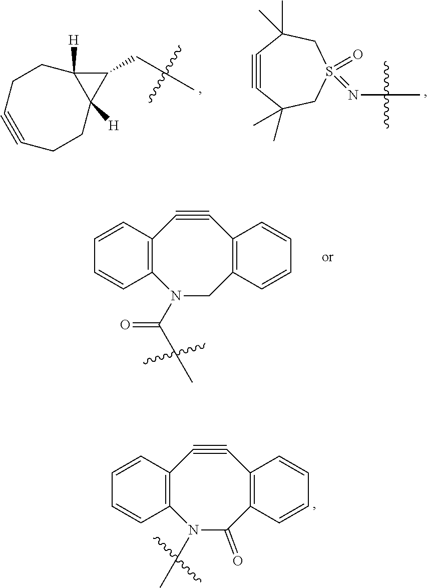

X₂ comprises

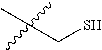 [SMILES: *CS]

and Y₂ comprises

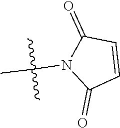 [SMILES: *N1C(=O)C=CC1=O]

and d) X₁ comprises

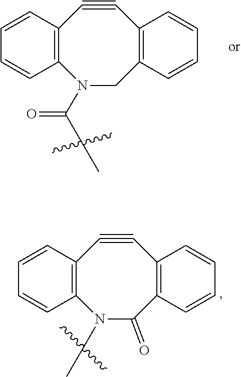

Y₁ comprises

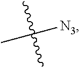 [SMILES: *N=[N+]=[N-]]

X₂ comprises

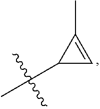 [SMILES: *C1C=C1C]

and Y₂ comprises

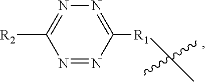

wherein R₁ is selected from the group consisting of C₁-C₂₂ alkylene group, C₅-C₂₂ (hetero)arylene group, C₆-C₂₂ alkyl(hetero)arylene group and C₆-C₂₂ (hetero)arylalkylene group, wherein the alkylene group, the (hetero)arylene group, the alkyl(hetero)arylene group or the (hetero)arylalkylene group is optionally substituted. R₂ is selected from the group consisting of hydrogen, halogen, C₁-C₂₂ alkyl group, C₅-C₂₂ (hetero)aryl group, C₆-C₂₂ alkyl(hetero)aryl group and C₆-C₂₂ (hetero)arylalkyl group, wherein the alkyl group, the (hetero)aryl group, the alkyl(hetero)aryl group or the (hetero)arylalkyl group is optionally substituted. For example, the alkylene group, the (hetero)arylene group, the alkyl(hetero)arylene group or the (hetero)arylalkylene group is optionally substituted by one or more halogen, —OH, —NH₂, —COOH or —CN. For example, the alkyl group, the (hetero)aryl group, the alkyl(hetero)aryl group or the (hetero)arylalkyl group is optionally substituted by one or more halogen, —OH, —NH₂, —COOH or —CN.

In some embodiments, the X₁ comprises

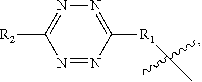

the Y₁ comprises

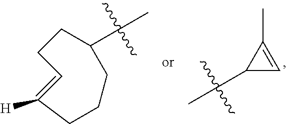

the X₂ comprises

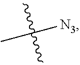 [SMILES: *N=[N+]=[N-]]

and the Y₂ comprises

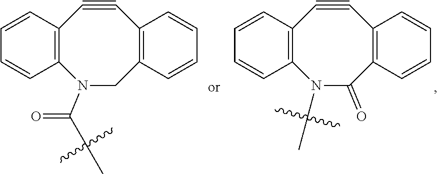

wherein each of R₁ and R₂ is defined as above.

In some embodiments, the GalX comprises a FL″, and FL″ is a spacer.

In some embodiments, the FL″ is selected from the group consisting of C₃-C₂₀₀ peptide, C₂-C₂₀₀ PEG, C₁-C₂₀₀ alkylene group, C₃-C₂₀₀ cycloalkylene group, C₂-C₂₀₀ alkenylene group, C₅-C₂₀₀ cycloalkenylene group, C₂-C₂₀₀ alkynylene group, C₅-C₂₀₀ cycloalkynylene group, C₂-C₂₄ (hetero)arylene group, C₃-C₂₀₀ (hetero)arylalkylene group, C₃-C₂₀₀ alkynyl(hetero)arylene group, their derivatives and any combination thereof, wherein said the peptide, the PEG, the alkylene group, the cycloalkylene group, the alkenylene group, the cycloalkenylene group, the alkynylene group, the cycloalkynylene group, the (hetero)arylene group, the (hetero)arylalkylene group, or the alkynyl(hetero)arylene group is optionally substituted by one or more Rs¹ and/or is optionally interrupted by one or more Rs², wherein Rs¹ is selected from the group consisting of halogen, —OH, —NH₂ and —COOH, Rs² is independently selected from the group consisting of —O—, —S—,

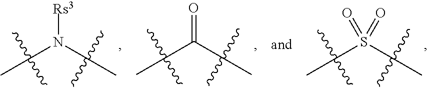

wherein Rs³ is selected from the group consisting of hydrogen, C₁-C₂₄ alkyl group, C₂-C₂₄ alkenyl group, C₂-C₂₄ alkynyl group and C₃-C₂₄ cycloalkyl group.

In some embodiments, the GalX comprises a L2, and L2 is a linker. In some embodiments, the L2 is a cleavable linker. In some embodiments, the L2 is an acid-labile linker, a redox-active linker, a photo-active linker and/or a proteolytically cleavable linker. For example, the L2 is a vc-PAB-linker, a disulfo linker, or a GGFG-linker.

For example, the GalX in Formula (1) is GalX₂. For example, the GalX in Formula (1) is GalX₂Y₂-(FL″)_{m″}-(L₂)_{n″}-P₂, wherein m″ is 0 or 1, and n″ is 0 or 1. For example, the GalX in Formula (1) is GalX₂Y₂-L₂-P₂. For example, the GalX in Formula (1) is GalX₂Y₂-FL″-L₂-P₂. For example, the GalX in Formula (1) is GalX₂Y₂-P₂.

In some embodiments, the GalX doesn't comprises a functional moiety capable of participating in a bioorthorgonal ligation reaction, and the GalX is represented by GalX₀. For example, the GalX in Formula (1) is GalX₀.

In some embodiments, the GalX doesn't comprises a functional moiety capable in participating in a bioorthorgonal ligation reaction. The functional moiety is selected from the group consisting of azido group, terminal alkynyl group, cyclic alkynyl group, tetrazinyl group, 1,2,4-trazinyl group, terminal alkenyl group, cyclic alkenyl group, ketone group, aldehyde group, hydroxyl amino group, sulfydryl group and N-maleimide group, and said GalX is represented by GalX₀.

In some embodiments, the

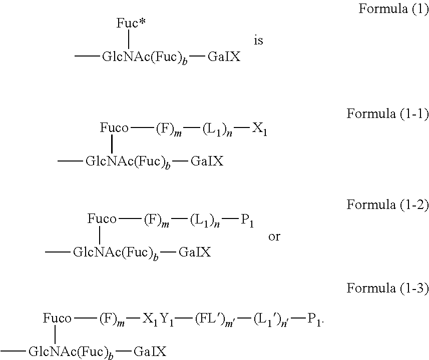

In some embodiments, the

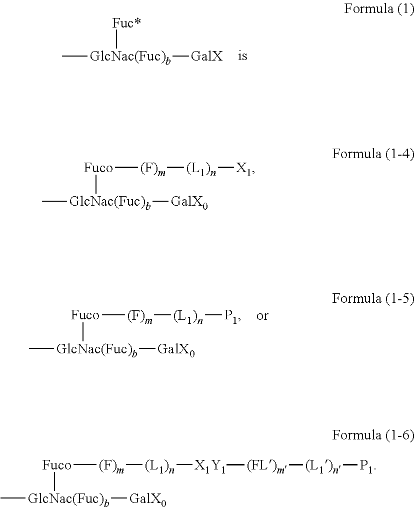

In some embodiments, the

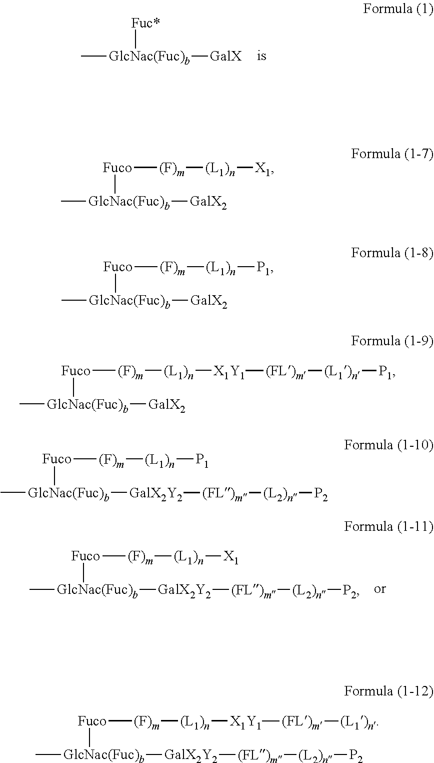

For example, the GalX in Formula (1) is selected from the group consisting of:

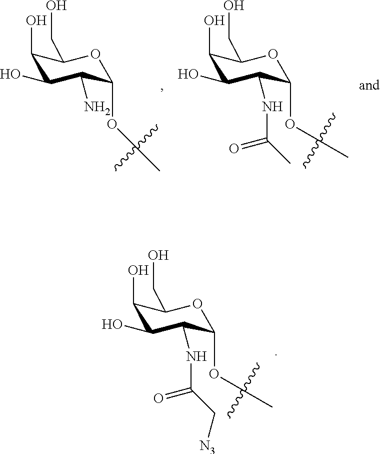

For example, the GalX₂ is

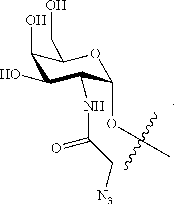

For example, the GalX₀ is selected from the group consisting of

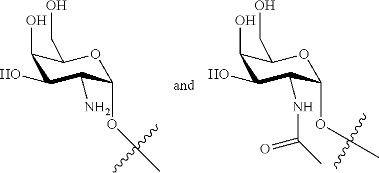

In some embodiments, the oligosaccharide comprises 1 to 20 Formula (1):

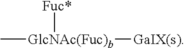

For example, the oligosaccharide comprises 2 Formula (1):

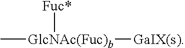

For example, the oligosaccharide comprises 4 Formula (1):

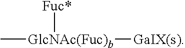

In some embodiments, the protein conjugate of the present closure comprises a structure of Formula (8): [symbol] wherein [symbol] is the GlcNAc, [symbol] is the Fuc linked to the GlcNAc through an α-1,6 linkage, GalX is linked to the GlcNAc through a GalXβ1,4GlcNAc linkage, Fuc* is linked to the GlcNAc through an Fuc*α1,3GlcNAc linkage, b is 0 or 1, and [symbol] is an antibody or a Fc-fusion protein.

In some embodiments, the protein conjugate of the present closure comprises a structure of [symbol] wherein [symbol] is a GlcNAc, [symbol] is the Fuc linked to the the GlcNAc through a α-1,6GlcNAc linkage, [symbol] is a mannose, GalX is linked to the GlcNAc through a GalXβ1,4GlcNAc linkage, Fuc* is linked to the GlcNAc through an Fuc*α1,3GlcNAc linkage, c is 0 or 1, and [symbol] is an antibody or Fc-fusion protein.

In some embodiments, the protein conjugate of the present disclosure has one or more of the following properties: (1) having a first MOI-to-antibody ratio (M₁AR), and the M₁AR is 2 or 4, (2) having a first MOI-to-antibody ratio (M₁AR) and a second MOI-to-antibody ratio (M₂AR), and the M₁AR is 2 and the M₂AR is 2, or, the M₁AR is 4 and the M₂AR is 4, (3) capable of binding to an antigen, (4) capable of binding to an antigen, with a similar binding affinity as its corresponding antibody, (5) stable in plasma (e.g. human plasma) for at least 1 day, as measured in mass spectrometry analysis, (6) the linkage between the Fuco of Fuc* and the GlcNAc in Formula (1) is stable in plasma (e.g. human plasma) for at least 1 day, as measured in mass spectrometry analysis, wherein b is 0 or 1, (7) having a high reactive activity, and (8) capable of inhibiting tumor growth and/or tumor cell proliferation.

In another aspect, the present disclosure provides a composition comprising the protein conjugate of the present disclosure.

In some embodiments, the composition has a first average MOI-to-antibody ratio (average M₁AR), wherein the average M₁AR is about 2. In some embodiments, the composition has a first average MOI-to-antibody ratio (average M₁AR) and a second average MOI-to-antibody ratio (average M₂AR), wherein the average M₁AR is about 2, and/or the average M₂AR is about 2.

In some embodiments, the composition has a first average MOI-to-antibody ratio (average M₁AR), wherein the average M₁AR is about 4. In some embodiments, the composition has a first average MOI-to-antibody ratio (average M₁AR) and a second average MOI-to-antibody ratio (average M₂AR), wherein the average M₁AR is about 4, and/or the average M₂AR is about 4.

In another aspect, the present disclosure provides a method for preparing the protein conjugate and/or the composition of the present disclosure of the present disclosure.

In another aspect, the present disclosure provides a method for preparing a protein conjugate, the method comprises step (a): contacting a fucose derivative donor Q-Fuc*′ with a protein comprising an oligosaccharide in the presence of a catalyst, wherein the oligosaccharide comprises Formula (10): -GlcNAc(Fuc)_{b}-GalX′ (also named as -(Fuc)_{b}(GalX′)GlcNAc)), to obtain a protein conjugate comprising Formula (11):

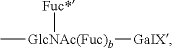

wherein the GlcNAc is directly or indirectly linked to an amino acid of the protein, the GalX′ is a substituted galactose, the Fuc is a fucose, b is 0 or 1, the Q-Fuc*′ is a molecule comprises Fuc*′, the Fuc*′ comprises a Fuco and a molecule of interest (MOI₁′). In some embodiments, the protein comprises an antigen binding moiety and/or a Fc fragment.

In some embodiments, the catalyst is a fucosyltransferase. In some embodiments, the catalyst is an α-1,3-fucosyltransferase. In some embodiments, the fucosyltransferase is derived from bacteria, nematodes, trematodes or mammal. In some embodiments, the fucosyltransferase is derived from bacteria.

In some embodiments, the fucosyltransferase is derived from Bacteroides fragilis

In some embodiments, the fucosyltransferase is derived from Helicobacter pylori. In some embodiments, the fucosyltransferase comprises an amino acid sequence as set forth in GenBank Accession No. AAB81031.1, GenBank Accession No. AAD07447.1, GenBank Accession No. AAD07710.1, GenBank Accession No. AAF35291.2, GenBank Accession No. AAB93985.1, and/or their functional variants or fragments thereof. For example, the fucosyltransferase comprises an amino acid sequence as set forth in GenBank Accession No. AAD07710.1, or a functional variant or fragment thereof. For example, the fucosyltransferase comprises an amino acid sequence as set forth in SEQ ID NO: 1 or SEQ ID NO: 2, or their functional variant or fragment thereof. For example, the fucosyltransferase may comprise an amino acid sequence with an identity of more than 80% (e.g., more than 88%, more than 88%, more than 90%, more than 95%, more than 96%, more than 97%, more than 98%, more than 99%, or more) of an amino acid sequence as described above.

In some embodiments, the oligosaccharide is a N-linked oligosaccharide. In some embodiments, the oligosaccharide is a O-linked oligosaccharide.

In some embodiments, the oligosaccharide is linked to an Asparagine (Asn) residue of the protein.

In some embodiments, the GlcNAc of Formula (10) is directly linked to an Asn residue of the protein, and b is 0. In some embodiments, the GlcNAc of Formula (10) is directly linked to an Asn residue of the protein, and b is 1. In some embodiments, the GlcNAc of Formula (11) is directly linked to an Asn residue of the protein, and b is 0. In some embodiments, the GlcNAc of Formula (11) is directly linked to an Asn residue of the protein, and b is 1. In some embodiments, the Asn residue is Asn297 on Fc domain of an antibody.

In some embodiments, the GlcNAc of Formula (10) is linked to a saccharide of the oligosaccharide, preferably b is 0. In some embodiments, the GlcNAc of Formula (10) is linked to a mannose of the oligosaccharide, preferably b is 0.

In some embodiments, the GlcNAc of Formula (11) is linked to a saccharide of the oligosaccharide, preferably b is 0. In some embodiments, the GlcNAc of Formula (11) is linked to a mannose of the oligosaccharide, preferably b is 0.

In some embodiments, the protein comprises a Fc fragment. In some embodiments, the protein is a Fc fusion protein. In some embodiments, the protein comprises a Fc fragment and the oligosaccharide is linked to the Fc fragment. In some embodiments, the oligosaccharide is linked to the CH₂ domain of the Fc fragment. In some embodiments, the oligosaccharide is linked to Asn297 of the Fc fragment, numbered according to the Kabat numbering system.

In some embodiments, the protein is an antibody.

In some embodiments, the antibody is a monoclonal antibody.

In some embodiments, the antibody is an IgG antibody. In some embodiments, the antibody is an IgA antibody. In some embodiments, the antibody is an IgE antibody. In some embodiments, the antibody is an IgM antibody.

In some embodiments, the antibody is a humanized antibody. In some embodiments, the antibody is a fully human antibody. In some embodiments, the antibody is a chimeric antibody.

In some embodiments, the optionally Fuc is linked to the GlcNAc through an α1,6 linkage.

In some embodiments, the GalX′ is linked to the GlcNAc through a GalX′β1,4GlcNAc linkage.

In some embodiments, the GalX′ is a substituted galactose, and is substituted on one or more positions selected from the C2 position, the C3 position, the C4 position and the C6 position of the galactose. For example, the hydroxyl group on one or more positions selected from the C2 position, the C3 position, the C4 position and the C6 position of the galactose, is substituted.

In some embodiments, the GalX′ is a substituted galactose, and is substituted on the C2 position and/or the C6 position. In some embodiments, the GalX′ is a substituted galactose, and is substituted on the C2 position. For example, the hydroxyl group on the C2 position of the galactose is substituted.

In some embodiments, the GalX′ is a substituted galactose, and is substituted on the C6 position. For example, the hydroxyl group on the C6 position of the galactose is substituted.

In some embodiments, the GalX′ is a not substituted with a saccharide. In some embodiments, the GalX′ is a monosaccharide.

In some embodiments, the GalX′ is GalX, and GalX is a galactose substituted by a substitution Rg and the Rg is according to Formula (5):

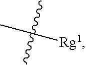

wherein Rg¹ is selected from the group consisting of hydrogen, halogen, —NH₂, —SH, —N₃, —COOH, —CN, C₁-C₂₄ alkyl group, C₃-C₂₄ cycloalkyl group, C₂-C₂₄ alkenyl group, C₅-C₂₄ cycloalkenyl group, C₂-C₂₄ alkynyl group, C₇-C₂₄ cycloalkynyl group, C₂-C₂₄ (hetero)aryl group, C₃-C₂₄ alkyl(hetero)aryl group, C₃-C₂₄ (hetero)arylalkyl group and any combination thereof, wherein the alkyl group, the cycloalkyl group, the alkenyl group, the cycloalkenyl group, the alkynyl group, the cycloalkynyl group, the (hetero)aryl group, the alkyl(hetero)aryl group, or the (hetero)arylalkyl group is optionally substituted by one or more Rs⁴ and/or is optionally interrupted by one or more Rs², wherein Rs⁴ is selected from the group of halogen, —OH, —NH₂, —SH, —N₃, —COOH and —CN, Rs² is selected from the group consisting of —O—,

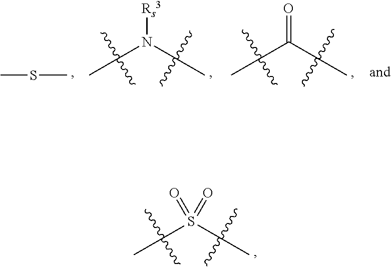

wherein Rs³ is selected from the group consisting of hydrogen, C₁-C₂₄ alkyl group, C₂-C₂₄ alkenyl group, C₂-C₂₄ alkynyl group and C₃-C₂₄ cycloalkyl group.

In some embodiments, the GalX′ is a galactose substituted by a substitution Rg and the Rg is according to Formula (6):

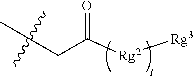

### or Formula (7):

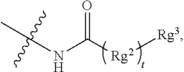

wherein t is 0 or 1, Rg² is selected from the group consisting of C₁-C₂₄ alkylene group, C₃-C₂₄ cycloalkylene group, C₂-C₂₄ alkenylene group, C₅-C₂₄ cycloalkenylene group, C₂-C₂₄ alkynylene group, C₇-C₂₄ cycloalkynylene group, C₂-C₂₄ (hetero)arylene group, C₃-C₂₄ alkyl(hetero)arylene group and C₃-C₂₄ (hetero)arylalkylene group, wherein the alkylene group, the cycloalkylene group, the alkenylene group, the cycloalkenylene group, the alkynylene group, the cycloalkynylene group, the (hetero)arylene group, the alkyl(hetero)arylene group or the (hetero)arylalkylene group is optionally substituted by one or more Rs⁴ and/or is optionally interrupted by one or more Rs², Rg³ is selected from the group consisting of hydrogen, halogen, —OH, —NH₂, —SH, —N₃, —COOH, —CN, C₁-C₂₄ alkyl group, C₃-C₂₄ cycloalkyl group, C₂-C₂₄ alkyne group, C₅-C₂₄ cycloalkyne group, C₂-C₂₄ alkynyl group, C₅-C₂₄ cycloalkynyl group, C₂-C₂₄ (hetero)aryl group and any combination thereof, wherein the C₁-C₂₄ alkyl group, the C₃-C₂₄ cycloalkyl group, the C₂-C₂₄ alkyne group, the C₅-C₂₄ cycloalkyne group, the C₂-C₂₄ alkynyl group, the C₅-C₂₄ cycloalkynyl group, or the C₂-C₂₄ (hetero)aryl group is optionally substituted by one or more Rs⁴, Rs⁴ is selected from the group of halogen —OH, —NH₂, —SH, —N₃, —COOH and —CN, Rs² is selected from the group consisting of —O—, —S—,

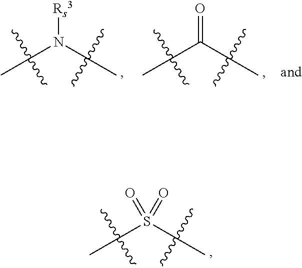

wherein Rs³ is selected from the group consisting of hydrogen, C₁-C₂₄ alkyl group, C₂-C₂₄ alkenyl group, C₂-C₂₄ alkynyl group and C₃-C₂₄ cycloalkyl group.

In some embodiments, the Q-Fuc*′ comprises a ribonucleotide diphosphate. In some embodiments, the Q-Fuc*′ comprises a uridine diphosphate (UDP), a guanosine diphosphate (GDP) or a cytidine diphosphate (CDP). In some embodiments, Q is UDP. In some embodiments, Q is GDP. In some embodiments, Q is CDP. In some embodiments, the Q-Fuc*′ comprises a uridine diphosphate (UDP), Q is UDP and Q-Fuc*′ is UDP-Fuc*′. In some embodiments, the Q-Fuc*′ comprises a cytidine diphosphate (CDP), Q is CDP and Q-Fuc*′ is CDP-Fuc*′. In some embodiments, the Q-Fuc*′ comprises a guanosine diphosphate (GDP), Q is GDP and is GDP-Fuc*′.

In some embodiments, the Q-Fuc*′ comprises a guanosine diphosphate (GDP) and is GDP-Fuc*′

In some embodiments, the MOI₁′ of Fuc*′ comprises an active moiety.

In some embodiments, the MOI₁′ comprises a chemically active moiety, an enzymatically active moiety, a biologically active moiety, and/or a pharmaceutically active moiety.

In some embodiments, the MOI₁′ comprises a P₁, and P₁ is a biologically and/or pharmaceutically active moiety.

In some embodiments, the method comprises step (a1): contacting the Q-Fuc*′ with the protein comprising Formula (10) -GlcNAc(Fuc)_{b}-GalX′ to obtain a protein conjugate comprising Formula (12)

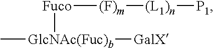

wherein Q-Fuc*′ is Q-Fuco-(F)ₘ-(L₁)ₙ-P₁, Fuc*′ is Fuco-(F)ₘ-(L₁)ₙ-P₁, Fuco is the fucose or fucose derivative of Fuc*′, F is a connector, m is 0 or 1, L₁ is a linker, and n is 0 or 1.

In some embodiments, Fuc*′ is Fuco-F-L₁-P₁. In some embodiments, Fuc*′ is Fuco-F-P₁. In some embodiments, Fuc*′ is Fuco-P₁.

In some embodiments, the comprises a chemically active moiety and/or an enzymatically active moiety.

In some embodiments, the MOI′ comprises a X₁, and X₁ is a functional group capable of participating in a ligation reaction.

In some embodiments, the method comprises step (a2): contacting the Q-Fuc*′ with the protein comprising Formula (10) -GlcNAc(Fuc)_{b}-GalX′ to obtain a protein conjugate comprising Formula (13)

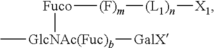

wherein Q-Fuc*′ is Q-Fuco-(F)ₘ-(L₁)ₙ-X₁, Fuc*′ is Fuco-(F)ₘ-(L₁)ₙ-X₁, Fuco is the fucose or fucose derivative of Fuc*′, F is a connector, m is 0 or 1, L₁ is a linker, and n is 0 or 1.

In some embodiments, Fuc*′ is Fuco-F-X₁. In some embodiments, Fuc*′ is Fuco-X₁.

In some embodiments, the method further comprises step (b): contacting the protein conjugate comprising Formula (13)

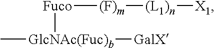

with a Y₁-(FL′)_{m′}-(L₁′)_{m′}-P₁, to obtain a protein conjugate comprising Formula (14)

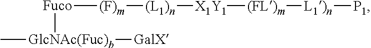

wherein, Y₁ is a functional group, X₁Y₁ is a remaining group after a ligation reaction between X₁ and Y₁, FL′ is a spacer, m′ is 0 or 1, L′ is a linker, n′ is 0 or 1, and P₁ is a biologically and/or pharmaceutically active moiety.

In some embodiments, m is 1, n is 0, m′ is 1 and n′ is 1. In some embodiments, m is 1, n is 0, m′ is 1 and n′ is 0.

In some embodiments, GalX′ comprises a X₂, and X₂ is a functional group capable of participating in a ligation reaction, and the GalX′ is represented by GalX₂.

In some embodiments, the method further comprises step (ci): contacting a protein conjugate comprising Formula (1-8)

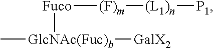

with Y₂-(FL″)_{m″}-(L₂)_{n″}-P₂, to obtain a protein conjugate comprising Formula (1-10):

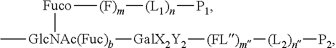

wherein, Y₂ is a functional group, X₂Y₂ is a remaining group after a ligation reaction between X₂ and Y₂, FL″ is a spacer, m″ is 0 or 1, L₂ is a linker, n″ is 0 or 1, P₂ is a biologically and/or pharmaceutically active moiety, and GalX₂ represents a GalX′ comprising a X₂, and X₂ is a functional group comprising a functional moiety capable of participating in a bioorthogonal ligation reaction.

In some embodiments, the method further comprises step (c2): contacting a protein conjugate comprising Formula (1-7)

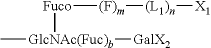

with Y₂(FL″)_{m″}-(L₂)_{n″}-P₂ to obtain a protein conjugate comprising Formula (1-11)

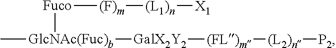

wherein Y₂ is a functional group, X₂Y₂ is a remaining group after a ligation reaction between X₂ and Y₂, FL″ is a spacer, m″ is 0 or 1, L₂ is a linker, n″ is 0 or 1, P₂ is a biologically and/or pharmaceutically active moiety, and GalX₂ represents a GalX′ comprising a X₂, and X₂ is a functional group comprising a functional moiety capable of participating in a bioorthogonal ligation reaction.

In some embodiments, the method further comprises step (d): contacting a protein conjugate comprising Formula (1-9):

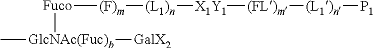

with Y₂-(FL″)_{m″}-(L₂)_{n″}-P₂ to obtain a protein conjugate comprising Formula (1-12)

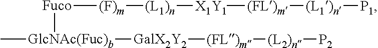

wherein Y₂ is a functional group, X₂Y₂ is a remaining group after a ligation reaction between X₂ and Y₂, FL″ is a spacer, m″ is 0 or 1, L₂ is a linker, n″ is 0 or 1, P₂ is a biologically and/or pharmaceutically active moiety, and GalX₂ represents a GalX′ comprising a X₂, and X₂ is a functional group comprising a functional moiety capable of participating in a bioorthogonal ligation reaction.

In some embodiments, the method further comprises a step (e): contacting the protein conjugate comprising Formula (1-11)

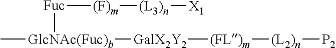

with Y₁-(FL′)_{m′}-(L₁′)_{n′}-P₁ to obtain a protein conjugate comprising Formula (1-12)

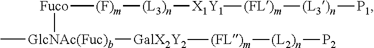

wherein Y₁ is a functional group, X₁Y₁ is a remaining group after a ligation reaction between X₁ and Y₁, FL′ is a spacer, m′ is 0 or 1, L′ is a linker, n′ is 0 or 1, and P₁ is a biologically and/or pharmaceutically active moiety. In some embodiments, m is 1 and n is 0.

In some embodiments, the X₁ comprises a functional moiety capable of participating in a bioorthogonal ligation reaction. For example, the X₁ may comprise one or more functional moieties.

In some embodiments, the X₁ comprises a functional moiety selected from the group consisting of azido group, terminal alkynyl group, cyclic alkynyl group, tetrazinyl group, 1,2,4-trazinyl group, terminal alkenyl group, cyclic alkenyl group, ketone group, aldehyde group, hydroxyl amino group, sulfydryl group, N-maleimide group and their functional derivatives.

In some embodiments, the X₁ comprises a functional moiety selected from the group consisting of

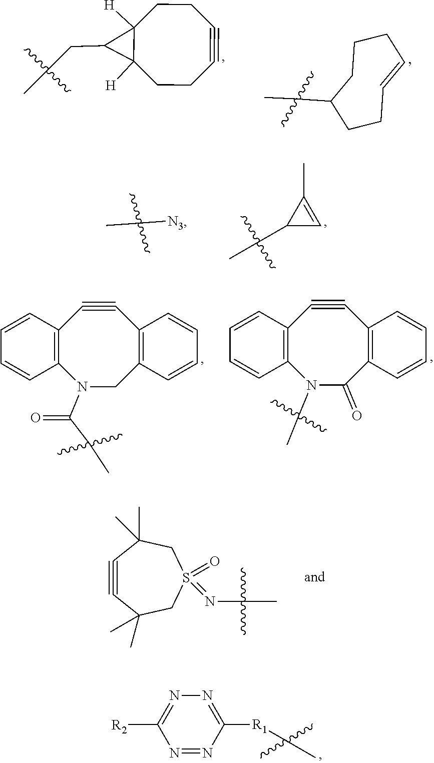

wherein R₁ is selected from the group consisting of C₁-C₂₂ alkylene group, C₅-C₂₂ (hetero)arylene group, C₆-C₂₂ alkyl(hetero)arylene group and C₆-C₂₂ (hetero)arylalkylene group, wherein the alkylene group, the (hetero)arylene group, the alkyl(hetero)arylene group or the (hetero)arylalkylene group is optionally substituted, R₂ is selected from the group consisting of hydrogen, halogen, C₁-C₂₂ alkyl group, C₅-C₂₂ (hetero)aryl group, C₆-C₂₂ alkyl(hetero)aryl group and C₆-C₂₂ (hetero)arylalkyl group, wherein the alkyl group, the (hetero)aryl group, the alkyl(hetero)aryl group or the (hetero)arylalkyl group is optionally substituted. For example, the alkylene group, the (hetero)arylene group, the alkyl(hetero)arylene group or the (hetero)arylalkylene group is optionally substituted by one or more halogen, —OH, —NH₂, —COOH or —CN. For example, the alkyl group, the (hetero)aryl group, the alkyl(hetero)aryl group or the (hetero)arylalkyl group is optionally substituted by one or more halogen, —OH, —NH₂, —COOH or —CN.

In some embodiments, the X₁ comprises a functional moiety selected from the group consisting of

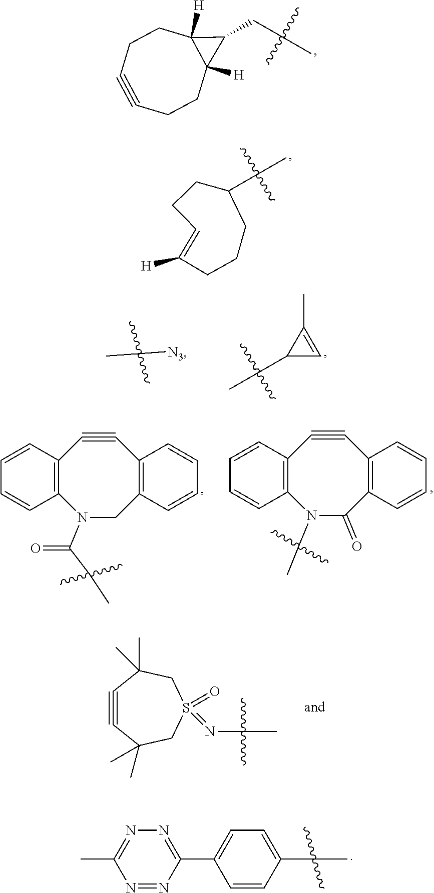

In some embodiments, the Y₁ comprises a functional moiety capable of reacting with the X₁ through a bioorthogonal ligation reaction.

In some embodiments, the Y₁ comprises a functional moiety selected from the group consisting of azido group, terminal alkynyl group, cyclic alkynyl group, tetrazinyl group, 1,2,4-trazinyl group, terminal alkenyl group, cyclic alkenyl group, ketone group, aldehyde group, hydroxyl amino group, sulfydryl group, N-maleimide group and their functional derivatives.

In some embodiments, the Y₁ comprises a functional moiety selected from the group consisting of

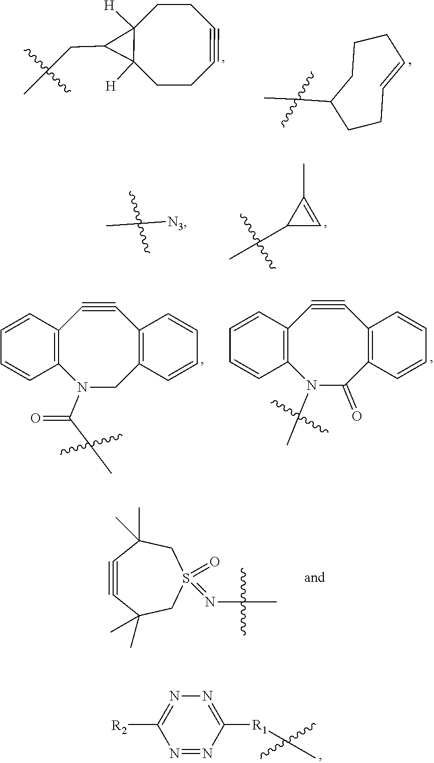

wherein R₁ is selected from the group consisting of C₁-C₂₂ alkylene group, C₅-C₂₂ (hetero)arylene group, C₆-C₂₂ alkyl(hetero)arylene group and C₆-C₂₂ (hetero)arylalkylene group, wherein the alkylene group, the (hetero)arylene group, the alkyl(hetero)arylene group or the (hetero)arylalkylene group is optionally substituted, R₂ is selected from the group consisting of hydrogen, halogen, C₁-C₂₂ alkyl group, C₅-C₂₂ (hetero)aryl group, C₆-C₂₂ alkyl(hetero)aryl group and C₆-C₂₂ (hetero)arylalkyl group, wherein the alkyl group, the (hetero)aryl group, the alkyl(hetero)aryl group or the (hetero)arylalkyl group is optionally substituted. For example, the alkylene group, the (hetero)arylene group, the alkyl(hetero)arylene group or the (hetero)arylalkylene group is optionally substituted by one or more halogen, —OH, —NH₂, —COOH or —CN. For example, the alkyl group, the (hetero)aryl group, the alkyl(hetero)aryl group or the (hetero)arylalkyl group is optionally substituted by one or more halogen, —OH, —NH₂, —COOH or —CN.

In some embodiments, the Y₁ comprises a functional moiety selected from the group consisting of

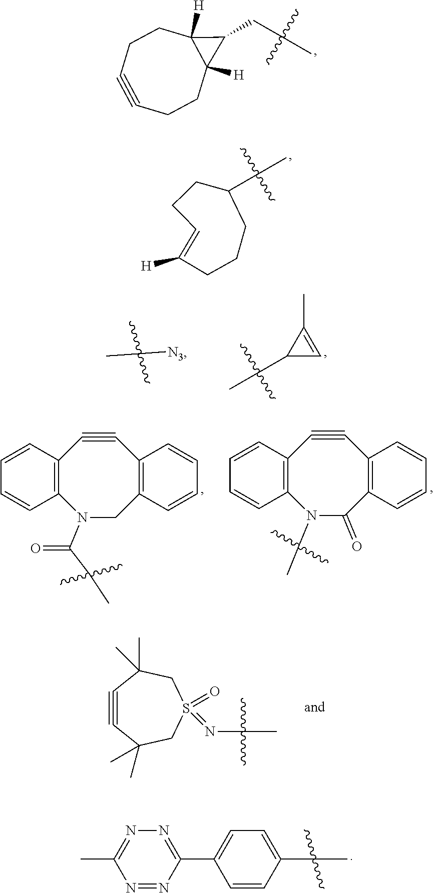

In some embodiments, the remaining group X₁Y₁ is selected from the group consisting of

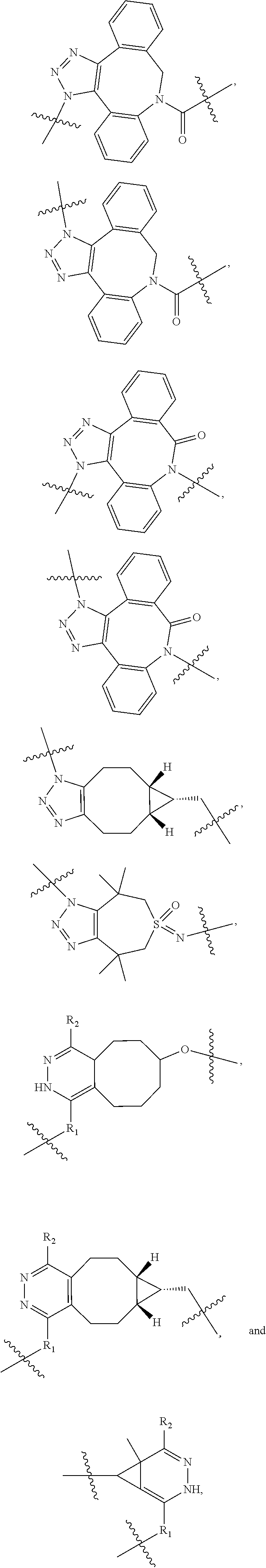

wherein R₁ is selected from the group consisting of C₁-C₂₂ alkylene group, C₅-C₂₂ (hetero)arylene group, C₆-C₂₂ alkyl(hetero)arylene group and C₆-C₂₂ (hetero)arylalkylene group, wherein the alkylene group, the (hetero)arylene group, the alkyl(hetero)arylene group or the (hetero)arylalkylene group is optionally substituted, R₂ is selected from the group consisting of hydrogen, halogen, C₁-C₂₂ alkyl group, C₅-C₂₂ (hetero)aryl group, C₆-C₂₂ alkyl(hetero)aryl group and C₆-C₂₂ (hetero)arylalkyl group, wherein the alkyl group, the (hetero)aryl group, the alkyl(hetero)aryl group or the (hetero)arylalkyl group is optionally substituted. For example, the alkylene group, the (hetero)arylene group, the alkyl(hetero)arylene group or the (hetero)arylalkylene group is optionally substituted by one or more halogen, —OH, —NH₂, —COOH or —CN. For example, the alkyl group, the (hetero)aryl group, the alkyl(hetero)aryl group or the (hetero)arylalkyl group is optionally substituted by one or more halogen, —OH, —NH₂, —COOH or —CN.

In some embodiments, the X₁ and the Y₁ comprise the functional moieties selected from the group consisting of: a) X₁ comprises

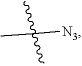 [SMILES: *N=[N+]=[N-]]

and Y₁ comprises

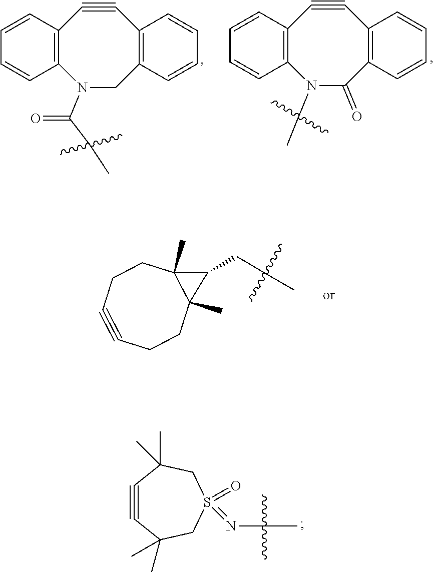

b) X₁ comprises

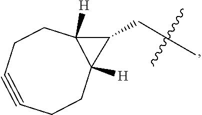 [SMILES: *C[C@H]1[C@H]2CCC#CCC[C@@H]12]

and Y₁ comprises

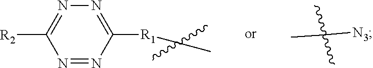

c) X₁ comprises

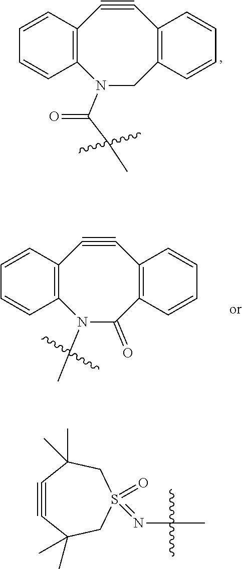 [SMILES: *C(=O)N1Cc2ccccc2C#Cc2ccccc21 ; *N1C(=O)c2ccccc2C#Cc2ccccc21 ; *N=S1(=O)CC(C)(C)C#CC(C)(C)C1]

Y₁ comprises

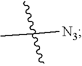 [SMILES: *N=[N+]=[N-]]

d) X₁ comprises

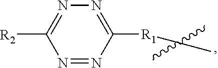

and Y₁ comprises

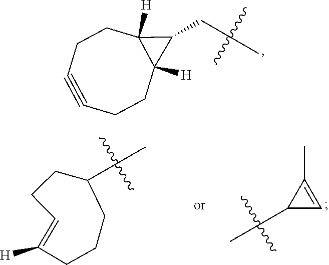

and e) X₁ comprises

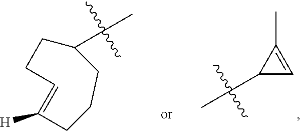

and Y₁ comprises

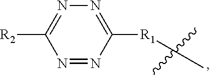

wherein R₁ is selected from the group consisting of C₁-C₂₂ alkylene group, C₅-C₂₂ (hetero)arylene group, C₆-C₂₂ alkyl(hetero)arylene group and C₆-C₂₂ (hetero)arylalkylene group, wherein the alkylene group, the (hetero)arylene group, the alkyl(hetero)arylene group or the (hetero)arylalkylene group is optionally substituted, R₂ is selected from the group consisting of hydrogen, halogen, C₁-C₂₂ alkyl group, C₅-C₂₂ (hetero)aryl group, C₆-C₂₂ alkyl(hetero)aryl group and C₆-C₂₂ (hetero)arylalkyl group, wherein the alkyl group, the (hetero)aryl group, the alkyl(hetero)aryl group or the (hetero)arylalkyl group is optionally substituted. For example, the alkylene group, the (hetero)arylene group, the alkyl(hetero)arylene group or the (hetero)arylalkylene group is optionally substituted by one or more halogen, —OH, —NH₂, —COOH or —CN. For example, the alkyl group, the (hetero)aryl group, the alkyl(hetero)aryl group or the (hetero)arylalkyl group is optionally substituted by one or more halogen, —OH, —NH₂, —COOH or —CN.

In some embodiments, the X₂ comprises a functional moiety selected from the group consisting of azido group, terminal alkynyl group, cyclic alkynyl group, tetrazinyl group, 1,2,4-trazinyl group, terminal alkenyl group, cyclic alkenyl group, ketone group, aldehyde group, hydroxyl amino group, sulfydryl group, N-maleimide group and their functional derivatives. For example, the X₂ comprises one or more functional moieties.

In some embodiments, the X₂ comprises a functional moiety selected from the group consisting of

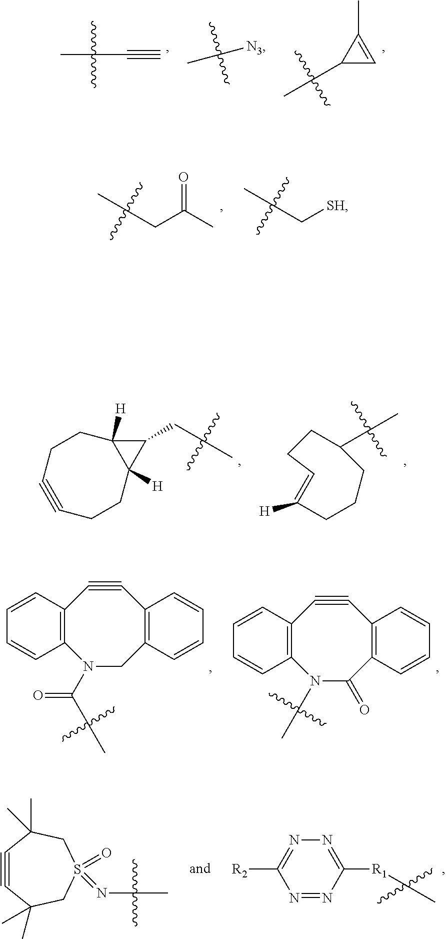

wherein R₁ is selected from the group consisting of C₁-C₂₂ alkylene group, C₅-C₂₂ (hetero)arylene group, C₆-C₂₂ alkyl(hetero)arylene group and C₆-C₂₂ (hetero)arylalkylene group, wherein the alkylene group, the (hetero)arylene group, the alkyl(hetero)arylene group or the (hetero)arylalkylene group is optionally substituted, R₂ is selected from the group consisting of hydrogen, halogen, C₁-C₂₂ alkyl group, C₅-C₂₂ (hetero)aryl group, C₆-C₂₂ alkyl(hetero)aryl group and C₆-C₂₂ (hetero)arylalkyl group, wherein the alkyl group, the (hetero)aryl group, the alkyl(hetero)aryl group or the (hetero)arylalkyl group is optionally substituted. For example, the alkylene group, the (hetero)arylene group, the alkyl(hetero)arylene group or the (hetero)arylalkylene group is optionally substituted by one or more halogen, —OH, —NH₂, —COOH or —CN. For example, the alkyl group, the (hetero)aryl group, the alkyl(hetero)aryl group or the (hetero)arylalkyl group is optionally substituted by one or more halogen, —OH, —NH₂, —COOH or —CN.

In some embodiments, the X₂ comprises a functional moiety selected from the group consisting of

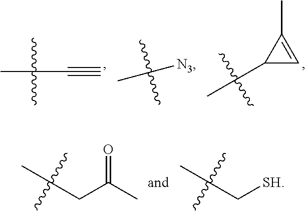

In some embodiments, the X₂ comprises

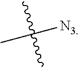 [SMILES: *N=[N+]=[N-]]

In some embodiments, the Y₂ comprises a functional moiety capable of reacting with X₂ through a ligation reaction.

In some embodiments, the Y₂ comprises a functional moiety selected from the group consisting of azido group, terminal alkynyl group, cyclic alkynyl group, tetrazinyl group, 1,2,4-trazinyl group, terminal alkenyl group, cyclic alkenyl group, ketone group, aldehyde group, hydroxyl amino group, sulfydryl group, N-maleimide group and their functional derivatives.

In some embodiments, the Y₂ comprises a functional moiety selected from the group consisting of

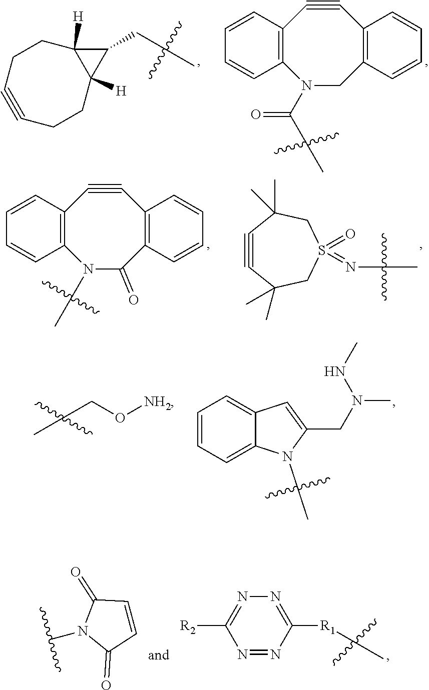

and N wherein R₁ is selected from the group consisting of C₁-C₂₂ alkylene group, C₅-C₂₂ (hetero)arylene group, C₆-C₂₂ alkyl(hetero)arylene group and C₆-C₂₂ (hetero)arylalkylene group, wherein the alkylene group, the (hetero)arylene group, the alkyl(hetero)arylene group or the (hetero)arylalkylene group is optionally substituted, R₂ is selected from the group consisting of hydrogen, halogen, C₁-C₂₂ alkyl group, C₅-C₂₂ (hetero)aryl group, C₆-C₂₂ alkyl(hetero)aryl group and C₆-C₂₂ (hetero)arylalkyl group, wherein the alkyl group, the (hetero)aryl group, the alkyl(hetero)aryl group or the (hetero)arylalkyl group is optionally substituted. For example, the alkylene group, the (hetero)arylene group, the alkyl(hetero)arylene group or the (hetero)arylalkylene group is optionally substituted by one or more halogen, —OH, —NH₂, —COOH or —CN. For example, the alkyl group, the (hetero)aryl group, the alkyl(hetero)aryl group or the (hetero)arylalkyl group is optionally substituted by one or more halogen, —OH, —NH₂, —COOH or —CN.

In some embodiments, the Y₂ comprises a functional moiety selected from the group consisting of

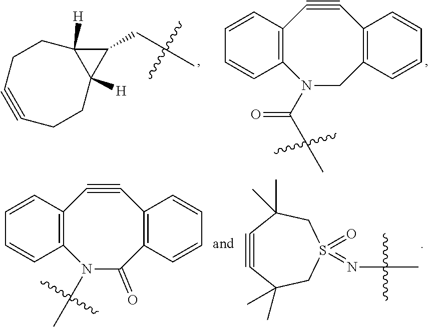 [SMILES: *C[C@H]1[C@H]2CCC#CCC[C@@H]12 ; *C(=O)N1Cc2ccccc2C#Cc2ccccc21 ; *N1C(=O)c2ccccc2C#Cc2ccccc21 ; *N=S1(=O)CC(C)(C)C#CC(C)(C)C1]

In some embodiments, the remaining group X₂Y₂ is selected from the group consisting of

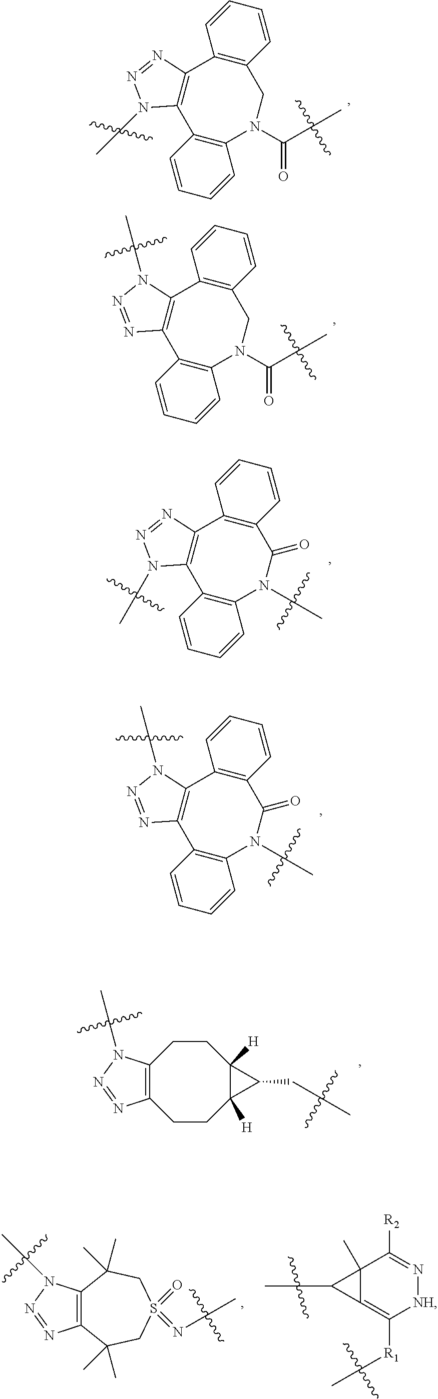

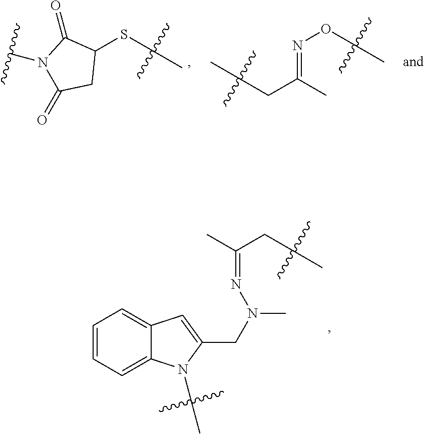

wherein R₁ is selected from the group consisting of C₁-C₂₂ alkylene group, C₅-C₂₂ (hetero)arylene group, C₆-C₂₂ alkyl(hetero)arylene group and C₆-C₂₂ (hetero)arylalkylene group, wherein the alkylene group, the (hetero)arylene group, the alkyl(hetero)arylene group or the (hetero)arylalkylene group is optionally substituted, R₂ is selected from the group consisting of hydrogen, halogen, C₁-C₂₂ alkyl group, C₅-C₂₂ (hetero)aryl group, C₆-C₂₂ alkyl(hetero)aryl group and C₆-C₂₂ (hetero)arylalkyl group, wherein the alkyl group, the (hetero)aryl group, the alkyl(hetero)aryl group or the (hetero)arylalkyl group is optionally substituted. For example, the alkylene group, the (hetero)arylene group, the alkyl(hetero)arylene group or the (hetero)arylalkylene group is optionally substituted by one or more halogen, —OH, —NH₂, —COOH or —CN. For example, the alkyl group, the (hetero)aryl group, the alkyl(hetero)aryl group or the (hetero)arylalkyl group is optionally substituted by one or more halogen, —OH, —NH₂, —COOH or —CN.

In some embodiments, the X₂ and the Y₂ comprise functional moieties selected from the group consisting of: a) X₂ comprises

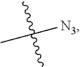 [SMILES: *N=[N+]=[N-]]

and Y₂ comprises

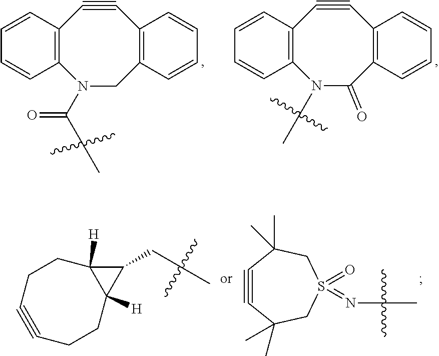

b) X₂ comprises

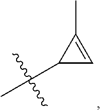 [SMILES: *C1C=C1C]

and Y₂ comprises

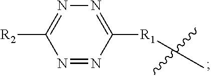

c) X₂ comprises

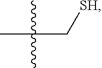 [SMILES: *CS]

and Y₂ comprises

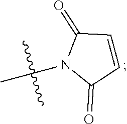 [SMILES: *N1C(=O)C=CC1=O]

and d) X₂ comprises

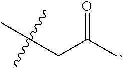 [SMILES: *CC(C)=O]

and Y₂ comprises

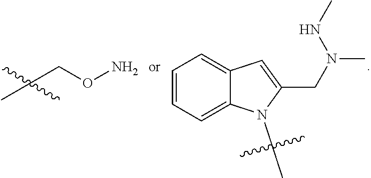

In some embodiments, the X₁ and the X₂ comprise the same functional moiety, and the Y₁ and the Y₂ comprise the same functional moiety. In some embodiments, Y₁-(FL′)_{m′}-(L′)_{n′}-P₁ and Y₂-(FL″)_{m″}-(L″)_{n″}-P₂ are the same molecule.

In some embodiments, the X₁ and the X₂ comprise different functional moieties, and the Y₁ and the Y₂ comprise the same functional moiety. In some embodiments, Y₁-(FL′)_{m′}-(L′)_{n′}-P₁ and Y₂-(FL″)_{m″}-(L″)_{n″}-P₂ are the same molecule.

In some embodiments, the X₁ comprises

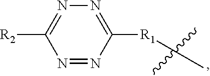

the X₂ comprises

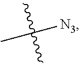 [SMILES: *N=[N+]=[N-]]

the Y₁ comprises

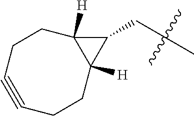 [SMILES: *C[C@H]1[C@H]2CCC#CCC[C@@H]12]

and the Y₂ comprises

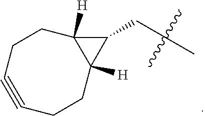 [SMILES: *C[C@H]1[C@H]2CCC#CCC[C@@H]12]

In some embodiments, the X₁ and the X₂ comprise different functional moieties, and the Y₁ and the Y₂ comprise different functional moieties.

In some embodiments, the reaction between the X₁ and the Y₁ substantially does not affect on the reaction between the X₂ and the Y₂.

In some embodiments, the X₁, the Y₁, the X₂ and the Y₂ comprise the functional moieties selected from the group consisting of: a) X₁ comprises

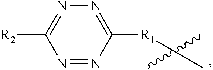

Y₁ comprises

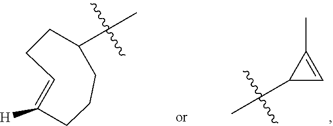

X₂ comprises

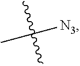 [SMILES: *N=[N+]=[N-]]

and Y₂ comprises

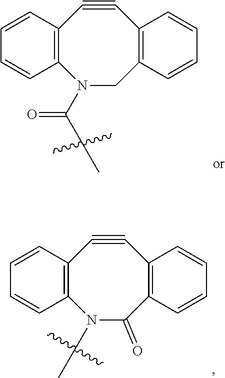 [SMILES: *C(=O)N1Cc2ccccc2C#Cc2ccccc21 ; *N1C(=O)c2ccccc2C#Cc2ccccc21]

b) X₁ comprises

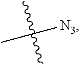 [SMILES: *N=[N+]=[N-]]

Y₁ comprises

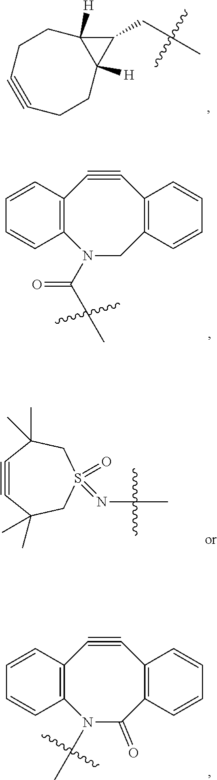 [SMILES: *C[C@H]1[C@H]2CCC#CCC[C@@H]12 ; *C(=O)N1Cc2ccccc2C#Cc2ccccc21 ; *N=S1(=O)CC(C)(C)C#CC(C)(C)C1 ; *N1C(=O)c2ccccc2C#Cc2ccccc21]

X₂ comprises

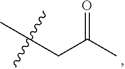 [SMILES: *CC(C)=O]

and Y₂ comprises

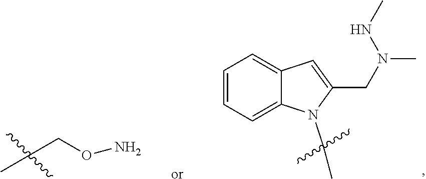

c) X₁ comprises

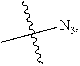 [SMILES: *N=[N+]=[N-]]

Y₁ comprises

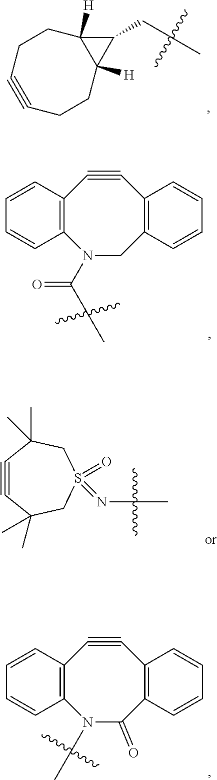 [SMILES: *C[C@H]1[C@H]2CCC#CCC[C@@H]12 ; *C(=O)N1Cc2ccccc2C#Cc2ccccc21 ; *N=S1(=O)CC(C)(C)C#CC(C)(C)C1 ; *N1C(=O)c2ccccc2C#Cc2ccccc21]

X₂ comprises

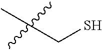 [SMILES: *CS]

and Y₂ comprises

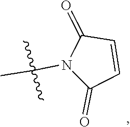 [SMILES: *N1C(=O)C=CC1=O]

and d) X₁ comprises

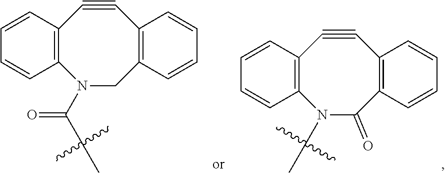

Y₁ comprises

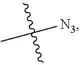 [SMILES: *N=[N+]=[N-]]

X₂ comprises

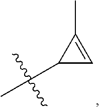 [SMILES: *C1C=C1C]

and Y₂ comprises

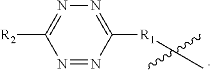

In some embodiments, the X₁ comprises

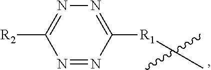

the Y₁ comprises

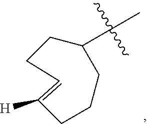

the X₂ comprises

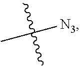 [SMILES: *N=[N+]=[N-]]

and the Y₂ comprises

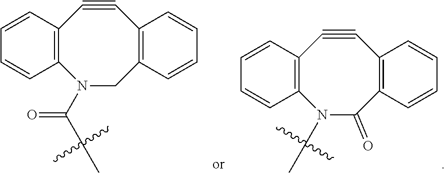

In some embodiments, the connector F is according to Formula (2): (J)_{q}-(FL)ₛ, J is a jointer, FL is a spacer, q is 0 or 1 and s is 0 or 1. In some embodiments, J is a chemical structure that connects the Fuco of Fuc*′ and the FL. In some embodiments, J is directly linked to the Fuco of Fuc*′. In some embodiments, the J is a

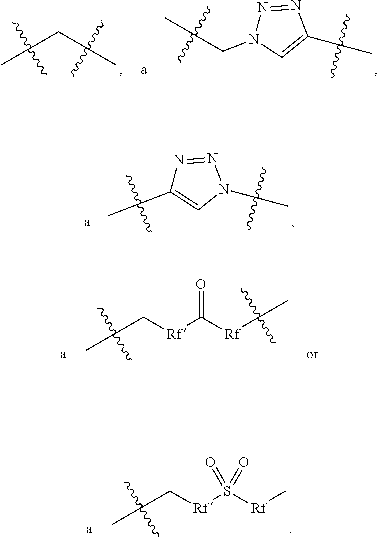

wherein Rf′ is selected from the group of —CH₂—, —NH— and —O—, and Rf is selected from the group of —CH₂—, —NH— and —O—. In some embodiments, the J is a

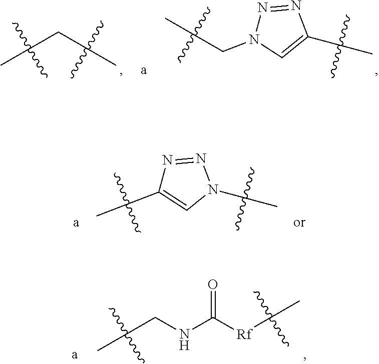

wherein Rf is a —CH₂—, a —NH— or a —O—. In some embodiments, the J is a

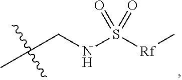

wherein Rf is a —CH₂—, a —NH— or a —O—. For example, the J is a

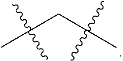 [SMILES: *C*]

For example, the J is a

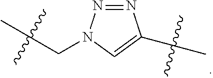 [SMILES: *Cn1cc(*)nn1]

For example, the J is a

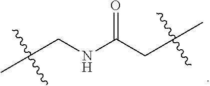 [SMILES: *CNC(=O)C*]

For example, the J is a

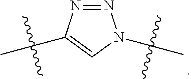 [SMILES: *c1cn(*)nn1]

For example, the J is a

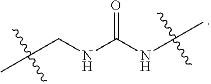 [SMILES: *CNC(=O)N*]

For example, the J is a

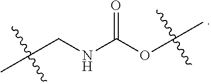 [SMILES: *CNC(=O)O*]

For example, the J is a

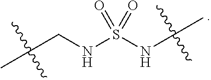 [SMILES: *CNS(=O)(=O)N*]

In some embodiments, F comprises J and FL, and F is J-FL. In some embodiments, F comprises J but does not comprise FL, and F is J.

In some embodiments, the spacer FL is selected from the group consisting of C₃-C₂₀₀ peptide, C₂-C₂₀₀ PEG, C₁-C₂₀₀ alkylene group, C₃-C₂₀₀ cycloalkylene group, C₂-C₂₀₀ alkenylene group, C₅-C₂₀₀ cycloalkenylene group, C₂-C₂₀₀ alkynylene group, C₅-C₂₀₀ cycloalkynylene group, C₂-C₂₄ (hetero)arylene group, C₃-C₂₀₀ (hetero)arylalkylene group, C₃-C₂₀₀ alkynyl(hetero)arylene group, their derivatives and any combination thereof, wherein said the peptide, the PEG, the alkylene group, the cycloalkylene group, the alkenylene group, the cycloalkenylene group, the alkynylene group, the cycloalkynylene group, the (hetero)arylene group, the (hetero)arylalkylene group, or the alkynyl(hetero)arylene group is optionally substituted by one or more Rs¹ and/or is optionally interrupted by one or more Rs², wherein Rs¹ is selected from the group consisting of halogen, —OH, —NH₂ and —COOH, Rs² is independently selected from the group consisting of —O—, —S—,

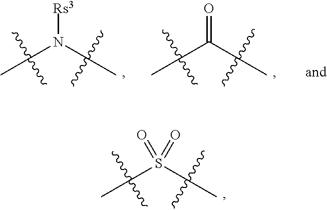

wherein Rs³ is selected from the group consisting of hydrogen, C₁-C₂₄ alkyl group, C₂-C₂₄ alkenyl group, C₂-C₂₄ alkynyl group and C₃-C₂₄ cycloalkyl group.

In some embodiments, the L₁ is a cleavable linker. In some embodiments, the L₁ is an acid-labile linker, a redox-active linker, a photo-active linker and/or a proteolytically cleavable linker. In some embodiments, the L₁ is a vc-PAB-linker, a disulfo linker, or a GGFG-linker.

In some embodiments, the spacer FL′ is selected from the group consisting of C₃-C₂₀₀ peptide, C₂-C₂₀₀ PEG, C₁-C₂₀₀ alkylene group, C₃-C₂₀₀ cycloalkylene group, C₂-C₂₀₀ alkenylene group, C₅-C₂₀₀ cycloalkenylene group, C₂-C₂₀₀ alkynylene group, C₅-C₂₀₀ cycloalkynylene group, C₂-C₂₄ (hetero)arylene group, C₃-C₂₀₀ (hetero)arylalkylene group, C₃-C₂₀₀ alkynyl(hetero)arylene group, their derivatives and any combination thereof, wherein said the peptide, the PEG, the alkylene group, the cycloalkylene group, the alkenylene group, the cycloalkenylene group, the alkynylene group, the cycloalkynylene group, the (hetero)arylene group, the (hetero)arylalkylene group, or the alkynyl(hetero)arylene group is optionally substituted by one or more Rs¹ and/or is optionally interrupted by one or more Rs², wherein Rs¹ is selected from the group consisting of halogen, —OH, —NH₂ and —COOH, Rs² is independently selected from the group consisting of —O—, —S—,

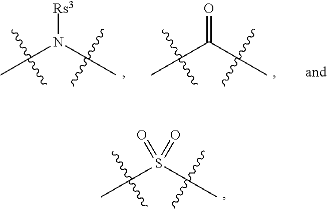

wherein Rs³ is selected from the group consisting of hydrogen, C₁-C₂₄ alkyl group, C₂-C₂₄ alkenyl group, C₂-C₂₄ alkynyl group and C₃-C₂₄ cycloalkyl group.

In some embodiments, the L₁′ is a cleavable linker. In some embodiments, the L₁′ is an acid-labile linker, a redox-active linker, a photo-active linker and/or a proteolytically cleavable linker. In some embodiments, the L₁′ is a vc-PAB-linker, a disulfo linker, or a GGFG-linker.

In some embodiments, the spacer FL″ is selected the group consisting of C₃-C₂₀₀ peptide, C₂-C₂₀₀ PEG, C₁-C₂₀₀ alkylene group, C₃-C₂₀₀ cycloalkylene group, C₂-C₂₀₀ alkenylene group, C₅-C₂₀₀ cycloalkenylene group, C₂-C₂₀₀ alkynylene group, C₅-C₂₀₀ cycloalkynylene group, C₂-C₂₄ (hetero)arylene group, C₃-C₂₀₀ (hetero)arylalkylene group, C₃-C₂₀₀ alkynyl(hetero)arylene group, their derivatives and any combination thereof, wherein said the peptide, the PEG, the alkylene group, the cycloalkylene group, the alkenylene group, the cycloalkenylene group, the alkynylene group, the cycloalkynylene group, the (hetero)arylene group, the (hetero)arylalkylene group, or the alkynyl(hetero)arylene group is optionally substituted by one or more Rs¹ and/or is optionally interrupted by one or more Rs², wherein Rs¹ is selected from the group consisting of halogen, —OH, —NH₂ and —COOH, Rs² is independently selected from the group consisting of —O—, —S—,

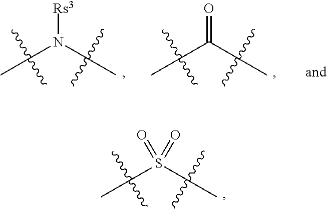

wherein Rs³ is selected from the group consisting of hydrogen, C₁-C₂₄ alkyl group, C₂-C₂₄ alkenyl group, C₂-C₂₄ alkynyl group and C₃-C₂₄ cycloalkyl group.

In some embodiments, the L₂ is a cleavable linker. In some embodiments, the L₂ is an acid-labile linker, a redox-active linker, a photo-active linker and/or a proteolytically cleavable linker. In some embodiments, the L₂ is a vc-PAB-linker, a disulfo linker, or a GGFG-linker.

In some embodiments, the P₁ comprises a cytotoxin, an agonist, an antagonist, an antiviral agent, an antibacterial agent, a radioisotope or a radionuclide, a metal chelator, a fluorescent dye, a biotin, an oligonucleotide, a peptide, a protein, or any combination thereof. In some embodiments, the P₁ is a pharmaceutically active moiety. In some embodiments, the P₁ comprises a cytotoxin, an agonist, an antagonist, an antiviral agent, an antibacterial agent, an oligonucleotide, a peptide or any combination thereof. In some embodiments, the P₁ comprises a cytotoxin. In some embodiments, the P₁ comprises a cytotoxin selected from the group consisting of a DNA or RNA damaging agent, a topoisomerase inhibitor and a microtubule inhibitor. In some embodiments, the P₁ comprises a cytotoxin selected from the group consisting of pyrrolobenzodiazepine, auristatin, maytansinoids, duocarmycin, tubulysin, enediyene, doxorubicin, pyrrole-based kinesin spindle protein inhibitor, calicheamicin, amanitin and camptothecin. In some embodiments, the P₁ comprises a cytotoxin selected from the group consisting of MMAE, MMAF, DXd, DM4 and seco-DUBA.

In some embodiments, the P₂ comprises a cytotoxin, an agonist, an antagonist, an antiviral agent, an antibacterial agent, a radioisotope or a radionuclide, a metal chelator, a fluorescent dye, a biotin, an oligonucleotide, a peptide, a protein, or any combination thereof. In some embodiments, the P₂ is a pharmaceutically active moiety. In some embodiments, the P₂ comprises a cytotoxin, an agonist, an antagonist, an antiviral agent, an antibacterial agent, an oligonucleotide, a peptide or any combination thereof. In some embodiments, the P₂ comprises a cytotoxin. In some embodiments, the P₂ comprises a cytotoxin selected from the group consisting of a DNA or RNA damaging agent, a topoisomerase inhibitor and a microtubule inhibitor. In some embodiments, the P₂ comprises a cytotoxin selected from the group consisting of pyrrolobenzodiazepine, auristatin, maytansinoids, duocarmycin, tubulysin, enediyene, doxorubicin, pyrrole-based kinesin spindle protein inhibitor, calicheamicin, amanitin and camptothecin. In some embodiments, the P₂ comprises a cytotoxin selected from the group consisting of MMAE, MMAF, DXd, DM4 and seco-DUBA.

In some embodiments, the the Fuco of the Fuc*′ is linked to the GlcNAc through an Fuc*′α1,3 linkage.

In some embodiments, the Fuco is according to Formula (3)

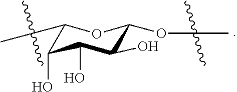

In some embodiments, the Fuc*′ is according to Formula (15)

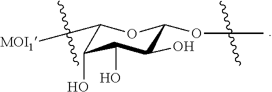

In the protein conjugate, the right part of Formula (3) and Formula (15) is linked to the GlcNAc. In the Q-Fuc*′, the right part of Formula (3) and Formula (15) is linked to the GlcNAc.

In some embodiments, the Q-Fuc*′ is selected from the group consisting of

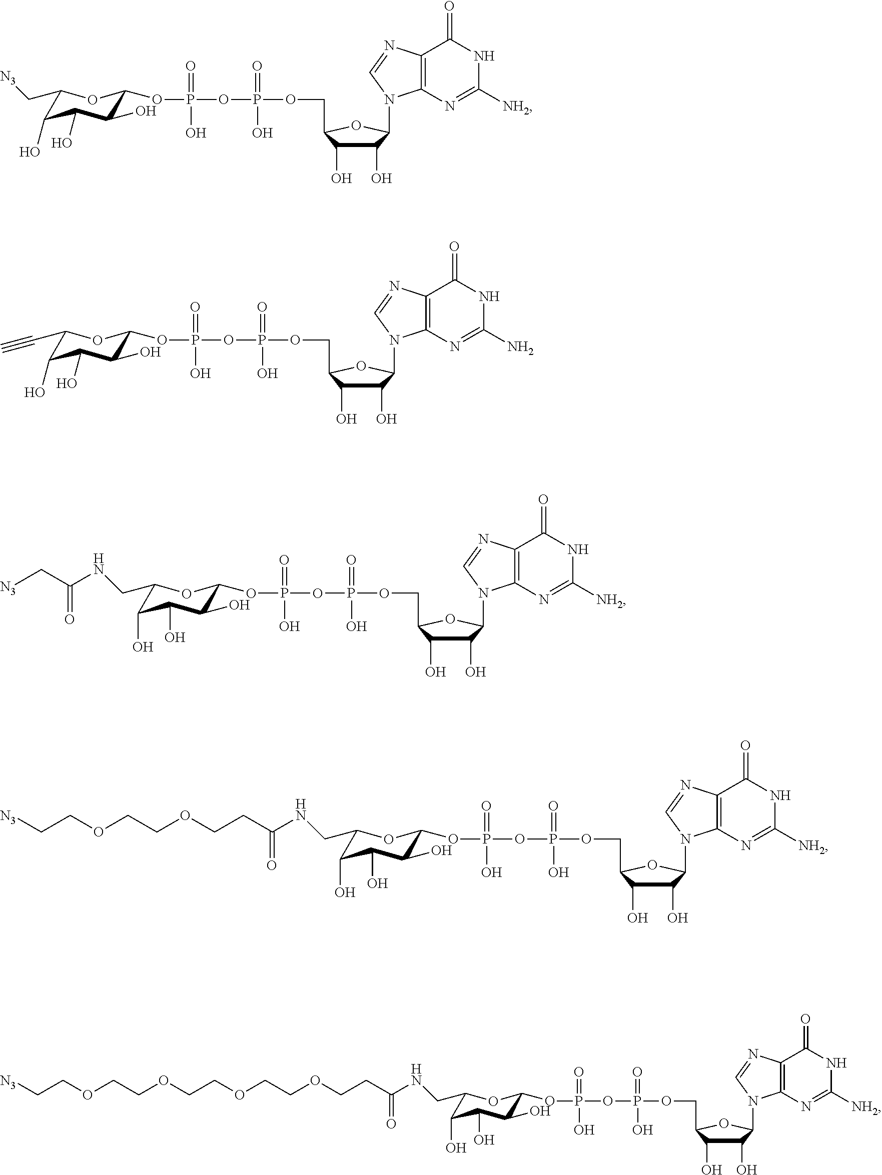

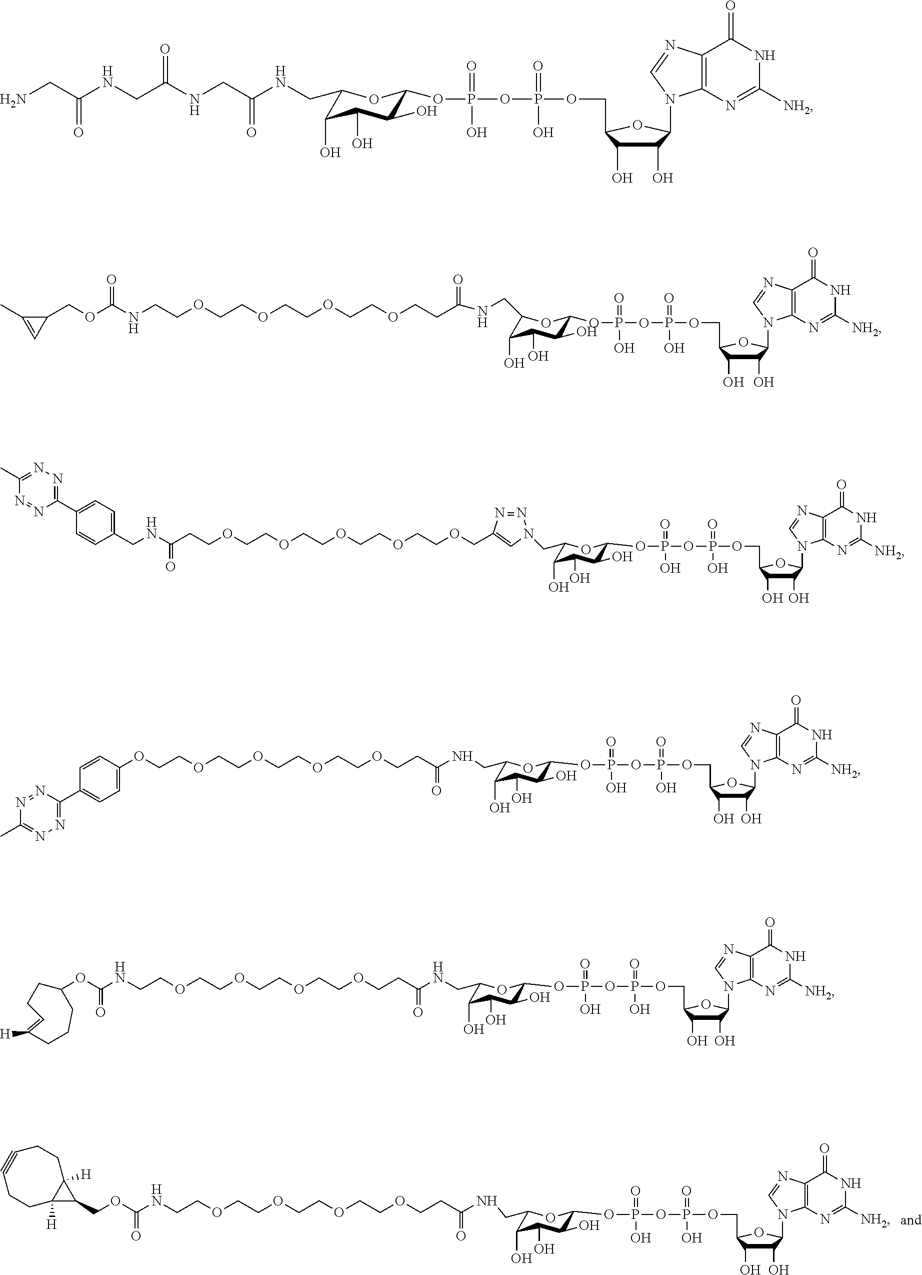

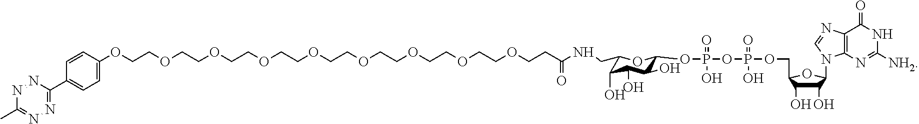

In some embodiments, the Q-Fuc*′ is selected from the group consisting of

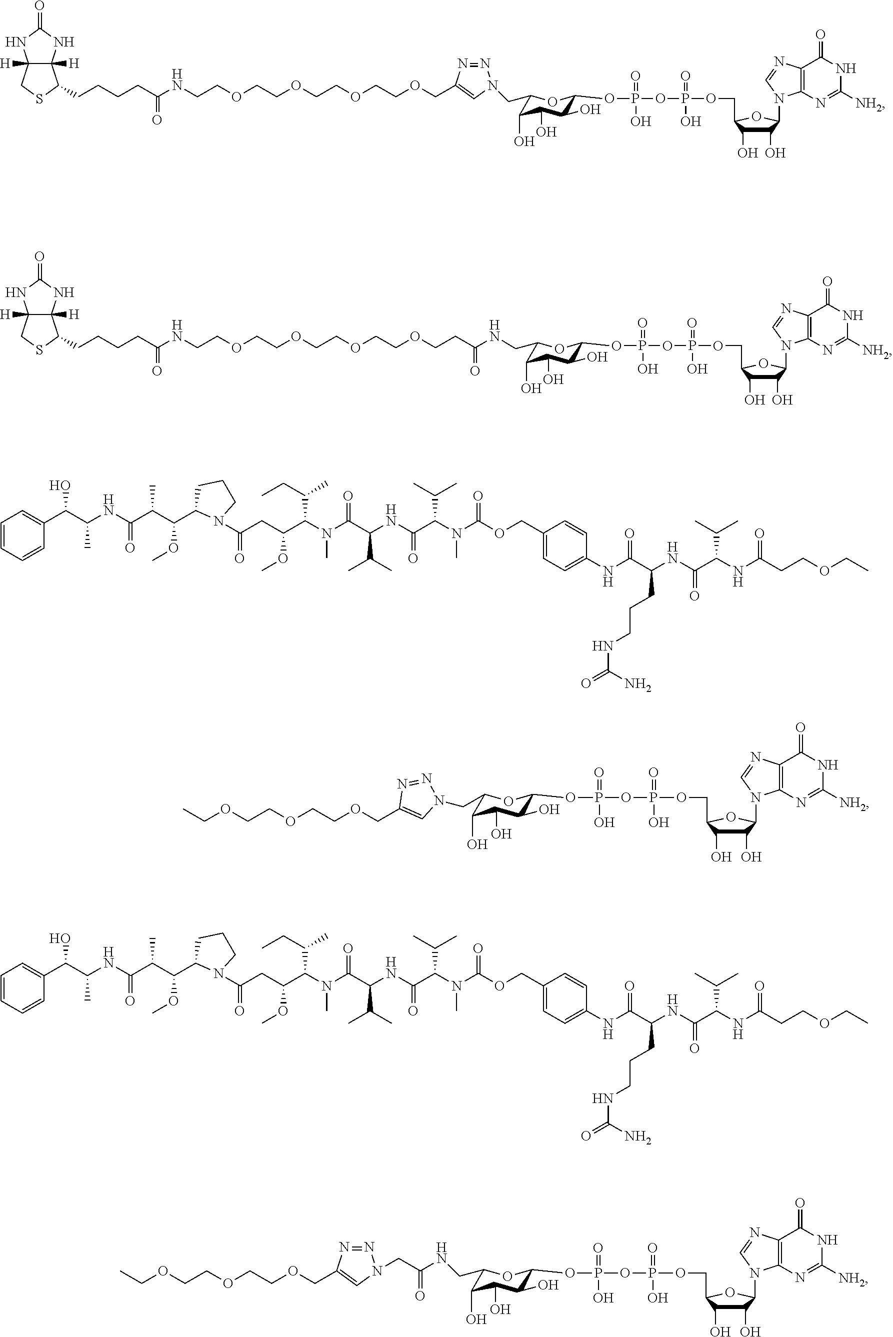

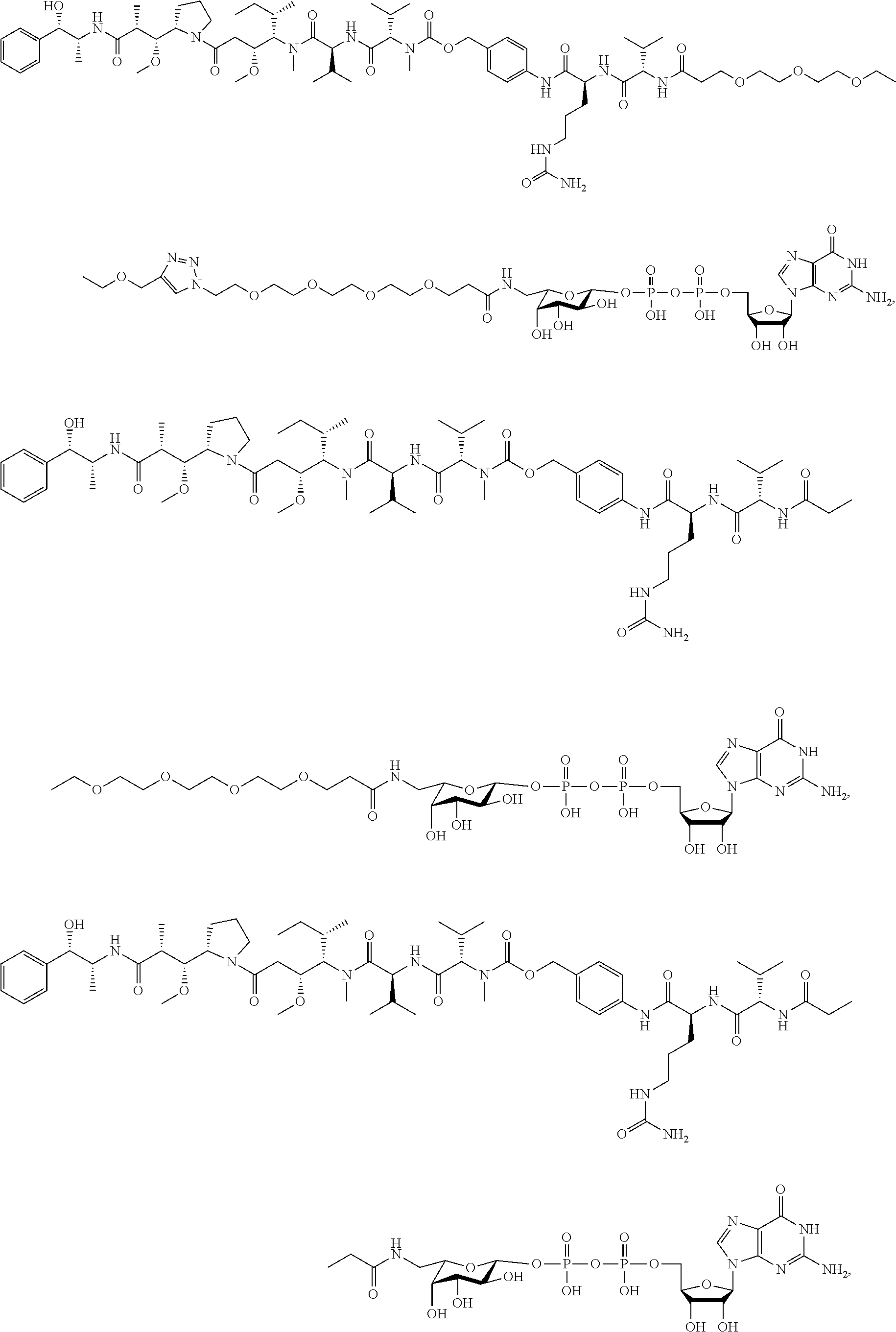

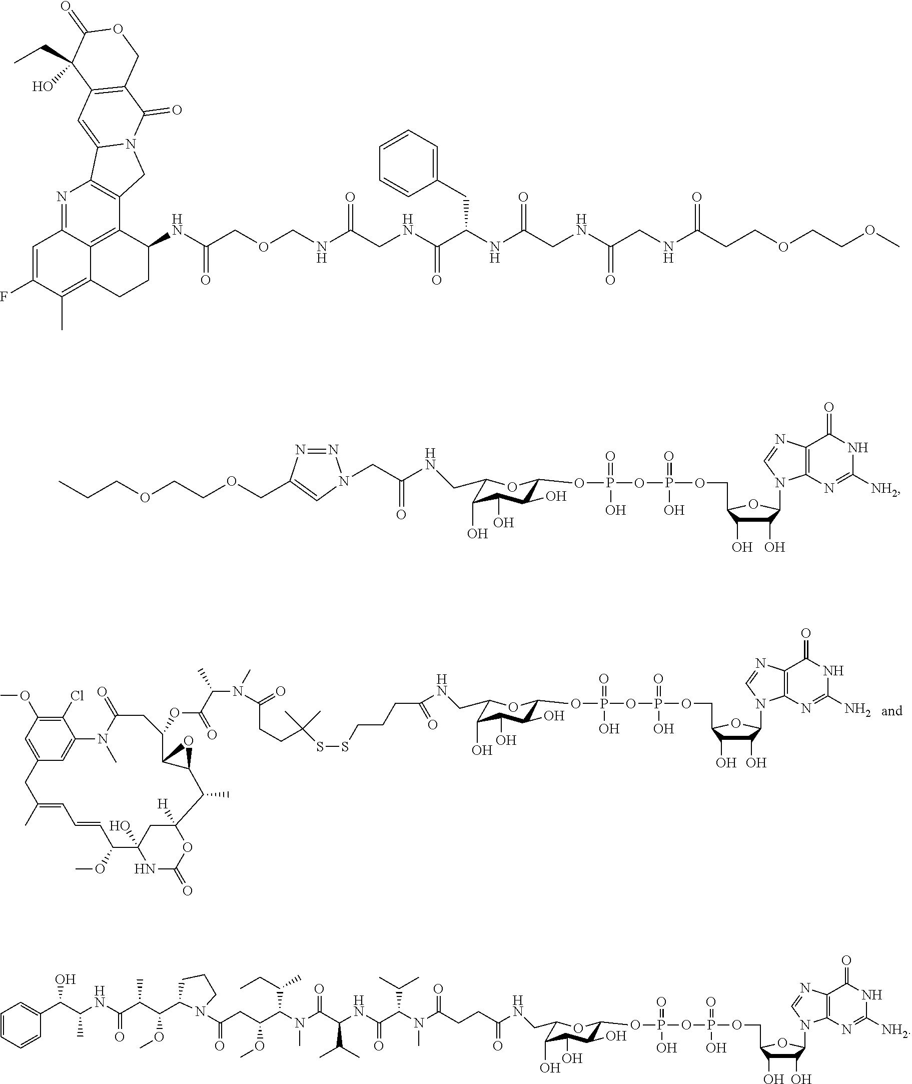

In some embodiments, the GalX′ is selected from the group consisting of

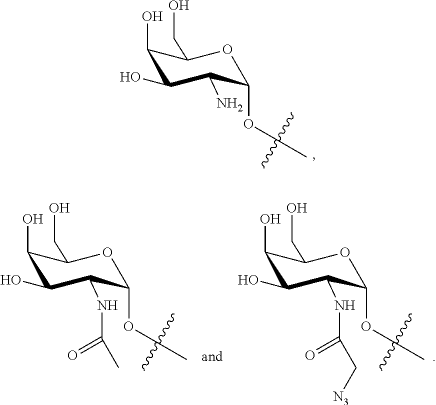

In some embodiments, the method further comprises a step (f): contacting a protein comprising an oligosaccharide comprising the -GlcNAc(Fuc)_{b} with a UDP-GalX′ in the presence of a catalyst, to obtain the protein comprising Formula (10): -GlcNAc(Fuc)_{b}-GalX′.

In some embodiments, the catalyst is a β1,4-galactosyltransferase, or a functional variant or fragment thereof. In some embodiments, the catalyst is a human β1,4-galactosyltransferase, a bovine β1,4-galactosyltransferase, or a functional variant or fragment thereof. In some embodiments, the catalyst comprises a catalytic domain of bovine β(1,4)-GalT1 with an mutation of Y289L, Y289N, Y289I, Y289F, Y289M, Y289V, Y289G, Y289I or Y289A, or a catalytic domain of human β(1,4)-GalT1 with an mutation of Y285L, Y285N, Y285I, Y285F, Y285M, Y285V, Y285G, Y285I or Y285A.

In some embodiments, the catalyst comprises an amino acid sequence as set forth in any one of SEQ ID NOs: 3-5.

In some embodiments, step (f) is performed before step (a). In some embodiments, step (f) is performed before step (a1). In some embodiments, step (f) is performed before step (a2).

In some embodiments, the method does not comprise a purification process between step (f) and step (a). In some embodiments, the method does not comprise a purification process between step (f) and step (a1). In some embodiments, the method does not comprise a purification process between step (f) and step (a2).

In some embodiments, step (f) and step (a) are performed in the same reaction vessel. In some embodiments, step (f) and step (a1) are performed in the same reaction vessel. In some embodiments, step (f) and step (a2) are performed in the same reaction vessel.

In some embodiments, step (f) and step (a) are performed simultaneously. In some embodiments, step (f) and step (a1) are performed simultaneously. In some embodiments, step (f) and step (a2) are performed simultaneously.

In some embodiments, the method further comprises a step (g): modifying a protein comprising an oligosaccharide to obtain a protein comprises a core -(Fucα1,6)_{b}GlcNAc, wherein b is 0 or 1. In some embodiments, step (g) is performed in the presence of an endoglycosidase or a functional variant or fragment thereof. In some embodiments, step (g) is performed in the presence of an EndoS or a functional variant or fragment thereof. In some embodiments, the EndoS comprises an amino acid sequence as set forth in SEQ ID NO: 6 or SEQ ID NO: 7.

In some embodiments, step (g) is performed before the step (f).

In some embodiments, the method further comprising a step (h): modifying a protein comprising the core -(Fucα1,6)₁GlcNAc to a protein comprises a core -GlcNAc.

In some embodiments, step (h) is performed in the presence of a core-α1,6 fucosidase or a functional variant or fragment thereof. In some embodiments, the core-α1,6 fucosidase is Alfc or a functional variant or fragment thereof. In some embodiments, the Alfc comprises an amino acid sequence as set forth in SEQ ID NO: 8 or SEQ ID NO: 9.

In some embodiments, the step (h) is performed behind step (g). In some embodiments, the step (h) is performed before the step (f).

In some embodiments, the step (g) and step (h) are performed simultaneously. In some embodiments, the step (g) and step (h) are performed in the same reaction vessel.

In some embodiments, the method does not comprise a purification process among step (a), step (f), step (g) and step (h). In some embodiments, the method does not comprise a purification process among step (a1), step (f), step (g) and step (h). In some embodiments, the method does not comprise a purification process among step (a2), step (f), step (g) and step (h).

In some embodiments, step (a), step (f), step (g) and step (h) are performed in the same reaction vessel. In some embodiments, step (a1), step (f), step (g) and step (h) are performed in the same reaction vessel. In some embodiments, step (a2), step (f), step (g) and step (h) are performed in the same reaction vessel.

In some embodiments, the protein conjugate comprises 1 to 20 Formula (10): -GlcNAc(Fuc)_{b}-GalX′. In some embodiments, the method comprises obtaining a protein conjugate comprising 1 to 20 Formula (11):

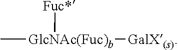

In some embodiments, the oligosaccharide comprises 2 Formula (10): -GlcNAc(Fuc)_{b}-GalX′. In some embodiments, the method comprises obtaining a protein conjugate comprising 2 Formula (11):

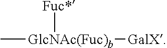

In some embodiments, the method comprises obtaining a protein conjugate according to Formula (8-1) [symbol] , wherein [symbol] is a GlcNAc, [symbol] is the Fuc linked to the GlcNAc through an α-1,6 linkage, GalX′ is a substituted galactose linked to the GlcNAc through a GalX′β1,4GlcNAc linkage, Fuc*′ is linked to the GlcNAc through an Fuc*′α1,3GlcNAc linkage, b is 0 or 1, and [symbol] is an antibody or a Fc-fusion protein.

In some embodiments, the oligosaccharide comprises 4 Formula (10): -GlcNAc(Fuc)_{b}-GalX′. In some embodiments, the method comprises obtaining a protein conjugate comprising 4 Formula (11):

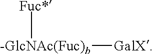

In some embodiments, the method comprises obtaining a protein conjugate according to Formula (9-1) [symbol] , wherein [symbol] is a GlcNAc, [symbol] is the Fuc linked to the GlcNAc through a α1,6GlcNAc linkage, [symbol] is a mannose, GalX′ is a substituted galactose linked to the GlcNAc through a GalX′β1,4GlcNAc linkage, Fuc*′ is linked to the GlcNAc through an Fuc*′α1,3GlcNAc linkage, c is 0 or 1, and [symbol] is an antibody or Fc-fusion protein.

In some embodiments, the method may start from a protein with heterogenous glycosylations (e.g. an antibody or Fc fusion protein with a heterogenous glycosylation forms). For example, the method may comprise the steps performed by the orders of step (g)-step (h)-step (f)-step (a). For example, the method may comprise the steps performed by the orders of step (g)-step (h)-step (f)-step (a1). For example, the method may comprise the steps performed by the orders of step (g)-step (h)-step (f)-step (a2). For example, the method may comprise the steps performed by the orders of step (g)-step (h)-step (f)-step (a1)-step (c1). For example, the method may comprise the steps performed by the orders of step (g)-step (h)-step (f)-step (a2)-step (b). For example, the method may comprise the steps performed by the orders of step (g)-step (h)-step (f)-step (a2)-step (b)-step (d). For example, the method may comprise the steps performed by the orders of step (g)-step (h)-step (f)-step (a2)-step (c2). For example, the method may comprise the steps performed by the orders of step (g)-step (h)-step (f)-step (a2)-step (c2)-step (e). For example, the method may comprise the steps performed by the orders of step (g)-step (f)-step (a). For example, the method may comprise the steps performed by the orders of step (g)-step (f)-step (a1). For example, the method may comprise the steps performed by the orders of step (g)-step (f)-step (a2). For example, the method may comprise the steps performed by the orders of step (g)-step (f)-step (a1)-step (c1). For example, the method may comprise the steps performed by the orders of step (g)-step (f)-step (a2)-step (b). For example, the method may comprise the steps performed by the orders of step (g)-step (f)-step (a2)-step (b)-step (d). For example, the method may comprise the steps performed by the orders of step (g)-step (f)-step (a2)-step (c2). For example, the method may comprise the steps performed by the orders of step (g)-step (f)-step (a2)-step (c2)-step (e).

In some embodiments, the method may start from a protein with an uniform glycosylation of G₀(F) (e.g. an antibody or Fc fusion protein with an glycosylation of G₀(F)). For example, the method may comprise the steps performed by the orders of step (f)-step (a). For example, the method may comprise the steps performed by the orders of step (f)-step (a1). For example, the method may comprise the steps performed by the orders of step (f)-step (a2). For example, the method may comprise the steps performed by the orders of step (f)-step (a1)-step (c1). For example, the method may comprise the steps performed by the orders of step (f)-step (a2)-step (b). For example, the method may comprise the steps performed by the orders of step (f)-step (a2)-step (b)-step (d). For example, the method may comprise the steps performed by the orders of step (f)-step (a2)-step (c2). For example, the method may comprise the steps performed by the orders of step (f)-step (a2)-step (c2)-step (e).

In another aspect, the present disclosure provides use of Q-Fuc*′ of the present disclosure in preparation of a protein conjugate.

In another aspect, the present disclosure provides a protein conjugate, which is obtained with the method of the present disclosure.

In another aspect, the present disclosure provides a method for preparing of a composition, the composition comprises the protein conjugate of the present disclosure.

In another aspect, the present disclosure provides a pharmaceutical composition comprising the protein conjugate of the present disclosure, and/or the composition of the present disclosure, and a pharmaceutically acceptable carrier.

In another aspect, the present disclosure provides a method for preventing or treating disease, comprising administrating the protein conjugate, the composition and/or the pharmaceutical composition of the present disclosure.

In another aspect, the present disclosure provides use of the protein conjugate, the composition and/or the pharmaceutical composition of the present disclosure in preparation of a medicament. In some embodiments, the medicament is used for preventing or treating disease.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWING

The novel features of the invention are set forth with particularity in the appended claims. A better understanding of the features and advantages of the present invention will be obtained by reference to the following detailed description that sets forth illustrative embodiments, in which the principles of the invention are employed, and the accompanying drawings (also “figure”, “Fig.” and “FIG.” herein), of which: FIG. 1 illustrates a preferred embodiment for preparation of a protein conjugate by transferring Fuc*′ (Fuco-MOI₁′) to a protein comprising a -GlcNAc(Fuc)_{0,1}-GalX′ using a GDP-Fuc*′ (GDP-Fuco-MOI₁′) and an α1,3-FucT. POI: protein of interest. MOI₁′: molecule of interest.

FIGS. 2A-2B illustrate the molecular structure of some exemplary GDP-Fuc*′.

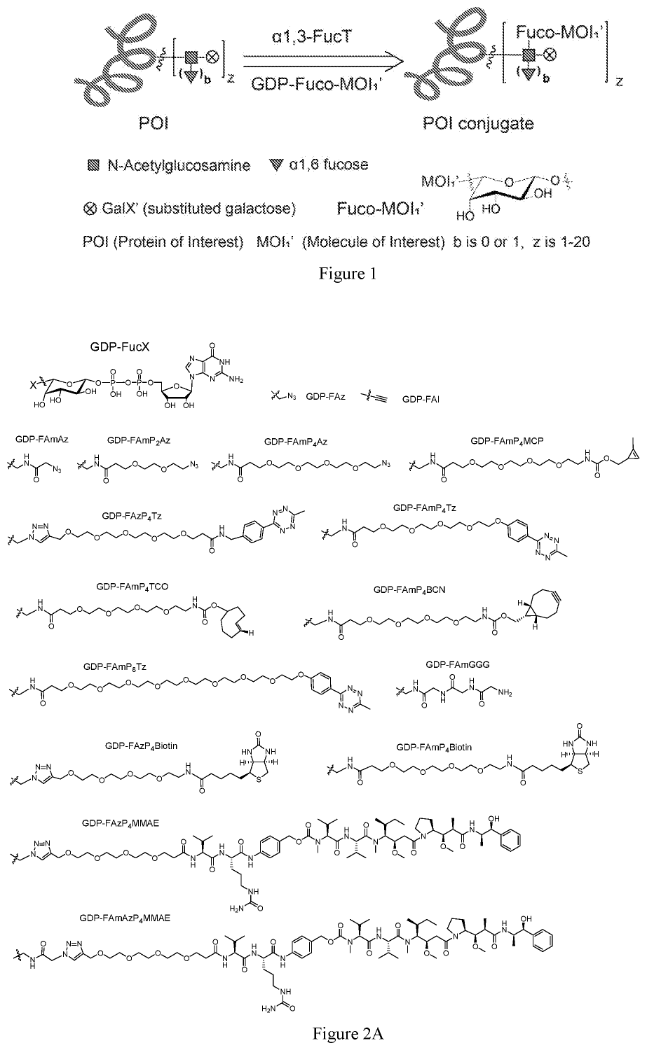

FIG. 3 illustrates a preferred embodiment for the preparation of antibody-(Fucα1,6)_{0.1}(GalX′β1,4)GlcNAc-Fuc*′ conjugates using the GDP-Fuc*′ (GDP-Fuco-MOI₁′) and an α1,3-FucT.

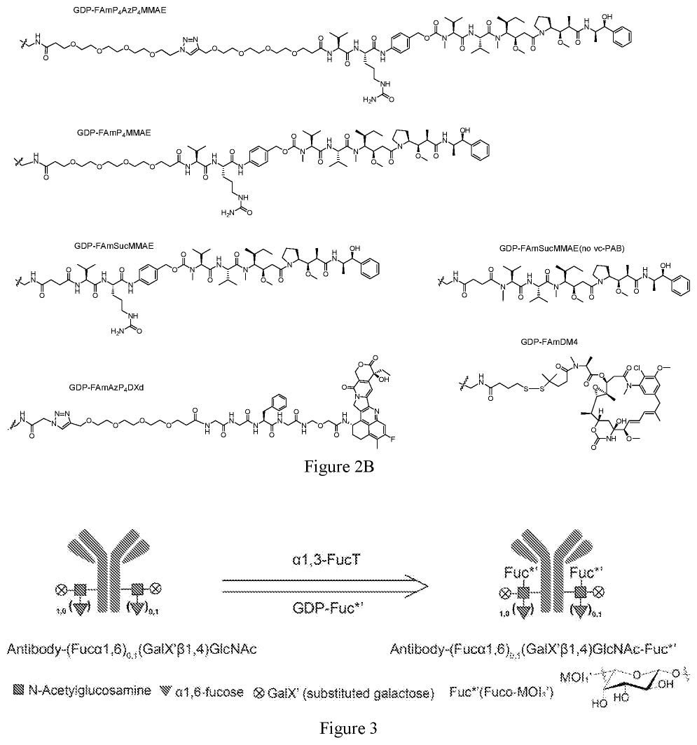

FIGS. 4A-4E illustrate the MS analysis of some exemplary antibody-(Fucα1,6)(GalNAcβ1,4)GlcNAc-Fuc*′ conjugates prepared by using the GDP-Fuc*′ and a Hp1,3-FucT.

FIGS. 5A-5P illustrate the MS analysis of some exemplary antibody-(GalNAcβ1,4)GlcNAc-Fuc*′ conjugates prepared by using the GDP-Fuc*′ and a Hp1,3-FucT FIGS. 6A-6E illustrate the MS analysis of some exemplary antibody-(GalNH₂β1,4)GlcNAc-Fuc*′ conjugates prepared using the GDP-Fuc*′ (GDP-Fuco-MOI₁′) and a Hp1,3-FucT.

FIG. 7 illustrates a preferred embodiment process for the preparation of antibody-(GalX′)₂(F)_{0,1}-Fuc*′ conjugates using GDP-Fuc*′ (GDP-Fuco-MOI₁′) and a Hp1,3-FucT.

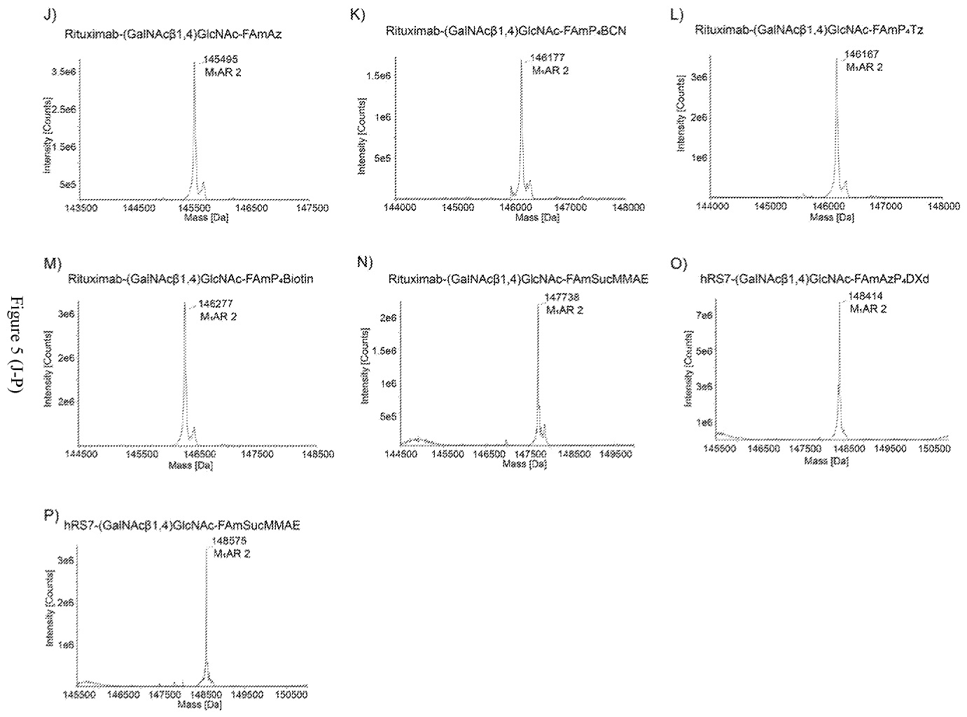

FIG. 8 illustrates the MS analysis of bevacizumab-(GalNAc)₂F, bevacizumab-(GalNAz)₂F, bevacizumab-(GalNAc)₂F-FAz, bevacizumab-(GalNAz)₂F-FAmAz, bevacizumab-(GalNAz)₂F-FAmP₄Biotin and bevacizumab-(GalNAz)₂F-FAmP₄Tz. The commercialized bevacizumab is mainly consisted of G₀F.

FIG. 9 illustrates a preferred embodiment of a “two-step” process for the preparation of an protein conjugate.

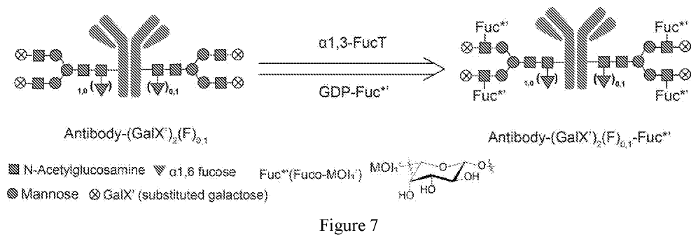

FIG. 10 illustrates the molecular structure of DBCO-PEG₄-vc-PAB-MMAE, TCO-PEG₄-vc-PAB-MMAE, DBCO-PEG₄-vc-PAB-MMAF, DBCO-Disulfo-Cy5 and DBCO-PEG₄-vc-PAB-seco-DUBA.

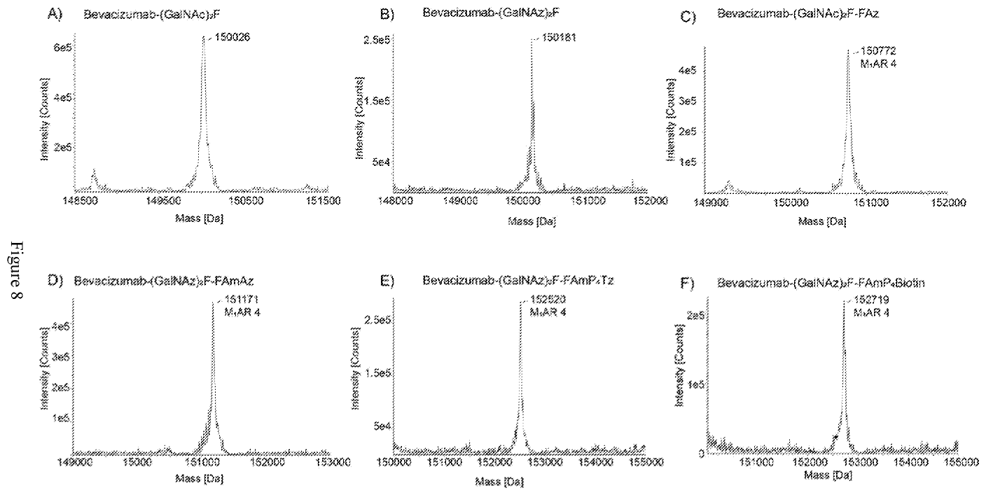

FIGS. 11A-11D illustrates the MS analysis of some exemplary antibody-(GalNAcβ1,4)GlcNAc-Drug conjugates generated from the “two-step” process.

FIG. 12 illustrates HIC-HPLC analysis of some exemplary antibody-drug conjugates.

FIG. 13 illustrates the in vitro cytotoxicity of some exemplary trastuzumab-MMAE conjugates on SK-Br-3 (Her2+) cell line, BT474(Her2+) cell lines and MVDA-MB-231(Her2-) cell line respectively.

FIG. 14 illustrates the in vitro cytotoxicity of an exemplary anti-Trop2-MMAE conjugate (hRS7-(GalNAcβ1,4)GlcNAc-FAmSucMMAE) on JIMT-1 (trop2 high expression) cell line and MDA-MB-231 (trop2 low expression) cell line respectively.

FIG. 15 illustrates preferred embodiments for dual-site-conjugation of a protein containing the GlcNAc(Fuc)_{0,1}-GalX₂ moiety FIG. 16 illustrates MS-analysis of some exemplary dual-site-conjugated antibody-conjugates prepared from the process described in FIG. 15A.

FIGS. 17A-17B illustrates MS-analysis of some exemplary dual-site-conjugated antibody-conjugates generated from the process described in FIG. 15B.

FIG. 18 illustrates the in vitro cytotoxicity of some dual-site-conjugated trastuzumab-drug conjugates on SKOV-3(Her2+) and NCI-N87(Her2+) cells.

FIG. 19 illustrates the binding to recombinant HER2 extracellular domain by trastuzumab and trastuzumab conjugates as analyzed by ELISA.

FIGS. 20A-20B illustrates the stability of some exemplary antibody-drug conjugates in human plasma.

FIG. 21 illustrates the catalytic efficiency of a Hp1,3-FucT on antibody-(GalNAzβ1,4)GlcNAc and antibody-(Fucα1,6)(GalNAβ1,4)GlcNAc in transferring GDP-FAzP₄Biotin or GDP-FAmP₄Biotin. Trastuzumab-(GalNAzβ1,4)GlcNAc (2 mg/mL) were treated with Hp1,3-FucT (0.5 mg/mL) in the presence of 1 mM GDP-FAmP₄Biotin or 1 mM GDP-FAzP₄Biotin for 2 h and measured by LC-MS. Trastuzumab-(Fucα1,6)(GalNAzβ1,4)GlcNAc (2 mg/mL) were treated with Hp1,3-FucT (0.5 mg/mL) in the presence of 1 mM GDP-FAmP₄Biotin for 2 h and measured by LC-MS. % of conversion=average MAR/2*100%.

FIG. 22 illustrates the G₀, G₀F, G₁, G₁F, G₂ and G₂F glycoforms and the (GalX′)₀, (GalX′)F, (GalX′)₁, (GalX′)₁F, (GalX′)₂ and (GalX′)₂F glycoforms of antibodies. The G₀(F) (or (GalX′)₀(F)) form lacks both galactose (Gal) residues or substituted galactose (GalX′) residues at the ends of the biantennary chains. The G₀(F) is the same as the (GalX′)₀(F). The G₁(F) (or (GalX′)₁(F)) are biantennary positional isomers carrying one Gal residue (or one GalX′ residue) attached to the mannose GlcNAc branch. In G₂(F) (or (GalX′)₂(F)), both branches carry a Gal residue (or GalX′ residue). In G₀F, G₁F, G₂F, (GalX′)F, GalX′)₁F and (GalX′)₂F the core-fucose were attached to the core-GlcNAc in an α-1,6 linkage.

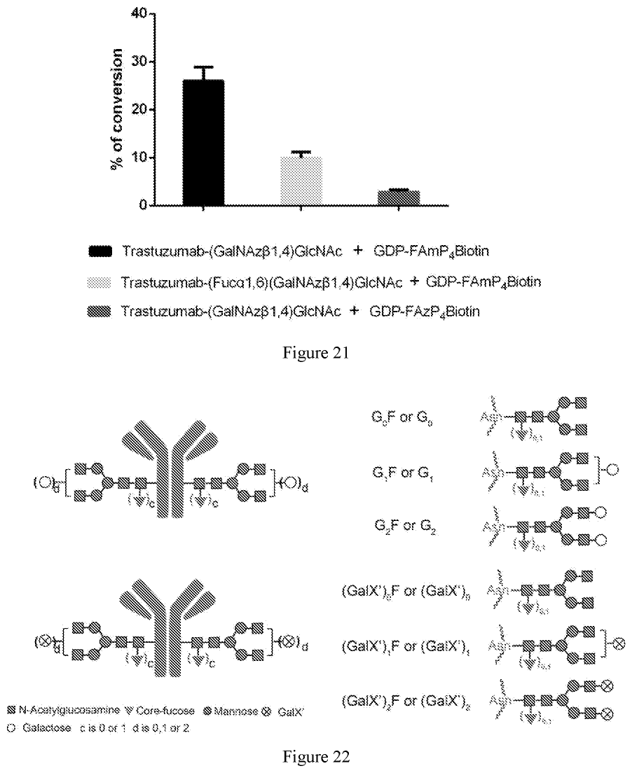

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

The term “conjugate”, as used herein, generally refers to any substance formed from the joining together of separate parts. In the conjugate, the separate parts may be joined at one or more active site with each other. Moreover, the separate parts may be covalently or non-covalently associated with, or linked to, each other and exhibit various stoichiometric molar ratios. The conjugate may comprise peptides, polypeptides, proteins, drugs, prodrugs, polymers, nucleic acid molecules, small molecules, binding agents, mimetic agents, synthetic drugs, inorganic molecules, organic molecules and radioisotopes.

The term “Fc fragment”, as used herein, generally refers to a portion of an antibody constant region. Traditionally, the term Fc domain refers to a protease (e.g., papain) cleavage product encompassing the paired CH₂, CH₃ and hinge regions of an antibody. In the context of this disclosure, the term Fc domain or Fc refers to any polypeptide (or nucleic acid encoding such a polypeptide), regardless of the means of production, that includes all or a portion of the CH₂, CH₃ and hinge regions of an immunoglobulin polypeptide.

The term “antigen binding fragment”, as used herein, generally refers to a peptide fragment capable of binding antigen. The antigen binding fragment may be a fragment of an immunoglobulin molecule. An antigen-binding fragment may comprise one light chain and part of a heavy chain with a single antigen-binding site. An antigen-binding fragment may be obtained by papain digestion of an immunoglobulin molecule. For example, an antigen-binding fragment may be composed of one constant and one variable domain of each of the heavy and the light chain. The variable domain may contain the paratope (the antigen-binding site), comprising a set of the complementarity determining regions, at the amino-terminal end of the immunoglobulin molecule. For example, the antigen binding fragment may be a Fab, a F(ab)₂, F(ab′), a F(ab′)₂, a ScFv, and/or a nanobody.

The term “antibody”, as used herein, generally refers to a polypeptide or a protein complex that specifically binds an epitope of an antigen or mimotope thereof. An antibody includes an intact antibody, or a binding fragment thereof. that competes with the intact antibody for specific binding and includes chimeric, humanized, fully human, and bispecific antibodies. Binding fragments include, but are not limited to, Fab, Fab′, F(ab′)₂, Fv, single-chain antibodies, nanobodies and disulfide-linked Fvs (sdFv) fragments. In some embodiments, an antibody is referred to as an immunoglobulin and include the various classes and isotypes, such as IgA (IgA1 and IgA2), IgD, IgE, IgM, and IgG (IgGl, IgG3 and IgG4) etc. In some embodiments the term “antibody” as used herein refers to polyclonal and monoclonal antibodies and functional fragments thereof. An antibody includes modified or derivatized antibody variants that retain the ability to specifically bind an epitope. Antibodies are capable of selectively binding to a target antigen or epitope. In some embodiments, the antibody is from any origin, such as mouse or human, including a chimeric antibody thereof. In some embodiments, the antibody is humanized.

The term “monoclonal antibody”, as used herein, generally refers to an antibody obtained from a population of substantially homogeneous antibodies, i.e., the individual antibodies comprising the population are identical except for possible naturally occurring mutations and/or post-translational modifications (e.g., isomerizations, amidations) that may be present in minor amounts.

The term “IgG”, as used herein, generally refers to various broad classes of polypeptides or proteins that can be distinguished biochemically. Those skilled in the art will appreciate that immunoglobulin heavy chains are classified as gamma, mu, alpha, delta, or epsilon, (γ, μ, α, δ, ε) with some subclasses among them (e.g., γ1-γ4 or α1-α2)). It is the nature of this chain that determines the “isotype” of the antibody as IgG, IgM, IgA IgG, or IgE, respectively. The immunoglobulin subclasses (subtypes) e.g., IgG1, IgG2, IgG3, IgG4, IgA1, IgA2, etc. are well characterized and are known to confer functional specialization. Human IgG is typically characterized by glycosylation at position Asn297 (numbering according to Kabat numbering system) in the heavy chain CH₂ region of the Fc region.

In the present disclosure, “Asn297”, or “N297”, can be used interchangeably, generally refers to the Asparagine at site 297 (numbered according to the Kabat numbering system, (Kabat et al., Sequences of Proteins of Immunological Interest, Vol. 1, 5th Ed. U.S. Public Health Service, National Institutes of Health. NIH Publication No. 91-3242; Copyright 1991)) of an antibody Fc fragment. Asn297 of an antibody or antibody fragment may be attached with one or more oligosaccharide.

The term “humanized antibody”, as used herein, generally refers to containing the antibody from some or all CDR of nonhuman animal antibody, and the framework of antibody and constant region contain the amino acid residue of derived from human antibody sequence.

The term “Fc-fusion protein”, as used herein, generally refers to a protein which are composed of the Fc domain of an immunoglobulin genetically linked to a peptide or protein of interest. In some embodiment, the protein conjugate of the present disclosure is a Fc-fusion protein conjugate.

The term “GlcNAc”, or “N-acetylglucosamine”, can be used interchangeably, generally refers to an amide derivative of the monosaccharide glucose.

Glycosylation generally refers to the reaction in which a carbohydrate, i.e., a glycosyl donor, is attached to a hydroxyl or other functional group of another molecule (a glycosyl acceptor). In some embodiments, glycosylation mainly refers in particular to the enzymatic process that attaches glycans to proteins, or other organic molecules. The glycosylation in protein can be modified in glycosylation linkage, glycosylation structure, glycosylation composition and/or glycosylation length. Glycosylation can comprise N-linked glycosylation, O-linked glycosylation, phosphoserine glycosylation, C-mannosylation, formation of GPI anchors (glypiation), and/or chemical glycosylation. Correspondingly, a glycosylated oligosaccharide of a protein can be a N-linked oligosaccharide, 0-linked oligosaccharide, phosphoserine oligosaccharide, C-mannosylated oligosaccharide, glypiated oligosaccharide, and/or chemical oligosaccharide.

The term “N-linked oligosaccharide”, as used herein, generally refers to the attachment of an oligosaccharide to a nitrogen atom. In some embodiments, the oligosaccharide may comprise a carbohydrate consisting of several sugar molecules, sometimes also referred to as glycan. In some embodiments, the nitrogen atom is an amide nitrogen of an amino acid residue of a protein, for example, an asparagine (Asn) of a protein.

The term “directly linked” as used herein, generally refers to that a moiety is linked to another moiety without any intermediate moiety or linker. For example, a GlcNAc is directly linked to an amino acid residue of an antibody generally refers to that the GlcNAc is bonded via a covalent bond to an amino acid residue of the antibody, for example, via an N-glycosidic bond to an amide nitrogen atom in a side chain of an amino acid (e.g., an asparagine amino acid) of the antibody. In the present disclosure, when a GlcNAc is “indirectly linked” to an amino acid of the protein, there are usually at least one monosaccharide moiety between the GlcNAc and the amino acid of the protein.

The term “Fuc*α1,3GlcNAc linkage”, as used herein, generally refers to a linkage between a Fuco of the Fuc* and a GlcNAc, which links the C1 of Fuco to the C3 of the GlcNAc. The term “Fuc*′α1,3GlcNAc linkage”, as used herein, generally refers to a linkage between a Fuco of the Fuc*′ and a GlcNAc, which links the C1 of Fuco to the C3 of the GlcNAc.

The term “GalXβ1,4GlcNAc linkage”, as used herein, generally refers to a linkage between a substituted galactose GalX and a GlcNAc, which links the C1 of GalX to the C4 of the GlcNAc. The term “GalX′β1,4GlcNAc linkage”, as used herein, generally refers to a linkage between a substituted galactose GalX′ and a GlcNAc, which links the C1 of GalX′ to the C4 of the GlcNAc.

The term “molecule of interest”, as used herein, generally refers to a molecule with a desired characteristic. The desired characteristic may be a physical characteristic or a chemical characteristic, for example, reactive activity, stability, solubility, binding activity, inhibiting activity, toxicity or degradability. A MOI may comprise any substances possessing a desired biological activity and/or a reactive functional group that may be used to incorporate a drug into the protein conjugate of the disclosure. For example, a MOI may comprise an active moiety. For example, the active moiety may be a therapeutical agent, a diagnosis agent, a pharmacological agent and/or a biological agent, e.g., a cytotoxin, a cytostatic agent, a radioisotope or radionuclide, a metal chelator, an oligonucleotide, an antibiotic, a fluorophore, a biotin tag, a peptide, a protein, or any combination thereof. In some cases, an active moiety could be a chemically active moiety. For example, a chemically active moiety may be a chemically functional moiety that could reacted with another chemically functional moiety to form a covalent bond. For example, a chemically active moiety may be able to participate in a ligation reaction. In some cases, an active moiety could be an enzymatically active moiety that could be reacted with complementary functional moiety to form a covalent bond in the presence of an enzyme. For example, an enzymatically active moiety may be an N-terminal peptide tag GGG which could react with a C terminal peptide tag in the presence of a sortase ligase to form a covalent bond.

The term “functional group” as used herein, generally refers to a group capable of reacting with another group. A functional group can be used to incorporate an agent (e.g., an agent without a reactive activity or with a low reactive activity) into a protein or a protein conjugate. For example, the agent may be a pharmaceutically active moiety (e.g. a cytotoxin). A functional group may be a chemical group or a residue having chemical and/or enzymatic reactivity. In some embodiments, a functional group may be a group capable of reacting in a ligation reaction. A functional group usually comprises a functional moiety, and the functional group may react with another group due to the functional moiety.

The term “ligation reaction” as used herein, generally refers to a chemical and/or an enzymatic reaction in which a molecule is capable of linked to another molecule. This binding may be driven by the functional group of the reactive molecules.

The term “bioorthogonal ligation reaction” as used herein, generally refers to a chemical reaction for making protein conjugate of the present disclosure that occurs specifically between a first functional moiety at specific positions on the protein (e.g. located on the oligosaccharide of the protein) and a second complementary functional moiety linked to a molecule to be introduced under a suitable condition. The first functional moiety and the second complementary functional moiety are a bioorthogonal ligation reaction pair. Generally, the first functional moiety at specific positions on the protein would be easily distinguished from other groups on the other part of the protein. Generally, the second complementary functional moiety would not react with the other parts of the protein except for the first functional moiety at specific positions. For example, an azido group is a functional moiety capable of participating in a bioorthogonal ligation reaction. A complementary DBCO or BCN groups could specifically react with the azido groups without cross-reacting with other groups on the protein. For another example, a —NH₂ group may not be a functional moiety capable of participating in a bioorthogonal ligation reaction in the present disclosure. As there's lots of —NH₂ groups on different sites of the protein, which is highly undistinguishable by using a N-hydroxysuccinimide ester reagent. The skilled person in the art will understand that if a —NH₂ group at specific positions on the protein would be easily distinguished from other —NH₂ groups on the other part of the protein, for example, under a certain condition, and then the —NH₂ group at specific positions on the protein may also be a functional moiety capable of participating in a bioorthogonal ligation reaction. A lot of chemically reactive functional moiety with suitable reactivity, chemoselectivity and/or biocompatibility can be used in a bioorthogonal ligation reaction. A group that capable of participating in a bioorthogonal ligation reaction could be selected from but not limited a functional moiety selected from the group consisting of azido groups, terminal alkynyl groups, cyclic alkynyl groups, tetrazinyl groups, 1,2,4-trazinyl groups, terminal alkenyl groups, cyclic alkenyl groups, ketone groups, aldehyde groups, hydroxyl amino groups, sulfydryl groups, N-maleimide groups and their functional derivatives (refer to Bertozzi C. R., et.al Angew. Chem. Int. Ed., 2009, 48, 6974; Chin J. W., et.al ACS Chem. Biol. 2014, 9, 16; van Del F. L., et.al Nat. Commun., 2014, 5, 5378; Prescher J. A., et.al Acc. Chem. Res. 2018, 51, 1073; Devaraj N K. ACS Cent. Sci. 2018, 4, 952; Liskamp R. M. J., et.al Chem. Sci., 2020, 11, 9011).

As used herein, the term “functional variant” of a parent polypeptide or protein generally refers to the polypeptide or protein having substantial or significant sequence identity or similarity to a parent polypeptide or protein, which functional variant retains at least one of the functions of the parent polypeptide or protein of which it is a variant. For example, a functional variant of an enzyme retains the enzymatic activity to a similar extent, the same extent, or to a higher extent, as the parent enzyme. In reference to parent the polypeptide or the protein, the functional variant can, for instance, be about 80% or more, about 90% or more, about 95% or more, about 96% or more, about 97% or more, about 98% or more, or about 99% or more identical in amino acid sequence to the parent polypeptide or protein. In some cases, the functional variant may be a polypeptide different from the parent a peptide or polypeptide at least one amino acid. For example, the functional variant may be a polypeptide different from the parent a polypeptide or protein by an addition, deletion or substitution of one or more amino acid, such as 1-200, 1-100, 1-50, 1-40, 1-30, 1-20, 1-15, 1-14, 1-13, 1-12, 1-11, 1-10, 1-9, 1-8, 1-7, 1-6, 1-5, 1-4, 1-3, or 1-2 amino acids.

As used herein, the term “functional fragment” of a parent peptide or polypeptide generally refers to a peptide or polypeptide (including, but not limited to, an enzyme), which contains at least 5 contiguous amino acid residues, at least 10 contiguous amino acid residues, at least 15 contiguous amino acid residues, at least 20 contiguous amino acid residues, at least 25 contiguous amino acid residues, at least 40 contiguous amino acid residues, at least 50 contiguous amino acid residues, at least 60 contiguous amino acid residues, at least 70 contiguous amino acid residues, at least 80 contiguous amino acid residues, at least 90 contiguous amino acid residues, at least 100 contiguous amino acid residues, at least 125 contiguous amino acid residues, at least 125 contiguous amino acid residues, at least 150 contiguous amino acid residues, at least 175 contiguous amino acid residues, at least 200 contiguous amino acid residues, at least 250 contiguous amino acid residues or at least 350 contiguous amino acid residues of the parent polypeptide or protein, wherein polypeptide or protein retains at least one of the functions of the parent peptide or polypeptide. For example, functional fragment” of a parent enzyme retains the enzymatic activity to a similar extent, the same extent, or to a higher extent, as the parent enzyme.

The term “fucosyltransferase”, as used herein, generally refers to an enzyme or a functional variant thereof. that can transfer a L-fucose sugar from a fucose donor substrate (such as, guanosine diphosphate-fucose) to an acceptor substrate. The acceptor substrate can be another sugar such as a sugar comprising a GlcNAc-Gal (LacNAc), as in the case of N-glycosylation, or in the case of 0-linked glycosylation. The example of fucosyltransferase may be an α-1,3 fucosyltransferase. The term “fucosyltransferase” may comprise any functional fragments (e.g. a catalytic domain thereof), or functional variants (e.g., mutant). The term “fucosyltransferase” may derived from various species, such as mammals (e.g., humans), bacteria, nematodes or trematodes. The term “fucosyltransferase” may derived from bacteria.

The term “Fuc” as used herein, generally refers to a fucose linked with a GlcNAc, wherein the GlcNAc is directly linked to an amino acid of a protein (e.g., an antibody or a fragment thereof). For example, the “Fuc” is linked with the GlcNAc through a α1,6 linkage. The Fuc is different with the Fuco of the Fuc* or Fuc*′ of the present disclosure. In the present disclosure, the “Fuco” represents the fucose or fucose derivative of the Fuc* or Fuc*′.

In the present disclosure, the term “connector”, represented by “F”, generally refers to a chemical structure that links an active moiety (e.g. X₁, P₁), or a linker (e.g. L₁), or a remaining group (X₁Y₁) to the Fuco of Fuc* (or, Fuc*′). The connector F is directly linked to the Fuco of Fuc*. The connector F is capable of (1) linking two parts together (2) tuning the distance between the two parts that the connector linked with; (3) tuning the hydrophilicity of the parts the connector linked with, and/or (4) tuning the conformation of the parts the connector linked with. In some embodiments, the F comprises a jointer, represented by “J”. In some embodiments, the F comprises a spacer, represented by “FL”. In some embodiments, the F comprises a jointer J and a spacer FL.

The term “jointer”, represented by “J”, as used herein, generally refers to a chemical structure connecting the Fuco of Fuc*(or, Fuc*′) and the spacer FL (when the F comprises a FL), or an active moiety (e.g. X₁, P₁) (when the F does not comprise a FL), or a linker (e.g. L₁) (when the F does not comprise a FL), or a remaining group (X₁Y₁) (when the F does not comprise a FL). In some embodiments, the jointer J may be directly linked the the Fuco of Fuc* (or, Fuc*′).

The term “spacer”, represented by “FL”, “FL′”, or FL″”, as used herein, generally refers to a chemical structure capable of (1) linking two part together; (2) tuning the distance between the two parts that the spacer linked with; (3) tuning the hydrophility of the parts the spacer linked with, and/or (4) tuning the conformation of the parts the spacer linked with.

The term “pharmaceutically active moiety”, as used herein, generally refers to any substance being pharmaceutically useful or having a pharmaceutical effect. In the present disclosure, a fluorescent label may not be a pharmaceutically active moiety. For example, a pharmaceutically active moiety may be an agent capable of alleviating, treating, preventing a disease, or delaying a disease process. The disease may be a disease associated with abnormal cell proliferation and/or cellular dysfunction. The disease may be a tumor and/or an immune disease.

A pharmaceutically active moiety may comprise a compound useful in the characterization of tumors or other medical condition, for example, diagnosis, characterization of the progression of a tumor, and assay of the factors secreted by tumor cells. For example, the pharmaceutically active moiety may be a radioisotope or radionuclide. For example, the pharmaceutically active moiety may be a PET imaging agent.

A pharmaceutically active moiety may be a cytotoxin. A cytotoxin may comprise any agents capable of damaging to cell proliferation and/or differentiation. A cytotoxin may have a cytotoxic effect on tumors including the depletion, elimination and/or the killing of tumor cells.

The term “corresponding antibody”, as used herein, generally refers to the antibody from which a protein conjugate can be obtained after some modifications, e.g., glycosylation modification, ligation reaction or conjugation, especially after performing the method of the present disclosure. A protein conjugate may be capable of binding to the same antigen or the same antigen epitome with its corresponding antibody. A corresponding antibody can be conjugated with a molecule of interest to become a protein conjugate. For a given protein conjugate, if the protein conjugate can be obtained from an antibody by one or more steps of glycosylation, deglycosylation, and conjugation with a molecule of interest, the antibody may be the corresponding antibody of the protein conjugate. The “corresponding antibody” and the protein conjugate may have different glycoforms. For example, the “corresponding antibody” may be an antibody comprising heterogenous glycoforms (e.g. a mixture of G₂(F), G₁(F) and G₀(F)).

The terms “comprise” and variations thereof. do not have a limiting meaning where these terms appear in the description and claims.

Unless otherwise specified, “a”, “an”, “the” and “at least one” are used interchangeably and mean one or more than one.

### Protein Conjugate

In one aspect, the present disclosure provides a protein conjugate, which comprises a protein and an oligosaccharide, wherein the oligosaccharide comprises Formula (1):

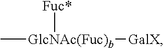

wherein, said GlcNAc is directly or indirectly linked to an amino acid of the protein, said GalX is a substituted galactose, said Fuc is a fucose, and b is 0 or 1, the Fuc* comprises a fucose or fucose derivative (Fuco) and a molecule of interest (MOI₁).

In another aspect, the present disclosure provides a method for preparing a protein conjugate, the method comprises step (a): contacting a fucose derivative donor Q-Fuc*′ with a protein comprising an oligosaccharide in the presence of a catalyst, wherein the oligosaccharide comprises Formula (10): -GlcNAc(Fuc)_{b}-GalX′, to obtain a protein conjugate comprising Formula (11):

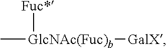

wherein the GlcNAc is directly or indirectly linked to an amino acid of the protein, the GalX′ is a substituted galactose, the Fuc is a fucose, b is 0 or 1, the Q-Fuc*′ is a molecule comprises Fuc*′, the Fuc*′ comprises a Fuco and a molecule of interest (MOI₁′).

The protein conjugate which comprises the oligosaccharide comprising Formula (1) can be obtained by the method comprising step (a) of the present disclosure. The protein conjugate which comprises the oligosaccharide comprising Formula (1) can be obtained by the method comprising step (a) and/or other steps (e.g., one or more ligation reactions between functional groups, galactosylation, and/or deglycosylation) mentioned in the present disclosure.

In the present disclosure, Formula (11) and Formula (1) may have the same structure. In the present disclosure, Formula (11) may be transformed into Formula (1) after one or more ligation reactions.

In the present disclosure, the protein conjugate comprising Formula (11) may be the same as the protein conjugate comprising Formula (1), or a intermediate product of preparation the protein conjugate comprising Formula (1). The GlcNAc of Formula (1) corresponds to the GlcNAc of Formula (11). The (Fuc)_{b} of Formula (1) corresponds to the (Fuc)_{b} of Formula (11), and both of (Fuc)_{b}. The Fuc* of Formula (1) corresponds to the Fuc*′ of Formula (11). The GalX of Formula (1) corresponds to the GalX′ of Formula (11).

For example, Formula (1) may not comprise a Fuc linked to the GlcNAc through an α1,6 linkage, and the oligosaccharide may comprise

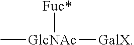

For example, Formula (1) may comprise a fucose linked to the GlcNAc through an α1,6 linkage, and the oligosaccharide may comprise

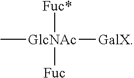

In some embodiments, the oligosaccharide is linked to an Asparagine (Asn) residue of said protein.

In some embodiments, the GlcNAc of Formula (1) and/or Formula (11) is directly linked to an amino acid of said protein. In some embodiments, the GlcNAc of Formula (1) and/or Formula (11) is directly linked to an Asn residue of said protein.

In some embodiments, the GlcNAc of Formula (1) and/or Formula (11) is indirectly linked to linked to an amino acid of the protein. For example, a saccharide may be between the GlcNAc of Formula (1) and/or Formula (11) and an amino acid of the protein. For example, one or more mannoses and GlcNAc may be between the GlcNAc of Formula (1) and/or Formula (11) and an amino acid of the protein. In some embodiments, when the GlcNAc of Formula (1) and/or Formula (11) is linked to a mannose, preferably b is 0.

In the present disclosure, the protein of the protein conjugate may comprise a Fc fragment. In the present disclosure, the oligosaccharide comprising Formula (1) and/or Formula (11) may be located in the Fc fragment. When the protein of the protein conjugate comprises a Fc fragment, the oligosaccharide comprising the Formula (1) and/or Formula (11) may be located in the CH₂ domain of the Fc fragment. For example, the oligosaccharide comprising Formula (1) and/or Formula (11) may be linked to the Asn297 of said Fc fragment, numbered according to the Kabat numbering system.

For example, the protein of the protein conjugate may be a Fc fusion protein. The protein of the protein conjugate may comprise a Fc fragment and a biologically active protein. For example, the biological active protein may be a therapeutic protein. For example, the biological active protein may be derived from a non-immunoglobulin. For example, the biological active protein may be a cytokine, a complement, and/or an antigen, or a fragment thereof.

In the present disclosure, the protein of the protein conjugate may comprise an antigen binding fragment. In the present disclosure, the oligosaccharide comprising Formula (1) and/or Formula (11) may located in the antigen binding fragment. For example, the protein of the protein conjugate may comprise nanobody, ScFv, Fab, F(ab)₂, F(ab′) and/or F(ab′)₂.

In the present disclosure, the protein of the protein conjugate may comprise a Fc fragment and an antigen binding fragment. In the present disclosure, the protein may be an antibody or a fragment thereof. In one aspect of the application, the antibody may recognize a target antigen. In some embodiments, the target antigen is a tumor antigen and may be localized to a tumor cell's surface. In some cases, the antibody bound to the target antigen can be internalized after binding to the tumor cell. When the antibody is covalently linked to a molecule of interest, the molecule of interest can be released into the cell after internalization. For example, when the functionalized antibody is linked to a cytotoxic drug, the cytotoxic drug can be released into the cell after internalization, resulting in cell death. In some cases, the target antigen displays differential expression between normal cells and tumor cells, displaying increased expression on tumor cells. For example, the target antigen may be selected from the group consisting of trop2, Her2, CD20 and VEGF.

In the present disclosure, the protein may be an antibody or a fragment thereof. For example, the antibody could be but not limited trastuzumab, bevacizumab, rituximab, durvalumab, pertuzumabetc, raxibacumab, dinutuximab, ixekizumab, labetuzumab, odesivimab. risankizumab, dinutuximab, adalimumab, cetuximab, daratumumab, tocilizumab, and etc. For example, the antibody may be trastuzumab, rituximab, bevacizumab or hRS7. For example, the heavy chain of trastuzumab may comprise the amino acid sequence as set forth in SEQ ID NO: 11, and the light chain of trastuzumab may comprise the amino acid sequence as set forth in SEQ ID NO: 10. For example, the heavy chain of rituximab may comprise the amino acid sequence as set forth in SEQ ID NO: 13, and the light chain of rituximab may comprise the amino acid sequence as set forth in SEQ ID NO 12. For example, the heavy chain of bevacizumab may comprise the amino acid sequence as set forth in SEQ ID NO: 15, and the light chain of bevacizumab may comprise the amino acid sequence as set forth in SEQ ID NO: 14. For example, the heavy chain of hRS7 may comprise the amino acid sequence as set forth in SEQ ID NO: 17, and the light chain of hRS7 may comprise the amino acid sequence as set forth in SEQ ID NO: 16.

In the present disclosure, the protein conjugate may have the similar binding affinity towards an antigen, compared to the corresponding antibody. For example, the protein may be an antibody, and the protein conjugate may have a comparable binding activity towards an antigen, compared to the corresponding antibody.

For example, the binding activity or binding affinity to an antigen of the protein conjugate in the present disclosure may be about 0.1% to about 100000% (e.g., about 1%-10000%, about 10%-1000%, or about 50%-200%) of the binding activity or binding affinity of the corresponding antibody. The binding activity to an antigen may be compared by a quantitative or a non-quantitative method. In some cases, the binding activity or binding affinity can be qualified. For example, by ELISA, under a condition that allows the protein conjugate and/or its corresponding antigen to bind to a target (e.g., an antigen), contact the protein conjugate and/or its corresponding antigen with the target (eg, antigen), determine whether a complex is formed between the protein conjugate and the target (e.g., an antigen), and determine whether a complex is formed between the corresponding and the target (e.g., an antigen). For example, the binding activity or binding affinity to a target (e.g., an antigen) may be quantified by a value. For example, the value is a Kd value. For example, the value is an OD value. For example, the value is an absorbance value. The binding affinity can be qualified by the value (e.g., OD value, KD value, or absorbance value) after statistical analysis, in which the binding affinity of the corresponding antibody may be set as 100%.

In the present disclosure, a variety of methods can be used to determine the binding activity of the protein conjugate and its corresponding antigen. The binding activity can be determined by, for example, ELISA, isothermal titration calorimetry, surface plasmon resonance, and/or biolayer interferometry. For example, the binding activity of the corresponding antibody can be set as 100%.

In the present disclosure, the Fuco of the Fuc* may link to the GlcNAc of Formula (1) through an Fuc*α1,3GlcNAc linkage.

In the present disclosure, the Fuc* may be Fuco-MOI₁, wherein said Fuco represents a fucose or fucose derivative. The Fuco may according to Formula (3)

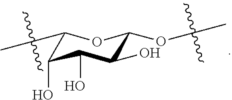

In the present disclosure, the MOI₁ may comprise an active moiety.

### X₁

In the present disclosure, the MOI₁ may comprise a chemically active moiety and/or an enzymatically active moiety. In the present disclosure, the active moiety of MOI₁ may comprises a X₁, and X₁ may be a functional group capable of participating in a ligation reaction. In some embodiment, the X₁ may be a functional group comprising a functional moiety capable of participating in a bioorthogonal ligation reaction.

For example, the X₁ may comprise a functional moiety selected from the group consisting of azido group, terminal alkynyl group, cyclic alkynyl group, tetrazinyl group, 1,2,4-trazinyl group, terminal alkenyl group, cyclic alkenyl group, ketone group, aldehyde group, hydroxyl amino group, sulfydryl group, N-maleimide group and their functional derivatives. The functional derivatives of the above functional moiety may retain the similar or higher reactivities of the functional moiety in a bioorthogonal ligation reaction. For example, the X₁ may comprise one or more functional moieties.

For example, the X₁ may comprise a functional moiety selected from the group consisting of,

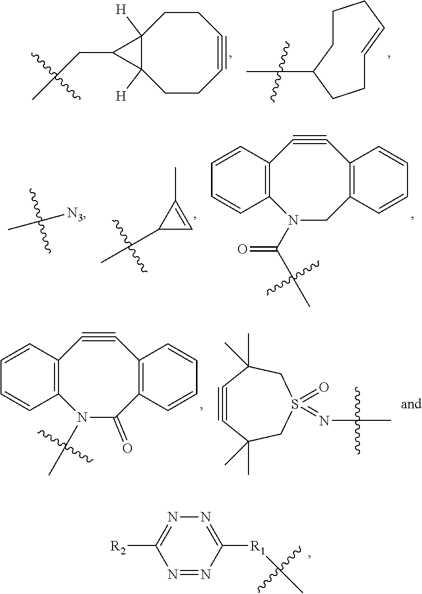

wherein R₁ is selected from the group consisting of C₁-C₂₂ alkylene group, C₅-C₂₂ (hetero)arylene group, C₆-C₂₂ alkyl(hetero)arylene group and C₆-C₂₂ (hetero)arylalkylene group, wherein the alkylene group, the (hetero)arylene group, the alkyl(hetero)arylene group or the (hetero)arylalkylene group is optionally substituted. R₂ is selected from the group consisting of hydrogen, halogen, C₁-C₂₂ alkyl group, C₅-C₂₂ (hetero)aryl group, C₆-C₂₂ alkyl(hetero)aryl group and C₆-C₂₂ (hetero)arylalkyl group, wherein the alkyl group, the (hetero)aryl group, the alkyl(hetero)aryl group or the (hetero)arylalkyl group is optionally substituted. For example, X₁ may comprise a functional moiety selected from the group consisting of

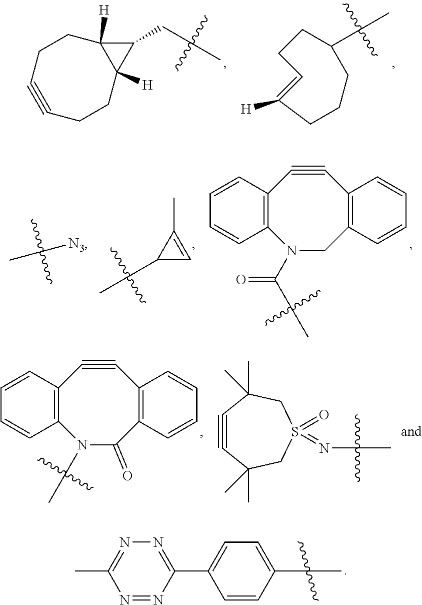

For example, the X₁ may comprise one or more functional moieties. For example, X₁ is

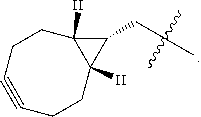 [SMILES: *C[C@H]1[C@H]2CCC#CCC[C@@H]12]

For example, X₁ is

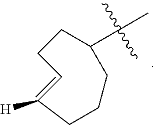

For example, X₁ is

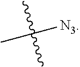 [SMILES: *N=[N+]=[N-]]

For example, X₁ is

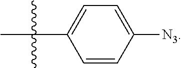 [SMILES: *c1ccc(N=[N+]=[N-])cc1]

For example, X₁ is

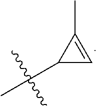 [SMILES: *C1C=C1C]

For example, X₁ is

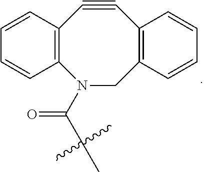 [SMILES: *C(=O)N1Cc2ccccc2C#Cc2ccccc21]

For example, X₁ is

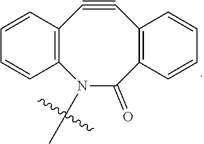 [SMILES: *N1C(=O)c2ccccc2C#Cc2ccccc21]

For example, X₁ is

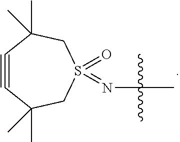 [SMILES: *N=S1(=O)CC(C)(C)C#CC(C)(C)C1]

For example, X₁ is

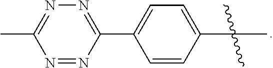 [SMILES: *c1ccc(-c2nnc(C)nn2)cc1]

In the present disclosure, the Fuc* and Fuc*′ may be Fuco-(F)ₘ-(L₁)ₙ-X₁, wherein F is a connector, L₁ is a linker, m is 0 or 1, n is 0 or 1 and X₁ is defined as above.

In the present disclosure, the X₁ may be induced to the protein by step (a2): contacting Q-Fuco-(F)ₘ-(L₁)ₙ-X₁ with the protein comprising Formula (10) -GlcNAc(Fuc)_{b}-GalX′ to obtain a protein conjugate comprising Formula (13)

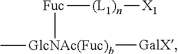

in the presence of a catalyst. For example, Fuc* and Fuc*′ may be Fuco-F-L₁-X₁. For example, Fuc* and Fuc*′ may be Fuco-F-X₁. For example, Fuc* and Fuc* may be Fuco-L₁-X₁. For example, the Fuc* and Fuc*′ may be Fuco-X₁.

### P₁

In the present disclosure, the MOI₁ may comprise a P₁, and P₁ is a biologically active moiety and/or a pharmaceutically active moiety. The P₁ itself may not participate in a ligation reaction. The P₁ may induce a biologically and/or pharmaceutically activity to the protein conjugate. In the present disclosure, the P₁ may comprise a cytotoxin, an agonist, an antagonist, an antiviral agent, an antibacterial agent, a radioisotope or a radionuclide, a metal chelator, a fluorescent dye, a biotin, an oligonucleotide, a peptide, a protein, or any combination thereof. For example, the P₁ may be a toxin, a cytokine, a growth factor, a radionuclide, a hormone, an anti-viral agent, an anti-bacterial agent, a fluorescent dye, an agent, a half-life increasing moiety, a solubility increasing moiety, a polymer-toxin conjugate, a nucleic acid, a biotin or streptavidin moiety, a vitamin, a target binding moiety, an anti-inflammatory agent or any combination thereof. For example, the P₁ may be a toxin, cytokine, a growth factor, a radionuclide, a hormone, an anti-viral agent, an anti-bacterial agent, a half-life increasing moiety, a solubility increasing moiety, a polymer-toxin conjugate, a nucleic acid, a vitamin, a target binding moiety, or an anti-inflammatory agent.

For example, the P₁ may be a pharmaceutically active moiety. For example, the P₁ may comprise a cytotoxin. For example, the P₁ may be a cytotoxin. For example, the P₁ may comprise one or more cytotoxin molecules. For example, the P₁ may be a cytotoxin selected from the group consisting of a DNA or RNA damaging agent, a topoisomerase inhibitor and a microtubule inhibitor. For example, the P₁ may be a cytotoxin selected from the group consisting of pyrrolobenzodiazepine, auristatin, maytansinoids, duocarmycin, tubulysin, enediyene, doxorubicin, pyrrole-based kinesin spindle protein inhibitor, calicheamicin, amanitin and camptothecin. For example, the P₁ may be a cytotoxin selected from the group consisting of a DNA or RNA damaging agent, a topoisomerase inhibitor and a microtubule inhibitor.

For example, the P₁ may be a cytotoxin selected from the group consisting of MMAE, MMAF, DXd, DM4 and seco-DUBA.

In the present disclosure, the Fuc* may be Fuco-(F)ₘ-(L₁)ₙ-P₁, wherein F is a connector, L₁ is a linker, m is 0 or 1, n is 0 or 1 and P₁ is defined as above.

In the present disclosure, the P₁ may be induced to the protein by step (a1): contacting Q-Fuco-(F)ₘ-(L₁)ₙ-P₁ with the protein comprising Formula (10) -GlcNAc(Fuc)_{b}-GalX′ to obtain a protein conjugate comprising Formula (12)

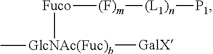

in the presence of a catalyst. For example, Fuc* and Fuc*′ may be Fuco-F-L₁-P₁. For example, Fuc* may be Fuco-F-P₁. For example, Fuc* may be Fuco-L₁-P₁. For example, Fuc* may be Fuco-F-L₁-P₁. For example, Fuc* may be Fuco-F-P₁. For example, Fuc* and Fuc*′ may be Fuco-L₁-P₁.

### Fucosylation (Transferring of Fuc*′)

In the present disclosure, the catalyst for fucosylation (e.g., in step (a), step (a1) and/or step (a2)) may be selected from the group of fucosyltransferase. In one embodiment, the Fuc*′ of the Q-Fuc*′ may be transferred to a -GlcNA(Fuc)_{b}-GalX. The fucosyltransferase may be an α-1,3-fucosyltransferase and/or a functional variant or fragment thereof. In one embodiment, the fucosyltransferase may be obtained from bacteria (e.g., Helicobacter pylori). In one embodiment, the α-1,3-fucosyltransferase is recombinantly prepared. In some embodiments, the fucosyltransferase is derived from Bacteroides fragilis. In some embodiments, the fucosyltransferase is derived from Helicobacter pylori. In some embodiments, wherein said fucosyltransferase comprises an amino acid sequence as set forth in GenBank accession no. AAB81031.1, GenBank accession no. AAD07447.1, GenBank Accession No. AAD07710.1, GenBank accession no. AAF35291.2, GenBank accession no. AAB93985.1, and/or their functional variant or fragment thereof. For example, the fucosyltransferase may comprise an amino acid sequence as set forth in GenBank Accession No. AAD07710.1, and/or a functional variant or fragment thereof. For example, a functional fragment of the amino acid sequence as set forth in GenBank Accession No. AAD07710.1 may be a catalytic domain (position 1 to position 364). For another example, a functional variant of the amino acid sequence as set forth in GenBank Accession No. AAD07710.1 may have a C₁₆₉S mutation. For another example, a functional variant of the amino acid sequence as set forth in GenBank Accession No. AAD07710.1 may have a fused C-terminal 6*His tag. For example, the fucosyltransferase comprise an amino acid sequence as set forth in GenBank Accession No. AAD07710.1, or a functional variant or fragment thereof. may have an amino acid sequence as set forth in SEQ ID NO 1 or SEQ ID NO 2. For example, the fucosyltransferase may comprise an amino acid sequence as set forth in SEQ ID NO: 1 or 2, or a functional variant or fragment thereof. For example, the fucosyltransferase may comprise an amino acid sequence as set forth in SEQ ID NO: 1 or 2 with a C₁₆₉S mutation.

In the present disclosure, said Q-Fuc*′ may comprise a donor and a Fuc*′. The donor may comprise uridine diphosphate (UDP), guanosine diphosphate (GDP) and/or cytidine diphosphate (CDP).

In the present disclosure, the Q-Fuc*′ may comprise a GDP, the fucose or fucoses derivative, optionally connector F, optionally the linker L₁, and an molecule of interest (e.g. a functional group X₁ or an active moiety P₁), In some embodiments, the Q-Fuc*′ is a GDP-Fuco-(F)ₘ-(L₁)ₙ-X₁. In some embodiments, the Q-Fuc*′ is a GDP-Fuco-(F)ₘ-(L₁)ₙ-P₁, wherein said Fuco is

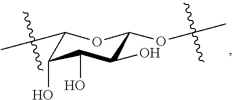

m is 0 or 1 and n is 0 or 1, the right part of the Fuco is linked to the GDP.

In the present disclosure, the contacting in fucosylation (e.g., in step (a), step (a1) and/or step (a2)) may be performed in a suitable buffer solution, such as for example phosphate, buffered saline (e.g. phosphate-buffered saline, tris-buffered saline), citrate, HEPES, tris, tris-HCl and glycine. Suitable buffers are known in the art. In some embodiments, the buffer solution is Tris-HCl buffer containing Mg²⁺.

The contacting in fucosylation (e.g., in step (a), step (a1) and/or step (a2)) may be performed at a temperature in the range of about 0 to about 50° C. In some embodiments, the method may be performed at a temperature in the range of about 5 to about 45° C. In some embodiments, the method may be performed at a temperature in the range of about 20 to about 40° C. In some embodiments, the method may be performed at a temperature in the range of about 20 to about 30° C. For example, the method may be performed at a temperature of about 37° C. For example, the method may be performed at a temperature of about 30° C.

The contacting in fucosylation (e.g., in step (a), step (a1) and/or step (a2)) may be performed at a pH in the range of about 4 to about 10. In some embodiments, the method may be performed at a pH in the range of about 5. to about 9. In some embodiments, the method may be performed at a pH in the range of about 6 to about 8. In some embodiments, the method may be performed at a pH in the range of about 7 to about 8, for example, in the range of about 7 to about 7.5.

### “One-Step” Process

In the present disclosure, when the Fuc* on the protein conjugates is prepared directly by a glycotranferring reaction by using a Q-Fuc*′ comprising a functional group or a biologically or pharmaceutically in the presence of an α1,3-fucotrasferase without a second ligation step, the process is named as the “one-step” process. In such cases, the Fuc* and Fuc*′ are the same. For example, the Q-Fuc*′ is a GDP-Fuco-(F)ₘ-(L₁)ₙ-P₁ or GDP-Fuco-(F)ₘ-(L₁)ₙ-X₁ and the Fuc* is Fuco-(F)ₘ-(L₁)ₙ-P₁ or Fuco-(F)ₘ-(L₁)ₙ-X₁.

### “Two-Step” Process

In the present disclosure, when the Fuc* on the protein conjugates is prepared by a glycotranferring reaction by using a Q-Fuc*′ in the presence of an α1,3-fucotrasferase, and followed by a second ligation reaction through a functional moiety, the process is named as the “two-step” process. In such cases, the Fuc* and Fuc*′ are different. The Fuc*′ may be an intermediate for making the Fuc*. For example, the Q-Fuc*′ is GDP-Fuco-(F)ₘ-(L₁)ₙ-X₁ and the Fuc* is Fuco-(F)ₘ-(L₁)ₙ-X₁Y₁-(FL′)_{m′}-(L₁′)_{n′}-P₁.

In some embodiments, the protein conjugate prepared from the “one-step” process may have better hydrophility.

Y₁ and X₁Y₁

In the present disclosure, the P₁ may be induced to the protein by a ligation reaction after fucosylation of a protein. For example, the P₁ may be induced to the protein by the step (a2) described above and a step (b): contacting said protein conjugate comprising Formula (13)

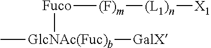

with a Y₁-(FL′)_{m′}-(L₁′)_{n′}-P₁, to obtain a protein conjugate comprising Formula (14)

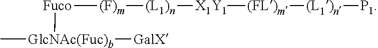

In these cases, the MOI₁ of Fuc* may comprise a X₁Y₁, and X₁Y₁ is a remaining group after a ligation reaction between the functional group X₁ and a functional group Y₁. In these cases, the Fuc* may be Fuco-(F)ₘ-(L₁)ₙ-X₁Y₁-(FL′)_{m′}-(L₁′)_{n′}-P₁, wherein F is a connector, L₁ is a linker, FL′ is a spacer, L₁′ is a linker, m is 0 or 1, n is 0 or 1, m′ is 0 or 1, n′ is 0 or 1, and P₁ is defined as above. For example, the Fuc* may be Fuco-X₁Y₁-FL′-L₁′-P₁. For example, the Fuc* may be Fuco-X₁Y₁-FL′-P₁. For example, the Fuc* may be Fuco-X₁Y₁-L₁′-P₁. For example, the Fuc* may be Fuco-F-X₁Y₁-L₁′-P₁. For example, the Fuc* may be Fuco-F-X₁Y₁-FL′-P₁. For example, the Fuc* may be Fuco-F-X₁Y₁-FL′-L₁′-P₁.

In the present disclosure, the Y₁ may comprise a functional moiety selected from the group consisting of azido group, terminal alkynyl group, cyclic alkynyl group, tetrazinyl group, 1,2,4-trazinyl group, terminal alkenyl group, cyclic alkenyl group, ketone group, aldehyde group, hydroxyl amino group, sulfydryl group, N-maleimide group and their functional derivatives. The functional derivatives of the above functional moiety may retain the similar or higher reactivities of the functional moiety in a bioorthogonal ligation reaction

In the present disclosure, the Y₁ may comprise a functional moiety selected from the group consisting of

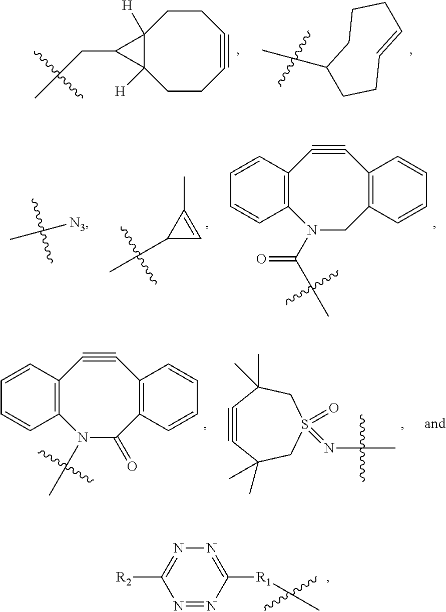

wherein each of R₁ is selected from the group consisting of C₁-C₂₂ alkylene group, C₅-C₂₂ (hetero)arylene group, C₆-C₂₂ alkyl(hetero)arylene group and C₆-C₂₂ (hetero)arylalkylene group, wherein the alkylene group, the (hetero)arylene group, the alkyl(hetero)arylene group or the (hetero)arylalkylene group is optionally substituted. R₂ is selected from the group consisting of hydrogen, halogen, C₁-C₂₂ alkyl group, C₅-C₂₂ (hetero)aryl group, C₆-C₂₂ alkyl(hetero)aryl group and C₆-C₂₂ (hetero)arylalkyl group, wherein the alkyl group, the (hetero)aryl group, the alkyl(hetero)aryl group or the (hetero)arylalkyl group is optionally substituted.

In the present disclosure, the Y₁ may comprise a functional moiety selected from the group consisting of

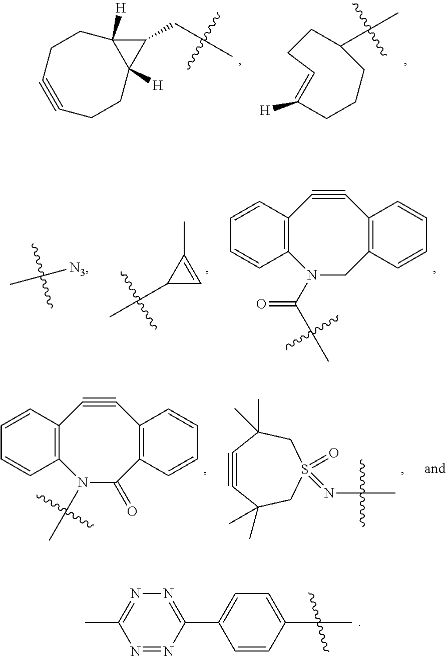

For example, Y₁ is

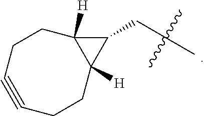 [SMILES: *C[C@H]1[C@H]2CCC#CCC[C@@H]12]

For example, Y₁ is

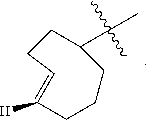

For example, Y₁ is

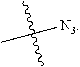 [SMILES: *N=[N+]=[N-]]

For example, Y₁ is

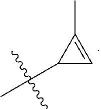 [SMILES: *C1C=C1C]

For example, Y₁ is

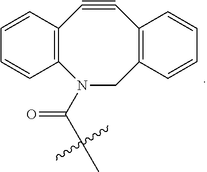 [SMILES: *C(=O)N1Cc2ccccc2C#Cc2ccccc21]

For example, Y₁ is

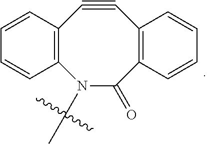 [SMILES: *N1C(=O)c2ccccc2C#Cc2ccccc21]

For example, Y₁ is

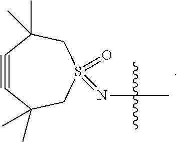 [SMILES: *N=S1(=O)CC(C)(C)C#CC(C)(C)C1]

For example, Y₁ is

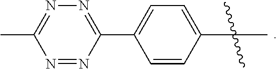 [SMILES: *c1ccc(-c2nnc(C)nn2)cc1]

For example, the X₁Y₁ may be selected from the group consisting of

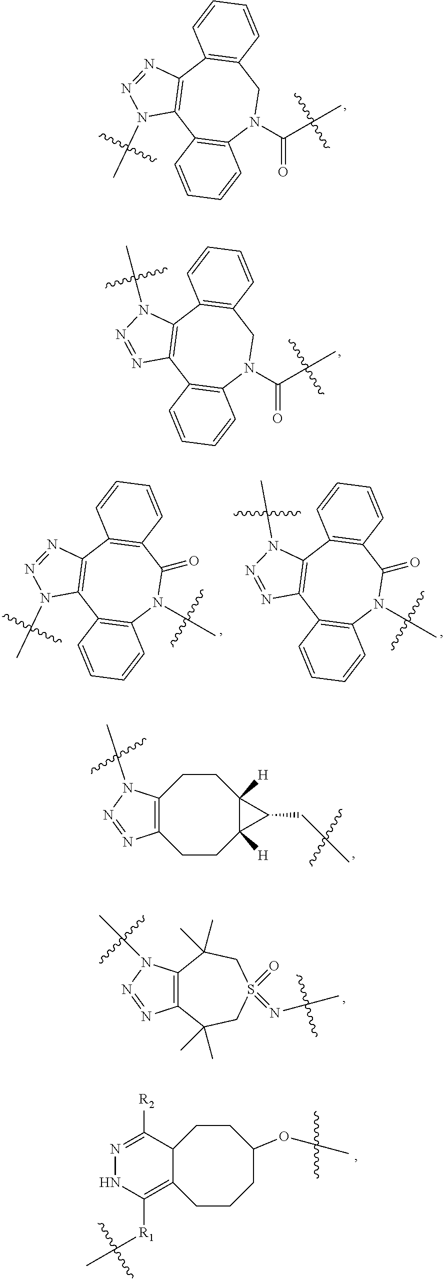

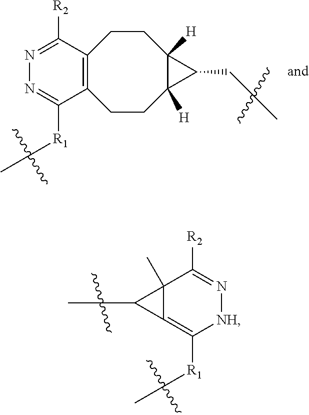

wherein R₁ is selected from the group consisting of C₁-C₂₂ alkylene group, C₅-C₂₂ (hetero)arylene group, C₆-C₂₂ alkyl(hetero)arylene group and C₆-C₂₂ (hetero)arylalkylene group, wherein the alkylene group, the (hetero)arylene group, the alkyl(hetero)arylene group or the (hetero)arylalkylene group is optionally substituted. R₂ is selected from the group consisting of hydrogen, halogen, C₁-C₂₂ alkyl group, C₅-C₂₂ (hetero)aryl group, C₆-C₂₂ alkyl(hetero)aryl group and C₆-C₂₂ (hetero)arylalkyl group, wherein the alkyl group, the (hetero)aryl group, the alkyl(hetero)aryl group or the (hetero)arylalkyl group is optionally substituted.

For example, the X₁ and the Y₁ may comprise the functional moieties selected from the group consisting of:
a) X₁ comprises

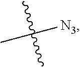 [SMILES: *N=[N+]=[N-]]

and Y₁ comprises

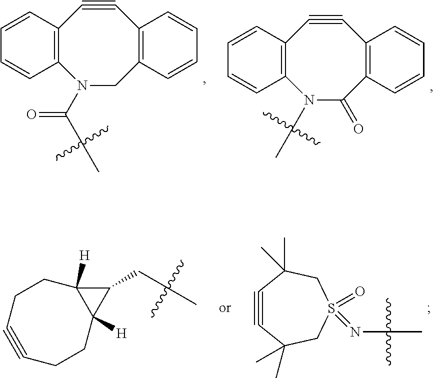

b) X₁ comprises

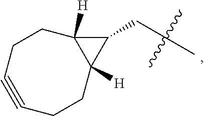 [SMILES: *C[C@H]1[C@H]2CCC#CCC[C@@H]12]

and Y₁ comprises

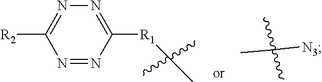

c) X₁ comprises

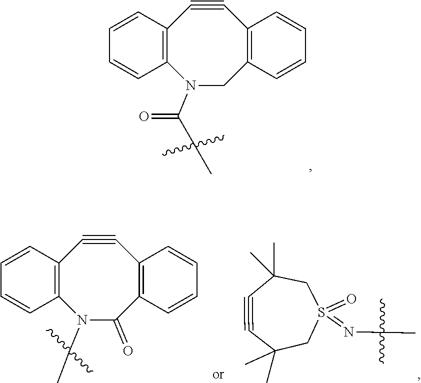 [SMILES: *C(=O)N1Cc2ccccc2C#Cc2ccccc21 ; *N1C(=O)c2ccccc2C#Cc2ccccc21 ; *N=S1(=O)CC(C)(C)C#CC(C)(C)C1]

and Y₁ comprises

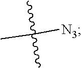 [SMILES: *N=[N+]=[N-]]

d) X₁ comprises

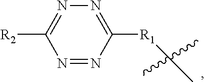

and Y₁ comprises

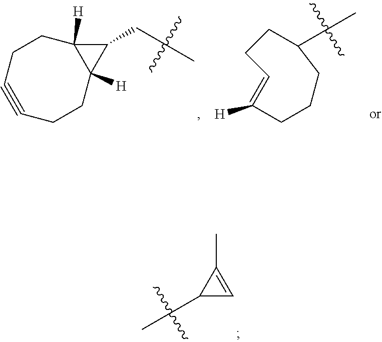

and e) X₁ comprises

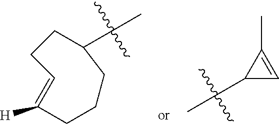

and Y₁ comprises

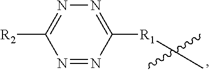

wherein each of R₁ and R₂ is defined as above.

### Connector, Jointer and Spacer

In the present disclosure, F is a connector which links X₁, P₁, L₁, or X₁Y₁ to the Fuco of the Fuc* or Fuc*′. The F may comprise a jointer J and/or a spacer FL. For example, J may be a

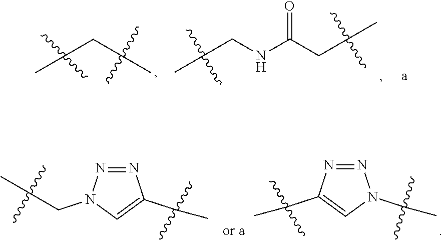

In the present disclosure, the FL may be capable of (1) tuning the distance between the Fuco and the X₁, P₁, L₁, or X₁Y₁ in the protein conjugate; (2) tuning the hydrophility of the protein conjugate and/or the GDP-Fuc*′ comprising the FL or (3) tuning the conformation of the Fuc* or Fuc*′ For example, a spacer derived from PEG may increase the hydrophility of the protein conjugate.

In the present disclosure, F may according to Formula (2): (J)_{q}-(FL)ₛ, wherein q is 0 or 1 and s is 0 or 1.

In the present disclosure, F may according to Formula (2): J-(FL)ₛ, wherein s is 0 or 1, J may be a

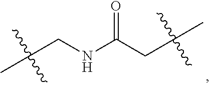 [SMILES: *CNC(=O)C*]

FL may be selected from the group consisting of C₃-C₂₀₀ peptide, C₂-C₂₀₀ PEG, C₁-C₂₀₀ alkylene group, C₃-C₂₀₀ cycloalkylene group, C₂-C₂₀₀ alkenylene group, C₅-C₂₀₀ cycloalkenylene group, C₂-C₂₀₀ alkynylene group, C₅-C₂₀₀ cycloalkynylene group, C₂-C₂₄ (hetero)arylene group, C₃-C₂₀₀ (hetero)arylalkylene group, C₃-C₂₀₀ alkynyl(hetero)arylene group, their derivatives and any combination thereof, wherein said the peptide, the PEG, the alkylene group, the cycloalkylene group, the alkenylene group, the cycloalkenylene group, the alkynylene group, the cycloalkynylene group, the (hetero)arylene group, the (hetero)arylalkylene group, or the alkynyl(hetero)arylene group is optionally substituted by one or more Rs¹ and/or is optionally interrupted by one or more Rs², wherein Rs¹ is selected from the group consisting of halogen, —OH, —NH₂ and —COOH, Rs² is independently selected from the group consisting of —O—, —S—,

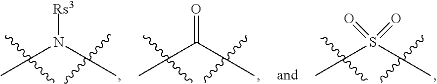

wherein Rs³ is selected from the group consisting of hydrogen, C₁-C₂₄ alkyl group, C₂-C₂₄ alkenyl group, C₂-C₂₄ alkynyl group and C₃-C₂₄ cycloalkyl group. For example, s is 1.

In the present disclosure, F may according to Formula (2): J-(FL)ₛ, wherein s is 0 or 1, J may be a

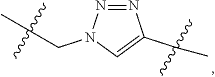 [SMILES: *Cn1cc(*)nn1]

FL may be selected from the group consisting of C₃-C₂₀₀ peptide, C₂-C₂₀₀ PEG, C₁-C₂₀₀ alkylene group, C₃-C₂₀₀ cycloalkylene group, C₂-C₂₀₀ alkenylene group, C₅-C₂₀₀ cycloalkenylene group, C₂-C₂₀₀ alkynylene group, C₅-C₂₀₀ cycloalkynylene group, C₂-C₂₄ (hetero)arylene group, C₃-C₂₀₀ (hetero)arylalkylene group, C₃-C₂₀₀ alkynyl(hetero)arylene group, their derivatives and any combination thereof, wherein said the peptide, the PEG, the alkylene group, the cycloalkylene group, the alkenylene group, the cycloalkenylene group, the alkynylene group, the cycloalkynylene group, the (hetero)arylene group, the (hetero)arylalkylene group, or the alkynyl(hetero)arylene group may be substituted by one or more Rs¹ and/or may be interrupted by one or more Rs², wherein Rs¹ is selected from the group consisting of halogen, —OH, —NH₂ and —COOH, R² is independently selected from the group consisting of —O—, —S—,

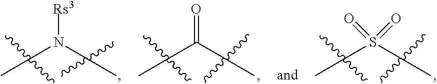

wherein Rs³ is selected from the group consisting of hydrogen, C₁-C₂₄ alkyl group, C₂-C₂₄ alkenyl group, C₂-C₂₄ alkynyl group and C₃-C₂₄ cycloalkyl group.

For example, the FL may have the structure selected from:

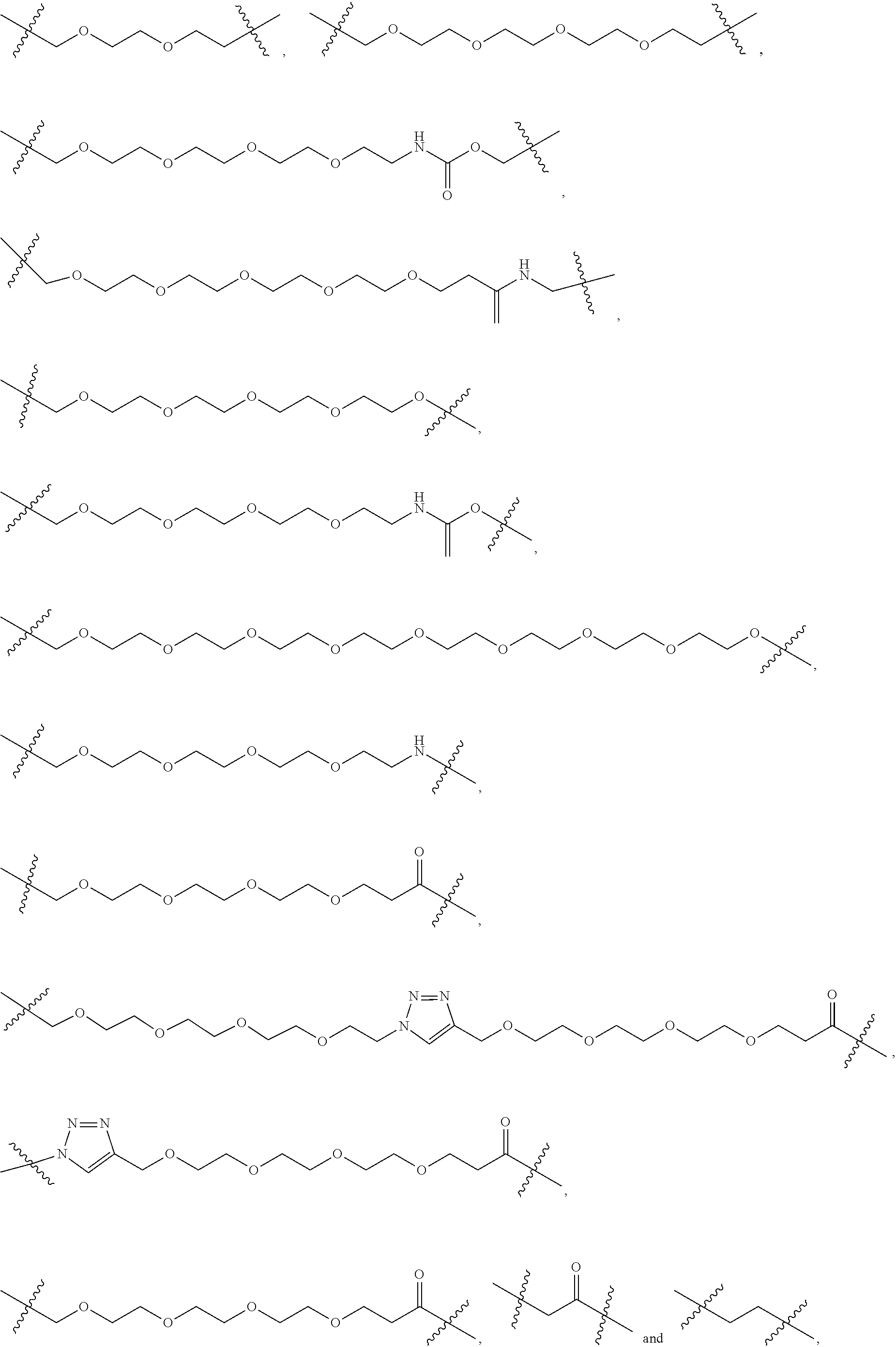

wherein the left part of the structure is directly linked to the jointer J.

For example, a FL may be an alkylene group

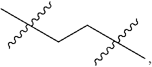 [SMILES: *CC*]

For another example, a FL of

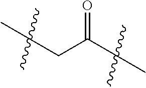 [SMILES: *CC(*)=O]

is an alkylene group interrupted by

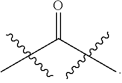 [SMILES: *C(*)=O]

For example, FL may be a

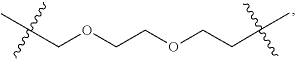 [SMILES: *CCOCCOC*]

For another example, a FL may be a PEG derivative (substituted PEG)

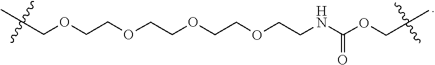 [SMILES: *COCCOCCOCCOCCNC(=O)OC*]

For another example, a FL may be a combination of substituted PEG and (hetero)arylene group

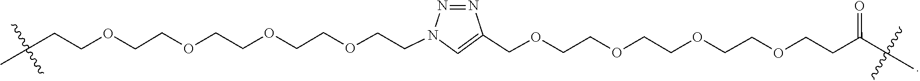 [SMILES: *CCOCCOCCOCCOCCn1cc(COCCOCCOCCOCCC(*)=O)nn1]

In the present disclosure, the structure of the jointer J may have influence on the catalytic efficiency of α-1,3-fucosyltransferase in transferring an active moiety (e.g. X₁ or P₁) to the GlcNAc of the -GlcNAc(Fuc)_{b}-GalX′ comprised by a protein, wherein b is 0 or 1.

For example, a GDP-Fuco-F-(L₁)ₙ-X₁ or a GDP-Fuco-F-(L₁)ₙ-P₁ with a jointer J of

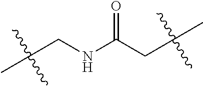 [SMILES: *CNC(=O)C*]

(the left terminus of the structure is directly linked to the Fuco) may be more efficiently to be transferred to an antibody or a Fc fusion protein comprising the -GlcNAc(Fuc)_{b}-GalX′ in the presence of an α-1,3-fucosyltransferase, wherein b is 0 or 1 and n is 0 or 1. For example, a GDP-Fuco-F-(L₁)ₙ-X₁ or a GDP-Fuco-F-(L₁)ₙ-P₁ with a jointer J of

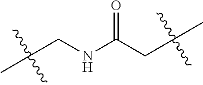 [SMILES: *CNC(=O)C*]

(the left terminus of the structure is directly linked to the Fuco) may be more efficiently to be transferred to an antibody or a Fc fusion protein comprising the -GlcNAc(Fuc)_{b}-GalX′ than those with a jointer of

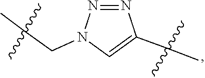

wherein b is 0 or 1 and n is 0 or 1.

In the present disclosure, in the structure of Fuco-(F)ₘ-(L₁)ₙ-X₁Y₁-(FL′)_{m′}-(L₁′)_{n′}-P₁ or Y₁-(FL′)_{m′}-(L₁′)_{n′}-P₁, when m′ is 1, FL′ is a spacer that connects the Y₁ and the L₁′, or the Y₁ and P₁. For example, the FL′ may be selected from the group consisting of C₃-C₂₀₀ peptide, C₂-C₂₀₀ PEG, C₁-C₂₀₀ alkylene group, C₃-C₂₀₀ cycloalkylene group, C₂-C₂₀₀ alkenylene group, C₅-C₂₀₀ cycloalkenylene group, C₂-C₂₀₀ alkynylene group, C₅-C₂₀₀ cycloalkynylene group, C₂-C₂₄ (hetero)arylene group, C₃-C₂₀₀ (hetero)arylalkylene group, C₃-C₂₀₀ alkynyl(hetero)arylene group, their derivatives and any combination thereof, wherein said the peptide, the PEG, the alkylene group, the cycloalkylene group, the alkenylene group, the cycloalkenylene group, the alkynylene group, the cycloalkynylene group, the (hetero)arylene group, the (hetero)arylalkylene group, or the alkynyl(hetero)arylene group is optionally substituted by one or more Rs¹ and/or is optionally interrupted by one or more Rs², wherein Rs¹ is selected from the group consisting of halogen, —OH, —NH₂ and —COOH, Rs² is independently selected from the group consisting of —O—, —S—,

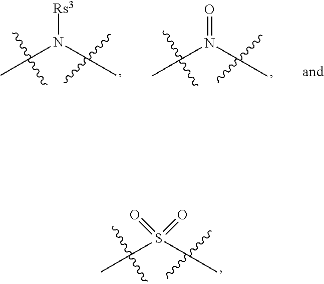

wherein Rs³ is selected from the group consisting of hydrogen, C₁-C₂₄ alkyl group, C₂-C₂₄ alkenyl group, C₂-C₂₄ alkynyl group and C₃-C₂₄ cycloalkyl group.

For example, the FL′ may have a structure selected from

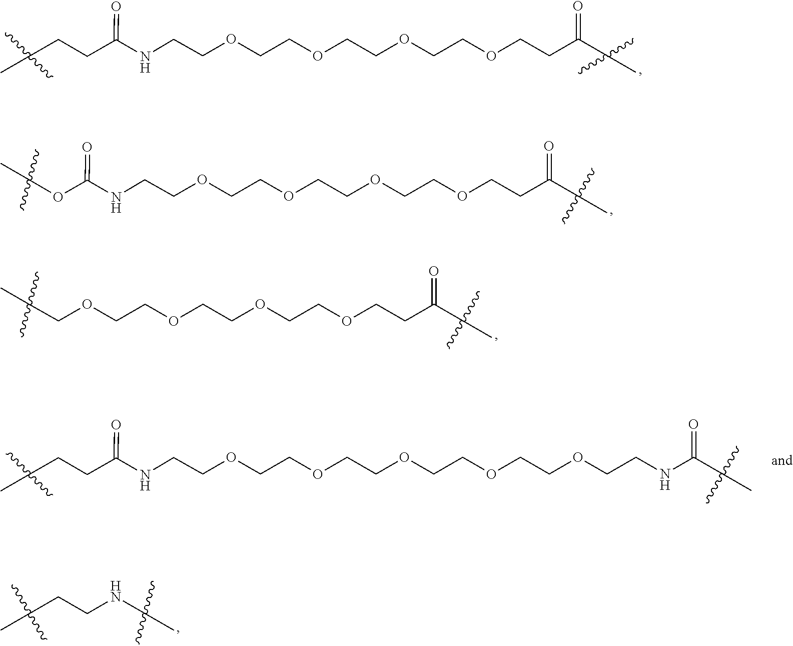 [SMILES: *CCC(=O)NCCOCCOCCOCCOCCC(*)=O ; *OC(=O)NCCOCCOCCOCCOCCC(*)=O ; *COCCOCCOCCOCCC(*)=O ; *CCC(=O)NCCOCCOCCOCCOCCOCCNC(*)=O ; *CCN*]

wherein the left part of the structure is directly linked to the Y₁.

### Linker

In the present disclosure, the MOI₁ of the Fuc* may comprise a L₁, and L₁ is a linker. In some circumstances, e.g., in a special range of pH, in a special range of temperature, or in presence of an enzyme, the linker may be cleaved, and the P₁ of MOI₁ can exert a biologically and/or pharmaceutically activity in vivo or in vitro, or the X₁ of MOI₁ can exert a chemically and/or enzymatically activity in vivo or in vitro, depended on where the protein of the protein conjugate is. For example, L₁ is a cleavable linker. A lot of type of cleavable linkers in the art can be used in the present disclosure. For example, the L₁ may be an acid-labile linker, a redox-active linker, a photo-active linker and/or a proteolytically cleavable linker. For example, the L₁ may be a vc-PAB linker, a GGFG linker or a disulfo linker.

In the present disclosure, when Fuc* is Fuco-(F)ₘ-(L₁)ₙ-X₁Y₁-(FL′)_{m′}-(L₁′)_{n′}-P₁, the MOI₁ may comprise a L₁′, and L₁′ is a linker. In some circumstances, e.g., in a special range of pH, in a special range of temperature, or in presence of an enzyme, the linker may be cleaved, and the P₁ of MOI₁ can exert a biologically and/or pharmaceutically activity in vivo or in vitro, depended on where the protein of the protein conjugate is. For example, L₁′ is a cleavable linker. A lot of type of cleavable linkers in the art can be used in the present disclosure. For example, the L₁′ may be an acid-labile linker, a redox-active linker, a photo-active linker and/or a proteolytically cleavable linker. For example, the L₁′ may be a vc-PAB linker, a GGFG linker or a disulfo linker.

In the present disclosure, the Fuco of Fuc* may be according to Formula (3):

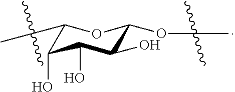

n the present disclosure, the Fuc* may be according to Formula (4):

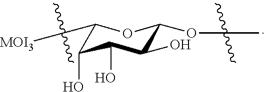

In the present disclosure, the C1 position of Fuco is linked to GlcNAc in a protein conjugate comprising Fuc*. The C1 position of Fuco may be linked to a GDP in GDP-Fuc*′. In some embodiment, in a “one-step” process, Fuc* and Fuc*′ are the same, Fuc* and Fuc*′ are Fuco-(F)ₘ-(L₁)ₙ-P₁ or Fuco-(F)ₘ-(L₁)ₙ-X₁. For Example, when m is 0, n is 0 and X₁ is

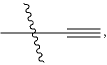 [SMILES: *C#C]

### Fuc* or Fuc*′ is

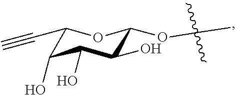

### GDP-Fuc*′ is

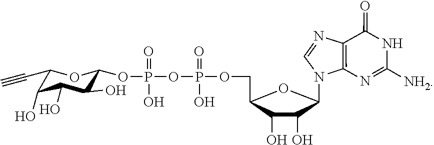

For another example, when m is 1, n is 0, F is

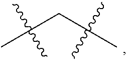 [SMILES: *C*]

X₁ is —N₃, Fuc* or Fuc*′ is

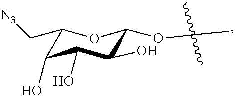

### GDP-Fuc*′ is

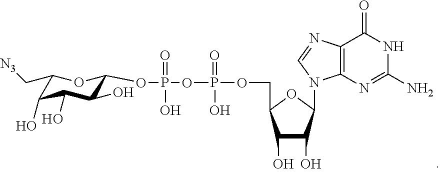

For another example, when m is 1, n is 0, F is

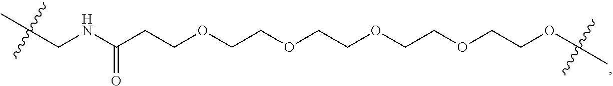 [SMILES: *CNC(=O)CCOCCOCCOCCOCCO*]

### Fuc* or Fuc*′ is

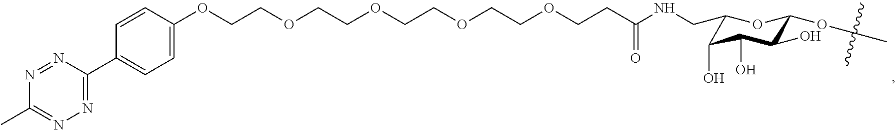

### GDP-Fuc*′ is

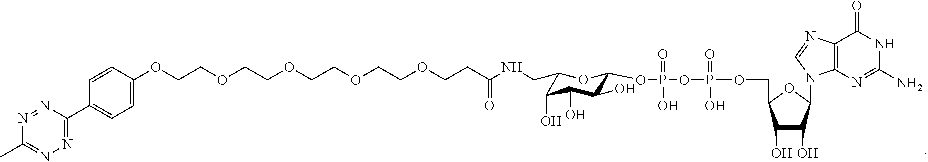

For another example, when m is 1, n is 1, F is

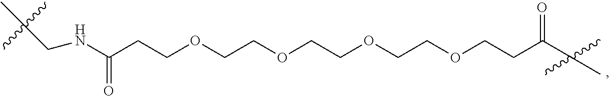 [SMILES: *CNC(=O)CCOCCOCCOCCOCCC(*)=O]

L₁ is a vc-PAB linker, P₁ is a MMAE, Fuc* or Fuc*′ is

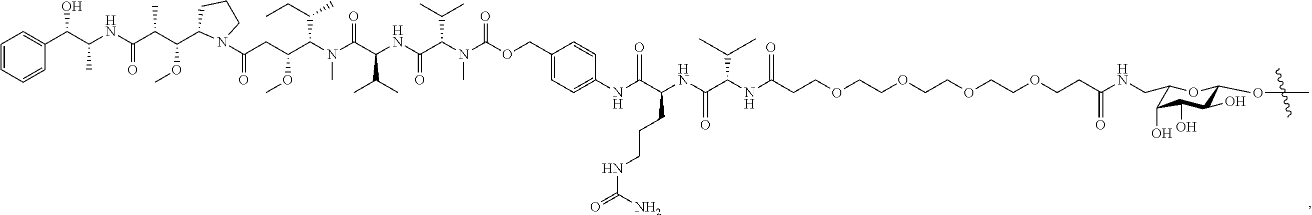

### GDP-Fuc*′ is

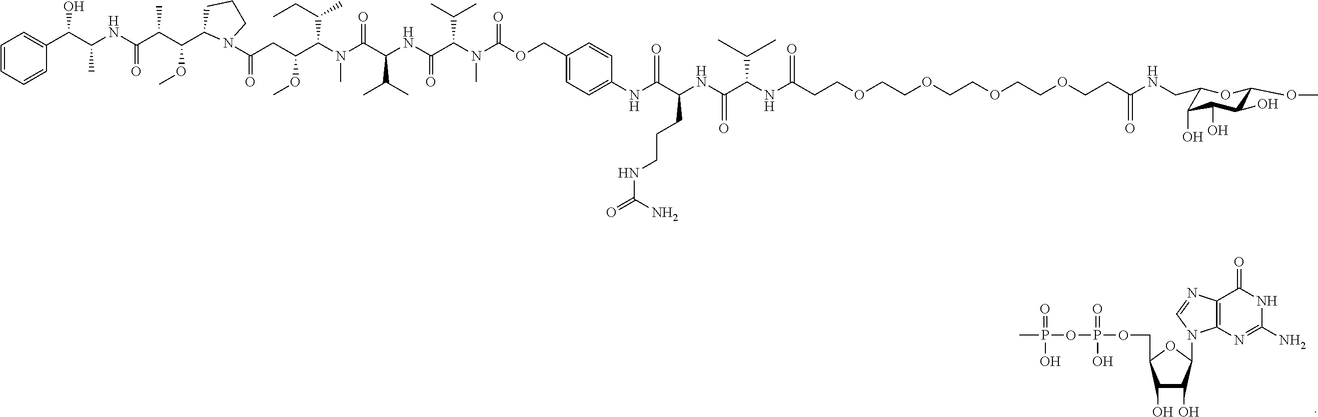

In some embodiment, Fuc* is Fuco-(F)ₘ-(L₁)ₙ-X₁Y₁-(FL′)_{m′}-(L₁′)_{n′}-P₁ and is generated from the “two-step” process (step (a2) and step (b)). For example, m is 1, n is 0, m′ is 1, L₁′ is 1, F is

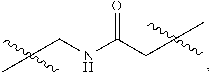 [SMILES: *CNC(=O)C*]

X₁Y₁ is

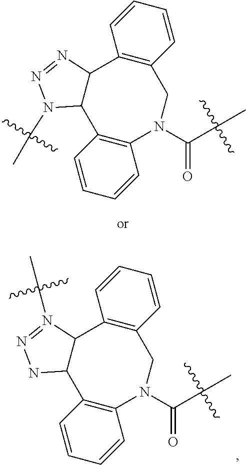

### FL′ is

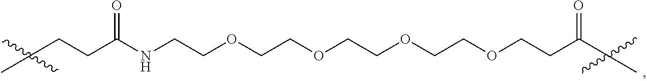 [SMILES: *CCC(=O)NCCOCCOCCOCCOCCC(*)=O]

L₁′ is vc-PAB linker and P₁ is MMAE, Fuc* is

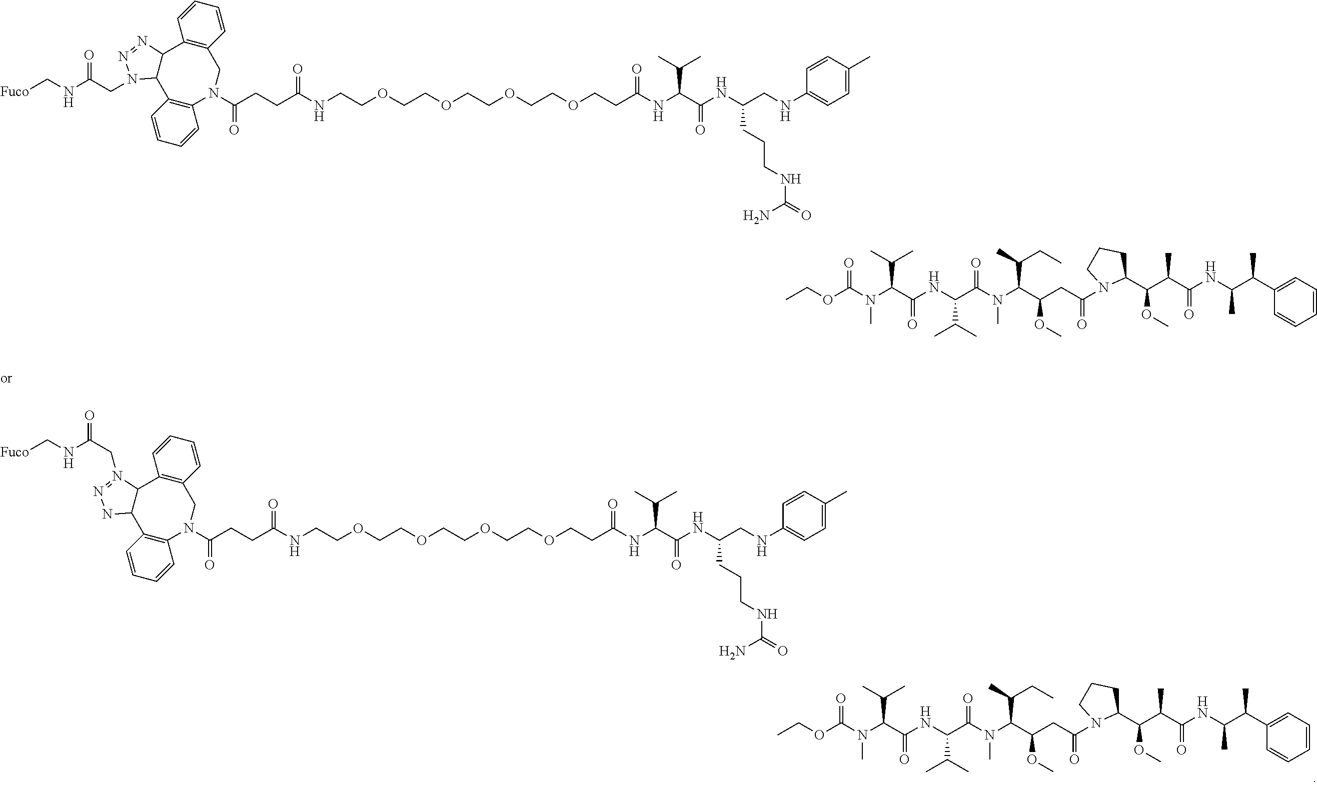

In the present disclosure, the C1 position of the GalX (or GalX′) is linked to a GlcNAc when the GalX (or GalX′) is in a protein conjugate. The C1 position of the GalX′ is linked to a UDP when the GalX′ is in a UDP-GalX′.

In the present disclosure, when the GalX is obtained directly through a glycotrasfering reaction using the UDP-GalX′ and a catalyst, GalX is GalX′. In some embodiments, GalX is GalX′, and GalX′ is GalX₀. For example, GalX₀ is

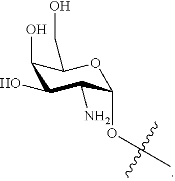

For example, GalX₀ is

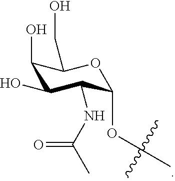

In some embodiments, GalX is GalX′, and GalX′ is GalX₂. For example, GalX₂ is

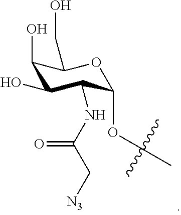

For example, GalX₂ is

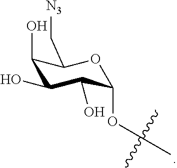

In the present disclosure, when the GalX is obtained through a glycotrasfering reaction using the UDP-GalX′, and followed by a ligation reaction. In such cases, the GalX and GalX′ are different. The GalX′ may be a intermediate for making the GalX. In some embodiments, GalX is GalX₂Y₂-(FL″)_{m″}-(L₂)_{n″}-P₂. generated by reacting GalX₂ with Y₂-(FL″)_{m″}-(L₂)_{n″}-P₂. For example, when m″ is 1, n″ is 1, FL″ is

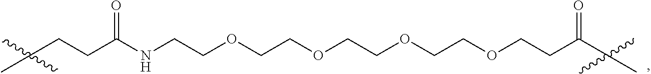 [SMILES: *CCC(=O)NCCOCCOCCOCCOCCC(*)=O]

X₂Y₂ is

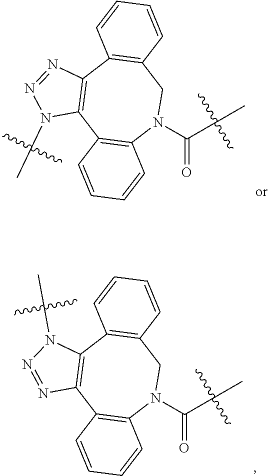 [SMILES: *C(=O)N1Cc2ccccc2-c2nnn(*)c2-c2ccccc21 ; *C(=O)N1Cc2ccccc2-c2c(nnn2*)-c2ccccc21]

L₂ is vc-PAB linker and P₂ is a MMAE, GalX is

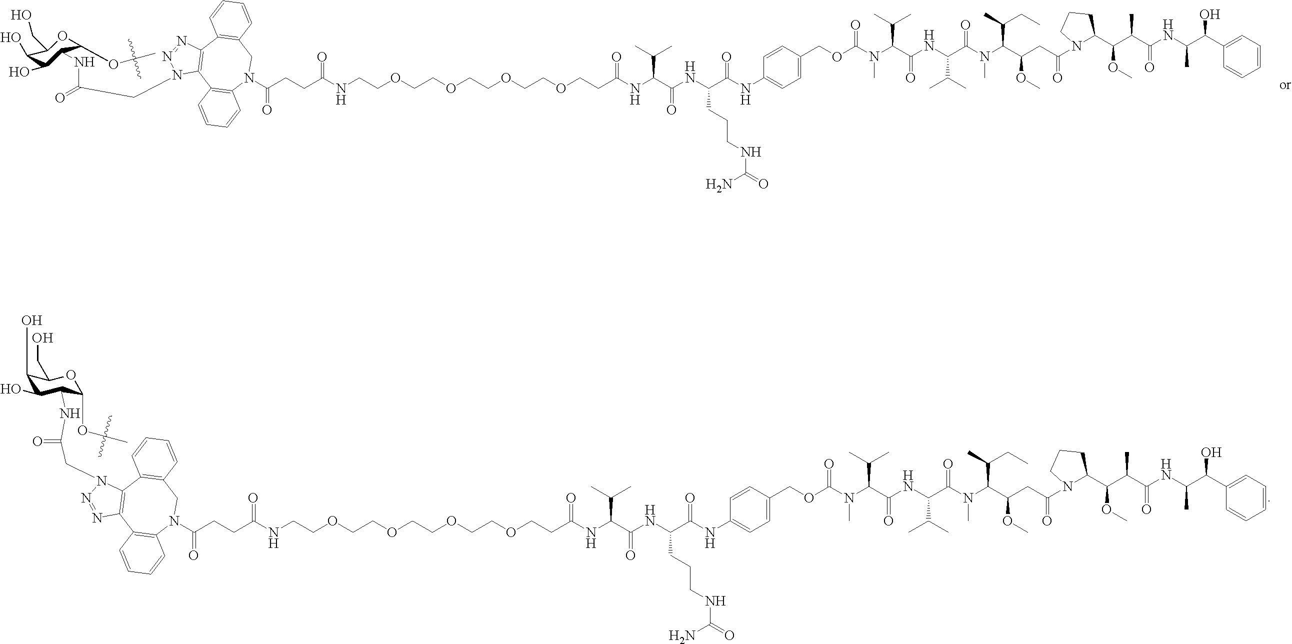

### GalX and GalX′

In the present disclosure, the GalX may be linked to the GlcNAc through a GalXβ1,4GlcNAc linkage. Correspondingly, the GalX′ may be linked to the GlcNAc through a GalX′β1,4GlcNAc linkage.

In the present disclosure, the GalX or the GalX′ may be a substituted galactose. Generally, a substituted galactose is not a natural galactose. In some embodiments, the galactose refers to a D-galactose.

In the present disclosure, the GalX or the GalX′ may be a substituted galactose, and may be substituted on one or more positions selected from the C2 position, the C3 position, the C4 position and the C6 position of the galactose. In some embodiments, the hydroxyl group on one or more positions selected from the C2 position, the C3 position, the C4 position and the C6 position of the galactose, is substituted.

In the present disclosure, the GalX or the GalX′ may be a substituted galactose, and may be substituted on the C2 position and/or the C6 position. For example, the GalX or the GalX′ may be a substituted galactose, wherein the hydroxyl group on the C2 position of the galactose may be substituted. For example, the GalX or the GalX′ may be a substituted galactose, wherein the hydroxyl group on the C6 position of the galactose may be substituted. For example, the GalX or the GalX′ may be a substituted galactose, wherein the hydroxyl group on the C3 position of the galactose may be substituted. For example, the GalX or the GalX′ may be a substituted galactose, wherein the hydroxyl group on the C4 position of the galactose may be substituted. For example, the GalX or the GalX′ may be a substituted galactose, wherein both of the hydroxyl groups on the C2 position and the C6 position of the galactose may be substituted.

In the present disclosure, the GalX or the GalX′ may be a monosaccharide. In the present disclosure, the GalX or the GalX′ may not be substituted by a monosaccharide. In the present disclosure, the GalX or the GalX′ may only linked with one saccharide (e.g., GlcNAc). In some embodiments, the GalX or GalX′ is substituted by a substitution Rg and the Rg is according to Formula (5):

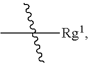

wherein Rg¹ is selected from the group consisting of hydrogen, halogen, —NH₂, —SH, —N₃, —COOH, —CN, C₁-C₂₄ alkyl group, C₃-C₂₄ cycloalkyl group, C₂-C₂₄ alkenyl group, C₅-C₂₄ cycloalkenyl group, C₂-C₂₄ alkynyl group, C₇-C₂₄ cycloalkynyl group, C₂-C₂₄ (hetero)aryl group, C₃-C₂₄ alkyl(hetero)aryl group, C₃-C₂₄ (hetero)arylalkyl group and any combination thereof, wherein the alkyl group, the cycloalkyl group, the alkenyl group, the cycloalkenyl group, the alkynyl group, the cycloalkynyl group, the (hetero)aryl group, the alkyl(hetero)aryl group, or the (hetero)arylalkyl group is optionally substituted by one or more Rs⁴ and/or is optionally interrupted by one or more Rs², wherein Rs⁴ is selected from the group of halogen, —OH, —NH₂, —SH, —N₃, —COOH and —CN, Rs² is selected from the group consisting of —O—, —S—,

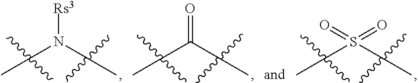

wherein Rs³ is selected from the group consisting of hydrogen, C₁-C₂₄ alkyl group, C₂-C₂₄ alkenyl group, C₂-C₂₄ alkynyl group and C₃-C₂₄ cycloalkyl group.

In some embodiments, the GalX or GalX′ is substituted by a substitution Rg and the Rg is according to Formula (6):

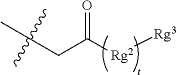

### or Formula (7):

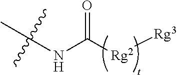

wherein t is 0 or 1, Rg² is selected from the group consisting of C₁-C₂₄ alkylene group, C₃-C₂₄ cycloalkylene group, C₂-C₂₄ alkenylene group, C₅-C₂₄ cycloalkenylene group, C₂-C₂₄ alkynylene group, C₇-C₂₄ cycloalkynylene group, C₂-C₂₄ (hetero)arylene group, C₃-C₂₄ alkyl(hetero)arylene group and C₃-C₂₄ (hetero)arylalkylene group, wherein the alkylene group, the cycloalkylene group, the alkenylene group, the cycloalkenylene group, the alkynylene group, the cycloalkynylene group, the (hetero)arylene group, the alkyl(hetero)arylene group or the (hetero)arylalkylene group is optionally substituted by one or more Rs⁴ and/or is optionally interrupted by one or more Rs², Rg³ is selected from the group consisting of hydrogen, halogen, —OH, —NH₂, —SH, —N₃, —COOH, —CN, C₁-C₂₄ alkyl group, C₃-C₂₄ cycloalkyl group, C₂-C₂₄ alkyne group, C₅-C₂₄ cycloalkyne group, C₂-C₂₄ alkynyl group, C₅-C₂₄ cycloalkynyl group, C₂-C₂₄ (hetero)aryl group and any combination thereof, wherein the C₁-C₂₄ alkyl group, the C₃-C₂₄ cycloalkyl group, the C₂-C₂₄ alkyne group, the C₅-C₂₄ cycloalkyne group, the C₂-C₂₄ alkynyl group, the C₅-C₂₄ cycloalkynyl group, or the C₂-C₂₄ (hetero)aryl group is optionally substituted by one or more Rs⁴, Rs⁴ is selected from the group of halogen, —OH, —NH₂, —SH, —N₃, —COOH and —CN, Rs² is selected from the group consisting of —O—, —S—,

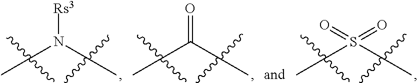

wherein Rs³ is selected from the group consisting of hydrogen, C₁-C₂₄ alkyl group, C₂-C₂₄ alkenyl group, C₂-C₂₄ alkynyl group and C₃-C₂₄ cycloalkyl group.

In the present disclosure, when the GalX is obtained directly through a glycotrasfering reaction using the UDP-GalX′ and a catalyst, the GalX is the same as GalX′.

In the present disclosure, when the GalX is obtained through a glycotrasfering reaction using the UDP-GalX′, and followed by a ligation reaction. In such cases, the GalX and GalX′ are different. The GalX′ may be an intermediate for making the GalX. For example, the GalX′ is GalX₂ and the GalX is GalX₂Y₂-(FL″)_{m″}-(L₁″)_{n″}-P₂.

### GalX₂

In the present disclosure, the GalX of Formula (1) may comprise a X₂, and X₂ is a functional group capable of participating in a ligation reaction. In this case, GalX is represented by GalX₂. In this case, Formula (10) -GlcNAc(Fuc)_{b}-GalX′ can be represented by Formula (10-1) -GlcNAc(Fuc)_{b}-GalX₂. In this case, Formula (13)

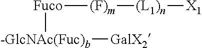

can be represented by Formula (1-7)

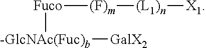

In this case, Formula (12)

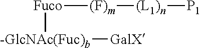

can be represented by Formula (1-8)

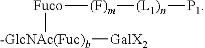

In this case, Formula (14)

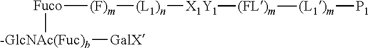

can be represented by Formula (1-9)

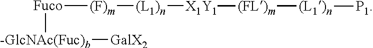

In some embodiments, the X₂ comprises a functional moiety selected from the group consisting of azido group, terminal alkynyl group, cyclic alkynyl group, tetrazinyl group, 1,2,4-trazinyl group, terminal alkenyl group, cyclic alkenyl group, ketone group, aldehyde group, hydroxyl amino group, sulfydryl group, N-maleimide group and their functional derivatives. In some embodiments, the X₂ comprises a functional moiety derived the group consisting of azido group, terminal alkynyl group, cyclic alkynyl group, tetrazinyl group, 1,2,4-trazinyl group, terminal alkenyl group, cyclic alkenyl group, ketone group, aldehyde group, hydroxyl amino group, sulfydryl group and N-maleimide group. The functional derivatives of the above functional moiety may retain the similar or higher reactivities of the functional moiety in a bioorthogonal ligation reaction.

In some embodiments, the X₂ comprises a functional moiety selected from the group consisting of

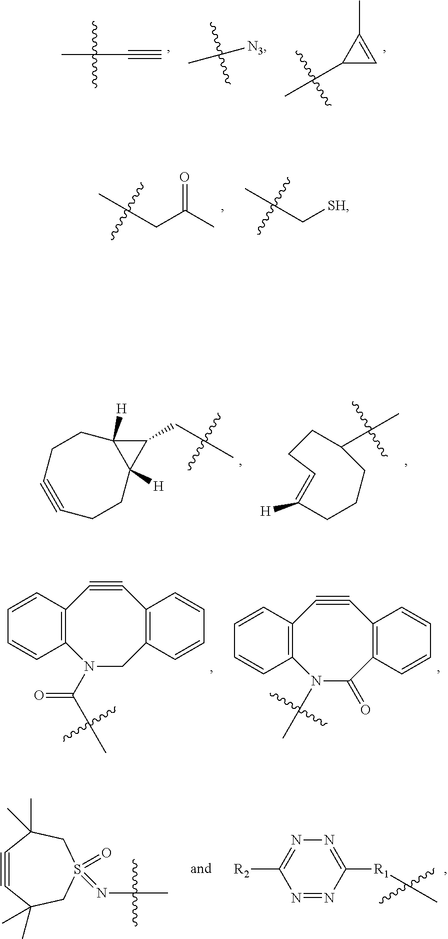

wherein R₁ is selected from the group consisting of C₁-C₂₂ alkylene group, C₅-C₂₂ (hetero)arylene group, C₆-C₂₂ alkyl(hetero)arylene group and C₆-C₂₂ (hetero)arylalkylene group, wherein the alkylene group, the (hetero)arylene group, the alkyl(hetero)arylene group or the (hetero)arylalkylene group is optionally substituted. R₂ is selected from the group consisting of hydrogen, halogen, C₁-C₂₂ alkyl group, C₅-C₂₂ (hetero)aryl group, C₆-C₂₂ alkyl(hetero)aryl group and C₆-C₂₂ (hetero)arylalkyl group, wherein the alkyl group, the (hetero)aryl group, the alkyl(hetero)aryl group or the (hetero)arylalkyl group is optionally substituted.

In some embodiments, the X₂ comprises a functional moiety selected from the group consisting of

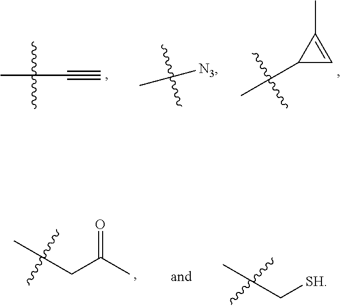

For example, X₂ is

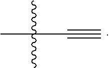 [SMILES: *C#C]

For example, X₂ is

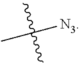 [SMILES: *N=[N+]=[N-]]

For example, X₂ is

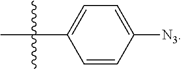 [SMILES: *c1ccc(N=[N+]=[N-])cc1]

For example, X₂ is

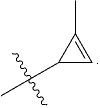 [SMILES: *C1C=C1C]

For example, X₂ is

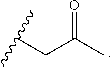 [SMILES: *CC(C)=O]

For example, X₂ is

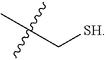 [SMILES: *CS]

### P₂

In the present disclosure, a biologically and/or a pharmaceutically active moiety P₂ may be induced to the protein conjugate by a ligation reaction between X₂ and a functional group Y₂.

For example, the P₂ may be induced to the protein by contacting Y₂-(FL″)_{m″}-(L₂)_{n″}-P₂ with GalX₂, wherein, FL″ is a spacer, L₂ is a linker, n″ is 0 or 1, and m″ is 0 or 1.

For example, the P₂ may be induced to the protein by step (ci): contacting a protein conjugate comprising Formula (1-8)

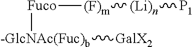

with Y₂-(FL″)_{m″}-(L₂)_{n″}-P₂, to obtain a protein conjugate comprising Formula (1-10):

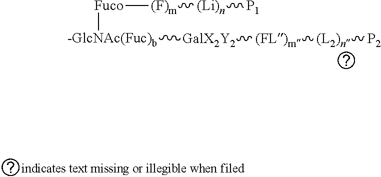

For example, the P₂ may be induced to the protein by step (c2): contacting a protein conjugate comprising Formula (1-7)

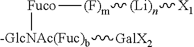

with Y₂-(FL″)_{m″}-(L₂)_{n″}-P₂ to obtain a protein conjugate comprising Formula (1-11)

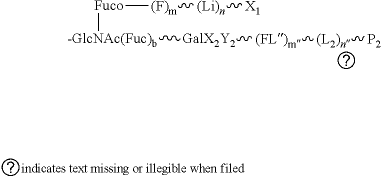

In this case, the P₁ can be induced to the protein conjugate by step (e): contacting said protein conjugate comprising Formula (1-11)

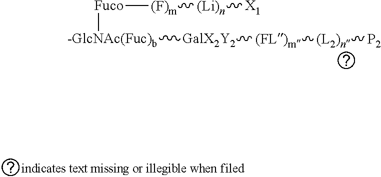

with Y₁-(FL′)_{m′}-(L₁′)_{n′}-P₁ to obtain a protein conjugate comprising Formula (1-12)

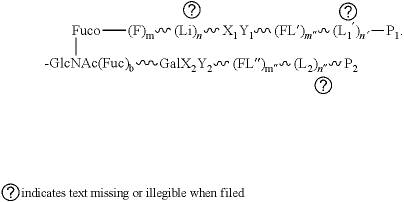

For example, the P₂ may be induced to the protein by step (d): contacting a protein conjugate comprising Formula (1-9)

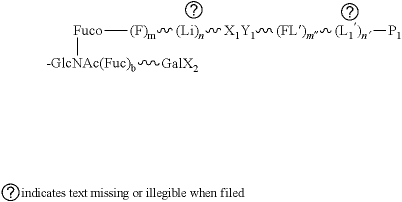

with Y₂-(FL″)_{m″}-(L₂)_{n″}-P₂ to obtain a protein conjugate comprising Formula (1-12)

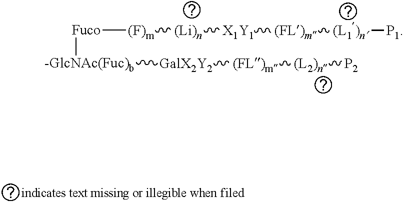

The “GalX₂Y₂” in any one of formulas of the present disclosure, generally refer to a substituted galactose comprising a remaining group X₂Y₂. “GalX₂Y₂” is obtained after the GalX₂ reacts with a molecule comprising the Y₂ (e.g. Y₂-(FL″)_{m″}-(L₂)_{n″}-P₂). The position of X₂Y₂ on “GalX₂Y₂” may be the same as the X₂ on “GalX₂”.

In the present disclosure, the P₂ itself may not participate in a ligation reaction. The P₂ may induce a biologically and/or pharmaceutically activity to the protein conjugate. In the present disclosure, the P₂ may comprise a cytotoxin, an agonist, an antagonist, an antiviral agent, an antibacterial agent, a radioisotope or a radionuclide, a metal chelator, a fluorescent dye, a biotin, an oligonucleotide, a peptide, a protein, or any combination thereof. For example, the P₂ may be a toxin, a cytokine, a growth factor, a radionuclide, a hormone, an anti-viral agent, an anti-bacterial agent, a fluorescent dye, an agent, a half-life increasing moiety, a solubility increasing moiety, a polymer-toxin conjugate, a nucleic acid, a biotin or streptavidin moiety, a vitamin, a target binding moiety, an anti-inflammatory agent or any combination thereof. For example, the P₂ may be a toxin, cytokine, a growth factor, a radionuclide, a hormone, an anti-viral agent, an anti-bacterial agent, a half-life increasing moiety, a solubility increasing moiety, a polymer-toxin conjugate, a nucleic acid, a vitamin, a target binding moiety, or an anti-inflammatory agent.

For example, the P₂ may be a pharmaceutically active moiety. For example, the P₂ may comprise a cytotoxin. For example, the P₂ may be a cytotoxin. For example, the P₂ may comprise one or more cytotoxin molecules. For example, the P₂ may be a cytotoxin selected from the group consisting of a nucleic acid (e.g., DNA or RNA) damaging agent, a topoisomerase inhibitor and a microtubule inhibitor. For example, the P₂ may be a cytotoxin selected from the group consisting of pyrrolobenzodiazepine, auristatin, maytansinoids, duocarmycin, tubulysin, enediyene, doxorubicin, pyrrole-based kinesin spindle protein inhibitor, calicheamicin, amanitin and camptothecin. For example, the P₂ may be a cytotoxin selected from the group consisting of a nucleic acid (e.g., DNA or RNA) damaging agent, a topoisomerase inhibitor and a microtubule inhibitor.

For example, the P₂ may be a cytotoxin selected from the group consisting of MMAE, MMAF, DXd, DM4 and seco-DUBA.

Y₂ and X₂Y₂

In the present disclosure, the Y₂ may comprise a functional moiety capable of participating in a bioorthogonal ligation reaction. In the present disclosure, the Y₂ may comprise a functional moiety selected from the group consisting of azido group, terminal alkynyl group, cyclic alkynyl group, tetrazinyl group, 1,2,4-trazinyl group, terminal alkenyl group, cyclic alkenyl group, ketone group, aldehyde group, hydroxyl amino group, sulfydryl group, N-maleimide group and their functional derivatives. The functional derivatives of the above functional moiety may retain the similar or higher reactivities of the functional moiety in a bioorthogonal ligation reaction.

In the present disclosure, the Y₂ may comprise a functional moiety selected from the group consisting of

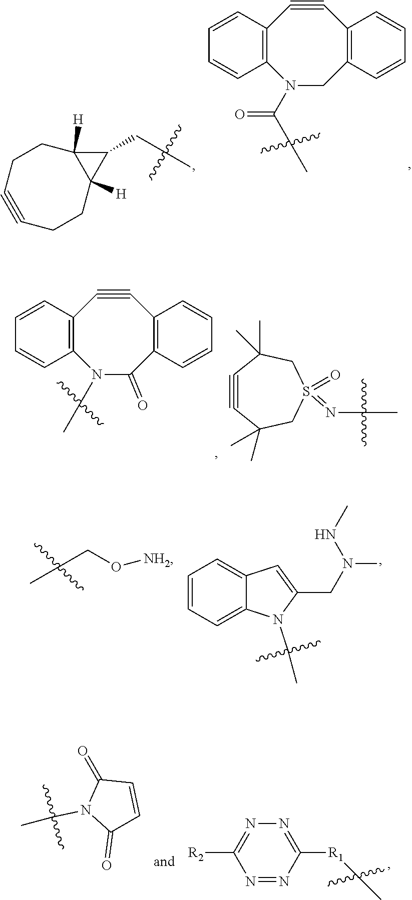

wherein R₁ is selected from the group consisting of C₁-C₂₂ alkylene group, C₅-C₂₂ (hetero)arylene group, C₆-C₂₂ alkyl(hetero)arylene group and C₆-C₂₂ (hetero)arylalkylene group, wherein the alkylene group, the (hetero)arylene group, the alkyl(hetero)arylene group or the (hetero)arylalkylene group is optionally substituted. R₂ is selected from the group consisting of hydrogen, halogen, C₁-C₂₂ alkyl group, C₅-C₂₂ (hetero)aryl group, C₆-C₂₂ alkyl(hetero)aryl group and C₆-C₂₂ (hetero)arylalkyl group, wherein the alkyl group, the (hetero)aryl group, the alkyl(hetero)aryl group or the (hetero)arylalkyl group is optionally substituted.

In the present disclosure, the Y₂ may comprise a functional moiety selected from the group consisting of

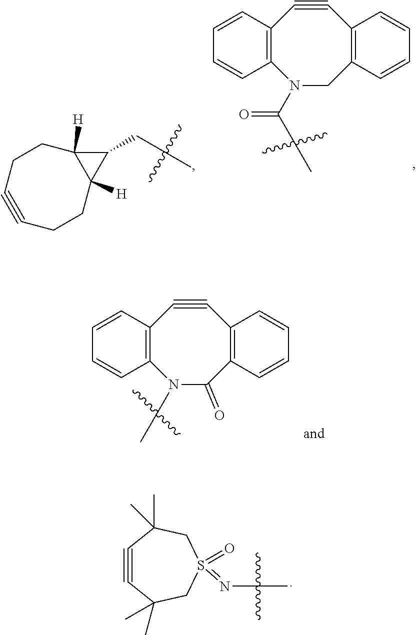

For example, Y₂ is

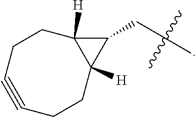

For example, Y₂ is

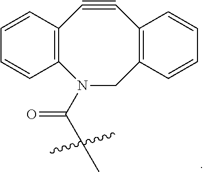 [SMILES: *C(=O)N1Cc2ccccc2C#Cc2ccccc21]

For example, Y₂ is

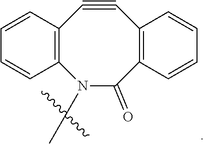 [SMILES: *N1C(=O)c2ccccc2C#Cc2ccccc21]

For example, Y₂ is

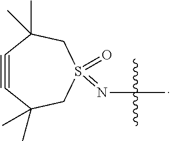

In the present disclosure, the X₂Y₂ may be selected from the group consisting of

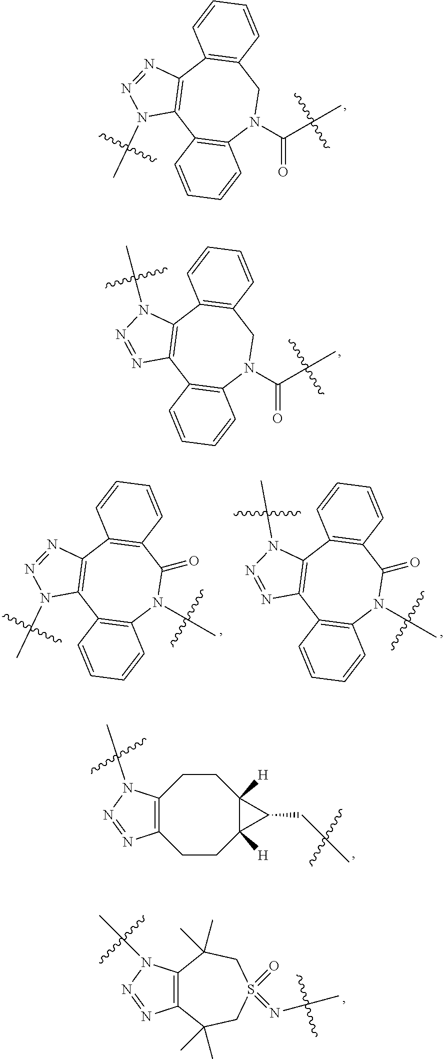

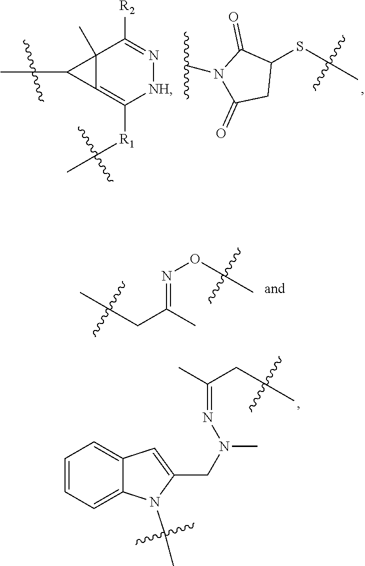

wherein R₁ is selected from the group consisting of C₁-C₂₂ alkylene group, C₅-C₂₂ (hetero)arylene group, C₆-C₂₂ alkyl(hetero)arylene group and C₆-C₂₂ (hetero)arylalkylene group, wherein the alkylene group, the (hetero)arylene group, the alkyl(hetero)arylene group or the (hetero)arylalkylene group is optionally substituted. R₂ is selected from the group consisting of hydrogen, halogen, C₁-C₂₂ alkyl group, C₅-C₂₂ (hetero)aryl group, C₆-C₂₂ alkyl(hetero)aryl group and C₆-C₂₂ (hetero)arylalkyl group, wherein the alkyl group, the (hetero)aryl group, the alkyl(hetero)aryl group or the (hetero)arylalkyl group is optionally substituted.

In the present disclosure, the X₂ and the Y₂ may comprise functional moieties selected from the group consisting of: a) X₂ comprises

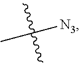 [SMILES: *N=[N+]=[N-]]

and Y₂ comprises

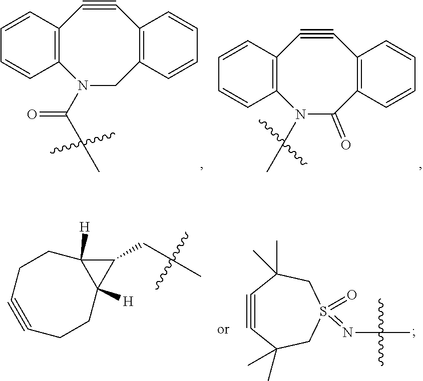

b) X₂ comprises

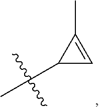 [SMILES: *C1C=C1C]

and Y₂ comprises

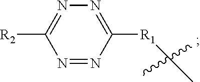

c) X₂ comprises

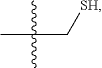 [SMILES: *CS]

and Y₂ comprises

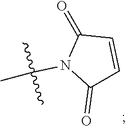 [SMILES: *N1C(=O)C=CC1=O]

and d) X₂ comprises

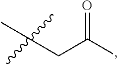

and Y₂ comprises

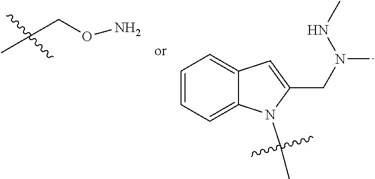

In the present disclosure, a protein conjugate comprising both an active moiety (e.g. P₁) in the Fuc* and an active moiety (e.g. P₂) in the GalX may be named as a dual-site-conjugates. And the process for making such a conjugate may be named as dual-site-conjugation process.

The dual-site-conjugated conjugates linked with to two active moieties (e.g., P₁ and P₂) would possess both of the activity of the active moiety P₁ and P₂, that may show improved functionalities compared to each of the single-conjugated conjugates.

In some embodiments, to obtain a protein conjugate comprising P₁ and P₂, the P₁ may be induced to the protein using Q-Fuco-(F)ₘ-(L₁)ₙ-P₁ by fucosylation (e.g., by step (a1)) and the P₂ may be induced to the protein by the ligation reaction between X₂ and Y₂ (e.g., by step (c1)).

For example, Formula (1-8)

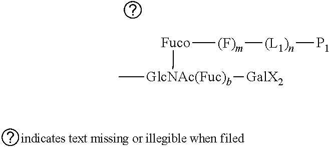

may be contacted with Y₂-(FL″)_{m″}-(L₂)_{n″}-P₂, to obtain a dual-site-conjugated protein conjugate comprising Formula (1-10):

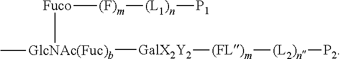

In some embodiments, to obtain a protein conjugate comprising P₁ and P₂, the P₁ may be induced to the protein using Q-Fuco-(F)ₘ-(L₁)ₙ-X₁ by fucosylation (e.g., by step (a2)) and followed by a ligation reaction between X₁ and Y₁ (e.g., by step (b), or step (e)), and the P₂ may be induced to the protein by the ligation reaction between X₂ and Y₂ (e.g., by step (c2) or step (d)). For example, the Formula (1-7)

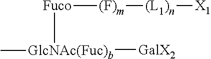

may be contacted with Y₁-(FL′)_{m′}-(L₁′)_{n′}-P₁ and/or Y₂-(FL″)_{m″}-(L₂)_{n″}-P₂. In these cases, the X₁ substantially does not react with the X₂.

For example, the X₁ and the X₂ may comprise the same functional moiety, and the Y₁ and the Y₂ may comprise the same functional moiety. Y₁-(FL′)_{m′}-(L′)_{n′}-P₁ and Y₂-(FL″)_{m″}-(L″)_{n″}-P₂ are the same molecule.

For example, the X₁ and the X₂ may comprise different functional moieties, and the Y₁ and the Y₂ may comprise the same functional moiety. For example, the X₁ may comprise

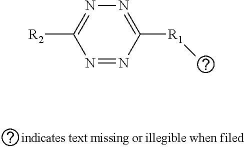

X₂ may comprise

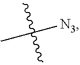 [SMILES: *N=[N+]=[N-]]

Y₁ may comprise

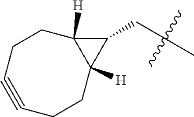 [SMILES: *C[C@H]1[C@H]2CCC#CCC[C@@H]12]

and Y₂ may comprise

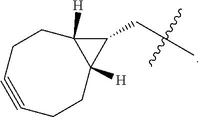 [SMILES: *C[C@H]1[C@H]2CCC#CCC[C@@H]12]

Y₁-(FL′)_{m′}-(L′)_{n′}-P₁ and Y₂-(FL″)_{m″}-(L″)_{n″}-P₂ are the same molecule.

For example, the X₁ and the X₂ may comprise different functional moieties, and the Y₁ and the Y₂ may comprise different functional moieties. For example, the reaction between the X₁ and Y₁ substantially may not affect on the reaction between the X₂ and the Y₂. For example, the X₁, the Y₁, the X₂ and the Y₂ may comprise functional moieties selected from the group consisting of: a) X₁ comprises

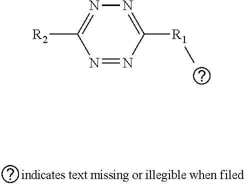

Y₁ comprises

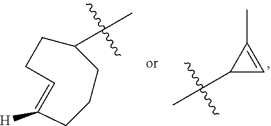

X₂ comprises

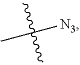 [SMILES: *N=[N+]=[N-]]

Y₂ comprises

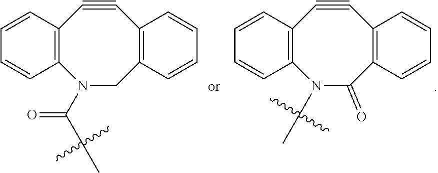

b) X₁ comprises

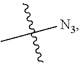 [SMILES: *N=[N+]=[N-]]

Y₁ comprises

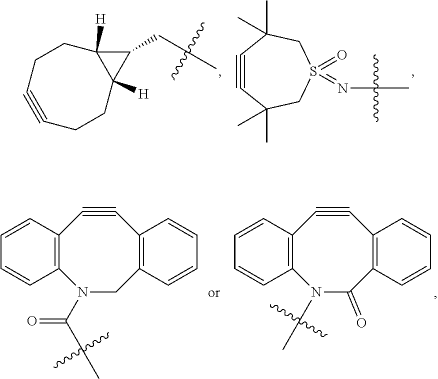

X₂ comprises

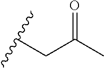 [SMILES: *CC(C)=O]

and Y₂ comprises

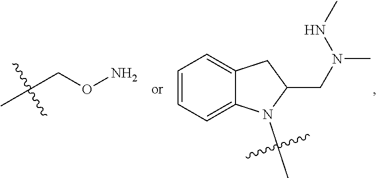

c) X₁ comprises

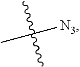 [SMILES: *N=[N+]=[N-]]

Y₁ comprises

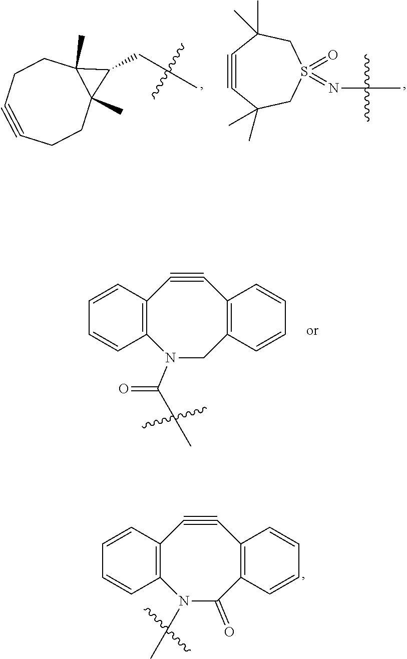 [SMILES: *C[C@@H]1[C@]2(C)CCC#CCC[C@]12C ; *N=S1(=O)CC(C)(C)C#CC(C)(C)C1 ; *C(=O)N1Cc2ccccc2C#Cc2ccccc21 ; *N1C(=O)c2ccccc2C#Cc2ccccc21]

X₂ comprises

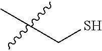 [SMILES: *CS]

and Y₂ comprises

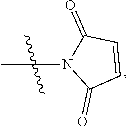 [SMILES: *N1C(=O)C=CC1=O]

and d) X₁ comprises

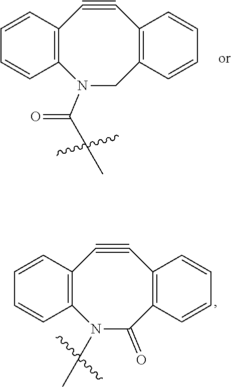 [SMILES: *C(=O)N1Cc2ccccc2C#Cc2ccccc21 ; *N1C(=O)c2ccccc2C#Cc2ccccc21]

Y₁ comprises

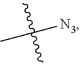 [SMILES: *N=[N+]=[N-]]

X₂ comprises

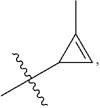 [SMILES: *C1C=C1C]

and Y₂ comprises

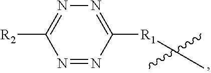

wherein R₁ is selected from the group consisting of C₁-C₂₂ alkylene group, C₅-C₂₂ (hetero)arylene group, C₆-C₂₂ alkyl(hetero)arylene group and C₆-C₂₂ (hetero)arylalkylene group, wherein the alkylene group, the (hetero)arylene group, the alkyl(hetero)arylene group or the (hetero)arylalkylene group is optionally substituted. R₂ is selected from the group consisting of hydrogen, halogen, C₁-C₂₂ alkyl group, C₅-C₂₂ (hetero)aryl group, C₆-C₂₂ alkyl(hetero)aryl group and C₆-C₂₂ (hetero)arylalkyl group, wherein the alkyl group, the (hetero)aryl group, the alkyl(hetero)aryl group or the (hetero)arylalkyl group is optionally substituted

For example, the X₁ comprises

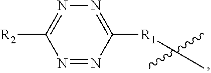

the Y₁ comprises

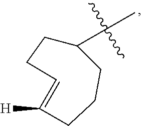

the X₂ comprises

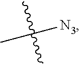 [SMILES: *N=[N+]=[N-]]

and the Y₂ comprises

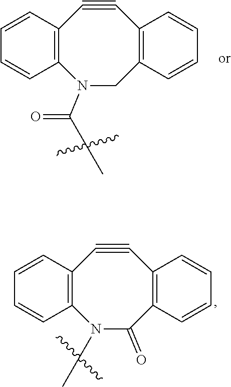 [SMILES: *C(=O)N1Cc2ccccc2C#Cc2ccccc21 ; *N1C(=O)c2ccccc2C#Cc2ccccc21]

wherein each of R₁ and R₂ is defined as above.

For example, the Formula (1-7)

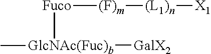

may be contacted first with Y₁-(FL′)_{m′}-(L₁′)_{n′}-P₁ and followed by contacting with Y₂-(FL″)_{m″}-(L₂)_{n″}-P₂ to obtain a dual-site-conjugated protein conjugate comprising Formula (1-12)

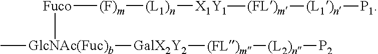

For example, the Formula (1-7)

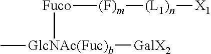

may be contacted first with Y₂-(FL″)_{m″}-(L₂)_{n″}-P₂ and followed by contacting with Y₁-(FL′)_{m′}-(L₁′)_{n′}-P₁ to obtain a dual-site-conjugated protein conjugate comprising Formula (1-12)

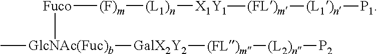

For example, the Formula (1-7)

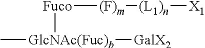

may be contacted simultaneously with Y₂-(FL″)_{m″}-(L₂)_{n″}-P₂ and Y₁-(FL′)_{m′}-(L₁′)_{n′}-P₁ to obtain a dual-site-conjugated protein conjugate comprising Formula (1-12)

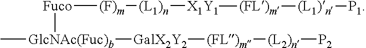

In the present disclosure, the FL″ may be selected from the group consisting of C₃-C₂₀₀ peptide, C₂-C₂₀₀ PEG, C₁-C₂₀₀ alkylene group, C₃-C₂₀₀ cycloalkylene group, C₂-C₂₀₀ alkenylene group, C₅-C₂₀₀ cycloalkenylene group, C₂-C₂₀₀ alkynylene group, C₅-C₂₀₀ cycloalkynylene group, C₂-C₂₄ (hetero)arylene group, C₃-C₂₀₀ (hetero)arylalkylene group, C₃-C₂₀₀ alkynyl(hetero)arylene group, their derivatives and any combination thereof, wherein said the peptide, the PEG, the alkylene group, the cycloalkylene group, the alkenylene group, the cycloalkenylene group, the alkynylene group, the cycloalkynylene group, the (hetero)arylene group, the (hetero)arylalkylene group, or the alkynyl(hetero)arylene group is optionally substituted by one or more Rs¹ and/or is optionally interrupted by one or more Rs², wherein Rs¹ is selected from the group consisting of halogen, —OH, —NH₂ and —COOH, Rs² is independently selected from the group consisting of —O—, —S—,

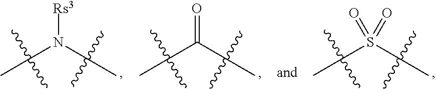

wherein Rs³ is selected from the group consisting of hydrogen, C₁-C₂₄ alkyl group, C₂-C₂₄ alkenyl group, C₂-C₂₄ alkynyl group and C₃-C₂₄ cycloalkyl group.

For example, the FL″ may have a structure selected from

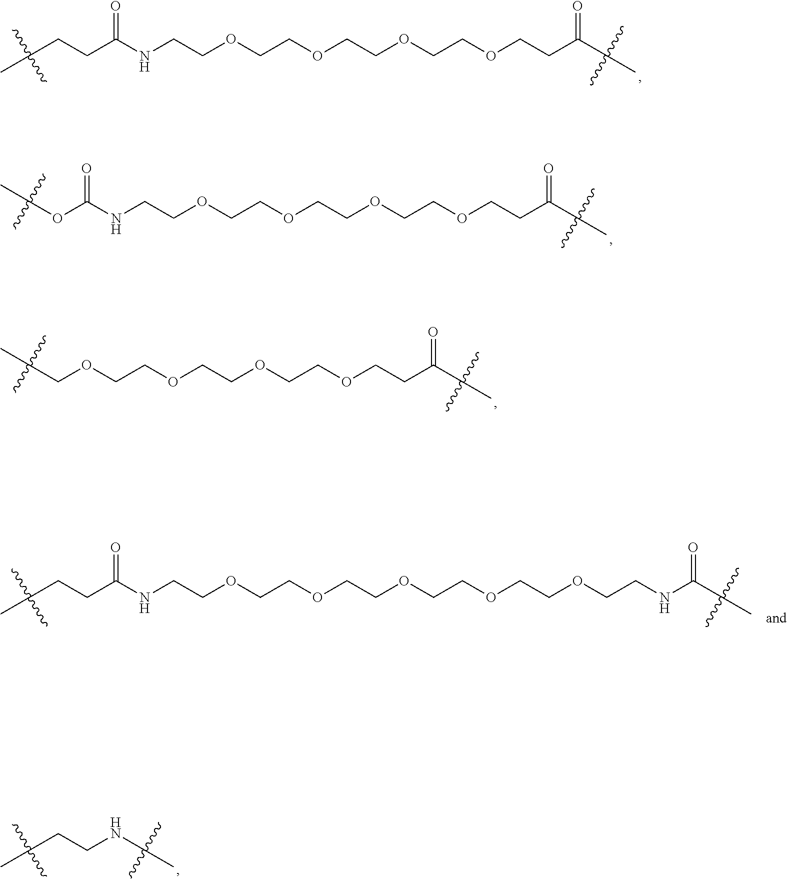 [SMILES: *CCC(=O)NCCOCCOCCOCCOCCC(*)=O ; *OC(=O)NCCOCCOCCOCCOCCC(*)=O ; *COCCOCCOCCOCCC(*)=O ; *CCC(=O)NCCOCCOCCOCCOCCOCCNC(*)=O ; *CCN*]

wherein the left part of the structure is directly linked to the Y₂.

In the present disclosure, the L₂ is a cleavable linker. In some circumstances, e.g., in a special range of pH, in a special range of temperature, or in presence of an enzyme, the linker may be cleaved, and the P₂ can exert a biologically and/or pharmaceutically activity in vivo or in vitro, depended on where the protein of the protein conjugate is. For example, L₂ is a cleavable linker. A lot of type of cleavable linkers in the art can be used in the present disclosure. For example, the L₂ may be an acid-labile linker, a redox-active linker, a photo-active linker and/or a proteolytically cleavable linker. For example, the L₂ may be a vc-PAB linker, a GGFG linker or a disulfo linker.

For example, in the above formulas, m″ is 1 and n″ is 1. For example, m″ is 0 and n″ is 1. For example, the GalX in Formula (1) is GalX₂Y₂-FL″-P₂. For example, the GalX in Formula (1) is GalX₂Y₂-FL″-L₂-P₂. For example, the GalX in Formula (1) is GalX₂Y₂-P₂.

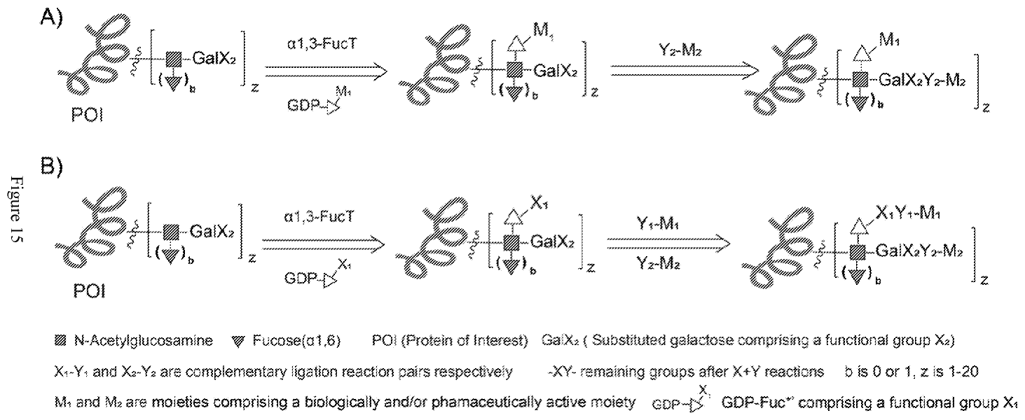

FIG. 15A shows a preferred embodiment for the preparation of a dual-site-conjugated conjugate.

FIG. 15B shows a preferred embodiment for the preparation of a dual-site-conjugates conjugate.

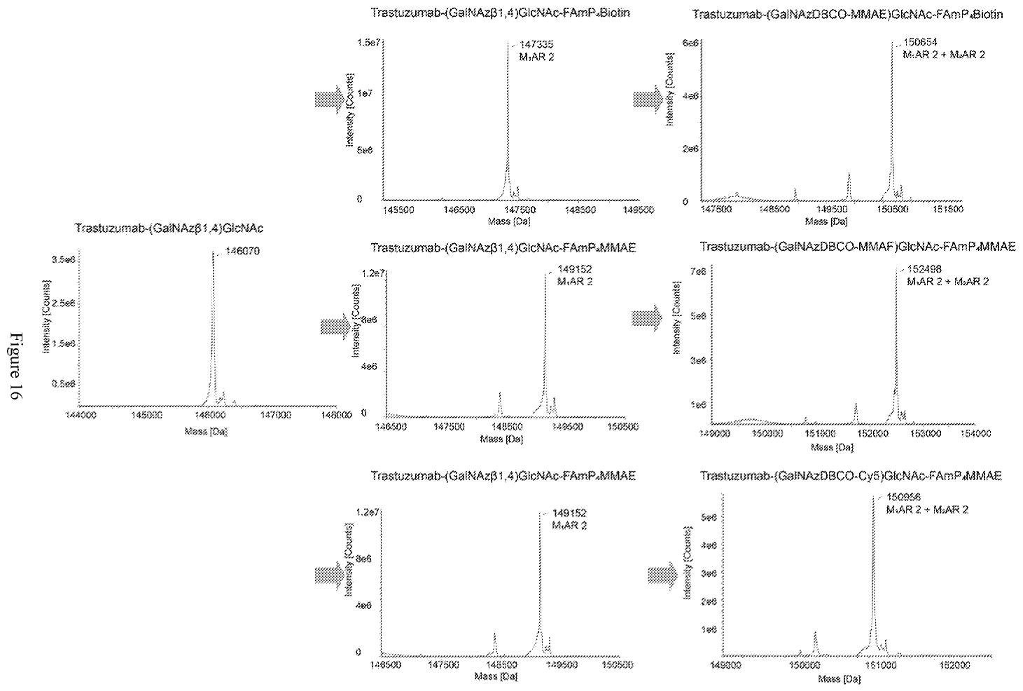

FIG. 16 and FIG. 17 shows the MS analysis of some exemplary dual-site-conjugated antibody conjugates.

### GalX₀

In the present disclosure, the GalX may not comprise a functional group capable of participating in a bioorthogonal ligation reaction. The GalX does not comprises a functional moiety capable of participating in a bioorthogonal ligation reaction may be represented by GalX₀. GalX₀ is a GalX not comprising a functional moiety capable of participating in a bioorthogonal ligation reaction, and said GalX is represented by GalX₀. In some embodiments, the GalX does not comprises a functional moiety capable of participating in a bioorthogonal ligation reaction selected from the group consisting of azido group, terminal alkynyl group, cyclic alkynyl group, tetrazinyl group, 1,2,4-trazinyl group, terminal alkenyl group, cyclic alkenyl group, ketone group, aldehyde group, hydroxyl amino group, sulfydryl group and N-maleimide group. For example, the GalX in Formula (1) is GalX₀. For example, the GalX₀ in Formula (1) may be a

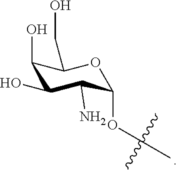

For example, the GalX₀ in Formula (1) may be a

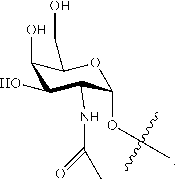

In the present disclosure, the method for preparation a protein conjugate may comprise buffer exchanging of the obtained protein conjugate into a buffer. For example, buffer exchanging of the obtained protein conjugate into a formulation buffer or a storage buffer. The buffer may comprise one or more pharmaceutically acceptable excipients. The excipient may help in improving the bioavailability or stability of the active pharmaceutical ingredient (e.g., the protein conjugate of the present disclosure) during its storage and use.

### Glycan Modification

In the present disclosure, the method further comprises a step (f): contacting a protein comprising an oligosaccharide comprising the -GlcNAc(Fuc)_{b} with a UDP-GalX′ in the presence of a catalyst, to obtain said protein comprising Formula (10): -GlcNAc(Fuc)_{b}-GalX′, wherein GalX′ is a substituted galactose, and b is 0 or 1. In the present disclosure, the catalyst may be a β1,4-galactosyltransferase, or a functional variant or fragment thereof. In some embodiments, the catalyst may be a human β1,4-galactosyltransferase, a bovine β1,4-galactosyltransferase, or a functional variant or fragment thereof. In some embodiments, the catalyst may comprise a catalytic domain of bovine β(1,4)-GalT1 with an mutation of Y289L, Y289N, Y289I, Y289F, Y289M, Y289V, Y289G, Y289I or Y289A, or a catalytic domain of human β(1,4)-GalT1 with an mutation of Y285L, Y285N, Y285I, Y285F, Y285M, Y285V, Y285G, Y285I or Y285A. In some embodiments, the catalyst may comprise an amino acid as set forth in any one of SEQ ID NOs: 3-5.

In some embodiments, the GalX′ is a C2 substituted galactose and is according to Formula 5-1

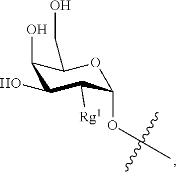

Rg¹ is defined as above. For example, GalX′ is GalNH₂ and has the structure of

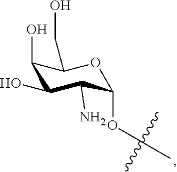

For another example, GalX′ has the structure of

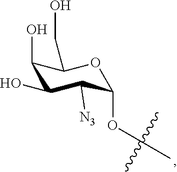

In some embodiments, the GalX′ is a C2 substituted galactose and is according to Formula 6-1

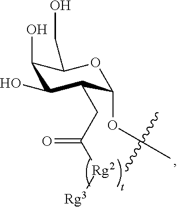

t is 0 or 1 and Rg² and Rg³ are defined as above. For another example, GalX′ has the structure of

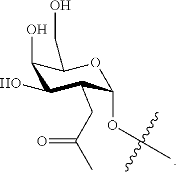

In some embodiments, the GalX′ is C₂ substituted galactose and is according to Formula (7-1)

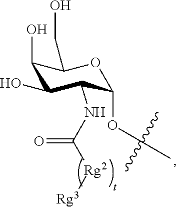

t is 0 or 1 and Rg² and Rg³ are defined as above. For example, GalX′ is GalNAc and has the structure of

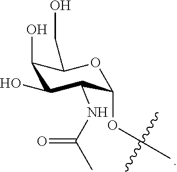

For another example, GalX′ is GalNAz and has the structure of

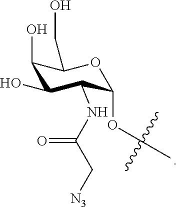

For another example, GalX′ has the structure of

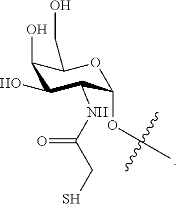

For another example, GalX′ has the structure of

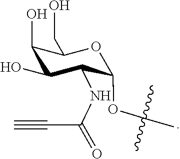

For another example, GalX′ has the structure of

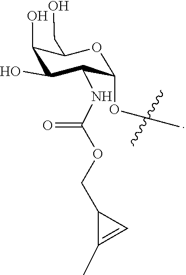

In some embodiments, the GalX′ is a C6 substituted galactose and is according to Formula (5-2)

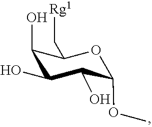

Rg¹ is defined as above. For example, GalX′ is Gal-6-Az and has the structure of

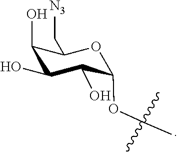

In some embodiments, the GalX′ is a C₆ substituted galactose and is according to Formula (6-2)

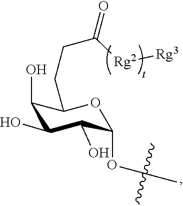

t is 0 or 1 and Rg² and Rg³ are defined as above. In some embodiments, the GalX′ is a C₆ substituted galactose and is according to Formula (7-2)

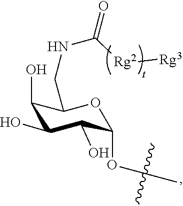

t is 0 or 1 and Rg² and Rg³ are defined as above.

In some embodiments, the GalX′ is a galactose substituted on C6 and C2. The substitutions at C6 and C2 are independently selected. For example, GlaX′ has the structure of

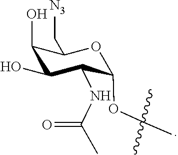

For example, GlaX′ has the structure of

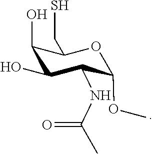

For example, GlaX′ has the structure of

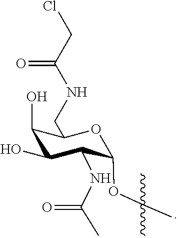

Variety structure of GalX′ and UDP-GalX′ are known in the art, e.g., US 2016/0235861 A1, US 2017/0226554 A1, US 2017/0009266 A1, those may be incorporated into the present disclosure.

For example, an antibody with heterogenous glycosylation forms of G₀(F), G₁(F), G₂(F) could be trimmed to an uniform antibody-G₀(F) by contacting with a β1,4-galactosidase. The antibody-G₀(F) could be transformed to the uniform antibody-(GalX′)₂(F) which contains four -GlcNAc-GalX′ moieties in an antibody molecule in the presence of a β1,4-galactosyltransferase and UDP-GalX′. For example, the GalX′ is a GalNAc, a GlaNAz or a GalNH₂. The antibody-(GalX′)₂(F) is according to the formula [symbol] , wherein [symbol] is a GlcNAc, [symbol] is a fucose linked to the said GlcNAc through an α1,6-linkage, [symbol] is a mannose, [symbol] is the GalX′ (substituted galactose) linked to the said GlcNAc through a GalX′ β1,4GlcNAc linkage, [symbol] is an antibody, c is 0 or 1.

For example, an antibody-GlcNAc(Fuc)_{b} could be transformed to an antibody-GlcNAc(Fuc)_{b}-GalX′ which contains two -GlcNAc(Fuc)_{b}-GalX′ moieties in an antibody molecule in the presence of a β1,4-galactosyltransferase. The antibody-GlcNAc(Fuc)_{b}-GalX (also name as antibody-(Fuc)_{b}(GalX′)GlcNAc) is according to the formula [symbol] , wherein [symbol] is the GlcNAc, [symbol] is an optional Fuc linked to the said GlcNAc through an α1,6-linkage, [symbol] is the GalX′ (substituted galactose) linked to the said GlcNAc through a GalX′β1,4GlcNAc linkage, [symbol] is an antibody, b is 0 or 1.

In the present disclosure, the contacting of galactosylation (step (f)) may be performed in a suitable buffer solution, such as for example phosphate, buffered saline (e.g. phosphate-buffered saline, tris-buffered saline), citrate, HEPES, tris, tris-HCl and glycine. Suitable buffers are known in the art.

In some embodiments, the buffer solution is Tris-HCl buffer containing Mn²⁺. The contacting of galactosylation (step (f)) may be performed at a temperature in the range of about 0 to about 50° C. In some embodiments, the method may be performed at a temperature in the range of about 5 to about 45° C. In some embodiments, the method may be performed at a temperature in the range of about 20 to about 40° C. In some embodiments, the method may be performed at a temperature in the range of about 25 to about 37° C. For example, the method may be performed at a temperature of about 30° C.

The contacting of galactosylation (step (f)) may be performed at a pH in the range of about 4 to about 10. In some embodiments, the method may be performed at a pH in the range of about 5. to about 9. In some embodiments, the method may be performed at a pH in the range of about 6 to about 8. In some embodiments, the method may be performed at a pH in the range of about 7 to about 8, for example, in the range of about 7 to about 7.5.

In the present disclosure, step (f) may be performed before step (a) (or step (a1), or step (a2)). In the present disclosure, the method may comprise a purification process between step (f) and step (a) (or step (a1), or step (a2)). In the present disclosure, the method may not comprise a purification process between step (a) (or step (a1), or step (a2)) and step (f). In the present disclosure, the step (a) (or step (a1), or step (a2)) and step (f) may be performed in the same reaction vessel. In the present disclosure, fucosyltransferase, Q-Fuco-(F)ₘ-(L₁)ₙ-X₁ or Q-Fuco-(F)ₘ-(L₁)ₙ-P₁ of step (a) (or step (a1), or step (a2)) and galactosyltransferase and UDP-galactose in step (f) may be in the same reaction vessel. In the present disclosure, step (a) (or step (a1), or step (a2)) and step (f) may be performed simultaneously. In the present disclosure, step (a) (or step (a1), or step (a2)) and step (f) may be performed at the same time. In the present disclosure, step (a) (or step (a1), or step (a2)) may be performed before step (f) was finished.

In the present disclosure, the method may further comprise a step (g) modifying a protein comprising an oligosaccharide to a protein comprising a core -(Fucα1,6) GlcNAc or core -GlcNAc, wherein in the core -(Fucα1,6)GlcNAc or core -GlcNAc, the GlcNAc is directly linked to an amino acid of the protein (the amino acid usually is a Asn), and the Fuc is linked to the GlcNAc through an α1,6 linkage. The GlcNAc of the core “-(Fucα1,6) GlcNAc” is directly linked to an amino acid of the protein. For example, the amino acid of the protein is an Asn. For example, the amino acid of the protein is Asn297. The endoglycosidase may cleave glycan chains from a glycoprotein (e.g. an antibody) and leave a core GlcNAc if the glycoprotein doesn't have an core α1,6 fucose linked to the core GlcNAc. The endoglycosidase may cleave glycan chains from a glycoprotein (e.g. an antibody or Fc-fusion protein) and leave a core -(Fucα1,6)GlcNAc if the glycoprotein have a core α1,6 fucose linked to the core GlcNAc. In the present disclosure, the endoglycosidase can modify the oligosaccharide of the antibody or Fc-fusion protein to a -GlcNAc or -(Fucα1,6)GlcNAc). In the present disclosure, the endoglycosidase may be an Endo S, Endo S2, Endo A, Endo F, Endo M, Endo D and Endo H or their functional mutants or variants, or any combination thereof. In the present disclosure, the endoglycosidase may be an EndoS. For example, the endoglycosidase may have an amino acid sequence as set forth in SEQ ID NO: 6 or 7, or a functional variant or fragment thereof.

For example, an antibody with heterogenous glycosylation forms may be trimmed to an uniform antibody-GlcNAc(Fuc) by using the endoglycosidase. The antibody-(Fucα1,6)GlcNAc (i.e. antibody-GlcNAc(Fuc)) is according to the formula [symbol] . Wherein [symbol] is a GlcNAc, [symbol] is a fucose linked the GlcNAc through an α1,6 linkage, [symbol] is an antibody or Fc-fusion protein, and the GlcNAc is linked to N297 position of the antibody. For example, an antibody with heterogenous glycosylation forms may be trimmed to a uniform antibody-GlcNAc by using the endoglycosidase. The antibody-GlcNAc is according to the formula [symbol] . Wherein [symbol] is a GlcNAc, [symbol] is an antibody or Fc-fusion protein, and the GlcNAc is linked to N297 position of the antibody.

In some embodiments, step (g) is performed before said step (f).

In the present disclosure, the method may further comprise a step (h) to remove the core α-1,6 fucose from the protein comprise a core -(Fucα1,6)GlcNAc to generate a protein comprising the core-GlcNAc. For example, step (h) may be performed in presence of a core-α1,6 fucosidase. For example, the core-α1,6 fucosidase may be a BfFucH, a fucosidase O, an Alfc, a BKF, a fucosidase O or their functional mutants or variants, or any combination thereof. For example, the core-α1,6 fucosidase may be Alfc. For example, the core-α1,6 fucosidase may have a protein sequence according to the SEQ ID NO: 8 or SEQ ID NO: 9, or a functional variant or fragment thereof.

For example, The antibody-GlcNAc(Fuc) could be further trimmed to antibody-GlcNAc by using the core-α1,6 fucosidase (e.g. Alfc). The antibody-GlcNAc is according to the formula [symbol] (e.g., the corresponding antibody), Wherein [symbol] is a GlcNAc, [symbol] is an antibody or Fc-fusion protein, and the GlcNAc is linked to N297 position of the antibody.

In the present disclosure, step (g) and/or step (h) may be performed in a suitable buffer solution, such as for example phosphate, buffered saline (e.g. phosphate-buffered saline, tris-buffered saline), citrate, HEPES, tris, tris-HCl and glycine. Suitable buffers are known in the art. In some embodiments, the buffer solution is PBS buffer.

Step (g) and/or step (h) may be performed at a temperature in the range of about 0 to about 50° C. In some embodiments, the method may be performed at a temperature in the range of about 5 to about 45° C. In some embodiments, the method may be performed at a temperature in the range of about 20 to about 40° C. In some embodiments, the method may be performed at a temperature in the range of about 20 to about 30° C. For example, the method may be performed at a temperature of about 37° C.

Step (g) and/or step (h) may be performed at a pH in the range of about 4 to about 10. In some embodiments, the method may be performed at a pH in the range of about 5. to about 9. In some embodiments, the method may be performed at a pH in the range of about 6 to about 8. In some embodiments, the method may be performed at a pH in the range of about 7 to about 8, for example, in the range of about 7 to about 7.5.

In some embodiments, step (h) is performed behind step (g) and before the step (f). In some embodiments, step (g) and step (h) are performed simultaneously. In some embodiments, step (g) and step (h) are performed in the same reaction vessel. In some embodiments, there does not comprise a purification process among step (a), step (f), step (g) and step (h). In some embodiments, there does not comprise a purification process among step (a1), step (f), step (g) and step (h). In some embodiments, there does not comprise a purification process among step (a2), step (f), step (g) and step (h). In some embodiments, step (a), step (f), step (g) and step (h) are performed in the same reaction vessel. In some embodiments, wherein step (a1), step (f), step (g) and step (h) are performed in the same reaction vessel. In some embodiments, step (a2), step (f), step (g) and step (h) are performed in the same reaction vessel.

In the present disclosure, a protein comprising a (GalX′β1,4)GlcNAc may be easier to be converted to a protein conjugate comprising (GalX′β1,4)GlcNAc-Fuc*′ than a protein comprising a (GalX′β1,4)(Fucα1,6)GlcNAc to a protein conjugate comprising a (GalX′β1,4)(Fucα1,6)GlcNAc-Fuc*′ by using a fucosyltrasferase and Q-Fuc*′. In some embodiments, the fucosyltrasferase displayed higher efficiency in transferring the Fuc*′ of GDP-Fuc*′ to (GalX′β1,4)GlcNAc than to the (GalX′β1,4)(Fucα1,6)GlcNAc, wherein the GlcNAc is directly linked to an amino acid of the protein.

M₁AR and M₂AR

In the present disclosure, the protein conjugate may have a first MOI-to-antibody ratio (M₁AR), which is a ratio of the molecule of interest in Fuc* to the protein (e.g., the antibody). In the present disclosure, the protein conjugate may have a second MOI-to-antibody ratio (M₂AR), which is a ratio of the molecule of interest in GalX to the protein (e.g., the antibody).

When the protein in the protein conjugate is an antibody, the term “first molecule of interest to antibody (M₁AR)” generally refers to the MOI₁-to-antibody ratio. M₁AR is the number of the MOI₁ comprised by the Fuc* in a protein conjugate (e.g., an antibody). The term “average M₁AR” generally refers to an average MOI₁-to-antibody ratio in a composition comprising two or more protein conjugates. In some embodiments, when the MOI₁ comprises a drug, e.g., a cytotoxin, the M₁AR can be represented by the “first drug to antibody ratio (D₁AR)”. A M₁OI may comprise one or more drugs. The value of DAR may be multiple of the value of MAR. or, the value of DAR may be equal to the value of MAR.

In the present disclosure, the value of M₁AR may be 2 or 4.

For example, when the protein conjugate is according to Formula (8): [symbol] b is 0 or 1. The value of M₁AR may be 2, and the value of D₁AR may be multiple of 2. When the composition comprises more conjugates having the Formula (8), the average M₁AR may be closed to 2.

For example, when the protein conjugate is according to Formula (9): [symbol] , c is 0 or 1. The value of M₁AR may be 4, and the value of D1AR may be multiple of 4. When the composition comprises more conjugates having the Formula (9), the average M₁AR may be closed to 4.

The M₁AR and/or D1AR may be measured by LC -MS or HIC-HPLC analysis.

In the present disclosure, in additional to the MOI, a second molecule of interest (or, an additional molecule of interest, MOI₂) may be induced to the protein conjugate. when the protein in the protein conjugate is an antibody, the term “second molecule of interest to antibody (M₂AR)” generally refers to the “second molecule of interest to antibody ratio”. In some embodiments, when the GalX comprises a functional group X₂ or a biologically and/or pharmaceutically active moiety P₂, the GalX could be the second molecule of interest. The (M₂AR) generally refers to the GalX to antibody ratio. The term “average M₂AR” generally refers to an average M₂AR in a composition comprising two or more protein conjugates of the present disclosure. In some embodiments, when the MOI₂ comprises a drug, e.g., a cytotoxin, the M₂AR can be represented by the “second drug to antibody ratio (D2AR)”. A MOI₂ may comprise one or more drugs. The value of D2AR may be multiple of the value of M₂AR or, the value of D2AR may be equal to the value of M₂AR.

In the present disclosure, the value of M₂AR may be 2 or 4

For example, when the protein conjugate is according to Formula (8): [symbol] b is 0 or 1. The value of M₂AR may be 2, and the value of D2AR may be multiple of 2. When the composition comprises more conjugates having the Formula (8), the average M₂AR may be closed to 2.

For example, when the protein conjugate is according to Formula (9): [symbol] , the value of M₂AR may be 4, and the value of D₂AR may be multiple of 4. When the composition comprises more conjugates having the Formula (9), the average M₂AR may be closed to 4.

The M₁AR, D₁AR, M₂AR, and/or D₂AR may be measured by LC-MS or HIC-HPLC analysis.

In the present disclosure, the protein conjugate may comprise 1 to 20 Formula (1):

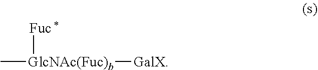

For example, the oligosaccharide may comprise 2 Formula (1):

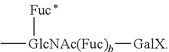

For example, the oligosaccharide may comprise 4 Formula (1):

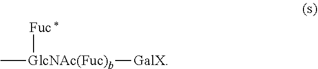

In the present disclosure, the protein conjugate may comprise a structure of [symbol] , wherein [symbol] is a GlcNAc, [symbol] is the Fuc linked to the GlcNAc through an α-1,6 linkage, GalX is linked to the GlcNAc through a GalX(β1,4GlcNAc linkage, Fuc* is linked to the GlcNAc through an Fuc*α1,3GlcNAc linkage, b is 0 or 1, and [symbol] is an antibody or a Fc-fusion protein. In these cases, the protein conjugate may be antibody-(Fucα1,6)_{0.1}(GalXβ1,4)GlcNAc-Fuc*.

In the present disclosure, the protein conjugate may have a M₁AR, and the M₁AR may be 2.

In the present disclosure, the protein conjugate may have a M₁AR and a M₂AR, wherein the M₁AR may be 2 and the M₂AR may be 2.

In the present disclosure, the protein conjugate may comprise a structure of [symbol] , wherein [symbol] is a GlcNAc, [symbol] is the Fuc linked to the said GlcNAc through a α-1,6GlcNAc linkage, [symbol] is a mannose, GalX is linked to the said GlcNAc through a GalXβ1,4GlcNAc linkage, Fuc* is linked to the GlcNAc through an Fuc*α1,3GlcNAc linkage, c is 0 or 1, and [symbol] is an antibody or Fc-fusion protein. In these cases, the protein conjugate may be an antibody-(GalX)₂(F)_{0,1}-Fuc* conjugate.

In the present disclosure, the protein conjugate may have a M₁AR, and the M₁AR may be 4

In the present disclosure, the protein conjugate may have a M₁AR and a M₂AR, wherein the M₁AR may be 4 and the M₂AR may be 4.

In the present disclosure, the protein conjugate may have one or more of the following properties: (1) having a first MOI-to-antibody ratio (M₁AR), and the M₁AR is 2 or 4, (2) having a first MOI-to-antibody ratio (M₁AR) and a second MOI-to-antibody ratio (M₂AR), and the M₁AR is 2 and the M₂AR is 2, or, the M₁AR is 4 and the M₂AR is 4, (3) capable of binding to an antigen, (4) capable of binding to an antigen, with a similar binding affinity as its corresponding antibody, (5) stable in plasma (e.g. human plasma) for at least 1 day, as measured in mass spectrometry analysis, (6) the linkage between the Fuco of Fuc* and the GlcNAc of the -GlcNAc(Fuc)_{b}-GalX are stable in plasma (e.g. human plasma) for at least 1 day, as measured in mass spectrometry analysis, wherein b is 0 or 1, (7) having a high reactive activity, and (8) capable of inhibiting tumor growth and/or tumor cell proliferation.

In another aspect, the present disclosure provides a composition comprising the protein conjugate of the present disclosure. In some embodiments, the composition has a first average MOI-to-antibody ratio (average M₁AR), wherein the average M₁AR may be about 2 (e.g., 1.9-2, 1.8-2, 1.7-2, 1.6-2, 1.5-2,1.2-2 or 1-2). For example, the average M₁AR may be 1.8-2. For example, the average M₁AR may be 1.6-2. For example, the average M₁AR may be 1.2-2. In some embodiments, the composition has a first average MOI-to-antibody ratio (average M₁AR) and a second average MOI-to-antibody ratio (average M₂AR), wherein the average M₁AR may be about 2 (e.g., 1.9-2, 1.8-2, 1.7-2, 1.6-2, 1.5-2 or 1-2), and/or the average M₂AR may be about 2 (e.g., 1.9-2, 1.8-2, 1.7-2, 1.6-2, 1.5-2 or 1-2). For example, the average M₁AR may be 1.8-2 and the average M₂AR may be 1.8-2. For example, the average M₁AR may be 1.6-2 and the average M₂AR may be 1.6-2. For example, the average M₁AR may be 1.2-2 and the average M₂AR may be 1.2-2.

In some embodiments, the composition has a first average MOI-to-antibody ratio (average M₁AR), wherein the average M₁AR may be 4 (e.g., 3.8-4, 3.6-4, 3.2-4, or 2.8-4). For example, the average M₁AR may be 3.6-4. For example, the average M₁AR may be 3.2-4. For example, the average M₁AR may be 2.8-4. In some embodiments, the composition has a first average MOI-to-antibody ratio (average M₁AR) and a second average MOI-to-antibody ratio (average M₂AR), wherein the average M₁AR may be about 4 (e.g., 3.8-4, 3.6-4, 3.2-4, or 2.8-4), and/or the average M₂AR may be about 4 (e.g., 3.8-4, 3.6-4, 3.2-4, or 2.8-4). For example, the average M₁AR may be 3.6-4 and the average M₂AR may be 3.6-4. For example, the average M₁AR may be 3.2-2 and the average M₂AR may be 3.2-4. For example, the average M₁AR may be 2.8-4 and the average M₂AR may be 2.8-4.

In another aspect, the present disclosure provides a protein conjugate, which is obtained from the method of the present disclosure.

In another aspect, the present disclosure provides a composition, which is obtained from the method of the present disclosure.

In another aspect, the present disclosure provides use of the Q-Fuc*′ of the present disclosure in preparation of said protein conjugate.

In another aspect, the present disclosure provides a pharmaceutical composition, comprising the protein conjugate and/or the composition of the present disclosure and optionally a pharmaceutically acceptable carrier.

In addition to the compositions and protein conjugates described above, the present invention also provides a number of methods that can be practiced utilizing the compounds and protein conjugates of the present disclosure. Methods for using the protein conjugate of the present disclosure may comprises: killing or inhibiting the growth or replication of a tumor cell or cancer cell, treating cancer, treating a pre-cancerous condition, killing or inhibiting the growth or replication of a cell that expresses an auto-immune antibody, treating an autoimmune disease, treating an infectious disease, preventing the multiplication of a tumor cell or cancer cell, preventing cancer, preventing the multiplication of a cell that expresses an auto-immune antibody, preventing an autoimmune disease, and preventing an infectious disease. These methods of use comprise administering to an animal such as a mammal or a human in need thereof. an effective amount of a protein conjugate.

Pharmaceutical compositions typically must be sterile and stable under the conditions of manufacture and storage. The pharmaceutical composition can be formulated as suitable for administration. The pharmaceutical composition can be formulated as a solution, emulsion, lyophilized formulation, microemulsion, liposome, or other ordered structure suitable to high drug concentration. As used herein, the term “pharmaceutically acceptable carrier” generally refers to a pharmaceutically acceptable adjuvant, excipient or stabilizer, which are non-toxic to the cells or subjects exposed to them at an administrated dose and concentration. Generally, the pharmaceutically acceptable carrier may be an aqueous solution. Examples of a pharmaceutically acceptable carrier may comprise a buffer, an antioxidant, a low molecular weight (less than about 10 residues) polypeptide, a protein, a hydrophilic polymer, a monosaccharide, a disaccharide and other carbohydrates, a chelating agent, a sugar alcohol, a salt-forming counterion, such as sodium; a nonionic surfactant, a preservative, a wetting agent, an emulsifying agent and/or a dispersing agent.

In another aspect, the present disclosure provides a method for preventing or treating disease, comprising administrating the protein conjugate, the composition and/or the pharmaceutical composition the of the present disclosure.

In another aspect, the present disclosure provides the use of the protein conjugate, the composition and/or the pharmaceutical composition, in preparation of a medicament for preventing or treating disease.

### EXAMPLES

The following examples are set forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Celsius, and pressure is at or near atmospheric. Standard abbreviations may be used, e.g., bp, base pair(s); kb, kilobase(s); pl, picoliter(s); s or sec, second(s); min, minute(s); h or hr, hour(s); aa, amino acid(s); nt, nucleotide(s); i.m., intramuscular(ly); i.p., intraperitoneal(ly); s.c., subcutaneous(ly); and the like.

### Example 1 Synthesis of GDP-FAz

GDP-FAz was synthesized according to the reported procedure (Wu P., et al., Proc. Natl. Acad. Sci. USA 2009, 106, 16096), and purified through a Bio-Gel P-2 Gel column (Biorad). HRMS (ESI−) calcd for C₁₆H₂₄N₈O₁₅P₂ (M−H⁺) 629.0764, found 629.0785.

### Example 2 Synthesis of GDP-FAm

To a clear solution of 100 mg (0.16 mmol) GDP-FAz in 8.75 mL MeOH/ddH₂O (1:1.5), 5 mg Pd/C (10%) was added. The air atmosphere was changed to H₂ by vacuum and refill, the H₂ pressure was kept at 0.28 MPa. The reaction was allowed for stirred 4 h and filtered through a 0.22 μM filter. Rotorvap and lyophilization give the product as a white solid (84.8 mg, yield 88%). HRMS (ESI−) calcd for C₁₆H₂₆N₆O₁₅P₂ (M−H⁺) 603.0859, found 603.0874. ¹H NMR (400 MHz, D₂O) δ 8.10 (s, 1H), 5.92 (d, J=6.1 Hz, 1H), 4.97 (t, J=7.6, 1H), 4.76-4.73 (m, 1H), 4.51 (dd, J=5.2, 3.4 Hz, 1H), 4.37-4.34 (m, 1H), 4.23-4.21 (m, 2H), 3.96 (dd, J=9.6, 2.4 Hz, 1H), 3.92-3.91 (m, 1H), 3.70 (dd, J=10.0, 3.3 Hz, 1H), 3.63 (dd, J=10.0, 7.6 Hz, 1H), 3.31 (dd, J=13.4, 9.6 Hz, 1H), 3.24 (dd, J=13.4, 3.1 Hz, 1H).

### Example 3 Synthesis of GDP-FAzP₄Biotin

400 μL GDP-FAz (50 mM in ddH₂O), 400 μL CuSO₄/BTTP (5 mM/10 mM in ddH₂O), 210 μL propargyl-PEG₄-Biotin (Click Chemistry Tools) (100 mM in MeOH), 40 μL ascorbate sodium (250 mM in ddH₂O) and 2.95 mL ddH₂O were mixed together. The reaction was allowed for stirring at r.t. for 5 h and monitored by TLC. Then, 2 mM BCS (bathocuproine sulphonate) was added to quench the reaction and the solvent was removed under reduced pressure. The crude product was further purified through a Prep-HPLC system to give the product as a white solid. (14.4 mg, yield 66%). HRMS (ESI−) calcd for C₃₇H₅₉N₁₁O₂₁P₂S (M−H⁺) 1086.3010, found 1086.3040. ¹H NMR (400 MHz, D₂O) δ 8.15 (s, 1H), 8.11 (s, 1H), 5.89 (d, J=6.0 Hz, 1H), 4.94-4.90 (m, 1H), 4.77-4.76 (m, 1H), 4.73-4.68 (m, 1H), 4.65 (d, J=3.1 Hz, 2H), 4.62-4.59 (m, 1H), 4.58-4.56 (m, 1H), 4.53-4.51 (m, 1H), 4.38 (dd, J=8.0, 4.4 Hz, 1H), 4.34-4.31 (m, 1H), 4.25-4.16 (m, 2H), 4.05-4.02 (m, 1H), 3.82 (s, 1H), 3.72-3.65 (m, 14H), 3.61 (t, J=5.3 Hz, 2H), 3.37 (t, J=5.2 Hz, 2H), 3.30-3.25 (m, 1H), 2.96 (dd, J=13.1, 5.0 Hz, 1H), 2.77-2.72 (m, 1H), 2.24 (t, J=7.3 Hz, 2H), 1.74-1.49 (m, 4H), 1.40-1.32 (m, 2H).

### Example 4 Synthesis of GDP-FAzP₄Tz

To a solution of 500 μL GDP-FAz (50 mM in ddH₂O) in ddH₂O/MeOH (1.45 mL/2.24 mL), 500 μL CuSO₄/BTTP (5 mM/10 mM in ddH₂O), 260 μL propargyl-PEG₄-Tz (Click Chemistry Tools) (100 mM in MeOH), and 50 μL ascorbate sodium (250 mM in ddH₂O) were added. The reaction was allowed for stirring at r.t. for 5 h and monitored by TLC. Then, 2 mM BCS was added to quench the reaction and the solvent was removed under reduced pressure. The crude product was further purified through a Prep-HPLC system to give the product as a pink solid (12.5 mg, yield 45%). HRMS (ESI−) calcd for C₄₀H₅₇N₁₃O₂₁P₂ (M−H⁺) 1116.3194, found 1116.3212.

### Example 5 Synthesis of GDP-FAzP₄MMAE

To a solution of 200 μL GDP-FAz (50 mM) in ddH₂O/MeOH (580 μL/790 μL), 200 μL CuSO₄/BTTP (5 mM/10 mM), 210 μL propargyl-PEG₄-vc-PAB-MMAE (Levena Biopharma) (50 mM in MeOH), and 20 μL ascorbate (250 mM in ddH₂O) were added. The reaction was allowed for stirring at r.t. for 5 h and monitored by TLC. Then, 2 mM BCS was added to quench the reaction and the solvent was removed under reduced pressure. The crude product was further purified through a Prep-HPLC system to give the product as a white solid (7.6 mg, 38%). HRMS (ESI−) calcd for C₈₆H₁₃₆N₁₈O₃₂P₂ (M-2H⁺)/2 996.4449, found 996.4463. ¹H NMR (400 MHz, D₂O) δ 8.19 (s, 1H), 8.09 (s, 1H), 7.49-7.47 (m, 2H), 7.38-7.29 (m, 6H), 7.21-7.12 (m, 1H), 5.88 (d, J=5.8 Hz, 1H), 5.25-5.09 (m, 1H), 5.08-4.96 (m, 1H), 4.91 (s, 1H), 4.76-4.75 (m, 1H), 4.70-4.66 (m, 2H), 4.62 (s, 2H), 4.59-4.54 (m, 1H), 4.52-4.50 (m, 1H), 4.47-4.45 (m, 2H), 4.33-4.32 (m, 2H), 4.20-4.15 (m, 5H), 4.03-4.00 (m, 1H), 3.80 (s, 1H), 3.74-3.71 (m, 2H), 3.68-3.58 (m, 16H), 3.49-3.39 (s, 1H), 3.35 (s, 1H), 3.31-3.27 (m, 5H), 3.18 (s, 1H), 3.07-3.06 (m, 3H), 2.92 (d, J=15.3 Hz, 3H), 2.84-2.79 (m, 1H), 2.62-2.37 (m, 4H), 2.20-1.99 (m, 3H), 1.85-1.77 (m, 5H), 1.65-1.51 (m, 4H), 1.37-1.22 (m, 4H), 1.21-1.12 (m, 2H), 1.08 (d, J=6.4 Hz, 2H), 0.96-0.67 (m, 26H), 0.52-0.51 (m, 1H).

### Example 6 Synthesis of GDP-FAmP₄Biotin

To a solution of 500 μL GDP-FAm (100 mM in ddH₂O) in 1.5 mL ddH₂O, 500 μL NaHCO₃ (200 mM), 1.95 mL THF and 550 μL NHS-PEG₄-Biotin (Click Chemistry Tools) (100 mM in THF) were added. The reaction was stirred at r.t. for 4 h and monitored by TLC. The solvent was removed under reduced pressure. The crude product was further purified through a Prep-HPLC system to give the product as a white solid (20.5 mg, 38%). HRMS (ESI−) calcd for C₃₇H₆₁N₉O₂₂P₂S (M−H⁺) 1076.3054, found 1076.3068. ¹H NMR (400 MHz, D₂O) δ 8.12 (s, 1H), 5.92 (d, J=6.1 Hz, 1H), 4.93 (t, J=7.8 Hz, 1H), 4.78-4.77 (m, 1H), 4.59 (dd, J=7.9, 4.6 Hz, 1H), 4.53 (dd, J=5.2, 3.4 Hz, 1H), 4.39 (dd, J=7.9, 4.4 Hz, 1H), 4.35-4.34 (m, 1H), 4.22 (dd, J=5.4, 3.4 Hz, 2H), 3.87 (d, J=3.1 Hz, 1H), 3.76 (t, J=6.3 Hz, 2H), 3.69-3.66 (m, 14H), 3.63-3.60 (m, 3H), 3.59-3.56 (m, 1H), 3.38 (t, J=5.3 Hz, 2H), 3.32-3.26 (m, 2H), 2.97 (dd, J=13.1, 5.0 Hz, 1H), 2.77 (d, J=13.0 Hz, 1H), 2.56 (t, J=6.2 Hz, 2H), 2.26 (t, J=7.3 Hz, 2H), 1.74-1.51 (m, 4H), 1.42-1.34 (m, 2H).

### Example 7 Synthesis of GDP-FAmP₄Tz

To a solution of 200 μL GDP-FAm (100 mM) in 600 uL ddH₂O were added 200 μL NaHCO₃ (200 mM), 780 μL THF and 220 μL NHS-PEG₄-Tz (Click Chemistry Tools) (100 mM in THF). The reaction was stirred at r.t. for 4 h and monitored by TLC. The solvent was removed under reduced pressure. The crude product was further purified through a Prep-HPLC system to give the product as a pink solid (9.9 mg, yield 48%). HRMS (ESI−) calcd for C₃₆H₅₂N₁₀O₂₁P₂ (M−H⁺) 1021.2711, found 1021.2725. ¹H NMR (400 MHz, D₂O) δ 8.17-8.13 (m, 2H), 8.00 (s, 1H), 7.06-7.02 (m, 2H), 5.76 (d, J=5.6 Hz, 1H), 4.91 (t, J=7.7 Hz, 1H), 4.67 (t, J=5.4 Hz, 1H), 4.49 (dd, J=5.1, 3.7 Hz, 1H), 4.30-4.28 (m, 1H), 4.27-4.25 (m, 2H), 4.21-4.19 (m, 2H), 3.95-3.93 (m, 2H), 3.84-3.83 (m, 1H), 3.80-3.78 (m, 2H), 3.74-3.71 (m, 2H), 3.70-3.57 (m, 13H), 3.51 (dd, J=14.1, 4.2 Hz, 1H), 3.24 (dd, J=14.0, 8.6 Hz, 1H), 3.00 (s, 3H), 2.49 (t, J=6.3 Hz, 2H).

### Example 8 Synthesis of GDP-FAmP₈Tz

To a solution of 200 μL GDP-FAm (100 mM) in 600 μL ddH₂O were added 200 μL NaHCO₃ (200 mM), 780 μL THF and 220 μL NHS-PEG₈-Tz (Xi'an Dianhua Biotechnology Co., Ltd) (100 mM in THF). The reaction was stirred at r.t. for 4 h and monitored by TLC. The solvent was removed under reduced pressure. The crude product was further purified through a Prep-HPLC system to give the product as a pink solid (9.2 mg, yield 38%). HRMS (ESI−) calcd for C₄₄H₆₈N₁₀O₂₅P₂ (M−2H⁺)/2 598.1844, found 598.1880. ¹H NMR (400 MHz, D₂O) δ 8.28-8.24 (m, 2H), 8.02 (s, 1H), 7.16-7.12 (m, 2H), 5.81 (d, J=6.0 Hz, 1H), 4.92 (t, J=7.8 Hz, 1H), 4.72 (t, J=5.6 Hz, 1H), 4.50 (dd, J=5.2 Hz, 3.5, 1H), 4.32-4.29 (m, 3H), 4.21-4.19 (m, 2H), 3.97-3.95 (m, 2H), 3.85 (d, J=3.0 Hz, 1H), 3.81-3.78 (m, 2H), 3.75-3.59 (m, 32H), 3.27 (dd, J=14.1, 8.7, 1H), 3.02 (s, 3H), 2.53 (t, J=6.2 Hz, 2H).

### Example 9 Synthesis of GDP-FAmP₄BCN

To a solution of 200 μL GDP-FAm (100 mM) in 600 μL ddH₂O were added 200 μL NaHCO₃ (200 mM), 560 μL THF and 440 μL NHS-PEG₄-BCN (Xi'an Dianhua Biotechnology Co., Ltd) (50 mM in THF). The reaction was stirred at r.t. for 4 h and monitored by TLC. The solvent was removed under reduced pressure. The crude product was further purified through a Prep-HPLC system to give the product as a white solid (7.4 mg, yield 36%). HRMS (ESI−) calcd for C₃₈H₅₉N₇O₂₂P₂ (M−2H⁺)/2 512.6522, found 512.6532. ¹H NMR (400 MHz, D₂O) δ 8.17 (s, 1H), 5.92 (d, J=5.9 Hz, 1H), 4.93 (t, J=7.8 Hz, 1H), 4.78-4.75 (m, 1H), 4.52 (dd, J=5.1, 3.5 Hz, 1H), 4.36-4.32 (m, 1H), 4.22 (dd, J=5.4, 3.4 Hz, 2H), 4.14 (d, J=8.2 Hz, 2H), 3.86 (d, J=2.3 Hz, 1H), 3.75 (t, J=6.3 Hz, 2H), 3.69-3.64 (m, 14H), 3.62-3.58 (m, 4H), 3.31 (t, J=5.3 Hz, 2H), 3.29-3.26 (m, 1H), 2.55 (t, J=6.2 Hz, 2H), 2.29-2.15 (m, 6H), 1.54-1.51 (m, 2H), 1.39-1.31 (m, 1H), 0.92 (t, J=9.8 Hz, 2H).

### Example 10 Synthesis of GDP-FAmP₄TCO

To a solution of 400 μL GDP-FAm (100 mM) in 1.4 mL ddH₂O were added 400 uL NaHCO₃ buffer (200 mM), 1.36 mL THF and 440 μL NHS-PEG₄-TCO (Click Chemistry Tools) (100 mM in THF). The reaction was stirred at r.t. for 4 h and monitored by TLC. The solvent was removed under reduced pressure. The crude product was further purified through a Prep-HPLC system to give the product as a white solid (8.2 mg, yield 20%). HRMS (ESI−) calcd for C₃₆H₅₉N₇₀₂₂P₂ (M−H⁺) 1002.3116, found 1002.3134.

### Example 11 Synthesis of GDP-FAmAz

To a solution of 200 μL GDP-FAm (100 mM) in 600 μL ddH₂O were added 200 μL NaHCO₃ (200 mM), 780 μL THF and 220 μL NHS-azide (Xi'an Dianhua Biotechnology Co., Ltd) (100 mM in THF). The reaction was stirred at r.t. for overnight and monitored by TLC. The solvent was removed under reduced pressure. The crude product was further purified through a Prep-HPLC system to give the product as a white solid (8.7 mg, yield 63%). HRMS (ESI−) calcd for C₁₈H₂₇N₉O₁₆P₂ (M−H⁺) 686.0978, found 686.1002. ¹H NMR (400 MHz, D₂O) δ 8.10 (s, 1H), 5.92 (d, J=6.0 Hz, 1H), 4.92 (t, J=7.9 Hz, 1H), 4.78-4.76 (m, 1H), 4.52 (dd, J=5.2, 3.4 Hz, 1H), 4.35-4.34 (m, 1H), 4.23-4.21 (m, 2H), 4.00 (s, 2H), 3.87 (d, J=3.2 Hz, 1H), 3.71 (dd, J=8.8, 3.9 Hz, 1H), 3.66 (dd, J=10.0, 3.3 Hz, 1H), 3.62-3.57 (m, 2H), 3.32 (dd, J=14.0, 8.6 Hz, 1H).

### Example 12 Synthesis of GDP-FAmP₂Az

To a solution of 200 μL GDP-FAm (100 mM) in 600 μL ddH₂O were added 200 μL NaHCO₃ buffer (200 mM), then 780 μL THF and 220 μL NHS-PEG₂-azide (Xi'an Dianhua Biotechnology Co., Ltd) (100 mM in THF) were added. The reaction was stirred at r.t. for overnight and monitored by TLC. The solvent was removed under reduced pressure. The crude product was further purified through a Prep-HPLC system to give the GDP-FAmP₂Az as a white solid (5.4 mg, yield 34%). HRMS (ESI−) calcd for C₂₃H₃₇N₉O₁₈P₂ (M−H⁺) 788.1659, found 788.1671. ¹H NMR (400 MHz, D₂O) δ 8.10 (s, 1H), 5.91 (d, J=6.0 Hz, 1H), 4.91 (t, J=7.3 Hz, 1H), 4.77-4.68 (m, 1H), 4.52 (t, J=3.8 Hz, 1H), 4.33-4.32 (m, 1H), 4.20-4.08 (m, 2H), 3.85 (d, J=3.1 Hz, 1H), 3.76-3.73 (m, 2H), 3.72-3.62 (m, 8H), 3.61-3.54 (m, 2H), 3.45 (t, J=4.8 Hz, 2H), 3.27 (dd, J=14.0, 8.5 Hz, 1H), 2.53 (t, J=6.0 Hz, 2H).

### Example 13 Synthesis of GDP-FAmP₄Az

To a solution of 200 μL GDP-FAm (100 mM) in 600 μL ddH₂O were added 200 μL NaHCO₃ buffer, then 780 μL THF and 220 μL NHS-PEG₄-azide (Xi'an Dianhua Biotechnology Co., Ltd) (100 mM in THF) were added. The reaction was stirred at r.t. for overnight and monitored by TLC. The solvent was removed under reduced pressure. The crude product was further purified through a Prep HPLC system to give the GDP-FAmP₄Az as a white solid (9.0 mg, yield 51%.). HRMS (ESI−) calcd for C₂₇H₄₅N₉O₂₀P₂ (M−H⁺) 876.2183, found 876.2187. ¹H NMR (400 MHz, D₂O) δ 8.14 (s, 1H), 5.90 (d, J=6.0 Hz, 1H), 4.90 (t, J=7.8 Hz, 1H), 4.75 (t, J=5.5 Hz, 1H), 4.50 (dd, J=5.0, 3.5 Hz, 1H), 4.33-4.32 (m, 1H), 4.21-4.19 (m, 2H), 3.84 (d, J=3.2 Hz, 1H), 3.73 (t, J=6.2, 2H), 3.70-3.61 (m, 16H), 3.60-3.53 (m, 2H), 3.47-3.45 (m, 2H), 3.26 (dd, J=14.0, 8.6 Hz, 1H), 2.53 (t, J=6.2 Hz, 2H).

### Example 14 Synthesis of GDP-FAmP₄MCP

To a solution of 220 μL GDP-FAm (100 mM) in 690 μL ddH₂O were added 200 μL NaHCO₃ (200 mM), 690 μL THF and 200 μL NHS-PEG₄-MCP (Xi'an Dianhua Biotechnology Co., Ltd) (100 mM in THF). The reaction was stirred at r.t. for 6 h and monitored by TLC. The solvent was removed under reduced pressure. The crude product was further purified through a Prep-HPLC system to give the desired product as a white solid (7.8 mg, 40%). HRMS (ESI−) calcd for C₃₃H₅₃N₇O₂₂P₂ (M−H⁺) 960.2646, found. 960.2638.

### Example 15 Synthesis of GDP-FAmGGG

Cbz-GGG-OH was synthesized according to the reported procedure (Miravet J. F., et al. Eur. J. Org. Chem. 2014,16, 3372).

Cbz-GGG-NHS. To a solution of 200 mg (0.62 mmol) Cbz-GGG-OH in 1.5 mL dry DMF were added 82 mg (0.71 mmol) NHS and 148 mg (0.82 mmol) EDC·HCl. The reaction was stirred at r.t. for 3 h and monitored by TLC. The solvent was removed under reduced pressure. The residue was resolved in 80 mL DCM, and washed by water, saturated NaHCO₃ solution and brine respectively. The organic layers were dried through Na₂SO₄ and concentrated to afford the crude for the next step without further purification.

GDP-FAmGGG-Cbz. The crude product (Cbz-GGG-NHS) obtained above were resolved in 5.4 mL H₂O followed by adding 40.5 mg (0.48 mmol) NaHCO₃ and 115.0 mg (0.19 mmol) GDP-FAm. The reaction was stirred at r.t. for overnight and monitored by TLC. The product was further purified through a Prep-HPLC system to give GDP-FAmGGG-Cbz as a white powder (135.6 mg. yield 24% in two steps).

GDP-FAmGGG. To a clear solution of 21 mg (0.023 mmol) GDP-FAmGGG-Cbz was added 2 mL H₂O and 15 mg Pd/C (10%). The air atmosphere was change to H₂ by vacuum and refill. The H₂ pressure was kept at 0.28 MPa. The reaction was stirred at r.t. for 1 h and filtered through a 0.22 μm filter. The product was further purified through a Prep-HPLC system to give the GDP-FAmGGG as a white powder (15.2 mg, yield 85%). HRMS (ESI−) calcd for C₂₂H₃₅N₉O₁₈P₂ (M−2H⁺)/2 386.5715, found 386.5717.

### Example 16 Synthesis of GDP-FAmP₄MMAE

To a solution of 200 uL GDP-FAm (100 mM) in 600 uL ddH₂O were added 200 uL NaHCO₃ (200 mM), then 560 uL THF and 440 uL OSu-PEG₄-vc-PAB-MMAE (Levena Biopharma) (50 mM in THF). The reaction was stirred at r.t. for 4 h and monitored by TLC. The solvent was removed under reduced pressure. The crude product was further purified through a Prep-HPLC system to give the desired product as a white solid (13.2 mg. 33%). HRMS (ESI−) calcd for C₈₆H₁₃₈N₁₆O₃₃P₂ (M−2H⁺)/2 991.4471, found 991.4502. ¹H NMR (400 MHz, D₂O) δ 8.19 (s, 1H), 7.48-7.42 (m, 2H), 7.41-7.28 (m, 6H), 7.22-7.16 (m, 1H), 5.90 (d, J=6.0 Hz, 1H), 5.28-5.14 (m, 1H), 5.03 (t, J=11.0 Hz, 1H), 4.90 (t, J=7.8 Hz, 1H), 4.64-4.58 (m, 1H), 4.51-4.49 (m, 1H), 4.44-4.38 (m, 2H), 4.34-4.28 (m, 2H), 4.21-4.16 (m, 3H), 4.11 (t, J=7.2 Hz, 2H), 3.83 (d, J=3.2 Hz, 1H), 3.75-3.70 (m, 4H), 3.69-3.64 (m, 3H), 3.63-3.56 (m, 14H), 3.34 (s, 1H), 3.30-3.25 (m, 6H), 3.19-3.14 (m, 2H), 3.11-3.06 (m, 4H), 2.95-2.83 (m, 4H), 2.62-2.44 (m, 6H), 2.16-1.97 (m, 4H), 1.92-1.71 (m, 6H), 1.65-1.43 (m, 5H), 1.35-1.21 (m, 4H), 1.17-1.14 (m, 3H), 1.06 (d, J=6.7 Hz, 2H), 0.96 (d, J=6.4 Hz, 2H), 0.93-0.89 (m, 8H), 0.83-0.78 (m, 10H), 0.71-0.66 (m, 2H), 0.50-0.48 (m, 2H).

### Example 17 Synthesis of GDP-FAmSucMMAE

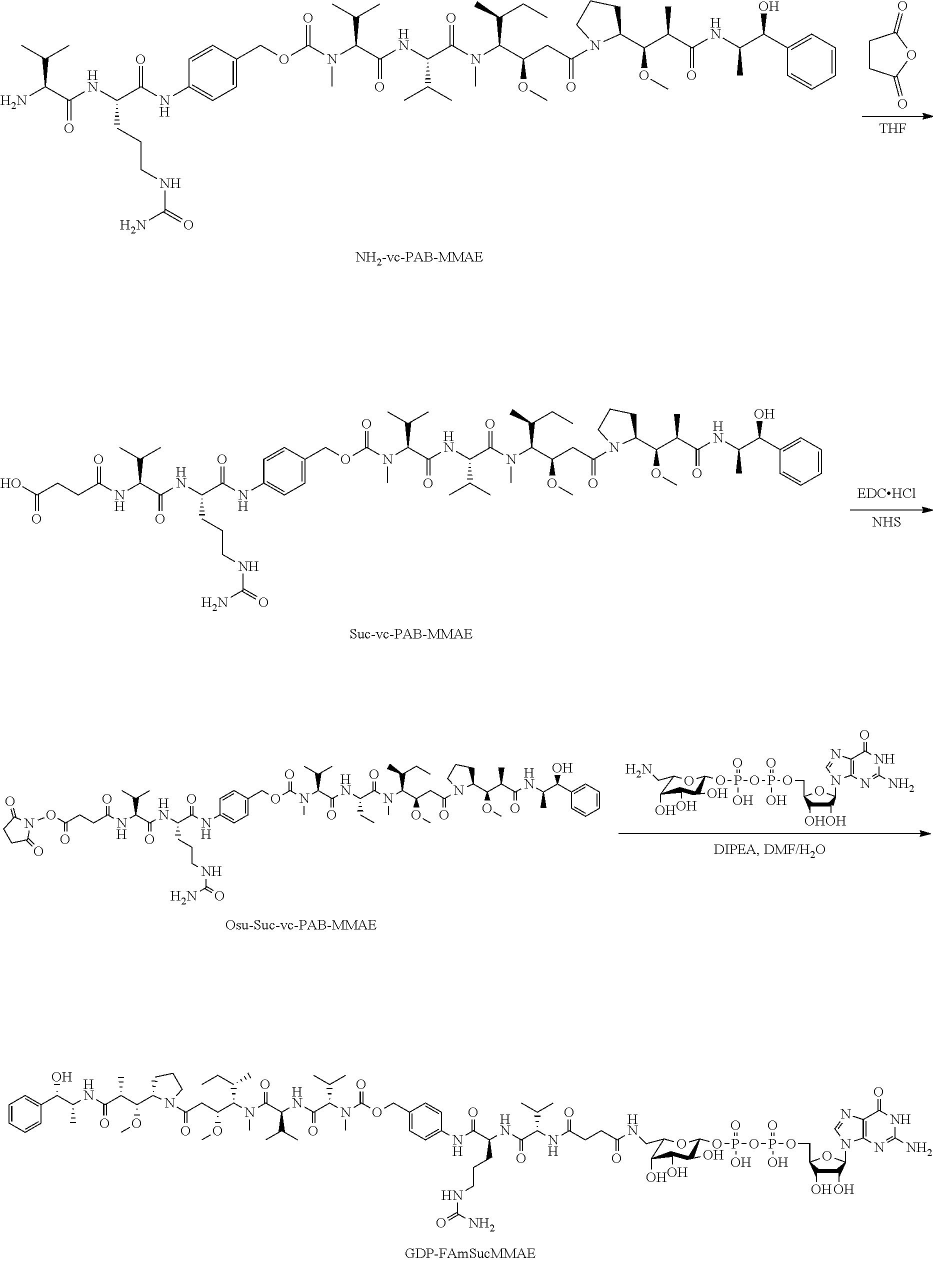

NH₂-vc-PAB-MMAE was synthesized according to the reported procedure (Tang, F., et al. Org. Biomol. Chem. 2016, 14, 9501).

Suc-vc-PAB-MMAE. To a solution of NH₂-vc-PAB-MMAE (833 mg, 0.74 mmol) in DMF (15 mL) and THF (15 mL) were added Succinic anhydride (120 mg, 1.12 mmol). The mixture was stirred at r.t. for 5 h and monitored by TLC. The product was further purified through a Prep-HPLC system to give the Suc-vc-PAB-MMAE as a white foam (683 mg, yield 75.3%). HRMS (ESI−) calcd for C₆₂H₉₈N₁₀O₁₅ (M−H⁺) 1221.7140, found 1221.7146.

OSu-Suc-vc-PAB-MMAE. To a solution of Suc-vc-PAB-MMAE (683 mg, 0.559 mmol) in DCM (10 mL) and THF (10 mL) were added NHS (644 mg, 5.596 mmol) and EDC·HCl (1284 mg, 6.698 mmol). The mixture was stirred at r.t. for 3 h and monitored by TLC. The product was further purified through a Prep-HPLC system to give the OSu-Suc-vc-PAB-MMAE as a white powder (565 mg, yield 76.6%). HRMS (ESI+) calcd for C₆₆H₁₀₁N₁₁O₁₇ (M+Na⁺) 1342.7269, found 1342.7283.

GDP-FAmSucMMAE. To a solution of GDP-FAm (190 mg, 0.315 mmol) in 30 mL ddH₂O were added 400 uL DIPEA, and then OSu-Suc-vc-PAB-MMAE (346 mg, 0.262 mmol) in 12 mL DMF were added. The mixture was stirred at r.t. for 5 h and monitored by TLC. The product was further purified through a Prep-HPLC system to give the GDP-FAmSucMMAE as a white powder (104.3 mg, yield 22.0%). HRMS (ESI−) calcd for C₇₈H₁₂₂N₁₆O₂₉P₂ (M−2H⁺)/2 903.3947, found 903.3959. ¹H NMR (400 MHz, D₂O) δ 8.16 (s, 1H), 7.48-7.40 (m, 3H), 7.37-7.28 (m, 5H), 7.22-7.14 (m, 1H), 5.9 (d, J=6.0 Hz, 1H), 5.30-5.14 (m, 1H), 5.06-5.00 (m, 1H), 4.76-4.68 (m, 2H), 4.63-4.59 (m, 1H), 4.51-4.49 (m, 1H), 4.46-4.30 (m, 4H), 4.21-4.04 (m, 6H), 3.76-3.63 (m, 2H), 3.57-3.53 (m, 2H), 3.45-3.14 (m, 14H), 3.11-3.05 (m, 4H), 2.94-2.90 (m, 3H), 2.66-2.44 (m, 6H), 2.16-2.01 (m, 3H), 1.94-1.76 (m, 4H), 1.68-1.47 (m, 4H), 1.36-1.14 (m, 8H), 1.06 (d, J=6.6 Hz, 2H), 0.97-0.89 (m, 10H), 0.85-0.77 (m, 10H), 0.66 (t, J=7.8 Hz, 2H), 0.48 (d, J=6.6 Hz, 1H).

### Example 18 Synthesis of GDP-FAmAzP₄MMAE

To a solution of 200 μL GDP-FAmAz (50 mM) in ddH₂O/MeOH (580 μL/790 μL), were added 200 μL CuSO₄/BTTP (5 mM/10 mM), 210 μL propargyl-PEG₄-vc-PAB-MMAE (Levena Biopharma) (50 mM in MeOH), and 20 μL ascorbate (250 mM in ddH₂O). The reaction as allowed for stirring at r.t. for 5 h and monitored by TLC. Then, 2 mM BCS was added to quench the reaction and the solvent was removed under reduced pressure. The crude product was further purified through a Prep-HPLC system to give the product as a white solid (16.8 mg, 82%). HRMS (ESI+) calcd for C₈₈H₁₃₉N₁₉O₃₃P₂ (M+2Na⁺)/2 1048.9521, Found 1048.9533.

### Example 19 Synthesis of GDP-FAmP₄AzP₄MMAE

To a solution of 200 μL GDP-FAmP₄Az (50 mM) in ddH₂O/MeOH (580 μL/790 μL), were added 200 μL CuSO₄/BTTP (5 mM/10 mM), 210 μL propargyl-PEG₄-vc-PAB-MMAE (Levena Biopharma) (50 mM in MeOH), and 20 μL ascorbate (250 mM in ddH₂O). The reaction as allowed for stirring at r.t. for 5 h and monitored by TLC. Then, 2 mM BCS was added to quench the reaction and the solvent was removed under reduced pressure. The crude product was further purified through a Prep-HPLC system to give the product as a white solid (17.0 mg, 76%). HRMS (ESI+) calcd for C₉₇H₁₅₇N₁₉O₃₇P₂(M+2Na+)/2 1144.0124, Found 1144.0086. ¹H NMR (400 MHz, D₂O) δ 8.16 (s, 1H), 8.04 (s, 1H), 7.49-7.42 (m, 3H), 7.39-7.30 (m, 5H), 7.25-7.21 (m, 1H), 5.91 (d, J=5.9 Hz, 1H), 5.30-5.17 (m, 1H), 5.05 (t, J=10.8 Hz, 1H), 4.95-4.90 (m, 1H), 4.76-4.70 (m, 1H), 4.65 (s, 2H), 4.59 (t, J=5.0 Hz, 2H), 4.52-4.49 (m, 1H), 4.46-4.39 (m, 2H), 4.36-4.32 (m, 1H), 4.26-4.18 (m, 3H), 4.14 (t, J=6.7 Hz, 2H), 3.93 (t, J=5.1 Hz, 2H), 3.85 (d, J=3.0 Hz, 1H), 3.76-3.55 (m, 35H), 3.47-3.42 (m, 1H), 3.37-3.36 (m, 1H), 3.32-3.24 (m, 6H), 3.21-3.17 (m, 1H), 3.13-3.06 (m, 4H), 2.97-2.90 (m, 3H), 2.85-2.67 (m, 1H), 2.66-2.38 (m, 6H), 2.35-1.99 (m, 4H), 1.93-1.76 (m, 5H), 1.65-1.51 (m, 5H), 1.31-1.20 (m, 4H), 1.18-1.14 (m, 2H), 1.08 (d, J=6.8 Hz, 2H), 0.99 (d, J=6.4 Hz, 2H), 0.94-0.90 (m, 8H), 0.87-0.78 (m, 12H), 0.73-0.67 (m, 2H), 0.52-0.51 (m, 1H).

### Example 20 Synthesis of GDP-FAmAzP₄DXd

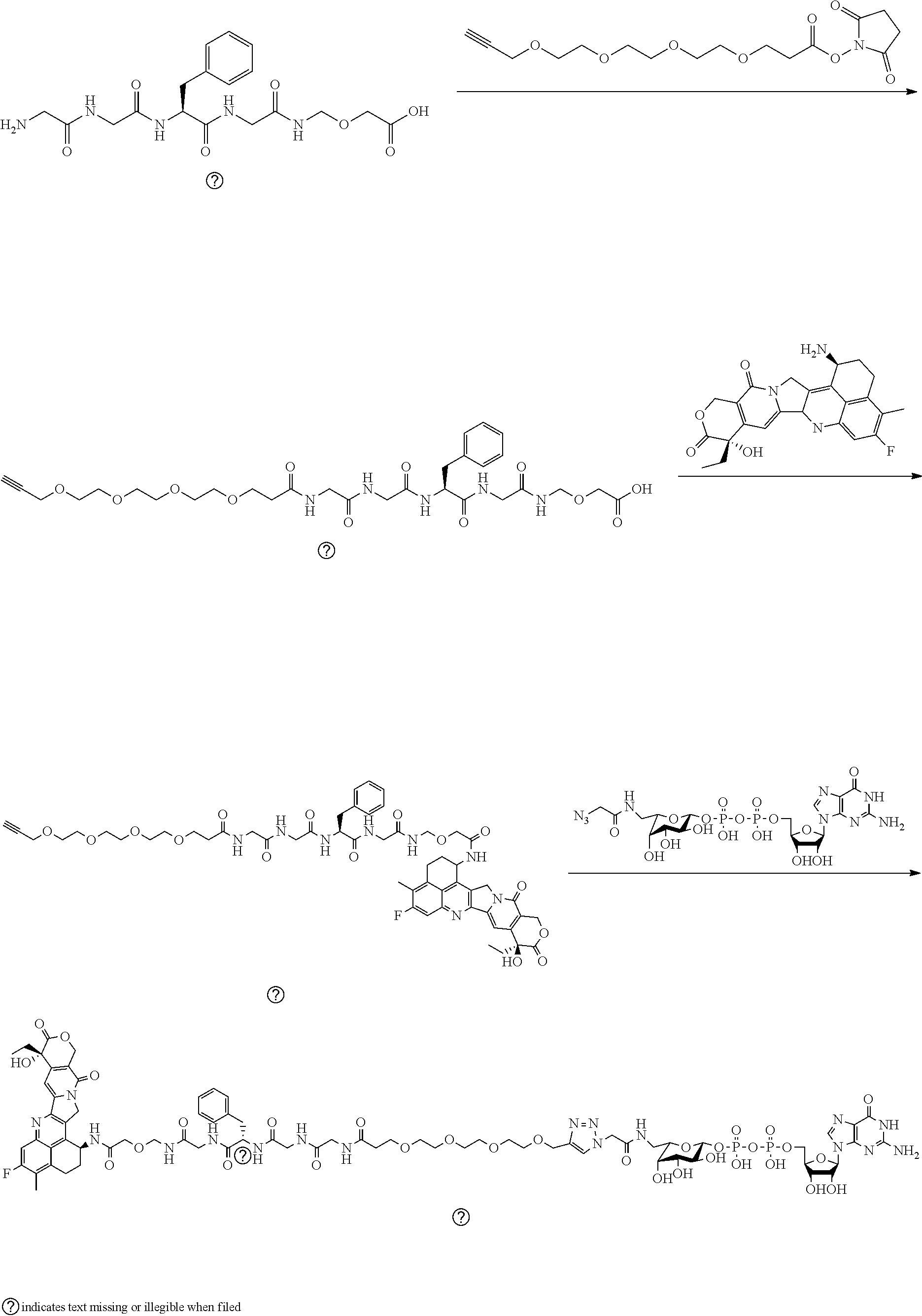

GGFG-Acid was synthesized according to the reported procedure (Yamaguchi, T., et al., EP3677589A1).

Propargyl-PEG₄-GGFG-Acid. To a solution of GGFG-Acid (98.4 mg, 0.23 mmol) in DMF (5 ml) were added DIPEA (0.2 ml) and propargyl-PEG₄-OSu (99.6 mg, 0.28 mmol). The mixture was stirred at r.t. for overnight and monitored by TLC. The crude product was further purified through a Prep-HPLC system to give the desired product as a white solid (114.8 mg, 75.0%). HRMS (ESI-) calcd for C₃₀H₄₃N₅O₁₂ (M−H⁺) 664.2835, found 664.2808.

Propargyl-PEG₄-GGFG-DXd. To a solution of propargyl-PEG₄-GGFG Acid (66.6 mg, 0.1 mmol) in DMF (5 ml) were added DIPEA (0.1 ml), Exatecan (43.5 mg, 0.1 mmol) and PyBOP (104.9 mg, 0.2 mmol). The mixture was stirred at r.t. for 2 h and monitored by TLC. The crude product was further purified through a Prep-HPLC system to give the desired product as a light-yellow solid (70.6 mg, 65.2%). HRMS (ESI+) calcd for C₅₄H₆₃FN₈O₁₅ (M+Na⁺) 1105.4289, found 1105.4255.

GDP-FAmAzP₄DXd. To a solution of 200 uL GDP-FAmAz (50 mM) in ddH₂O/MeOH (580 μL/790 uL), were added 200 μL CuSO₄/BTTP (5 mM/10 mM), 210 μL propargyl-PEG₄-GGFG-DXd (50 mM in MeOH), and 20 μL ascorbate (250 mM in ddH₂O). The reaction was allowed for stirring at r.t. for 5 h and monitored by TLC. Then, 2 mM BCS was added to quench the reaction and the solvent was removed under reduced pressure. The crude product was further purified through a Prep-HPLC system to give the product as a white solid (12.1 mg, 68.5%). HRMS (ESI-) calcd for C₇₂H₉₀FN₁₇O₃₁P₂ (M−2H⁺)/2 883.7651, found 883.7651. 1H NMR (400 MHZ, D20) 8 7.97 (s, 2H), 7.21 (s, 1H), 7.11-7.03 (m, 4H), 6.86 (d, J=7.0 Hz, 2H), 5.68 (d, J=5.7 Hz, 1H), 5.59-5.55 (m, 1H), 5.45-5.40 (m, 1H), 5.29-5.25 (m, 1H), 5.20 (s, 1H), 4.95-4.91 (m, 2H), 4.70-4.53 (m, 7H), 4.46 (t, J=4.1 Hz, 1H), 4.37-4.31 (m, 2H), 4.26-4.17 (m, 4H), 3.86-3.57 (m, 26H), 3.34-3.28 (m, 1H), 3.10-3.06 (m, 1H), 2.97-2.88 (m, 1H), 2.78-2.73 (m, 1H), 2.56-2.50 (m, 3H), 2.39-2.29 (m, 1H), 2.09 (s, 3H), 1.93-1.82 (m, 2H), 0.94 (t, J=7.3, 3H).

### Example 21 GDP-FAmDM4

To a solution of 200 μL GDP-FAm (100 mM) was added 600 μL ddH₂O, 200 μL NaHCO₃ (200 mM), 560 μL THF and 440 μL OSu-SPDB-DM4 (Levena Biopharma) (50 mM in THF). The reaction was stirred at r.t. for 4 h and monitored by TLC. The solvent was removed under reduced pressure. The crude product was further purified through a Prep-HPLC system to give the desired product as a white solid (9.2 mg. 620%). HRMS (ESI−) calcd for C₅₈H₈₄ClN₉O₂₆P₂S2 (M−2H⁺)/2 740.6994, Found: 740.7004. ¹H NMR (400 MHz, D₂O) δ 8.20 (s, 1H), 7.13 (s, 1H), 6.62-6.54 (m, 3H), 5.90 (d, J=5.6 Hz, 1H), 5.65-5.60 (m, 1H), 5.38-5.37 (m, 1H), 4.93 (t, J=7.8 Hz, 1H), 4.71 (t, J=5.4 Hz, 1H), 4.60-4.57 (m, 1H), 4.52-4.50 (m, 1H), 4.31 (s, 1H), 4.25-4.22 (m, 3H), 3.94 (s, 3H), 3.87-3.86 (m, 1H), 3.70-3.48 (m, 7H), 3.35 (s, 3H), 3.30-3.26 (m, 1H), 3.23-3.18 (m, 4H), 3.08 (d, J=9.4 Hz, 1H), 2.83 (s, 3H), 2.69-2.62 (m, 1H), 2.52-2.49 (m, 3H), 2.39-2.37 (m, 1H), 2.26-2.23 (m, 3H), 1.88-1.79 (m, 2H), 1.74-1.67 (m, 2H), 1.59-1.51 (m, 4H), 1.36-1.33 (m, 1H), 1.26 (d, J=6.6 Hz, 3H), 1.21-1.19 (m, 6H), 1.11 (s, 3H), 0.78 (s, 3H).

### Example 22 Synthesis of GDP-FAmSucMMAE (No vc-PAB)

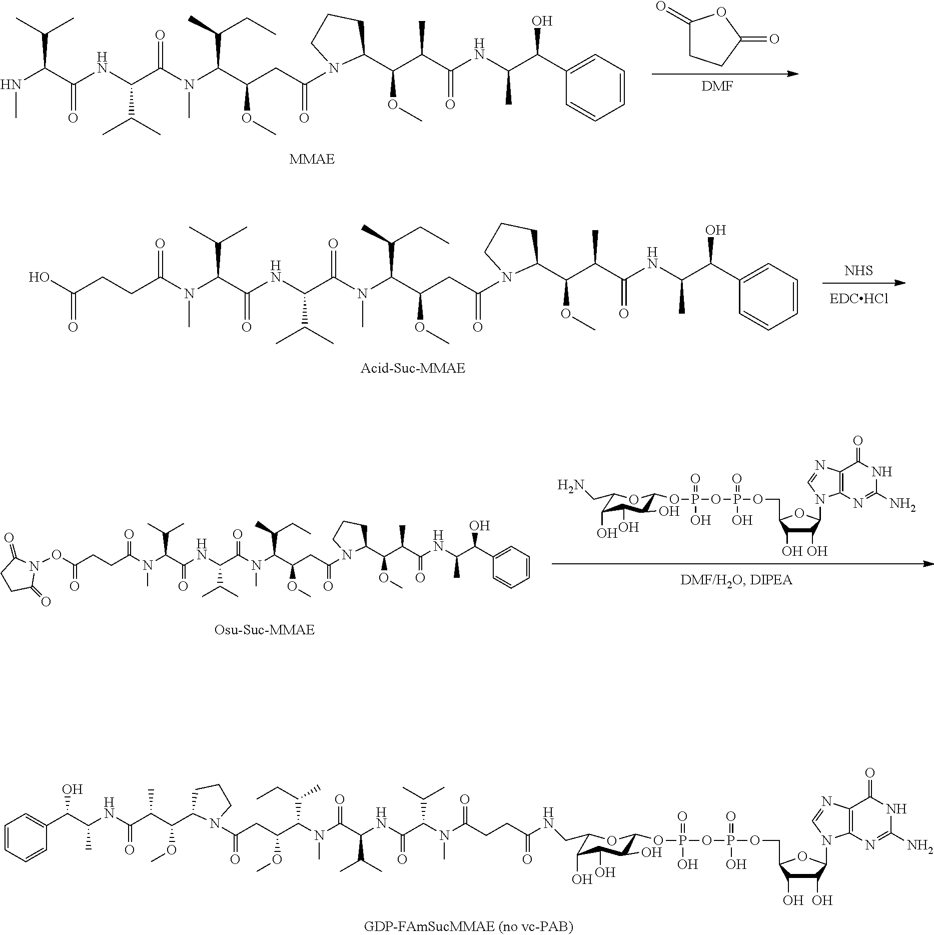

GDP-FAmSucMMAE (no cleavable linker) was synthesized according to the route list above. Acid-Suc-MMAE. To a solution of MMAE (59 mg, 0.082 mmol) in DMF (4 mL) was added Succinic anhydride (24.7 mg, 0.25 mmol). The mixture was stirred at r.t. overnight and monitored by TLC. The product was further purified by Prep-HPLC system to give the Acid-Suc-MMAE as a white powder (52 mg, yield 77.4%).

OSu-Suc-MMAE. To a solution of Acid-Suc-MMAE (80.5 mg, 0.098 mmol) in DCM (4 mL) was added NHS (45.3 mg, 0.394 mmol) and EDC·HCl (113.2 mg, 0.59 mmol). The mixture was stirred at r.t. for 3 h and monitored by TLC. The product was further purified by Prep-HPLC system to give the OSu-Suc-MMAE as a white powder (68 mg, yield 75.6%).

GDP-FAmSucMMAE (no vc-PAB). To a solution of GDP-FAm (30 mg, 0.05 mmol) in 3 mL ddH₂O was added 26 μL DIPEA, and then OSu-Suc-MMAE (90.9 mg, 0.099 mmol) in 3 mL DMF was added. The mixture was stirred at r.t. overnight and monitored by TLC. The product was further purified by Prep-HPLC system to give the GDP-FAmSucMMAE (no cleavable linker) as a white powder (10 mg, yield 14.3%). ¹H NMR (400 MHz, D₂O) δ 8.17 (s, 1H), 7.31-7.22 (m, 5H), 5.85 (d, J=6.0 Hz, 1H), 4.84 (t, J=8.0 Hz, 1H), 4.65-4.55 (m, 1H), 4.53-4.35 (m, 4H), 4.30-4.20 (m, 2H), 4.15-3.98 (m, 4H), 3.78-3.77 (m, 1H), 3.63-3.58 (m, 2H), 3.56-3.55 (m, 1H), 3.53-3.51 (m, 1H), 3.35-2.30 (m, 1H), 3.27-3.12 (m, 10H), 3.06-3.04 (m, 2H), 2.98-2.96 (m, 2H), 2.86-2.79 (m, 1H), 2.72-2.57 (m, 3H), 2.50-2.35 (m, 4H), 2.26-1.92 (m, 4H), 1.75-1.51 (m, 4H), 1.46-1.48 (m, 1H), 1.22-1.21 (m, 2H), 1.16-1.14 (m, 1H), 1.07-1.06 (m, 1H), 1.00-0.98 (m, 2H), 0.93-0.70 (m, 20H).

### Example 23 Synthesis of TCO-PEG₄-vc-PAB-MMAE

To a solution of NH₂-vc-PAB-MMAE (30.0 mg, 0.027 mmol) in DMF (1.5 mL) were added DIPEA (100 μL) and NHS-PEG₄-TCO (Xi'an Dianhua Biotechnology Co., Ltd) (16.5 mg, 0.032 mmol). The mixture was stirred at r.t. for overnight and monitored by TLC. The product was further purified through a Prep-HPLC system to give the TCO-PEG₄-vc-PAB-MMAE as a white powder (21.7 mg, yield 53%). HRMS (ESI−) calcd for C₇₈H₁₂₇N₁₁O₁₉ (M−H⁺) 1520.9237, found 1520.9277.

### Example 24 Synthesis of DBCO-PEG₄-vc-PAB-seco-DUBA (24-12)

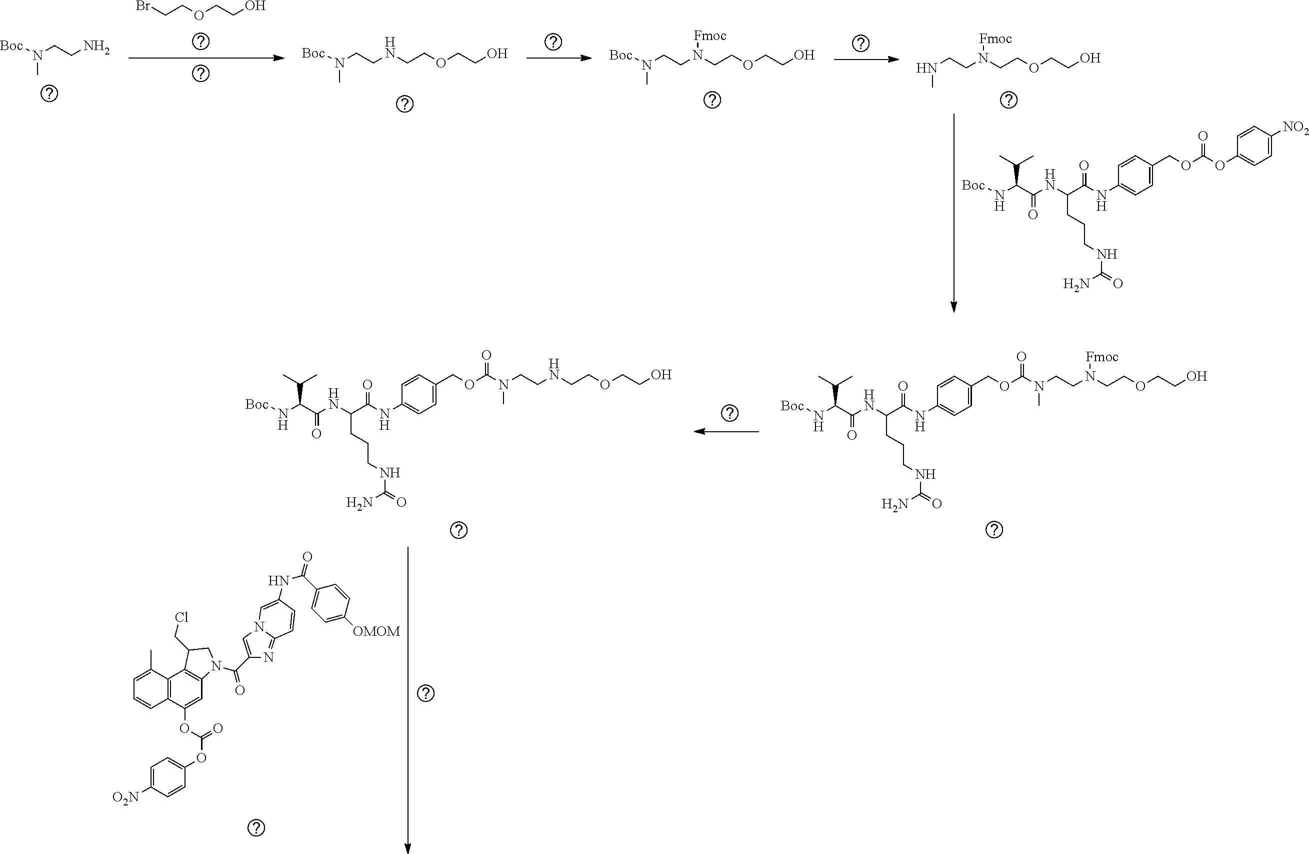

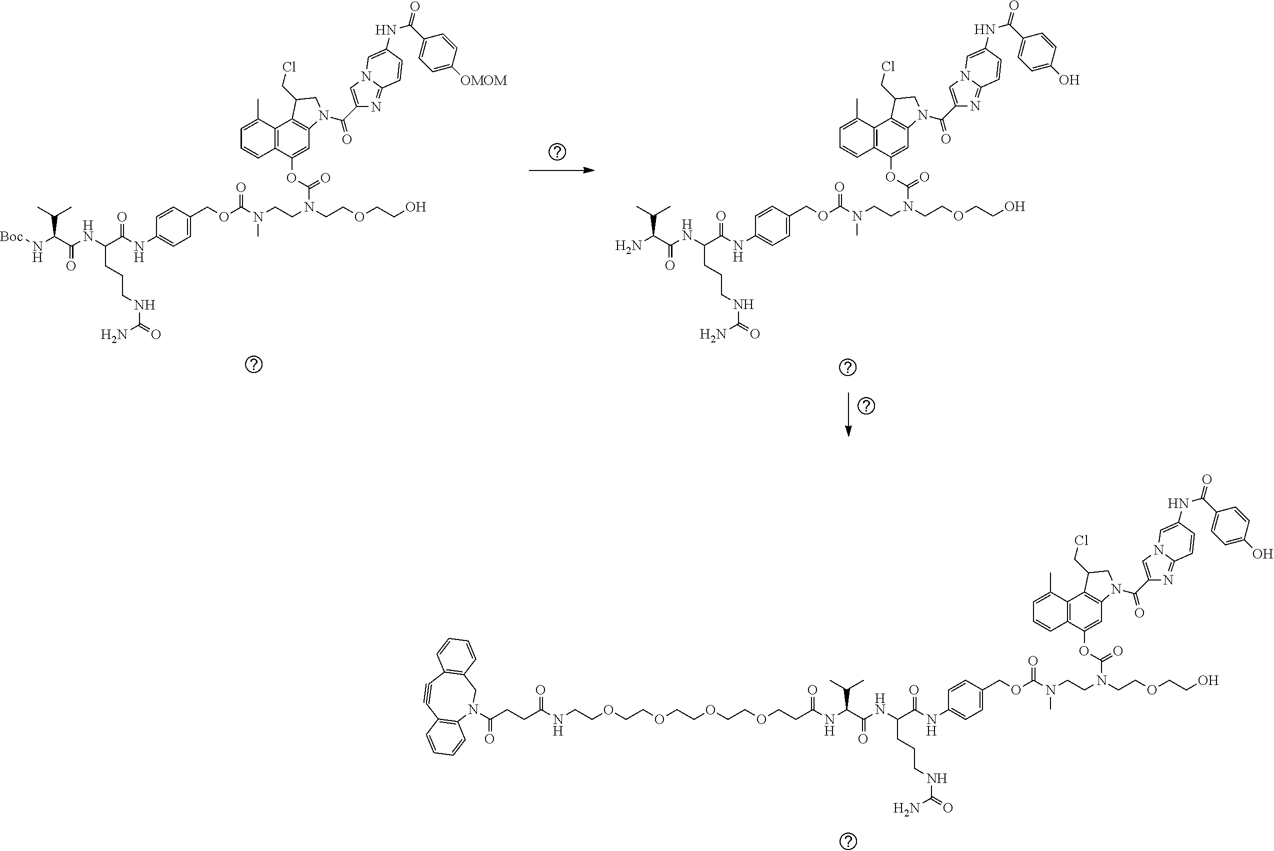

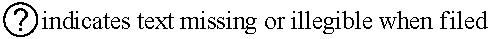

DBCO-PEG₄-vc-PAB-seco-DUBA was synthesized according to the route listed above. Tert-butyl(2-((2-(2-hydroxyethoxy)ethyl)amino)ethyl)(methyl)carbamate (24-3). To a solution of tert-butyl(2-aminoethyl)(methyl)carbamate (24-1) (5.2 g, 30 mmol) in THF (60 mL) were added 5 g TEA. Then 2-(2-bromoethoxy)ethanol (24-2) (1.7 g, 10 mmol) was dropped to the mixture stepwise. The mixture was stirred at r.t. for 5 h and monitored by TLC. The solvent was removed under reduced pressure to give the crude product 24-3.

Tert-butyl(2-((((9H-fluoren-9-yl)methoxy)carbonyl)(2-(2-hydroxyethoxy)ethyl)amino)ethyl)(methyl)carbamate (24-4). To a solution of all of the crude product 24-3 in THF (30 mL) and NaOH aq. (80 mL, 1N) were added Fmoc-Cl (7.8 g, 30.2 mmol) at r.t. The mixture was stirred at r.t. for 1 h and monitored by TLC. The solvent was then removed under reduced pressure. The residue was dissolved in 200 ml ethyl acetate and wash with washed with saturated NaHCO₃ solution and water respectively, followed by dried with Na₂SO₄. The crude product was further purified through a column chromatography to yield the 24-4 (3.5 g, yield 72% in two steps) as a light-yellow liquid. (9H-fluoren-9-yl)methyl(2-(2-hydroxyethoxy)ethyl)(2-(methylamino)ethyl)carbamate (24-5). To a solution of 24-4 (3.4 g, 7.0 mmol) in DCM (15 mL) were added 20 ml TFA. The mixture was stirred at r.t. for 4 h and monitored by TLC. The solvent was removed under reduced pressure. The crude product was further purified through a column chromatography to yield 24-5 (0.88 g, yield 33%) as a colorless liquid. HRMS (ESI+) calcd for C₂₂H₂₈N₂O₄ (M+H⁺) 385.2122, found 385.2109. 4-((S)-2-((S)-2-((tert-butoxycarbonyl)amino)-3-methylbutanamido)-5-ureidopentanamido)benzyl (2-((2-(2-hydroxyethoxy)ethyl)amino)ethyl)(methyl) carbamate (24-8). To a solution of 24-6 Boc-vc-PAB-PNP (Tsbiochem) (1.5 g, 2.3 mmol) in DMF (5 mL) were added DIPEA (1.3 mL) and 24-5 (880 mg, 2.3 mmol). The mixture was stirred at r.t. for overnight and monitored by TLC. Then 3 ml piperidine was added to the mixture and stirred for another 4 h. The product was further purified through a Prep-HPLC system to give the 24-8 as a white solid (736 mg. yield 48%). HRMS (ESI−) calcd for C₃₁H₅₃N₇O₉ (M−H⁺) 666.3832, found 666.3837.

4-((S)-2-((S)-2-((tert-butoxycarbonyl)amino)-3-methylbutanamido)-5-ureidopentanamido)benzyl(2-(((((S)-1-(chloromethyl)-3-(6-(4-(methoxymethoxy)benzamido)imidazo[1,2-a]pyridine-2-carbonyl)-9-methyl-2,3-dihydro-1H-benzo[e]indol-5-yl)oxy)carbonyl)(2-(2-hydroxyethoxy)ethyl)amino)ethyl)(methyl)carbamate (24-10). The PNP-seco-DUBA (24-9) was synthesized according to the reported procedure (Beusker P. H., et al., Mol. Pharmaceutics 2015, 12, 1813). To a solution of 24-9 (125 mg, 0.17 mmol) in DMF (5 mL) were added 130 μL TEA and 136 mg 24-8 (0.20 mmol). The mixture was stirred at r.t. for overnight and monitored by TLC. The product was further purified through a Prep-HPLC system to give the 24-10 as a white solid (71 mg. yield 33%). HRMS (ESI−) calcd for C₆₃H₇₈ClN₁₁O₁₅ (M−H⁺) 1262.5295, found 1262.5287.

4-((S)-2-((S)-2-amino-3-methylbutanamido)-5-ureidopentanamido)benzyl(2-(((((S)-1-(chloromethyl)-3-(6-(4-hydroxybenzamido)imidazo[1,2-a]pyridine-2-carbonyl)-9-methyl-2,3-dihydro-1H-benzo[e]indol-5-yl)oxy)carbonyl)(2-(2-hydroxyethoxy)ethyl)amino)ethyl)(methyl)carbamate (24-11). To a solution of 24-10 (71 mg, 0.056 mmol) in DCM (2 mL) were added 3 ml TFA. The mixture was stirred at r.t. for 3 h and monitored by TLC. The solvent was removed under reduced pressure to afford the 24-11 as a crude product (58 mg) without further purification.

DBCO-PEG₄-vc-PAB-seco-DUBA (24-12). To a solution of the crude product 24-11 (25 mg) in DMF (1.5 mL) were added 100 μL TEA and 20 mg NHS-PEG₄-DBCO ester (Xi'an Dianhua Biotechnology Co., Ltd) (0.03 mmol). The mixture was stirred at room temperature overnight and monitored by TLC. The product was further purified through a Prep-HPLC system. Concentration and lyophilization to give 24-12 as a white powder (15.2 mg). HRMS (ESI−) calcd for C₈₆H₁₀₀ClN₁₃O₁₉ (M−H⁺) 1653.6908, found 1653.6948.

### Example 25 Preparation of Antibody-(Fucα1,6)GlcNAc

Antibodies (trastuzumab, bevacizumab or rituximab) (10 mg/mL) were incubated with EndoS (SEQ ID NO: 6) (0.05 mg/mL) in 50 mM Tris-HCl buffer (pH 7.5) at 30° C. for 1 h. The reaction mixture was purified with protein A resin to give the antibody-(Fucα1,6)GlcNAc. Mass spectral analysis showed the complete conversion to trastuzumab-(Fucα1,6)GlcNAc (found as 145867 Da), bevacizumab-(Fucα1,6)GlcNAc (found as 147012 Da) and rituximab-(Fucα1,6)GlcNAc (found as 144887 Da) respectively.

The amino acid sequence of heavy chain of trastuzumab is set forth in SEQ ID NO: 11, the amino acid sequence of light chain of trastuzumab is set forth in SEQ ID NO: 10. The amino acid sequence of heavy chain of bevacizumab is set forth in SEQ ID NO: 15, the amino acid sequence of light chain of bevacizumab is set forth in SEQ ID NO: 14. The amino acid of heavy chain of sequence rituximab is set forth in SEQ ID NO: 13, the amino acid sequence of light chain of rituximab is set forth in SEQ ID NO: 12.

### Example 26 Preparation of Antibody-(Fucα1,6)(GalX′β1,4)GlcNAc

Antibody-(Fucα1,6)GlcNAc (10 mg/mL) was incubated with UDP-GalX′ (UDP-GalNAc or UDP-GalNAz) (5 mM) and bovine β1,4-GalT1(Y289L) (SEQ ID NO: 3) (0.5 mg/mL) in 50 mM Tris-HCl buffer (pH 7.5) with 10 mM MnCl₂ for overnight to 36 h at 30° C. The reaction mixture was purified with protein A resin to give the antibody-(Fucα1,6)(GalX′β1,4)GlcNAc. Mass spectral analysis showed the complete conversion to trastuzumab-(Fucα1,6)(GalNAcβ1,4)GlcNAc (found as 146276 Da), bevacizumab-(Fucα1,6)(GalNAcβ1,4)GlcNAc (found as 147419 Da), rituximab-(Fucα1,6)(GalNAcβ1,4)GlcNAc (found as 145296 Da), and trastuzumab-(Fucα1,6)(GalNAzβ1,4)GlcNAc (found as 146357 Da) respectively.

### Example 27 Preparation of Antibody-(Fucα1,6)(GalNAcβ1,4)GlcNAc-Fuc*′

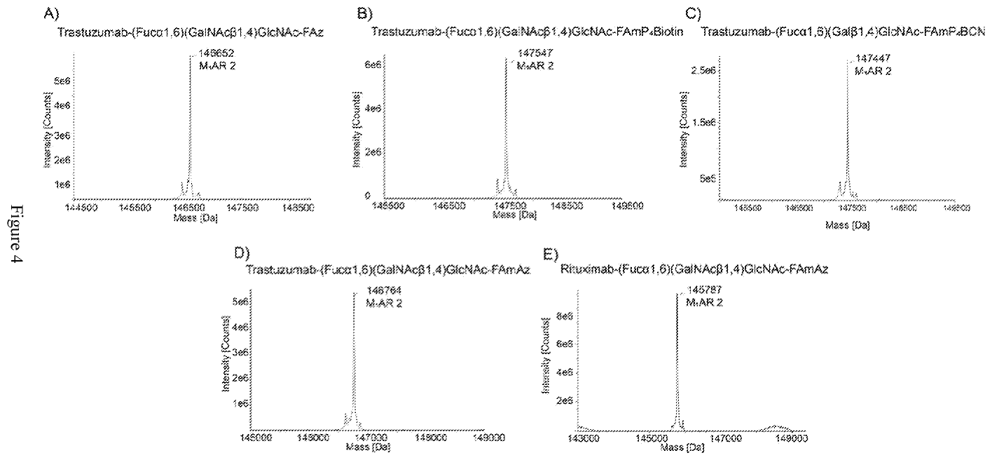

Antibody-(Fucα1,6)(GalNAcβ1,4)GlcNAc (8 mg/mL) was incubated with GDP-Fuc*′ (GDP-FAz, GDP-FAmAz, GDP-FAmP₄BCN or GDP-FAmP₄Biotin) (5 mM) and Hp1,3-FucT (SEQ ID NO: 1) (0.5 mg/mL) in 50 mM Tris-HCl buffer (pH 7.5) with 20 mM MgCl₂ at 37° C. for overnight to 72 h. The reaction mixture was purified with protein A resin to give the antibody-(Fucα1,6)(GalNAcβ1,4)GlcNAc-Fuc*′ conjugates. Mass spectral analysis showed the formation of one major peak corresponding to trastuzumab-(Fucα1,6)(GalNAcβ1,4)GlcNAc-FAz (found as 146652 Da, M₁AR 2), trastuzumab-(Fucα1,6)(GalNAcβ1,4)GlcNAc-FAmP₄Biotin (found as 147547, M₁AR 2), trastuzumab-(Fucα1,6)(GalNAcβ1,4)GlcNAc-FAmP₄BCN (found as 147447, M₁AR 2), trastuzumab-(Fucα1,6)(GalNAcβ1,4)GlcNAc-FAmAz (found as 146764 Da, M₁AR 2), and rituximab-(Fucα1,6)(GalNAcβ1,4)GlcNAc-FAmAz (found as 145787 Da, M₁AR 2) (FIG. 4) respectively.

### Example 28 Preparation of Antibody-GlcNAc

Antibodies (10 mg/mL) were incubated with EndoS (0.05 mg/mL) and Alfc (SEQ ID NO: 8) (1.5 mg/mL) in 50 mM Tris-HCl buffer (pH 7.5) at 37° C. for 24 h. The reaction mixture was purified with protein A resin to give the Antibody-GlcNAc. Mass spectral analysis showed the complete conversion to trastuzumab-GlcNAc (found as 145582 Da), hRS7-GlcNAc (found as 145426 Da) and rituximab-GlcNAc (found as 144599 Da), respectively.

The amino acid sequence of heavy chain of hRS7 is set forth in SEQ ID NO: 17, the amino acid sequence of light chain of hRS7 is set forth in SEQ ID NO: 16.

### Example 29 Preparation of Antibody-(GalX′β1,4)GlcNAc

Antibody-GlcNAc (10 mg/mL) was incubated with UDP-GalX′ (UDP-GalNAc, UDP-GalNAz or UDP-GalNH₂) (5 mM) and bovine β1,4-GalT1(Y289L) (0.3 mg/mL) in 50 mM Tris-HCl buffer (pH 7.5) with 10 mM MnCl₂ for overnight to 72 h at 30° C. The reaction mixture was purified with protein A resin to give the antibody-(GalX′β1,4)GlcNAc. Mass spectral analysis showed the complete conversion to trastuzumab-(GalNAcβ1,4)GlcNAc (found as 145986 Da), trastuzumab-(GalNH₂β1,4)GlcNAc (found as 145904 Da), trastuzumab-(GalNAzβ1,4)GlcNAc (found as 146070 Da), rituximab-(GalNAcβ1,4)GlcNAc (found as 145009 Da) and hRS7-(GalNAcβ1,4)GlcNAc (found as 145830 Da) respectively.

### Example 30 Preparation of Antibody-(GalNAcβ1,4)GlcNAc-Fuc*′

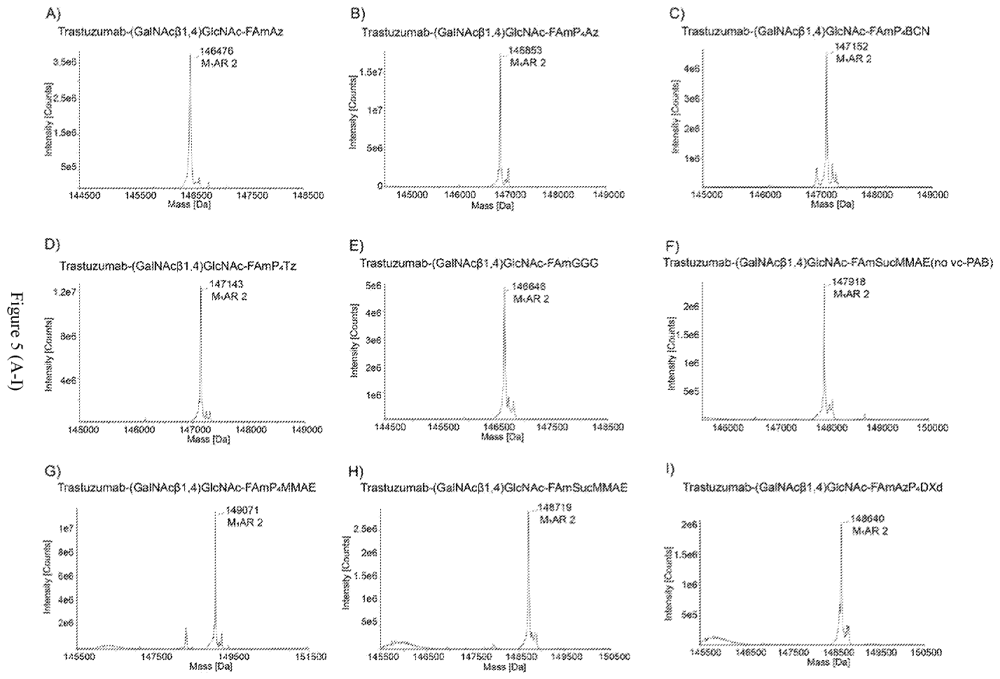

Antibody-(GalNAcβ1,4)GlcNAc (8 mg/mL) was incubated with GDP-Fuc*′ (GDP-FAmAz, GDP-FAmP₄Az, GDP-FAmP₄BCN, GDP-FAmP₄Tz, GDP-FAmGGG, GDP-FAmP₄MMAE, GDP-FAmAzP₄DXd, GDP-FAmSucMMAE, or GDP-FAmSucMMAE (no vc-PAB)) (5 mM) and Hp1,3-FucT (0.5 mg/mL) in 50 mM Tris-HCl buffer (pH 7.5) with 20 mM MgCl₂ at 30° C. for overnight to 48 h respectively. The reaction mixture was purified with protein A resin to give the antibody-(GalNAcβ1,4)GlcNAc-Fuc*′ conjugates. Mass spectral analysis showed the formation of one major peak corresponding to trastuzumab-(GalNAcβ1,4)GlcNAc-FAmAz (found as 146476 Da, M₁AR 2), trastuzumab-(GalNAcβ1,4)GlcNAc-FAmP₄Az (found as 146853 Da, M₁AR 2), trastuzumab-(GalNAcβ1,4)GlcNAc-FAmP₄BCN (found as 147152 Da, M₁AR 2), trastuzumab-(GalNAcβ1,4)GlcNAc-FAmP₄Tz (found as 147143 Da, M₁AR 2), trastuzumab-(GalNAcβ1,4)GlcNAc-FAmGGG (found as 146646 Da, M₁AR 2), trastuzumab-(GalNAcβ1,4)GlcNAc-FAmSucMMAE (no vc-PAB) (found as 147918 Da, M₁AR 2), trastuzumab-(GalNAcβ1,4)GlcNAc-FAmP₄MMAE (found as 149071 Da, M₁AR 2), trastuzumab-(GalNAcβ1,4)GlcNAc-FAmSucMMAE (found as 148719 Da, M₁AR 2), trastuzumab-(GalNAcβ1,4)GlcNAc-FAmAzP₄DXd (found as 148640 Da, M₁AR 2), rituximab-(GalNAcβ1,4)GlcNAc-FAmAz (found as 145495 Da, M₁AR 2), rituximab-(GalNAcβ1,4)GlcNAc-FAmP₄BCN (found as 146177 Da, M₁AR 2), rituximab-(GalNAcβ1,4)GlcNAc-FAmP₄Tz (found as 146167 Da, M₁AR 2), rituximab-(GalNAcβ1,4)GlcNAc-FAmP₄Biotin (found as 146277 Da, M₁AR 2), rituximab-(GalNAcβ1,4)GlcNAc-FAmSucMMAE (found as 147738 Da, M₁AR 2), hRS7-(GalNAcβ1,4)GlcNAc-FAmAzP₄DXd (found as 148414 Da, M₁AR 2) and hRS7-(GalNAcβ1,4)GlcNAc-FAmSucMMAE (found as 148575 Da, M₁AR 2) respectively (FIG. 5). All the compositions of conjugates have an average M₁AR of 1.8-2.0, respectively.

In terms of trastuzumab-(GalNAcβ1,4)GlcNAc-FAmP₄MMAE, mass spectral analysis showed the formation of a major peak corresponding to the trastuzumab-(GalNAcβ1,4)GlcNAc-FAmP₄MMAE and a minor peak (found as 148312 Da) due to fragmentation of the vc-PAB linker during mass spectrometry, similar fragments appeared in the following antibody-drug conjugates which containing the vc-PAB linkers (FIG. 5).

### Example 31 Preparation of Trastuzumab-(GalNH₂β1,4)GlcNAc-Fuc*′

Trastuzumab-(GalNH₂β1,4)GlcNAc (8 mg/mL) was incubated with GDP-Fuc*′ (GDP-FAz, GDP-FAmP₄Biotin, GDP-FAzP₄Biotin, GDP-FAmP₄Tz or GDP-FAmSucMMAE) (5 mM) and Hp1,3-FucT (0.5 mg/mL) in 50 mM Tris-HCl buffer (pH 7.5) with 20 mM MgCl₂ at 30° C. for overnight to 48 h. The reaction mixture was purified with protein A resin to give the antibody conjugates. Mass spectral analysis showed the formation of one major peak corresponding to trastuzumab-(GalNH₂β1,4)GlcNAc-FAz (found as 146276 Da, M₁AR 2), trastuzumab-(GalNH₂β1,4)GlcNAc-FAmP₄Biotin (found as 147173 Da, M₁AR 2), trastuzumab-(GalNH₂β1,4)GlcNAc-FAzP₄Biotin (found as 147186 Da, M₁AR 2), trastuzumab-(GalNH₂β1,4)GlcNAc-FAmP₄Tz (found as 147061 Da, M₁AR 2), trastuzumab-(GalNH₂β1,4)GlcNAc-FAmSucMMAE (found as 148634 Da, M₁AR 2) respectively (FIG. 6).

### Example 32 Preparation of Trastuzumab-(GalNH₂β1,4)GlcNAc-FAmDM4

Trastuzumab-(GalNH₂β1,4)GlcNAc (8 mg/mL) was incubated with GDP-FAmDM4 (2 mM) and Hp1,3-FucT (0.5 mg/mL) in 50 mM Tris-HCl buffer (pH 7.5) with 20 mM MgCl₂ at 30° C. for 48 h. The reaction mixture was purified with protein A resin to give the antibody conjugates. Mass spectral analysis showed the formation of one major peak corresponding to trastuzumab-(GalNH₂β1,4)GlcNAc-FAmDM4 (found as 147983 Da, M₁AR 2).

### Example 33 Preparation of bevacizumab-(GalNAc)₂F and bevacizumab-(GalNAz)₂F

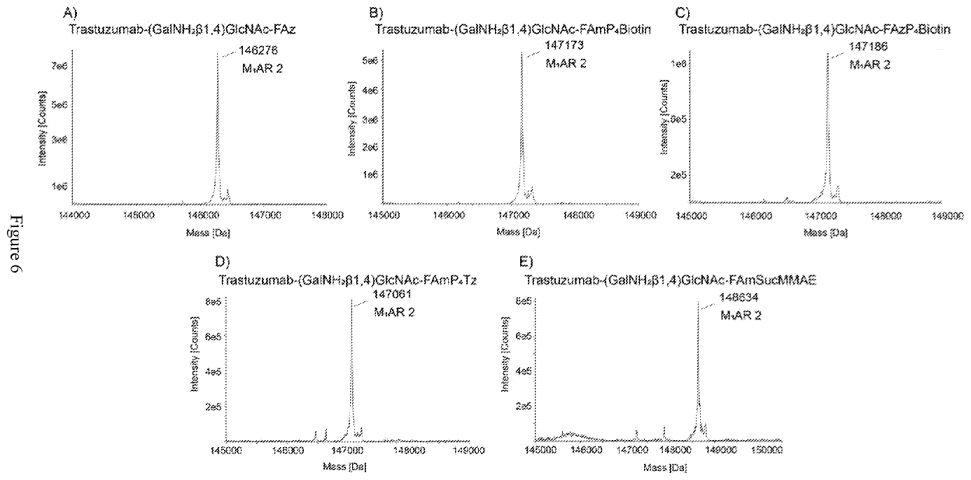

Bevacizumab (mainly consisted of G₀F) (10 mg/mL) was incubated with UDP-GalNAc or UDP-GalNAz (5 mM) and bovine β1,4-GalT1(Y289L) (0.5 mg/mL) in 50 mM Tris-HCl buffer (pH 7.5) with 10 mM MnCl₂ for 40 h at 30° C. The reaction mixture was purified with protein A resin to give the product. Mass spectral analysis showed the formation of one major peak corresponding to bevacizumab-(GalNAc)₂F (found as 150026 Da) and bevacizumab-(GalNAz)₂F (found as 150181 Da)(FIG. 8).

### Example 34 Preparation of bevacizumab-(GalNAc)₂F-Fuc*′ and bevacizumab-(GalNAz)₂F-Fuc*′

Bevacizumab-GalNAc₂F or bevacizumab-(GalNAz)₂F (8 mg/mL) were incubated with GDP-Fuc*′ (GDP-FAz, GDP-FAmAz, GDP-FAmBiotin or GDP-FAmP₄Tz) (5 mM) and Hp1,3-FucT (0.5 mg/mL) in 50 mM Tris-HCl buffer (pH 7.5) with 20 mM MgCl₂ at 30° C. for overnight to 48 h. The reaction mixture was purified with protein A resin to give the products. Mass spectral analysis showed the formation of one major peak corresponding to bevacizumab-GalNAc₂F-FAz (found as 150772 Da, M₁AR 4), bevacizumab-(GalNAz)₂F-FAmAz (found as 151171 Da, M₁AR 4), bevacizumab-(GalNAz)₂F-FAmBiotin (found 152719 as Da, M₁AR 4) and bevacizumab-(GalNAz)₂F-FAmP₄Tz (found as 152520 Da, M₁AR 4) (FIG. 8).

### Example 35 Preparation of Trastuzumab-(GalNAcβ1,4)GlcNAc-FAmAzDBCO-MMAE

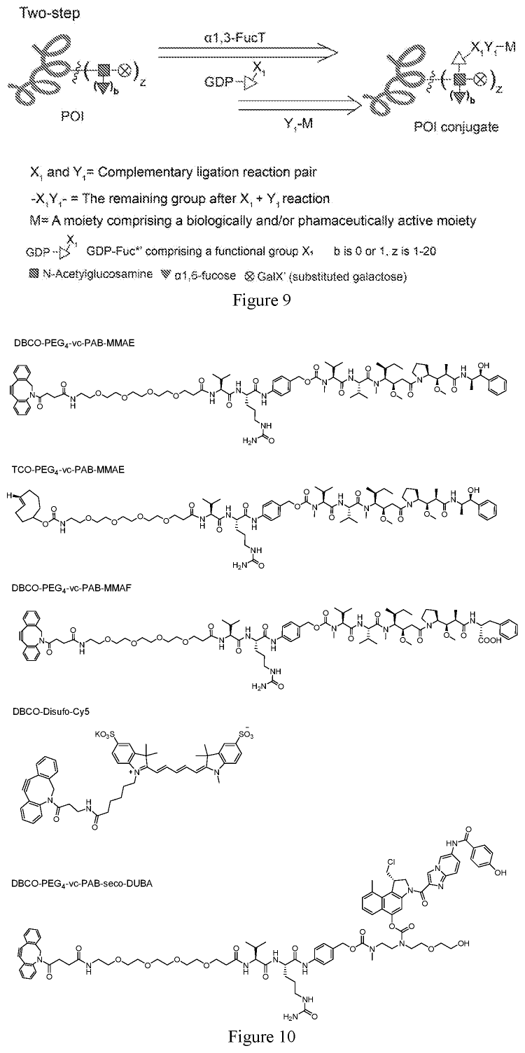

Trastuzumab-(GalNAcβ1,4)GlcNAc-FAmAz (5 mg/mL) was incubated with DBCO-PEG₄-vc-PAB-MMAE (Levena Biopharma) (200 μM) in PBS (pH 7.4) with 8% DMSO at r.t. for 4 hours. The reaction mixture was purified with protein A resin to give the trastuzumab-(GalNAcβ1,4)GlcNAc-FAmAzDBCO-MMAE. Mass spectral analysis showed one major peak (found as 149790 Da, M₁AR 2) with two MMAE added to one trastuzumab-(GalNAcβ1,4)GlcNAc-FAmAz molecule. The composition of conjugate has an average M₁AR of 1.8-2.0 (FIG. 11A).

### Example 36 Preparation of Trastuzumab-(GalNAcβ1,4)GlcNAc-FAmP₄AzDBCO-seco-DUBA

Trastuzumab-(GalNAcβ1,4)GlcNAc-FAmP₄Az (5 mg/mL) was incubated withDBCO-PEG₄-vc-PAB-seco-DUBA (200 μM) in PBS (pH 7.4) with 15% DMSO at r.t. for 16 hours. The reaction mixture was purified with protein A resin to give the trastuzumab-(GalNAcβ1,4)GlcNAc-FAmP₄AzDBCO-seco-DUBA. Mass spectral analysis showed one major peak (found as 150163 Da, M₁AR 2) with two seco-DUBA added to one trastuzumab-(GalNAcβ1,4)GlcNAc-FAmP₄Az molecule. The composition of conjugate has an average M₁AR of 1.8-2.0 (FIG. 11B).

### Example 37 Preparation of Trastuzumab-(GalNAcβ1,4)GlcNAc-FAmP₄TzTCO-MMAE

Trastuzumab-(GalNAcβ1,4)GlcNAc-FAmP₄Tz (5 mg/mL) was incubated with TCO-PEG₄-vc-PAB-MMAE (200 μM) in PBS (pH 7.4) with 8% DMSO at r.t. for 1 h. The reaction mixture was purified with protein A resin to give the Trastuzumab-(GalNAcβ1,4)GlcNAc-FAmP₄TzTCO-MMAE. Mass spectral analysis showed the generation of one major peak corresponding to trastuzumab-(GalNAcβ1,4)GlcNAc-FAmP₄TzTCO-MMAE (found as 150133 Da, M₁AR 2), one minor peak (found as 149376 Da) due to the fragmentation of vc-PAB linker and one more minor peak (found as 148719 Da) due to the fragmentation of TCO linker during MS spectrometry, similar fragments appear in the following antibody-drug conjugates which containing the TCO moiety. The composition of conjugate has an average M₁AR of 1.8-2.0 (FIG. 11C).

### Example 38 Preparation of rituximab-(GalNAcβ1,4)GlcNAc-FAmAzDBCO-MMAE

Rituximab-(GalNAcβ1,4)GlcNAc-FAmAz (5 mg/mL) was subjected to the process described in example 35. Mass spectral analysis showed one major peak (148808 Da) with two MMAE added to one rituximab-(GalNAcβ1,4)GlcNAc-FAmAz molecule.

### Example 39 Preparation of Trastuzumab-(Fucα1,6)(GalNAcβ1,4)GlcNAc-FAzDBCO-MMAE

Trastuzumab-(Fucα1,6)(GalNAcβ1,4)GlcNAc-FAz (5 mg/mL) was incubated with DBCO-PEG₄-vc-PAB-MMAE (200 μM) in PBS (pH 7.4) with 8% DMSO at r.t. for 16 hours. The reaction mixture was purified with protein A resin to give the trastuzumab-(Fucα1,6)(GalNAcβ1,4)GlcNAc-FAzDBCO-MMAE. Mass spectral analysis showed one major peak (found as 149967 Da, M₁AR 2) with two MMAE added to one trastuzumab-(Fucα1,6)(GalNAcβ1,4)GlcNAc-FAz molecule. The composition of conjugate has an average M₁AR of 1.8˜2.0.

### Example 40 In Vitro Efficacy of Transtuzmab-Drug Conjugates

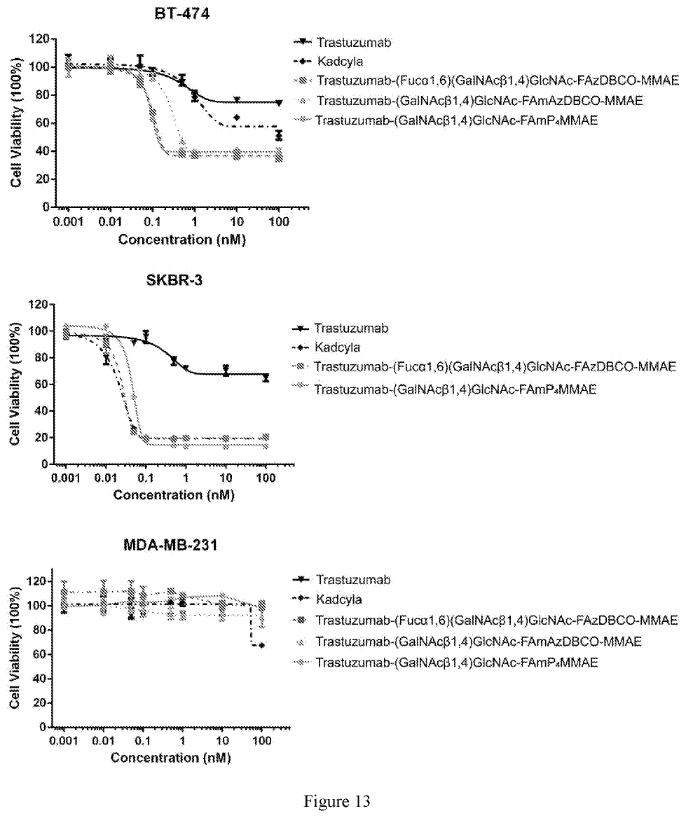

BT-474(Her2+) and SK-Br-3 (Her2+) were cultured in RPMI 1640 medium supplemented with 10% FBS (Gibco). MDA-MB-231 (Her2−) cells were DMEM (Gibco) supplemented with 10% FBS (Gibco). The cells were plated in 96-well plates with 5000 cells per well and were incubated for 24 hours at 37° C. and 5% CO₂. After removing of the culture medium, samples trastuzumab, kadcyla, trastuzumab-(GalNAcβ1,4)GlcNAc-FAmAzDBCO-MMAE, trastuzumab-(GalNAcβ1,4)GlcNAc-FAmP₄MMAE or trastuzumab-(Fucα1,6)(GalNAcβ1,4)GlcNAc-FAzDBCO-MMAE was added to the culturing medium to a series of final concentrations (100 nM, 10 nM, 1 nM, 0.5 nM, 0.1 nM, 0.05 nM, 0.01 nM, 0.001 nM and 0 nM) and added to the plates respectively. The cells were incubated for 72 h at 37° C. and 5% CO₂ and subjected to a CellTiter-Glo® Luminescent Cell Viability Assay (Promega) to measure the cell viability. The trastuzumab conjugates showed high potency towards Her2 positive cell lines, but not of the Her2 negative cell line MDA-MB-231 (FIG. 13).

### Example 41 In vitro efficacy of hRS7-(GalNAcβ1,4)GlcNAc-FAmSucMMAE

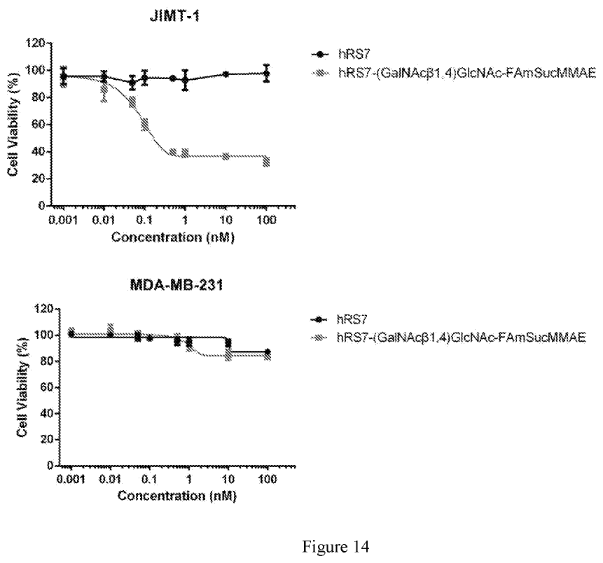

JIMT-1 (trop2 high expression) and MDA-MB-231 (trop2 low expression) cells were cultured in DMEM (Gibco) supplemented with 10% FBS (Gibco). The cells were plated in 96-well plates with 5000 cells per well and were incubated for 24 hours at 37° C. and 5% CO2. After removing of the culture medium, samples hRS7 and hRS7-(GalNAcβ1,4)GlcNAc-FAmSucMMAE were added to the culturing medium to a series of final concentrations (100 nM, 10 nM, 1 nM, 0.5 nM, 0.1 nM, 0.05 nM, 0.01 nM, 0.001 nM and 0 nM) and added to the plates respectively. The cells were incubated for 72 h at 37° C. and 5% CO₂ and subjected to a CellTiter-Glo® Luminescent Cell Viability Assay (Promega) to measure the cell viability. The hRS7-(GalNAcβ1,4)GlcNAc-FAmSucMMAE showed high potent towards trop2-high expressing cell line JIMT-1, but not the trop2-low expressing cell line MDA-MB-231 (FIG. 14).

### Example 42 Preparation of Antibody-(GalNAzβ1,4)GlcNAc-Fuc*′

Antibody-(GalNAzβ1,4)GlcNAc (8 mg/mL) was incubated with GDP-Fuc*′ (GDP-FAmAz, GDP-FAmP₄Biotin, GDP-FAmP₈Tz, GDP-FAmP₄MMAE) (5 mM) and Hp1,3-FucT (0.3 mg/mL) in 50 mM Tris-HCl buffer (pH 7.5) with 20 mM MgCl₂ at 30° C. for overnight to 48 h respectively. The reaction mixture was purified with protein A resin to give the antibody-(GalNAββ1,4)GlcNAc-Fuc*′ conjugates. Mass spectral analysis showed the generation of one major peak corresponding to trastuzumab-(GalNAzβ1,4)GlcNAc-FAmP₄Biotin (found as 147335 Da, M₁AR 2), trastuzumab-(GalNAzβ1,4)GlcNAc-FAmP₄MMAE (found as 149152 Da, M₁AR 2), trastuzumab-(GalNAzβ1,4)GlcNAc-FAmAz (found as 146551 Da, M₁AR 2), trastuzumab-(GalNAzβ1,4)GlcNAc-FAmP₈Tz (found as 147578 Da, M₁AR 2) respectively (FIG. 16 and FIG. 17).

### Example 43 Preparation of Trastuzumab-(Fucα1,6)(GalNAzβ1,4)GlcNAc-FAmP₄Tz

Trastuzumab-(Fucα1,6)(GalNAzβ1,4)GlcNAc (8 mg/mL) was incubated with GDP-FAmP₄Tz (5 mM) and Hp1,3-FucT (0.5 mg/mL) in 50 mM Tris-HCl buffer (pH 7.5) with 20 mM MgCl₂ at 37° C. for 72 h. The reaction mixture was purified with protein A resin to give the product. Mass spectral analysis showed the formation of one major peak corresponding to trastuzumab-(Fucα1,6)(GalNAzβ1,4)GlcNAc-FAmP₄Tz (found as 147520 Da, M₁AR 2) (FIG. 17B).

### Example 44 “One-Pot” Synthesis of rituximab-(GalNAzβ1,4)GlcNAc-FucAmP₄Tz

Rituximab (8 mg/mL) were incubated with EndoS (0.05 mg/mL) and Alfc (1.5 mg/mL) in 50 mM Tris-HCl buffer (pH 7.5) at 30° C. After 24 h, the reaction mixtures were added with UDP-GalNAz (5 mM), bovine β(1,4)-GalT1(Y289L) (0.5 mg/mL), GDP-FAmP₄Tz (5 mM) and Hp1,3-FucT (0.5 mg/mL), MgCl₂ (20 mM) and MnCl₂ (10 mM) followed by incubating at 30° C. for 40 h. The modified antibody was purified with protein A resin to give the rituximab-(GalNAzβ1,4)GlcNAc-FAmP₄Tz (found as 146244 Da, M₁AR 2) (FIG. 17B).

### Example 45 Preparation of Trastuzumab-(GalNAzDBCO-MMAE)GlcNAc-FAmP₄Biotin

Trastuzumab-(GalNAβ1,4)GlcNAc-FAmP₄Biotin (5 mg/mL) was incubated with DBCO-PEG₄-vc-PAB-MMAE (200 μM) in PBS (pH 7.4) with 8% DMSO r.t. for 4 h. The reaction mixture was purified with protein A resin to give the product. Mass spectral analysis showed the formation of one major peak corresponding to trastuzumab-(GalNAzDBCO-MMAE)GlcNAc-FAmP₄Biotin (found as 150654 Da, M₁AR 2 and M₂AR 2). The composition of conjugate has an average M₁AR of 1.8-2.0 and an average M₂AR of 1.8-2.0 (FIG. 16).

### Example 46 Preparation of Trastuzumab-(GalNAzDBCO-MMAF)GlcNAc-FAmP₄MMAE

Trastuzumab-(GalNAβ1,4)GlcNAc-FAmP₄MMAE (5 mg/mL) was incubated with DBCO-PEG₄-vc-PAB-MMAF (Levena Biopharma) (200 μM) in PBS (pH 7.4) with 8% DMSO at r.t. for 4 hours. The reaction mixture was purified with protein A resin to give the product. Mass spectral analysis showed the formation of one major peak corresponding to the trastuzumab-(GalNAzDBCO-MMAF)GlcNAc-FAmP₄MMAE (found as 152498 Da, M₁AR 2 and M₂AR 2). The composition of conjugate has an average M₁AR of 1.8-2.0 and an average M₂AR of 1.8-2.0 (FIG. 16).

### Example 47 Preparation of Trastuzumab-(GalNAzDBCO-Cy5)GlcNAc-FAmP₄MMAE

Trastuzumab-(GalNAzβ1,4)GlcNAc-FAmP₄MMAE (5 mg/mL) was incubated with DBCO-Disulfo-Cy5 (200 μM) (Xi'an ruixi Biological Technology) in PBS (pH 7.4) with 8% DMSO r.t. for 4 h. Mass spectral analysis showed the generation of one major peak corresponding to the trastuzumab-(GalNAzDBCO-Cy5)GlcNAc-FAmP₄MMAE (found as 150956 Da, M₁AR 2 and M₂AR 2)(FIG. 16).

### Example 48 Preparation of Trastuzumab-(GalNAzDBCO-MMAE)GlcNAc-FAmAzDBCO-MMAE

Trastuzumab-(GalNAβ1,4)GlcNAc-FAmAz (5 mg/mL) was incubated with DBCO-PEG₄-vc-PAB-MMAE (300 μM) in PBS (pH 7.4) with 8% DMSO r.t. for 6 hours. The reaction mixture was purified with protein A resin to give the conjugate. Mass spectral analysis showed the formation of one major peak corresponding to trastuzumab-(GalNAzDBCO-MMAE)GlcNAc-FAmAzDBCO-MMAE (found as 153185 Da, M₁AR 2 and M₂AR 2). (FIG. 17A).

### Example 49 Preparation of Trastuzumab-(Fucα1,6)(GalNAzDBCO-Cy5)GlcNAc-FAmP₄TzTCO-MMAE

Trastuzumab-(Fucα1,6)(GalNAβ1,4)GlcNAc-FAmP₄Tz (5 mg/mL) was incubated with DBCO-Disulfo-Cy5 (200 μM) and TCO-PEG₄-vc-PAB-MMAE (200 μM) in PBS (pH 7.4) with 8% DMSO at r.t. for 4 hours. The reaction mixture was purified with protein A resin to give the product. Mass spectral analysis showed the formation of one major peak corresponding to trastuzumab-(Fucα1,6)(GalNAzDBCO-Cy5)GlcNAc-FAmP₄TzTCO-MMAE (found as 152308 Da, M₁AR 2 and M₂AR 2) with two MM AE and two Cy5 added to one trastuzumab-(Fucα1,6)(GalNAzβ1,4)GlcNAc-FAmP₄Tz molecule (FIG. 17B).

### Example 50 Preparation of rituximab-(GalNAzDBCO-Cy5)GlcNAc-FAmP₄TzTCO-MMAE

Rituximab-(GalNAzβ1,4)GlcNAc-FAmP₄Tz (5 mg/mL) was subjected to the process described in example 49. Mass spectral analysis showed the formation of one major peak corresponding to the rituximab-(GalNAzDBCO-Cy5)GlcNAc-FAmP₄TzTCO-MMAE (found as 151035 Da, M₁AR 2 and M₂AR 2) with two MMAE and two Cy5 added to one rituximab-(GalNAzβ1,4)GlcNAc-FAmP₄Tz molecule (FIG. 17B).

### Example 51 Preparation of Trastuzumab-(GalNAzDBCO-MMAF)GlcNAc-FAmP₈TzTCO-MMAE

Trastuzumab-(GalNAzβ1,4)GlcNAc-FAmP₈Tz (5 mg/mL) was incubated with DBCO-PEG₄-vc-PAB-MMAF (200 μM) and TCO-vc-PAB-MMAE (200 μM) in PBS (pH 7.4) with 8% DMSO at r.t. for 4 hours. The reaction mixture was purified with protein A resin to give the trastuzumab-(GalNAzDBCO-MMAF)GlcNAc-FAmP₈TzTCO-MMAE. Mass spectral analysis showed the generation of one major peak (found 153913 Da, M₁AR 2 and M₂AR 2) with two MMAE and two MMAF added to one trastuzumab-(GalNAzβ1,4)GlcNAc-FAmP₈Tz molecule (FIG. 17B).

### Example 52 Preparation of Trastuzumab-(GalNAzDBCO-seco-DUBA)GlcNAc-FAmP₈TzTCO-MMAE

Trastuzumab-(GalNAβ1,4)GlcNAc-FAmP₈Tz (5 mg/mL) was incubated with DBCO-PEG₄-vc-PAB-seco-DUBA (200 μM) and TCO-vc-pAB-MMAE (200 μM) in PBS (pH 7.4) with 8% DMSO at r.t. for overnight. The reaction mixture was purified with protein A resin to give the trastuzumab-(GalNAzDBCO-seco-DuBA)GlcNAc-FAmP₈TzTCO-MMAE. Mass spectral analysis showed the generation of one major peak (found as 153878 Da, M₁AR 2 and M₂AR 2) with two MMAE and two seco-DUBA added to one trastuzumab-(GalNAzβ1,4)GlcNAc-FAmP₈Tz molecule (FIG. 17B).

### Example 53 Preparation of bevacizumab-(GalNAzDBCO-MMAE)₂F-FAmAzDBCO-MMAE

Bevacizumab-(GalNAz)₂F-FAmAz (5 mg/mL) was incubated with DBCO-PEG₄-vc-PAB-MMAE (400 μM) in PBS (pH 7.4) with 8% DMSO at r.t. for overnight. The reaction mixture was purified with protein A resin to give the product. Mass spectral analysis showed the generation of one major peak corresponding to bevacizumab-(GalNAzDBCO-MMAE)₂F-FAmAzDBCO-MMAE (found as 164428 Da, M₁AR 4 and M₂AR 4) with eight MMAE added to one bevacizumab-(GalNAz)₂F-FAmAz molecule

### Example 54 “One-pot” synthesis of trastuzumab-(Galβ1,4)GlcNAc-Fuc*′

Trastuzumab (8 mg/mL) were incubated with EndoS (0.05 mg/mL) and Alfc (1.5 mg/mL) in 50 mM Tris-HCl buffer (pH 7.5) at 30° C. After 24 h, the reaction mixtures were added with UDP-galactose (final concentration 5 mM), bovine β(1,4)-GalT1(Y289L) (final concentration 0.5 mg/mL), GDP-FAmAz or GDP-FAmP₄Az (final concentration 5 mM) and Hp1,3-FucT (final concentration 0.5 mg/mL), MgCl₂ (final concentration 20 mM) and MnCl₂ (final concentration 10 mM) followed by incubating at 30° C. for 24 h. The modified antibody was purified with protein A resin to give the trastuzumab-(Galβ1,4)GlcNAc-FAmP₄Az (found as 146771 Da, MAR2) trastuzumab-(Galβ1,4)GlcNAc-FAmAz (found as 146387 Da, MAR2) respectively.

### Example 55 Preparation of Trastuzumab-(Galβ1,4)GlcNAc-FAmAzDBCO-MMAE and trastuzumab-(Galβ1,4)GlcNAc-FAmAzDBCO-MMAF

Trastuzumab-(Galβ1,4)GlcNAc-FAmAz (5 mg/mL) was incubated with DBCO-PEG₄-vc-PAB-MMAE (200 μM) or DBCO-PEG₄-vc-PAB-MMAF (200 μM) in PBS with 8% DMSO at r.t. for 4 hours. The reaction mixture was purified with protein A resin to give the products. Mass spectral analysis showed the generation of one major peak corresponding to trastuzumab-(Gal β1,4)GlcNAc-FAmAzDBCO-MMAE (found as 149708 Da, M₁AR 2) and trastuzumab-(Gal R_{1,4})GlcNAc-FAmAzDBCO-MMAF (found as 149736 Da, M₁AR 2) respectively.

### Example 56 Preparation of Trastuzumab-(Galβ1,4)GlcNAc-FAmP₄AzDBCO-seco-DUBA

Trastuzumab-(Galβ1,4)GlcNAc-FAmP₄Az (5 mg/mL) was incubated with DBCO-PEG₄-vc-PAB-seco-DUBA (200 μM) in PBS (pH 7.4) with 15% DMSO at r.t. for 16 hours. The reaction mixture was purified with protein A resin to give the trastuzumab-(GalNAcβ1,4)GlcNAc-FAmP₄AzDBCO-seco-DUBA. Mass spectral analysis showed the formation of one major peak corresponding to the trastuzumab-(Galβ1,4)GlcNAc-FAmP₄AzDBCO-seco-DUBA (found as 150082 Da, M₁AR 2).

### Example 57 In Vitro Efficacy of Dual-Conjugated ADC

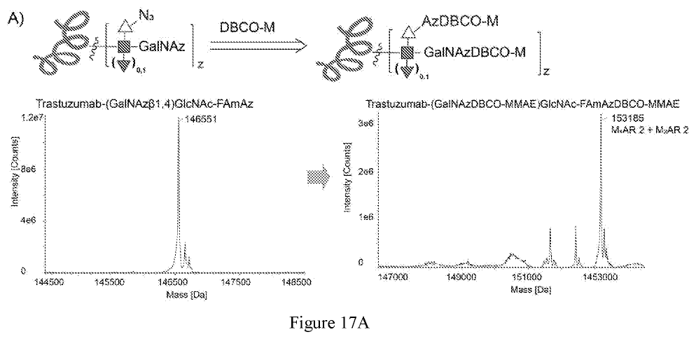

SKOV-3(Her2⁺) and NCI-N₈₇ (Her2⁺) cells were cultured in RPMI 1640 medium (Gibco) supplemented with 10% FBS (Gibico). The cells were plated in 96-well plates with 5000 cells per well and were incubated for 24 hours at 37° C. and 5% CO₂. After removing of the culture medium, the antibody samples trastuzumab-(GalNAzDBCO-MMAF)GlcNAc-FAmP₈TzTCO-MMAE (for SKOV-3 group), trastuzumab-(Galβ1,4)GlcNAc-FAmAzDBCO-MMAE (for SKOV-3 and NCI-N87 group), trastuzumab-(Galβ1,4)GlcNAc-FAmAzDBCO-MMAF (for SKOV-3 group), trastuzumab-(GalNAzDBCO-seco-DUBA)GlcNAc-FAmP₈TzTCO-MMAE (for NCI-N87 group) or trastuzumab-(Galβ1,4)GlcNAc-FAmP₄AzDBCO-seco-DUBA (for NCI-N87 group) were added to the culturing medium to a series of final concentrations (100 nM, 10 nM, 1 nM, 0.5 nM, 0.1 nM, 0.05 nM, 0.01 nM, 0.001 nM and 0 nM) and added to the plates respectively. Then the cells were incubated for 72 h at 37° C. and 5% CO₂ and subjected to a CellTiter-Glo® Luminescent Cell Viability Assay (Promega) to measure the cell viability. The dual-drug conjugate trastuzumab-(GalNAzDBCO-MMAF)GlcNAc-FAmP₈TzTCO-MMAE showed higher efficacy towards SKOV-3 cells compared to that of the single-drug conjugates trastuzumab-(Galβ1,4)GlcNAc-FAmAzDBCO-MMAE and trastuzumab-(Galβ1,4)GlcNAc-FAmAzDBCO-MMAF (FIG. 18). The dual-drug conjugate trastuzumab-(GalNAzDBCO-seco-DUBA)GlcNAc-FAmP₈TzTCO-MMAE showed similar efficacy towards NCI-N87 cells compared to the MMAE-conjugate trastuzumab-(Galβ1,4)GlcNAc-FAmAzDBCO-MMAE, while showed higher efficacy towards NCI-N87 cells compared to the seco-DUBA conjugate trastuzumab-(Galβ1,4)GlcNAc-FAmP₄AzDBCO-seco-DUBA (FIG. 18)

### Example 58 In Vitro Plasma Stability Assay

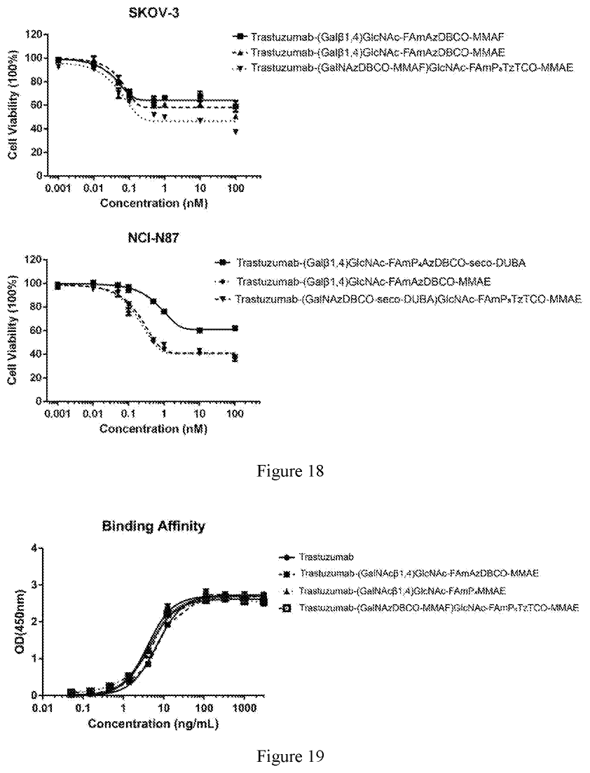

Human plasma was co-treated with protein A resin for 1 h at r.t. then centrifuged at 200 g for 5 min to removal the IgG. The depleted IgG plasma was filter sterilized by 0.22 μM filter. The trastuzumab-(Fucα1,6)(GalNAcβ1,4)GlcNAc-FAzDBCO-MMAE or trastuzumab-(GalNAcβ1,4)GlcNAc-FAmSucMMAE was incubated with the plasma to a final concentration of 100 μg/mL at 37° C. and 5% CO₂ in an incubator. Samples were taken at 0, 2, 6, 8 days and purified with protein A followed by MS analysis. Mass spectral analysis showed the peak corresponding to the trastuzumab-(Fucα1,6)(GalNAcβ1,4)GlcNAc-FAzDBCO-MMAE or trastuzumab-(GalNAcβ1,4)GlcNAc-FAmSucMMAE did not decrease in time. Meanwhile, no degradation peaks could be detected, indicating that the sample was stable in human plasma for at least 8 days (FIG. 20)

### Example 59 HIC-HPLC Assay

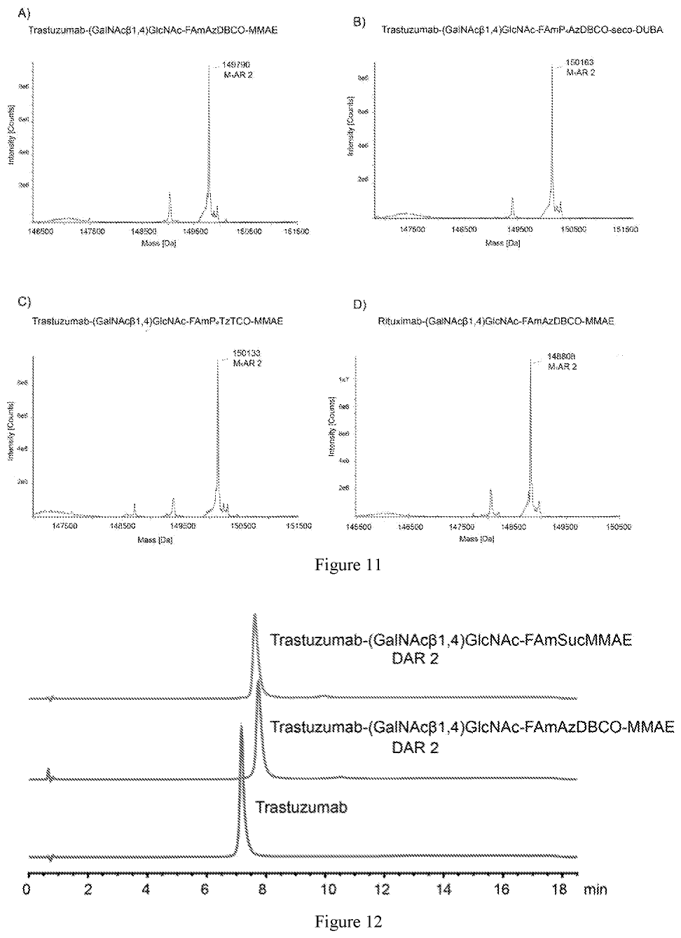

Some trastuzumab-drugs were evaluated by HIC-HPLC analysis using the Agilent 1260 HPLC system with a TSKgel Butyl-NPR column (4.6 mm×35 mm, 2.5 μm; TOSOH; Tokyo, Japan) under the following conditions: (1) buffer A: 20 mM sodium phosphate, 1.5 M ammonium sulfate (pH 6.9); (2) buffer B: 75% (v/v) 20 mM sodium phosphate, 25% (v/v) isopropanol (pH 6.9); (3) flow rate: 0.4 mL/min; (4) gradient: from 100% buffer A to 100% buffer B (over 1-13 min); and (5) column temperature was 25° C. HIC-HPLC analysis showed the high homogeneity of trastuzumab-drugs (FIG. 12).

### Example 60 Binding Affinity Assay

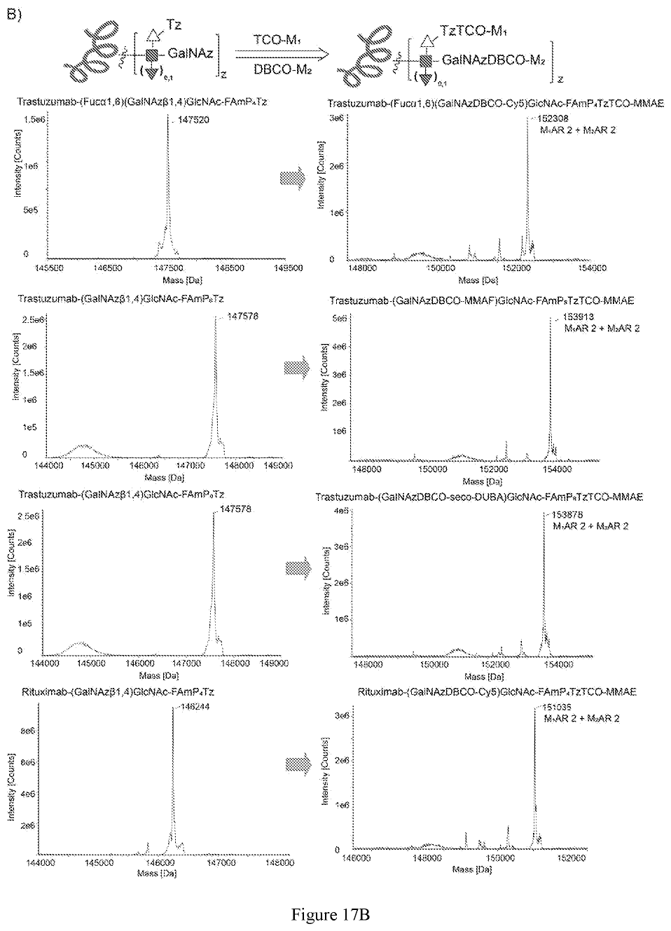

Recombinant Her2 extracellular domains (HER2, novoprotein) was diluted to a final concentration of 250 ng/mL with coating buffer and plated on 96-well plates (100 μL/well) at 4° C. for overnight. After removing the coating solution, the plates were blocked with 3% (v/v) bovine serum albumin in PBS for 2 h at 37° C. After washing with PBST (PBS containing 0.03% tween-20) for 3 times, trastuzumab, trastuzumab-(GalNAcβ1,4)GlcNAc-FAmP₄MMAE, trastuzumab-(GalNAcβ1,4)GlcNAc-FAmAzDBCO-MMAE and trastuzumab-(GalNAzDBCO-MMAF)GlcNAc-FAmP₈TzTCO-MMAE were added to PBST (with 1% (v/v) bovine serum albumin in PBS) to a series of final concentrations (3000 ng/mL, 1000 ng/mL, 333.33 ng/mL, 111.11 ng/mL, 37.04 ng/mL, 12.35 ng/mL, 4.12 ng/mL, 1.37 ng/mL, 0.46 ng/mL, 0.15 ng/mL, 0.05 ng/mL, 0 ng/mL) and added to the plates respectively. After incubating for 1.5 h, the plates were washed 3 times with PBST, then horseradish peroxidase (HRP)-conjugated goat anti-human IgG antibody was added to each well and incubated for 1 h at 37° C. Finally, each well was washed with PBST for 3 times, and then tetramethyl benzidine substrate was cotreated to produce color for visualization. The reaction in each well was stopped by adding 100 μL of 3 M HCl after 15 min of incubation. The absorbance was read at 450 nm on a Synergy™ LX plate reader. The result showed a similar HER2-binding affinity between trastuzumab and trastuzumab conjugates (FIG. 19).

### Example 61 Comparison of the catalytic efficiency of Hp1,3-FucT on antibody-(GalNAzβ1,4)GlcNAc and antibody-(Fucα1,6)(GalNAzβ1,4)GlcNAc in transferring using GDP-FAzP₄Biotin or GDP-FAmP₄Bioti

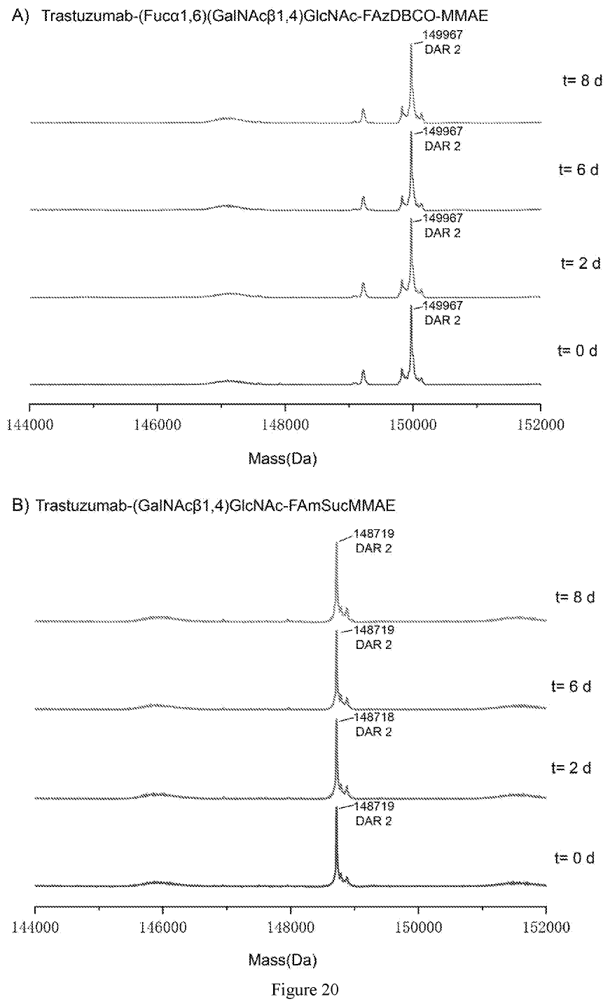

Trastuzumab-(GalNAzβ1,4)GlcNAc (2 mg/mL) was incubated with GDP-FAzP₄Biotin (1 mM) or GDP-FAmP₄Biotin (1 mM) and Hp1,3-FucT (0.5 mg/mL) in 50 mM Tris-HCl buffer (pH 7.5) with 5 mM MgCl₂ at 30° C. for 2 hours. Trastuzumab-(Fucα1,6)(GalNAzβ1,4)GlcNAc (2 mg/mL) was incubated with GDP-FAmP₄Biotin (1 mM) and Hp1,3-FucT (0.5 mg/mL) in 50 mM Tris-HCl buffer (pH 7.5) with 5 mM MgCl₂ at 30° C. for 2 hours. The reaction mixtures were purified with protein A resin and analyzed by LC-MS respectively, and % of conversion=average MAR/2*100%. The results showed that Hp1,3-FucT displayed higher catalytic efficiency towards the GDP-FAmX derivatives than the GDP-FAzX derivatives in transferring active molecule to the trastuzumab-(GalNAzβ1,4)GlcNAc. The results also showed that Hp1,3-FucT display higher catalytic efficiency towards trastuzumab-(GalNAzβ1,4)GlcNAc than trastuzumab-(Fucα1,6)(GalNAzβ1,4)GlcNAc (FIG. 21).

### Example 62 Intact Protein Mass Analysis

For LC-MS analysis, the purified proteins were analyzed on an Xevo G2-XS QTOF MS System (Waters Corporation) equipped with an electrospray ionization (ESI) source in conjunction with Waters Acuqity UPLC I-Class plus. Separation and desalting were carried out on a waters ACQUITY UPLC Protein BEH C4 Column (300 Å, 1.7 μm, 2.1 mm×100 mm). Mobile phase A was 0.1% formic acid in water and mobile phase B was acetonitrile with 0.1% formic acid. A constant flow rate of 0.2 ml/min was used. Data were analysed using Waters Unify software. Mass spectral deconvolution was performed using a Unify software (version 1.9.4, Waters Corporation).

### Example 63 Cloning, expression and purification of bovine β-1,4-GalT1(Y289L), EndoS, α-1,6-fucosidase (AlfC) and Hp1,3-FucT

The cloning, expression and purification of bovine β1,4-GalT1(Y289L)(SEQ ID NO: 3), Streptococcus pyogenes EndoS (SEQ ID NO: 6), Lactobacillus casei α-1,6-fucosidase (AlfC) (SEQ ID NO: 8) and Helicobacter pylori α1,3 fucosyltransferase (Hp1,3-FucT) (SEQ ID NO: 1) were performed according to the reported procedure by Qasba, P. K et al. (Prot. Expr. Fur. 2003, 30, 219) (J. Biol. Chem. 2002, 277, 20833.), by Collin, M. et al. (EMBO J. 2001, 20, 3046; Infect. Immun. 2001, 69, 7187), by Wang L., et al. (Methods Mol. Biol. 2018, 19, 367), and by Wu P. (Proc. Natl. Acad. Sci. USA 2009, 106, 16096) respectively.

### Example 64 Cloning, Expression and Purification of hRS7

The sequence of hRS7 antibody light chain and heavy chain were referenced to the patent (U.S. Pat. No. 7,238,785 B2). The gene encoding the light chain and the heavy chain of hRS7 were synthesized and clone into a PPT5 vector respectively by Genescript. Then, FreeStyle 293F cells were grown to a density of ˜2.5×10⁶ cells/ml and transfected by direct addition of 0.37 μg/ml and 0.66 μg/ml of the light chain and heavy chain expression plasmid DNA, and 2.2 μg/ml polyethylenimine (linear 25 kDa PEI, Polysciences, Inc, Warrington, PA) to the suspension cultures. The cultures were diluted 1:1 with Freestyle 293 expression medium containing 4.4 mM valproic acid (2.2 mM final) 24 h after transfection, and protein production was continued for another 4-5 d at 37° C. After protein production, the antibodies were purified through the protein A agarose following the manufacturer's instructions.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

1-123. (canceled)

124. A method for preparing a protein conjugate, comprising:
contacting a fucose derivative donor Q-Fuc*′ with a protein comprising an oligosaccharide in a presence of a catalyst, wherein said oligosaccharide comprises Formula (10): -GlcNAc(Fuc)_{b}-GalX′, to obtain a protein conjugate comprising Formula (11): 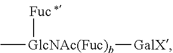
wherein:
said GlcNAc is directly or indirectly linked to an amino acid of said protein;
said GalX′ is a substituted galactose;
said Fuc is a fucose, b is 0 or 1;
said Fuc*′ comprises a fucose or fucose derivative (Fuco) and a molecule of interest (MOI₁′),
wherein a structure of Fuco is according to Formula (3): 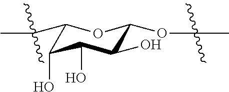
and
a structure of Fuc*′ is according to Formula (15): 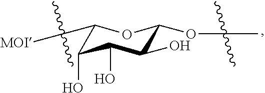
MOI′ comprises an active moiety,
said Q is a guanosine diphosphate (GDP), a uridine diphosphate (UDP), and/or a cytidine diphosphate (CDP), and
said protein comprises an antigen binding moiety and/or a Fc fragment.

125. (canceled)

126. The method of claim 124, wherein said catalyst is a fucosyltransferase or a functional variant or fragment thereof.

127-146. (canceled)

147. The method of claim 124, wherein a hydroxyl group on one or more positions selected from a C2 position, a C3 position, a C4 position and a C6 position of the substituted galactose, is substituted.

148. (canceled)

149. The method of claim 124, wherein said GalX′ is a monosaccharide.

150-157. (canceled)

158. The method of claim 124, comprising: contacting the Q-Fuc*′ with the protein comprising the oligosaccharide comprising Formula (10) -GlcNAc(Fuc)_{b}-GalX′ to obtain a protein conjugate comprising Formula (12) 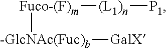
wherein
Q-Fuc*′ is Q-Fuco-(F)ₘ-(L₁)ₙ-P₁,
Fuc*′ is Fuco-(F)ₘ-(L₁)ₙ-P₁,
Fuco is the fucose or fucose derivative of Fuc*′,
F is a connector and according to Formula (2): (J)_{q}-(FL)ₛ, J is a jointer, FL is a spacer, q is 0 or 1, s is 0 or 1, m is 0 or 1,
L₁ is a linker, and n is 0 or 1,
P₁ is a biologically and/or pharmaceutically active moiety.

159-162. (canceled)

163. The method of claim 124, comprising: contacting the Q-Fuc*′ with the protein comprising the oligosaccharide comprising Formula (10) -GlcNAc(Fuc)_{b}-GalX′ to obtain a protein conjugate comprising Formula (13), 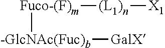
wherein
Q-Fuc*′ is Q-Fuco-(F)ₘ-(L₁)ₙ-X₁,
Fuc*′ is Fuco-(F)ₘ-(L₁)ₙ-X₁,
Fuco is the fucose or fucose derivative of Fuc*′,
F is a connector and according to Formula (2): (J)_{q}-(FL)ₛ, J is a jointer, FL is a spacer, q is 0 or 1, s is 0 or 1, m is 0 or 1,
L₁ is a linker, and n is 0 or 1,
X₁ is a functional group capable of participating in a bioorthogonal ligation reaction.

164. (canceled)

165. The method of claim 163, further comprising: contacting said protein conjugate comprising Formula (13) 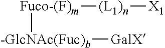
with a Y₁-(FL′)_{m′}-(L₁′)_{n′}-P₁, to obtain a protein conjugate comprising Formula (14) 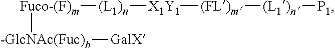
wherein,
Y₁ comprises a functional moiety capable of reacting with said X₁ through a bioorthogonal ligation reaction,
X₁Y₁ is a remaining group after a ligation reaction between X₁ and Y₁,
FL′ is a spacer, m′ is 0 or 1,
L′ is a linker, n′ is 0 or 1, and
P₁ is a biologically and/or pharmaceutically active moiety.

166-168. (canceled)

169. The method of claim 158, further comprising: contacting a protein conjugate comprising Formula (1-8) 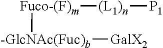
with Y₂-(FL″)_{m″}-(L₂)_{n″}-P₂, to obtain a protein conjugate comprising Formula (1-10): 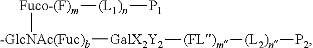
wherein,
GalX′ comprises a functional group X₂ which comprises a functional moiety capable of participating in a bioorthogonal ligation reaction, and said GalX′ is represented by GalX₂,
Y₂ is a functional group,
X₂Y₂ is a remaining group after a ligation reaction between X₂ and Y₂,
FL″ is a spacer, m″ is 0 or 1,
L₂ is a linker, n″ is 0 or 1, and
P₂ is a biologically and/or pharmaceutically active moiety.

170. The method of claim 163, further comprising: contacting a protein conjugate comprising Formula (1-7) 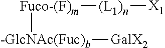
with Y₂-(FL″)_{m″}-(L₂)_{n″}-P₂ to obtain a protein conjugate comprising Formula (1-11) 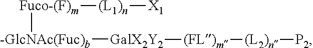
wherein
GalX′ comprises a functional group X₂ which comprises a functional moiety capable of participating in a bioorthogonal ligation reaction, and said GalX′ is represented by GalX₂,
Y₂ is a functional group,
X₂Y₂ is a remaining group after a ligation reaction between X₂ and Y₂,
FL″ is a spacer, m″ is 0 or 1,
L₂ is a linker, n″ is 0 or 1, and
P₂ is a biologically and/or pharmaceutically active moiety.

171. The method of claim 165, further comprising: contacting a protein conjugate comprising Formula (1-9): 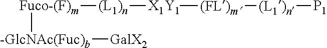
with Y₂-(FL″)_{m″}-(L₂)_{n″}-P₂ to obtain a protein conjugate comprising Formula (1-12) 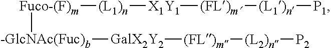
wherein
GalX′ comprises a functional group X: which comprises a functional moiety capable of participating in a bioorthogonal ligation reaction, and said GalX′ is represented by GalX₂,
Y₂ is a functional group,
X₂Y₂ is a remaining group after a ligation reaction between X₂ and Y₂,
FL″ is a spacer, m″ is 0 or 1,
L₂ is a linker, n″ is 0 or 1, and
P₂ is a biologically and/or pharmaceutically active moiety.

172. The method of claim 170, further comprising: contacting said protein conjugate comprising Formula (1-11) 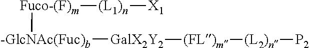
with Y₁-(FL′)_{m′}-(L₁′)_{n′}-P₁ to obtain a protein conjugate comprising Formula (1-12) 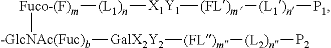
wherein
Y₁ comprises a functional moiety capable of reacting with said X₁ through a bioorthogonal ligation reaction,
X₁Y₁ is a remaining group after a ligation reaction between X₁ and Y₁,
FL′ is a spacer, m′ is 0 or 1,
L′ is a linker, n′ is 0 or 1, and
P₁ is a biologically and/or pharmaceutically active moiety.

173-202. (canceled)

203. The method of claim 158, wherein said J is a 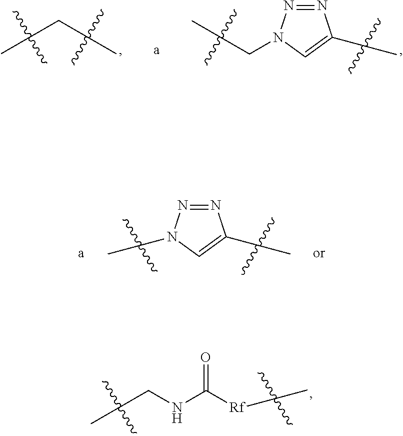
wherein Rf is —CH₂—, —NH— or —O—.

204. (canceled)

205. The method of claim 158, wherein J is a

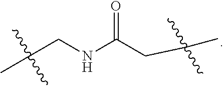 [SMILES: *CNC(=O)C*]

206-234. (canceled)

235. The method of claim 124, wherein said Q-Fuc*′ is selected from the group consisting of

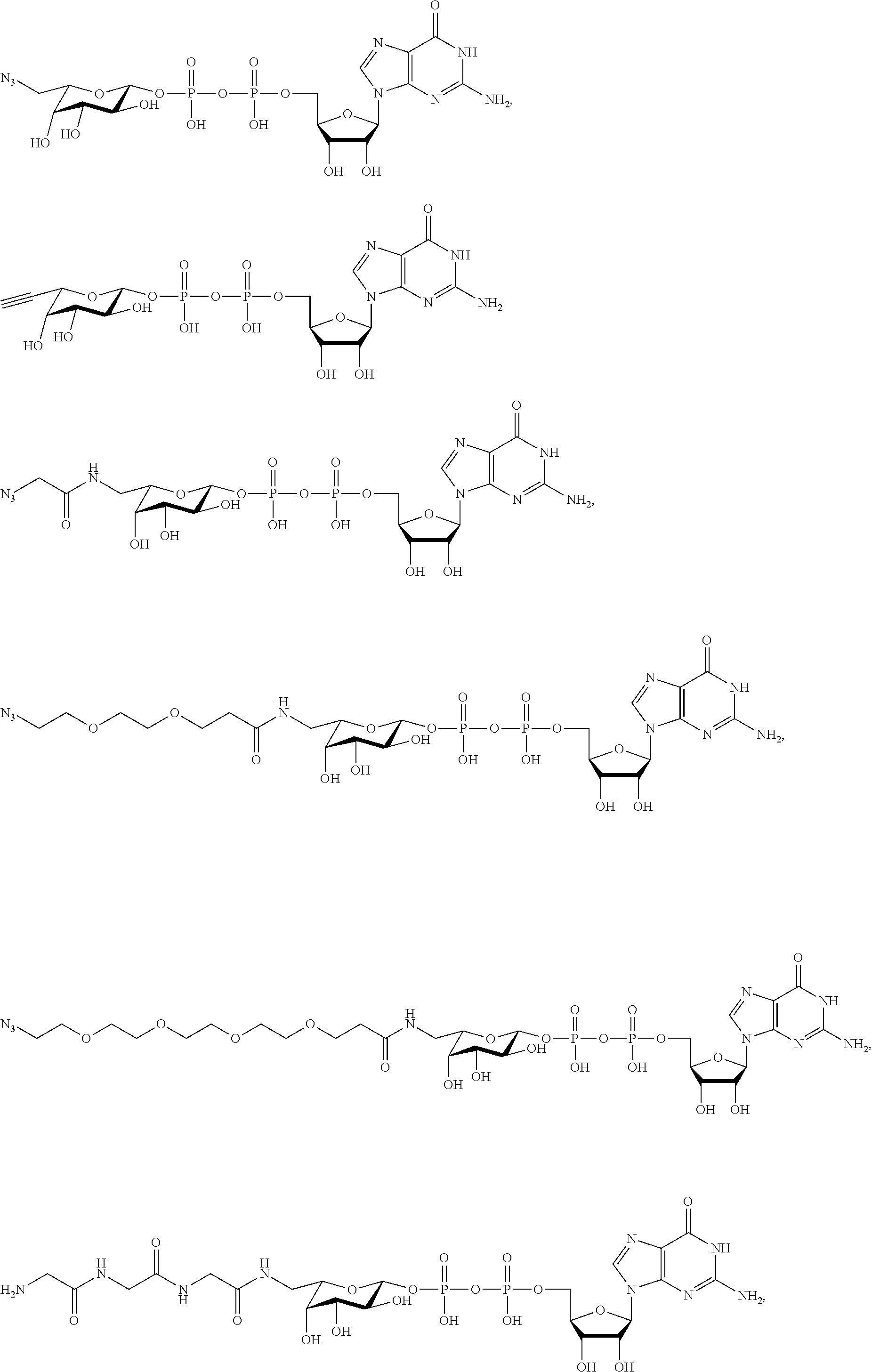

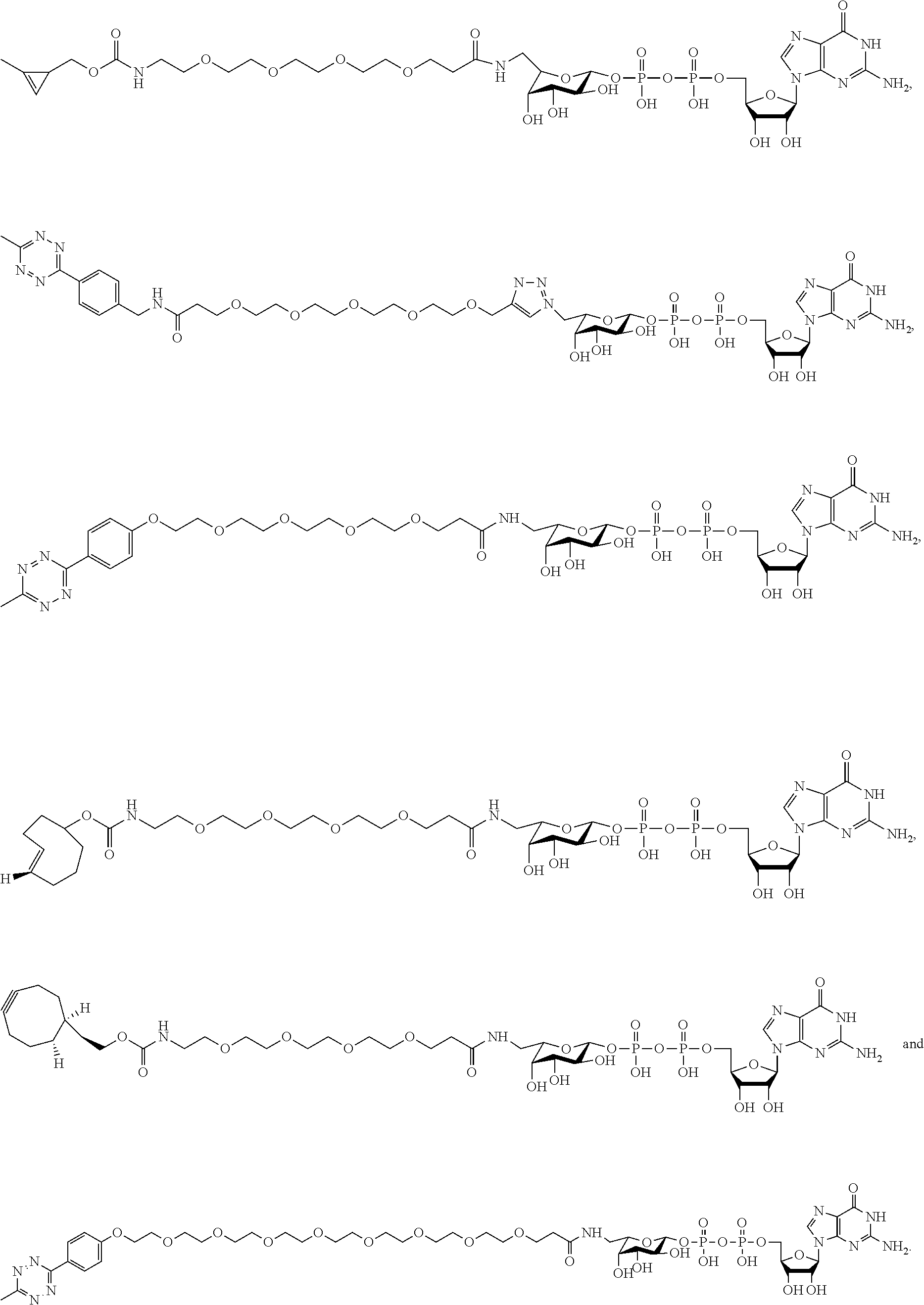

236. The method of claim 124, wherein said Q-Fuc*′ is selected from the group consisting of

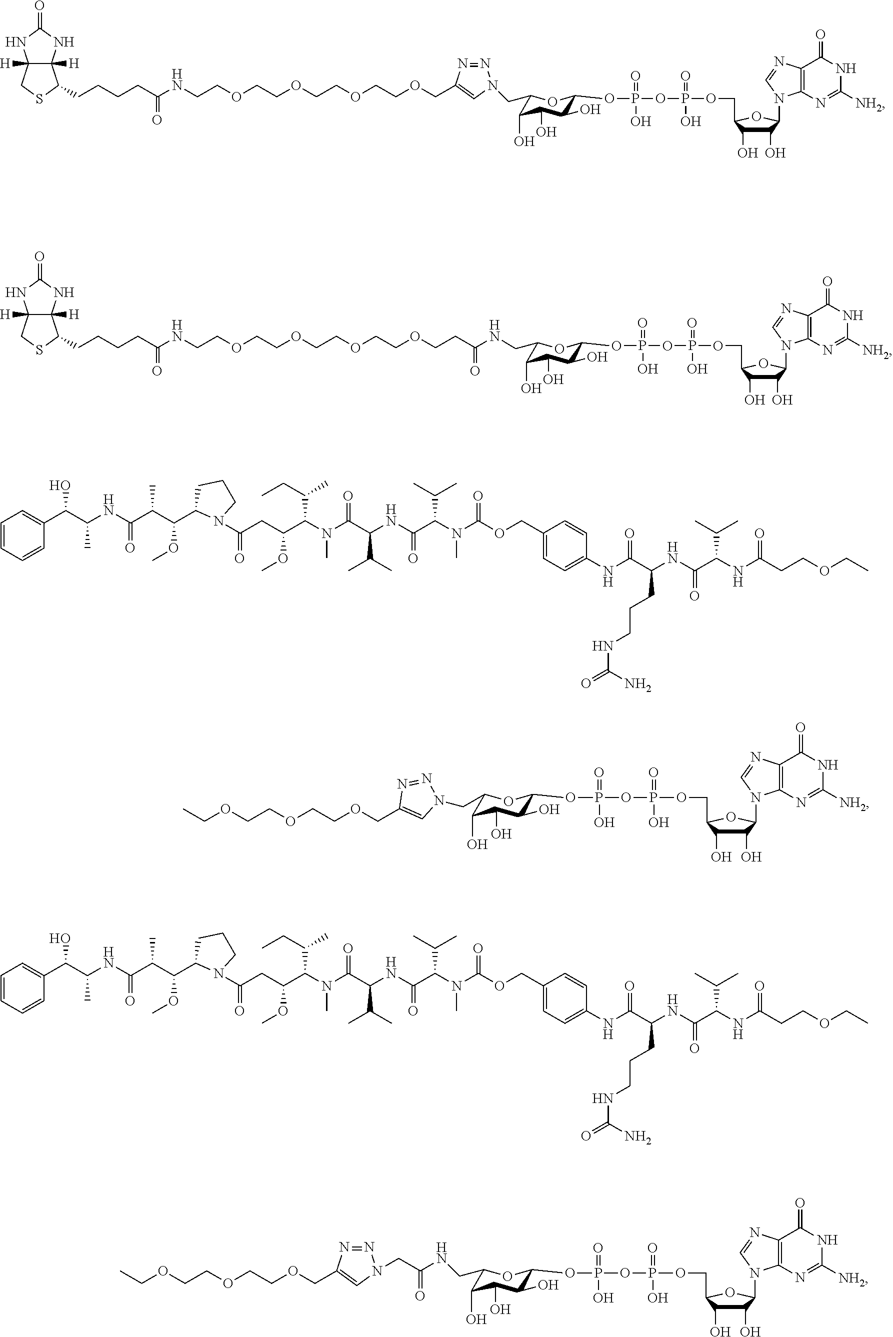

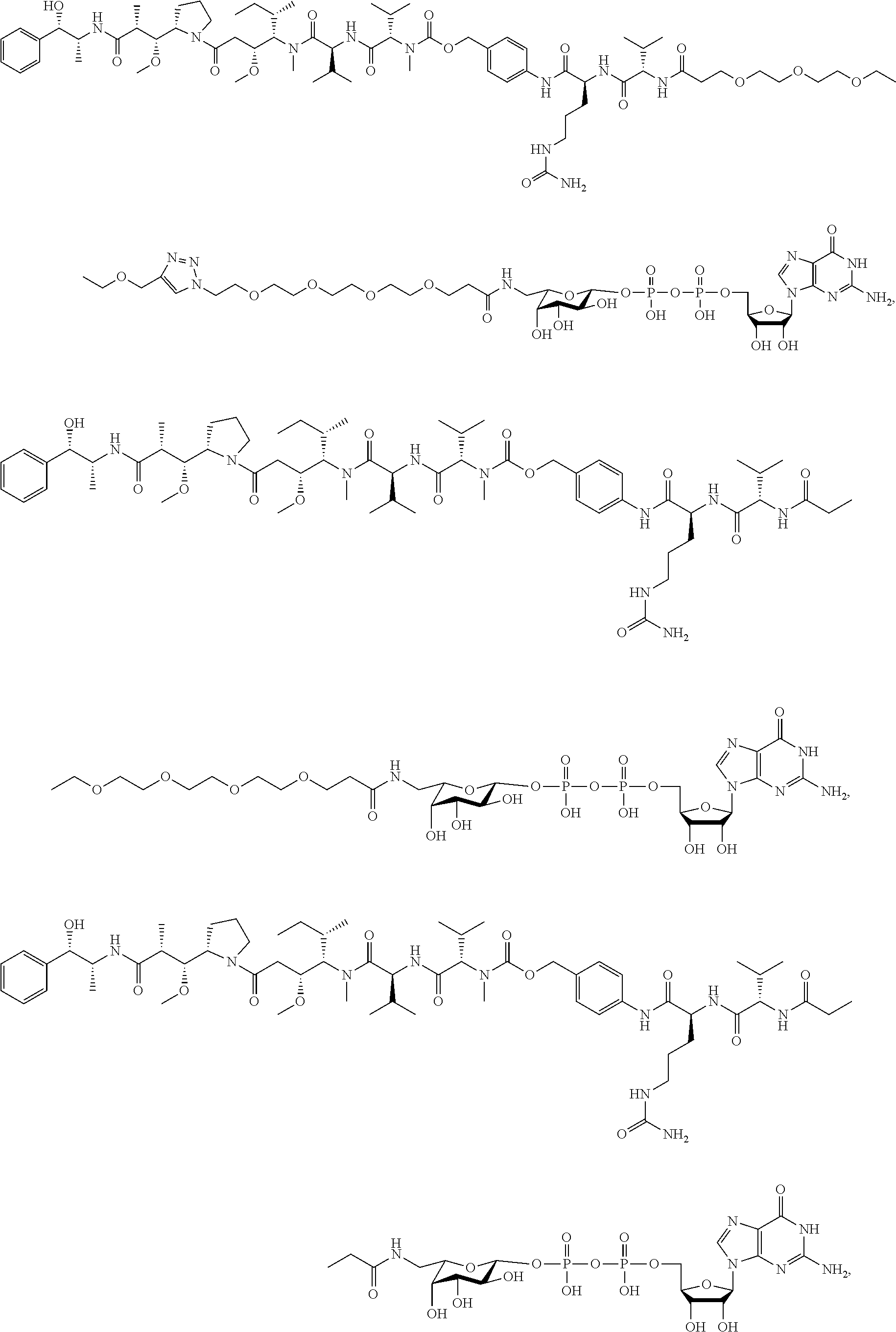

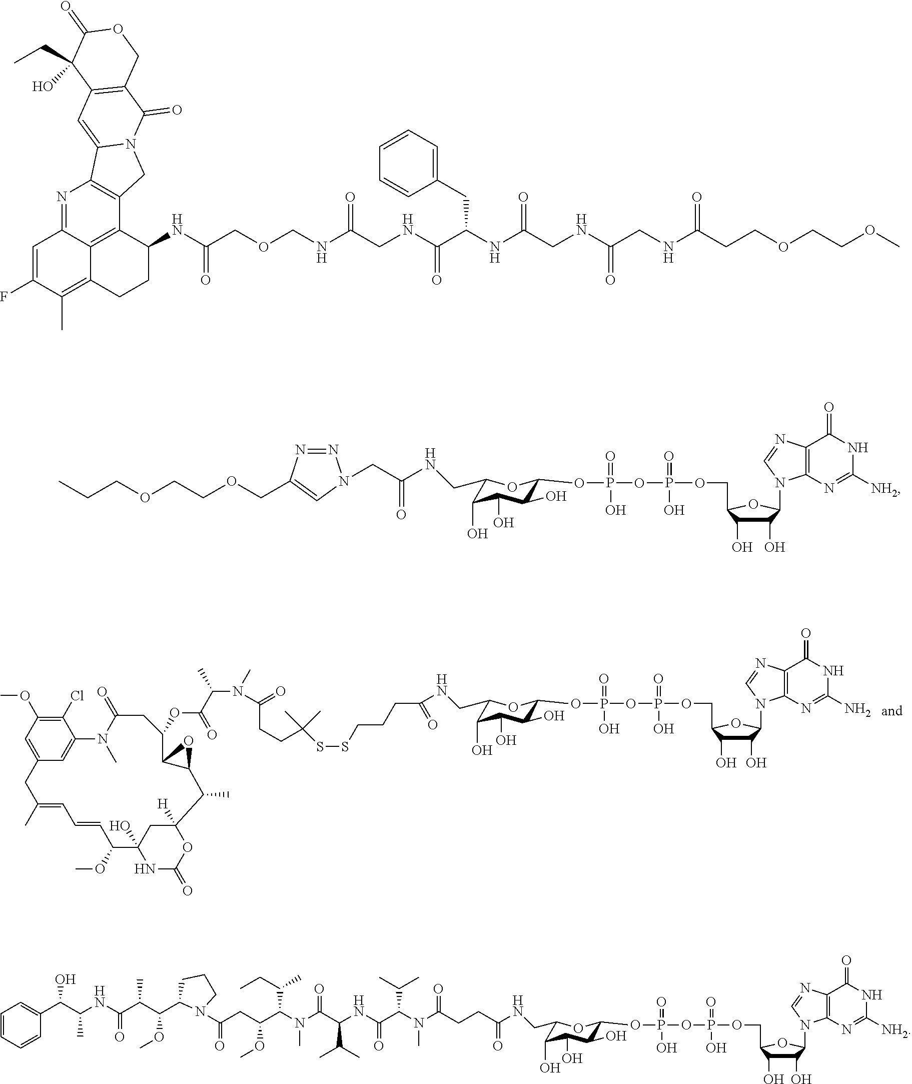

237. The method of claim 124, wherein said GalX′ is selected from the group consisting of

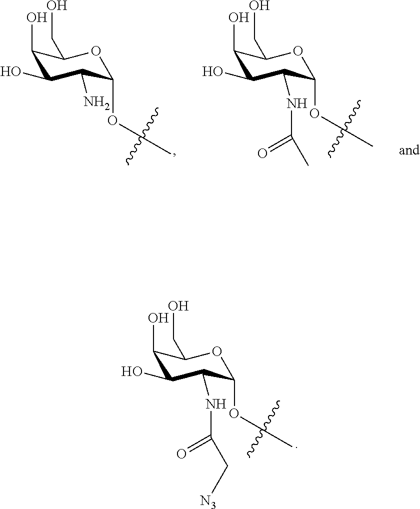

238. The method of claim 124, further comprising: contacting a protein comprising an oligosaccharide comprising the -GlcNAc(Fuc)ₕ with a UDP-GalX′ in a presence of a catalyst, to obtain said protein comprising Formula (10): -GlcNAc(Fuc)_{b}-GalX′.

239-252. (canceled)

253. The method of claim 238, further comprising: modifying a protein comprising an oligosaccharide to obtain a protein comprising a -(Fucα1,6)_{b}GlcNAc, wherein b is 0 or 1.

254-272. (canceled)

273. The method of claim 124, wherein the protein conjugate is according to Formula (8-1) [symbol] , wherein [symbol] is a GlcNAc, [symbol] is the Fuc linked to the GlcNAc through an α-1,6 linkage, GalX′ is the substituted galactose linked to the GlcNAc through a GalX′β1,4GlcNAc linkage, Fuc*′ is linked to the GlcNAc through an Fuc*′α1,3GlcNAc linkage, b is 0 or 1, [symbol] is an antibody or a Fc-fusion protein, and the [symbol] is directly linked to an asparagine residue in said protein.

274. (canceled)

275. The method of claim 124, wherein the protein conjugate is according to Formula (9-1) [symbol] , wherein [symbol] is a GlcNAc, [symbol] is the Fuc linked to the GlcNAc through a α1,6GlcNAc linkage, [symbol] is a mannose, GalX′ is the substituted galactose linked to the GlcNAc through a GalX′β1,4GlcNAc linkage, Fuc*′ is linked to the GlcNAc through an Fuc*′α1,3GlcNAc linkage, c is 0 or 1, [symbol] is an antibody or Fc-fusion protein, and the [symbol] of [symbol] is directly linked to an adjacent mannose in a glycan chain.

276. (canceled)

277. A protein conjugate, which is obtained with the method according to claim 124.

278-280. (canceled)

281. A method for preventing or treating disease, comprising administering the protein conjugate of claim 277.

282. (canceled)
